(19) United States
(12) Patent Application Publication
(10) Pub. No.: US 2025/0084041 A1
(43) Pub. Date: Mar. 13, 2025

(54) ANTIMICROBIAL COMPOUNDS AND METHODS

(71) Applicants: Curza Global, LLC, Salt Lake City, UT (US); The University of Utah Research Foundation, Salt Lake City, UT (US)

(72) Inventors: Paul R. Sebahar, Sandy, UT (US); Ryan E. Looper, Salt Lake City, UT (US); Seth Grant, Payson, UT (US); Hariprasada R. Kanna Reddy, Salt Lake City, UT (US); Ben Isaac C. Tresco, Cambridge, MA (US); Travis J. Haussener, Salt Lake City, UT (US); Daniel Feodore Zigar, Salt Lake City, UT (US); Charles A. Testa, North Salt Lake, UT (US)

(21) Appl. No.: 18/725,391

(22) PCT Filed: Dec. 28, 2022
(86) PCT No.: PCT/US2022/082474

§ 371 Date: Jun. 28, 2024

Related U.S. Application Data
(60) Provisional application No. 63/295,128, filed on Dec. 30, 2021.

(51) Int. Cl. C07D 239/47; A61K 31/513; A61K 31/55; C07D 401/10; C07D 403/10

(52) U.S. Cl. CPC C07D 239/47; A61K 31/513; A61K 31/55; C07D 401/10; C07D 403/10

(57) ABSTRACT

The invention is directed to compounds that are active as antibacterial agents. The invention compounds are active against gram-positive and gram-negative bacteria and can be used to treat infections caused by gram-positive and gram-negative bacteria. Also disclosed are processes and intermediates for making the compounds.

### CROSS-REFERENCE TO RELATED APPLICATIONS

This application claims priority to U.S. Provisional Application Ser. No. 63/295,128, filed Dec. 30, 2021, the entire contents of which are incorporated herein by reference.

### STATEMENT OF GOVERNMENT INTEREST

This invention was made with government support under 1R44AI152665-01 awarded by the Small Businesses Innovation Research (SBIR) program agencies. The government has certain rights in the invention.

### FIELD OF THE INVENTION

The present disclosure relates to compounds that are active as antibacterial agents. The present disclosure also relates to methods of treating bacterial infections with the present compounds.

### BACKGROUND OF THE INVENTION

Antibacterial resistance is a worldwide problem. Both gram-positive and gram-negative bacteria are increasingly becoming resistant to antibiotics.

Gram-positive bacteria such as methicillin resistant Staphylococcus aureus (MRSA) are resistant to most antibiotics that are related to penicillin. MRSA strains are commonly involved in infections acquired in health care facilities and can cause infections in greater communities.

Gram-negative bacteria are believed to be more resistant to antibiotics than Gram-positive bacteria, because of the impermeability of their cell walls. According to the National Institutes of Health (NIH), Gram-negative bacteria can cause many types of infections and are spread to humans in a variety of ways. Several species, including Escherichia coli, are common causes of foodborne disease. Vibrio cholerae, the bacteria responsible for cholera, is a waterborne pathogen. Gram-negative bacteria can also cause respiratory infections, such as certain types of pneumonia, and sexually transmitted diseases, including gonorrhea. Yersinia pestis, the Gram-negative bacterium responsible for plague, is transmitted to people through the bite of an infected insect or handling an infected animal. See www.niaid.nih.gov/research/gram-negative-bacteria (last visited Dec. 22, 2020).

Certain types of Gram-negative bacteria have become increasingly resistant to available antibiotic drugs. Some strains are now resistant to many, most, or all available treatments resulting in increased illness and death from bacterial infections and contributing to escalating healthcare costs. Examples of Gram-negative bacteria that have demonstrated drug resistance include: E. coli, which causes the majority of urinary tract infections; Acinetobacter baumanii, which causes disease mainly in healthcare settings; Pseudomonas aeruginosa, which causes bloodstream infections and pneumonia in hospitalized patients and is a common cause of pneumonia in patients with cystic fibrosis; Klebsiella pneumoniae, which causes many types of healthcare-associated infections, including pneumonia, urinary tract infections, and bloodstream infections; and Neisseria gonorrhoeae, which causes the sexually transmitted disease gonorrhea and is the second most commonly reported infectious disease in the United States.

As a result, new drugs to combat Gram-positive and Gram-negative bacterial infections are needed.

### SUMMARY OF THE INVENTION

These and other needs are met by the present invention which provides in one aspect a compound of formula I:

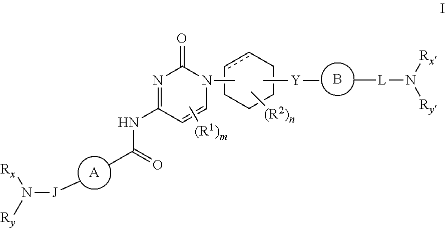

or a single stereoisomer or mixture of stereoisomers thereof, or a pharmaceutically acceptable salt thereof, wherein:
ring A is a 3-8 membered monocyclic heterocycloalkylene optionally substituted with up to three substituents independently selected from the group consisting of C₁-C₆ alkyl, C₁-C₆ alkoxy, halo, CN, C₁-C₆ haloalkyl, phenyl, OH, NH₂, NH(C₁-C₆ alkyl), N(C₁-C₆ alkyl)₂, COOH, COO(C₁-C₆ alkyl), CONH₂, CONH(C₁-C₆ alkyl), CON(C₁-C₆ alkyl)₂, and oxo;
J is C₁-C₆ alkylene or C₃-C₈ cycloalkylene, either of which is optionally substituted with halo, OH, or C₁-C₆ alkoxy, wherein up to two methylene units of the C₁-C₆ alkylene are optionally and independently replaced with O, S, SO, SO₂, or C═O;
Y is a bond or C₁-C₆ alkylene optionally substituted with OH, NH₂, CN, halo, or C₁-C₆alkoxy, wherein up to two methylene units of the C₁-C₆ alkylene are optionally and independently replaced by O, NH, N—(C₁-C₆ alkyl), N—(C₁-C₆ hydroxyalkyl), N—(C₁-C₆ haloalkyl), N—(C₁₋₆ alkylene-C₃₋₈ cycloalkyl), N—(C₃₋₈ cycloalkyl), NH(C═O), N—(C₁₋₆ alkyl)(C═O), or (C═O);
ring B is a 3-8 membered monocyclic cycloalkylene, 3-8 membered monocyclic heterocycloalkylene, 6-12 membered bicyclic cycloalkylene, or a 6-12 membered bicyclic heterocycloalkylene, each of which is optionally substituted with up to three substituents independently selected from the group consisting of C₁-C₆ alkyl, C₁-C₆ alkoxy, halo, CN, C₁-C₆ haloalkyl, OH, COOH, COO(C₁-C₆ alkyl), CONH₂, CONH(C₁-C₆ alkyl), CON(C₁-C₆ alkyl)₂, and C₁-C₆ hydroxyalkyl;
L is a bond or C₁-C₆ alkylene, wherein up to two methylene units of the C₁-C₆ alkylene are optionally and independently replaced with O, NH, (C═O), NH(C═O), N—(C₁₋₆ alkyl)(C═O), (C═NH), NH(C═N), or N—(C₁₋₆ alkyl);
R¹ and R² are each independently selected from the group consisting of C₁-C₆ alkyl, halo, CN, OH, NH₂, O(C₁-C₆ haloalkyl), NH(C₁-C₆ alkyl), N(C₁-C₆ alkyl)₂, —COO(C₁-C₆ alkyl), CONH₂, C₁-C₆ haloalkyl, C₁-C₆ alkoxy, and C₁-C₆ haloalkoxy;
Rₓ, R_{y}, R_{x′}, and R_{y′} are each independently H, C₁-C₆ alkyl, or an amino protecting group;
m and n are each independently 0, 1, 2, or 3; and
[symbol] represents a single bond or a double bond.

In another aspect, the invention provides methods of using compounds of formula I or a pharmaceutically acceptable salt thereof for the treatment of bacterial infections.

In another aspect, the invention provides pharmaceutical compositions comprising a compound of formula I or a pharmaceutically acceptable salt thereof and a pharmaceutically acceptable carrier.

In a further aspect, the invention provides processes for making compounds of formula I or a pharmaceutically acceptable salt thereof, as well as compound intermediates used in the processes, as depicted in the synthetic schemes.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety, including U.S. Pat. Publ. No. 2013/0090326. In case of conflict, the present specification, including these definitions, will control.

The terms “a,” “an,” and “the” as used herein not only include aspects with one member, but also include aspects with more than one member.

The term “about” as used herein means “approximately” and is used to modify a numerical value indicating a defined range around that value. If “X” were the value, “about X” would generally indicate a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” When the quantity “X” only includes whole-integer values (e.g., “X carbons”), “about X” indicates from (X−1) to (X+1). In this case, “about X” as used herein specifically indicates at least the values X, X−1, and X+1.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 110%” is equivalent to “about 7%, about 9%, or about 11%.”

The symbol “—” means a single bond, “═” means a double bond, “ [symbol] ” means a triple bond, “ [symbol] ” means a single or a double bond.

As used herein, a wavy line drawn on a structure can be used to show the attachment point of the structure, such as

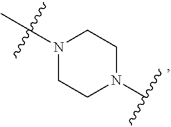 [SMILES: *N1CCN(*)CC1]

wherein “ [symbol] ” indicates points of attachment.

A “*” in a chemical structure is used to identify a chiral center.

The term “acyl” as used herein includes an alkanoyl, aroyl, heterocycloyl, or heteroaroyl group as defined herein. Examples of acyl groups include, but are not limited to, acetyl, benzoyl, and nicotinoyl.

The term “alkanoyl” as used herein includes an alkyl-C(O)— group wherein the alkyl group is as defined herein. Examples of alkanoyl groups include, but are not limited to, acetyl and propanoyl.

The term “agent” as used herein includes a compound or mixture of compounds that, when added to a composition, tend to produce a particular effect on the composition's properties. For example, a composition comprising a thickening agent is likely to be more viscous than an otherwise identical comparative composition that lacks the thickening agent.

The term “alkyl” as used herein includes an aliphatic hydrocarbon chain that may be straight chain or branched. The chain may contain an indicated number of carbon atoms: For example, C₁-C₁₀ indicates that the group may have from 1 to 10 (inclusive) carbon atoms in it. If not otherwise indicated, an alkyl group contains from 1 to about 20 carbon atoms. In some aspects, alkyl groups have 1 to about 10 carbon atoms. In some aspects, alkyl groups (“lower alkyl”) have 1 to 8, 1 to 6, or 1 to 3 carbon atoms in the chain. Examples may include, but are not limited to, methyl, ethyl, propyl, isopropyl (iPr), 1-butyl, 2-butyl, isobutyl (iBu), tert-butyl, pentyl, 2-methylbutyl, 1,1-dimethylpropyl, hexyl, heptyl, octyl, nonyl, decyl, docecyl, cyclopentyl, or cyclohexyl.

An alkyl group can be unsubstituted or optionally substituted. When optionally substituted, one or more hydrogen atoms of the alkyl group (e.g., from 1 to 4, from 1 to 2, or 1) may be replaced with a moiety independently selected from the group consisting of fluoro, hydroxy, alkoxy, amino, alkylamino, acylamino, thio, and alkylthio. In some aspects, the alkyl group is unsubstituted or not optionally substituted.

“Alkylene” as used herein includes an alkyl group that is substituted at two points. An example is methylene (—CH₂—), propylene (—CH₂CH₂CH₂—), and the like.

The term “alkenyl” as used herein includes a straight or branched chain hydrocarbon containing at least one carbon-carbon double bond. The chain may contain an indicated number of carbon atoms. For example, “C₁-C₁₂ alkenyl” indicates that the group may have from 1 to 12 (inclusive) carbon atoms and at least one carbon-carbon double bond. When the indicated number of carbon atoms is 1, then the Cᵢ alkenyl is double bonded to a carbon (i.e., a carbon equivalent to an oxo group). In certain aspects, the chain includes 1 to 12, about 2 to 15, about 2 to 12, about 2 to 8, or about 2 to 6 carbon atoms. An alkenyl group can be preferably one stereoisomer (i.e., cis- or, alternatively, trans-). Examples of an alkenyl group may include, but are not limited to, ethenyl (i.e., vinyl), allyl, propenyl, butenyl, crotyl, pentenyl, hexenyl, heptenyl, octenyl, nonenyl, decenyl, dodecenyl, cyclopentenyl, cyclohexenyl, 2-isopentenyl, allenyl, butadienyl, pentadienyl, 3-(1,4-pentadienyl), and hexadienyl.

An alkenyl group can be unsubstituted or optionally substituted. When optionally substituted, one or more hydrogen atoms of the alkenyl group (e.g., from 1 to 4, from 1 to 2, or 1) may be replaced with a moiety independently selected from the group consisting of fluoro, hydroxy, alkoxy, amino, alkylamino, acylamino, thio, and alkylthio, with the proviso that no hydrogen atom substituent on the carbon-carbon double bond is replaced by a hydroxy, amino, or thio group. In some aspects, the alkenyl group is unsubstituted or not optionally substituted.

“Alkenylene” as used herein includes an alkenyl group that is substituted at two points. An example is but-2-enylene (—CH₂CH═CHCH₂—) and the like.

The term “alkynyl” as used herein includes a straight, branched, or cyclic hydrocarbon containing at least one carbon-carbon triple bond. Examples may include, but are not limited to, ethynyl, propargyl, propynyl, butynyl, pentynyl, hexynyl, heptynyl, octynyl, nonynyl, decynyl, or decynyl.

An alkynyl group can be unsubstituted or optionally substituted. When optionally substituted, one or more hydrogen atoms of the alkynyl group (e.g., from 1 to 4, from 1 to 2, or 1) may be replaced with a moiety independently selected from the group consisting of fluoro, hydroxy, alkoxy, amino, alkylamino, acylamino, thio, and alkylthio, with the proviso that no sp-hybridized hydrogen atom substituent is replaced by a hydroxy, amino, or thio group. In some aspects, the alkynyl group is unsubstituted or not optionally substituted.

“Alkynylene” as used herein includes an alkynyl group that is substituted at two points. An example is 2-butynylene (—CH₂CCCH₂—) and the like.

The term “alkoxy” as used herein includes a straight or branched chain saturated or unsaturated hydrocarbon containing at least one oxygen atom in an ether group (e.g., EtO—). The chain may contain an indicated number of carbon atoms. For example, “C₁-C₁₂ alkoxy” indicates that the group may have from 1 to 12 (inclusive) carbon atoms and at least one oxygen atom. Examples of a C₁-C₁₂ alkoxy group include, but are not limited to, methoxy, ethoxy, isopropoxy, butoxy, n-pentoxy, isopentoxy, neopentoxy, and hexoxy.

An alkoxy group can be unsubstituted or optionally substituted. When optionally substituted, one or more hydrogen atoms of the alkoxy group (e.g., from 1 to 4, from 1 to 2, or 1) may be replaced with a moiety independently selected from the group consisting of fluoro, hydroxy, alkoxy, amino, alkylamino, acylamino, thio, and alkylthio, with the proviso that no hydrogen atom alpha to the ether oxygen is replaced by a hydroxy, amino, or thio group. In some aspects, the alkoxy group is unsubstituted or not optionally substituted.

The term “aryl” as used herein includes cyclic aromatic carbon ring systems containing from 6 to 18 carbons. Examples of an aryl group include, but are not limited to, phenyl, naphthyl, anthracenyl, tetracenyl, biphenyl and phenanthrenyl.

The term “cycloalkyl” as used herein includes non-aromatic saturated monocyclic or multicyclic ring system that may contain an indicated number of carbon atoms. For example, C₃-C₁₂ indicates that the group may have from 3 to 12 (inclusive) carbon atoms in it. If not otherwise indicated, a cycloalkyl group includes about 3 to about 20 carbon atoms. In some aspects, cyclo alkyl groups have 3 to about 12 carbon atoms in the group. In some aspects, cycloalkyl groups have 3 to about 7 carbon atoms in the group. Examples may include, but are not limited to, cyclopropyl, cyclobutyl, cyclopentyl, cyclohexyl, 4,4-dimethylcyclohexyl, and cycloheptyl. The term “cycloalkyl” also includes multicyclic rings such as a bicyclic cycloalkyl, or a tricyclic cycloalkyl which may be in a fused, bridged, or spiro orientation.

The term “cycloalkylene” as used herein includes a cycloalkyl group that is substituted at two points.

The terms “disorder” and “disease” are used herein interchangeably for a condition in a subject. A disorder is a disturbance or derangement that affects the normal function of the body of a subject. A disease is a pathological condition of an organ, a body part, or a system resulting from various causes, such as infection, genetic defect, or environmental stress that is characterized by an identifiable group of symptoms. A disorder or disease can refer to a biofilm-related disorder or disorder caused by a planktonic bacterial phenotype that is characterized by a disease-related growth of bacteria.

The term “effective amount” or “effective dose” as used herein includes an amount sufficient to achieve the desired result and accordingly will depend on the ingredient and its desired result. Nonetheless, once the desired effect is identified, determining the effective amount is within the skill of a person skilled in the art.

As used herein, “fluoroalkyl” includes an alkyl group wherein the alkyl group includes one or more fluoro-substituents. Examples include, but are not limited to, trifluoromethyl.

As used herein, “geminal” substitution includes two or more substituents that are directly attached to the same atom. An example is 3,3-dimethyl substitution on a cyclohexyl or spirocyclohexyl ring.

As used herein, “halo” or “halogen” includes fluoro, chloro, bromo, and iodo.

As used herein, “heterocycloalkyl” includes a non-aromatic saturated ring of about 3 to about 12 ring atoms (e.g., 5 to about 10 ring atoms, 3 to about 8 ring atoms, or 3 to about 6 ring atoms), in which one or more of the atoms in the ring system is an element or elements other than carbon, e.g., nitrogen, oxygen or sulfur. A heterocycloalkyl group optionally comprises at least one sp²-hybridized atom (e.g., a ring incorporating a carbonyl, endocyclic olefin, or exocyclic olefin). In some embodiments, a nitrogen or sulfur atom of the heterocycloalkyl is optionally oxidized to the corresponding N-oxide, S-oxide or S,S-dioxide. The monocyclic heterocycle means a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycloalkyl include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyridazin-3(2H)-onyl, pyridin-2(1H)-onyl, pyrrolinyl, pyrrolidinyl, tetrahydrofuranyl, tetrahydropyranyl, tetrahydropyridinyl, tetrahydropyrimidinyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl.

The term “heterocycloalkyl” also includes multicyclic rings such as a bicyclic heterocycle, or a tricyclic heterocycle which may be in a fused, bridged, or spiro orientation. The bicyclic heterocycle may be a monocyclic heterocycle fused to a monocyclic cycloalkyl, or a monocyclic heterocycle fused to a monocyclic cycloalkenyl, or a monocyclic heterocycle fused to a monocyclic heterocycle, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, 3-azabicyclo[3.1.0]hexane, 3-azabicyclo[4.1.0]heptane, 3-azabicyclo[3.2.0]heptane, (3aR,6aS)-hexahydro-1H-2λ₂-cyclopenta[c]pyrrole, (3aR,7aS)-octahydro-2λ₂-isoindole.

Tricyclic heterocycles may be exemplified by a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms.

A heterocycloalkyl group can be unsubstituted or optionally substituted. When optionally substituted, one or more hydrogen atoms of the group (e.g., from 1 to 4, from 1 to 2, or 1) may be replaced with a moiety independently selected from the group consisting of fluoro, hydroxy, alkoxy, amino, alkylamino, acylamino, thio, and alkylthio. In some aspects, a substituted heterocycyl group can incorporate an exo- or endocyclic alkene (e.g., cyclohex-2-en-1-yl). In some aspects, the heterocycloalkyl group is unsubstituted or not optionally substituted.

The monocyclic, bicyclic, and tricyclic heterocycles may be connected to the parent molecular moiety through any carbon atom or any nitrogen atom contained within the rings, and can be unsubstituted or substituted.

The term “heterocycloalkylene” as used herein includes a heterocycloalkyl group that is substituted at two points.

As used herein, the term “hydroxyalkyl” includes an alkyl group where at least one hydrogen substituent has been replaced with an alcohol (—OH) group. In certain aspects, the hydroxyalkyl group has one alcohol group. In certain aspects, the hydroxyalkyl group has one or two alcohol groups, each on a different carbon atom. In certain aspects, the hydroxyalkyl group has 1, 2, 3, 4, 5, or 6 alcohol groups. Examples may include, but are not limited to, hydroxymethyl, 2-hydroxyethyl, and 1-hydroxyethyl.

When any two substituent groups or any two instances of the same substituent group are “independently selected” from a list of alternatives, the groups may be the same or different. For example, if R^{a} and R^{b} are independently selected from the group consisting of alkyl, fluoro, amino, and hydroxyalkyl, then a molecule with two R^{a} groups and two R^{b} groups could have all groups be an alkyl group (e.g., four different alkyl groups).

Alternatively, the first R^{a} could be alkyl, the second R^{a} could be fluoro, the first R^{b} could be hydroxyalkyl, and the second R^{b} could be amino (or any other substituents taken from the group). Alternatively, both R^{a} and the first R^{b} could be fluoro, while the second R^{b} could be alkyl (i.e., some pairs of substituent groups may be the same, while other pairs may be different).

“Amino protecting group” is a protecting group that is suitable for preventing undesired reactions at an amino nitrogen. Representative amino-protecting groups include, but are not limited to, formyl; acyl groups, for example alkanoyl groups, such as acetyl and trifluoroacetyl; alkoxycarbonyl groups, such as tert-butoxycarbonyl (Boc); arylmethoxycarbonyl groups, such as benzyloxycarbonyl (Cbz) and 9-fluorenylmethoxycarbonyl (Fmoc); arylmethyl groups, such as benzyl (Bn), trityl (Tr), and 1,1-di-(4′-methoxyphenyl)methyl; and the like.

“Hydroxyl protecting group” is a protecting group that is suitable for preventing undesired reactions at a hydroxyl oxygen. Representative hydroxy-protecting groups include, but are not limited to, acyl groups, for example alkanoyl groups, such as acetyl; arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBDMS); and the like.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

“Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, orotic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

As used herein, “or” should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

As used herein, “spiro bicyclic cycloalkyl” includes a cycloalkyl in which geminal substituents on a carbon atom are replaced to join in forming a 1,1-substituted ring. For example, but without limitation, for a —C(R¹)(R²)— group that was part of a longer carbon chain, if R¹ and R² joined to form a cyclopropyl ring incorporating the carbon to which R¹ and R² were bonded, this would be a spiro bicyclic cycloalkyl group (i.e., spirocyclopropyl).

The term “spiro bicyclic cycloalkylene” as used herein includes a spiro bicyclic cycloalkyl group that is substituted at two points.

As used herein, “spiro bicyclic heterocycloalkyl” includes a heterocycloalkyl in which geminal substituents on a carbon atom are replaced to join in forming a 1,1-substituted ring. For example, but without limitation, for a —C(R¹)(R²)— group that was part of a longer carbon chain, if R¹ and R² joined to form a pyrrolidine ring incorporating the carbon to which R¹ and R² were bonded, this would be a spiro bicyclic heterocycloalkyl group.

The term “Spiro bicyclic heterocycloalkylene” as used herein includes a spiro bicyclic heterocycloalkyl group that is substituted at two points.

Some compounds disclosed herein are characterized by the presence of amino functional groups. One of ordinary skill would therefore understand that compounds can be isolated as salts wherein the amino functional group nitrogen is quarternized.

As used herein, the term “treat,” “treating,” or “treatment” includes administering or applying a composition (e.g., a composition described herein) in an amount, manner (e.g., schedule of administration), and mode (e.g., route of administration) that is effective to improve a disorder or a symptom thereof, or to retard, or to slow the progression of a disorder or a symptom thereof. Such improvements can include, but are not limited to, alleviation or amelioration of one or more symptoms or conditions, diminishment of the extent of a disease, stabilizing (i.e., not worsening) the state of disease, delaying or slowing of disease progression, amelioration or palliation of the disease state, diminishment of the reoccurrence of disease, and remission, whether partial or total and whether detectable or undetectable.

### EMBODIMENTS

### Compounds

In a first aspect, the disclosure provides a compound of Formula I:

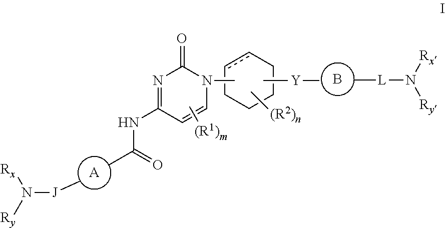

or a single stereoisomer or mixture of stereoisomers thereof, or a pharmaceutically acceptable salt thereof, wherein:
ring A is a 3-8 membered monocyclic heterocycloalkylene optionally substituted with up to three substituents independently selected from the group consisting of C₁-C₆ alkyl, C₁-C₆ alkoxy, halo, CN, C₁-C₆ haloalkyl, phenyl, OH, NH₂, NH(C₁-C₆ alkyl), N(C₁-C₆ alkyl)₂, COOH, COO(C₁-C₆ alkyl), CONH₂, CONH(C₁-C₆ alkyl), CON(C₁-C₆ alkyl)₂, and oxo;
J is C₁-C₆ alkylene or C₃-C₈ cycloalkylene, either of which is optionally substituted with halo, OH, or C₁-C₆ alkoxy, wherein up to two methylene units of the C₁-C₆ alkylene are optionally and independently replaced with O, S, SO, SO₂, or C═O;
Y is a bond or C₁-C₆ alkylene optionally substituted with OH, NH₂, CN, halo, or C₁-C₆ alkoxy, wherein up to two methylene units of the C₁-C₆ alkylene are optionally and independently replaced by O, NH, N—(C₁-C₆ alkyl), N—(C₁-C₆ hydroxyalkyl), N—(C₁-C₆ haloalkyl), N—(C₁₋₆ alkylene-C₃₋₈ cycloalkyl), N—(C₃₋₈ cycloalkyl), NH(C═O), N—(C₁₋₆ alkyl)(C═O), or (C═O);
ring B is a 3-8 membered monocyclic cycloalkylene, 3-8 membered monocyclic heterocycloalkylene, 6-12 membered bicyclic cycloalkylene, or a 6-12 membered bicyclic heterocycloalkylene, each of which is optionally substituted with up to three substituents independently selected from the group consisting of C₁-C₆ alkyl, C₁-C₆ alkoxy, halo, CN, C₁-C₆ haloalkyl, OH, COOH, COO(C₁-C₆ alkyl), CONH₂, CONH(C₁-C₆ alkyl), CON(C₁-C₆ alkyl)₂, and C₁-C₆ hydroxyalkyl;
L is a bond or C₁-C₆ alkylene, wherein up to two methylene units of the C₁-C₆ alkylene are optionally and independently replaced with O, NH, (C═O), NH(C═O), N—(C₁₋₆ alkyl)(C═O), (C═NH), NH(C═N), or N—(C₁₋₆ alkyl);
R¹ and R² are each independently selected from the group consisting of C₁-C₆ alkyl, halo, CN, OH, NH₂, O(C₁-C₆ haloalkyl), NH(C₁-C₆ alkyl), N(C₁-C₆ alkyl)₂, —COO(C₁-C₆ alkyl), CONH₂, C₁-C₆ haloalkyl, C₁-C₆ alkoxy, and C₁-C₆ haloalkoxy;
Rₓ, R_{y}, R_{x′}, and R_{y′} are each independently H, C₁-C₆ alkyl, or an amino protecting group;
m and n are each independently 0, 1, 2, or 3; and
[symbol] represents a single bond or a double bond.

In one embodiment of a compound of formula I, or a single stereoisomer or mixture of stereoisomers thereof, or a pharmaceutically acceptable salt thereof, ring A is a 5-6 membered monocyclic heterocycloalkylene optionally substituted with up to three substituents selected from the group consisting of C₁-C₆ alkyl, C₁-C₆ alkoxy, halo, CN, C₁-C₆ haloalkyl, phenyl, OH, NH₂, and oxo.

In another embodiment of a compound of formula I, or a single stereoisomer or mixture of stereoisomers thereof, or a pharmaceutically acceptable salt thereof, ring A is

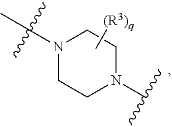

wherein each R³ is independently selected from the group consisting of C₁-C₆ alkyl, C₁-C₆ alkoxy, halo, CN, C₁-C₆ haloalkyl, phenyl, OH, NH₂, and oxo, wherein q is 0, 1, or 2.

In another embodiment of a compound of formula I, or a single stereoisomer or mixture of stereoisomers thereof, or a pharmaceutically acceptable salt thereof, ring A is

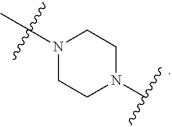 [SMILES: *N1CCN(*)CC1]

In another embodiment of a compound of formula I, or a single stereoisomer or mixture of stereoisomers thereof, or a pharmaceutically acceptable salt thereof, J is C₁-C₆ alkylene optionally substituted with halo, OH, or C₁-C₆ alkoxy, wherein up to two methylene units of the C₁-C₆ alkylene are optionally and independently replaced with O, S, SO, SO₂, or C═O.

In another embodiment of a compound of formula I, or a single stereoisomer or mixture of stereoisomers thereof, or a pharmaceutically acceptable salt thereof, J is C₁-C₆ alkylene optionally substituted with OH, wherein one methylene unit of the C₁-C₆ alkylene is optionally replaced with C═O.

In another embodiment of a compound of formula I, or a single stereoisomer or mixture of stereoisomers thereof, or a pharmaceutically acceptable salt thereof, J is C₁-C₆ alkylene, wherein one methylene unit of the C₁-C₆ alkylene is replaced with C═O.

In another embodiment of a compound of formula I, or a single stereoisomer or mixture of stereoisomers thereof, or a pharmaceutically acceptable salt thereof, J is

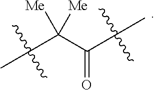 [SMILES: *C(=O)C(*)(C)C]

In another embodiment of a compound of formula I, or a single stereoisomer or mixture of stereoisomers thereof, or a pharmaceutically acceptable salt thereof, Rₓ and R_{y} are H. In another embodiment, Rₓ is H and R_{y} is C₁-C₆ alkyl. In another embodiment, Rₓ is H and R_{y} is an amino protecting group. In another embodiment, Rₓ is H and R_{y} is a Boc group. In another embodiment, Rₓ and R_{y} are each independently C₁-C₆ alkyl.

In another embodiment of a compound of formula I, or a single stereoisomer or mixture of stereoisomers thereof, or a pharmaceutically acceptable salt thereof,

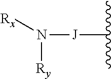

is

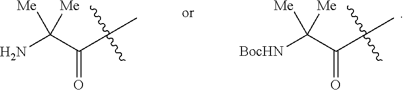 [SMILES: *C(=O)C(C)(C)N ; *C(=O)C(C)(C)NC(=O)OC(C)(C)C]

In another embodiment,

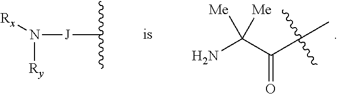

In another embodiment of a compound of formula I, or a single stereoisomer or mixture of stereoisomers thereof, or a pharmaceutically acceptable salt thereof,

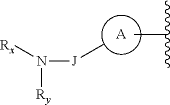

is

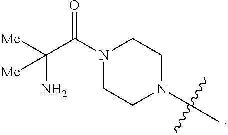 [SMILES: *N1CCN(C(=O)C(C)(C)N)CC1]

In another embodiment of a compound of formula I, or a single stereoisomer or mixture of stereoisomers thereof, or a pharmaceutically acceptable salt thereof,

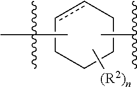

is

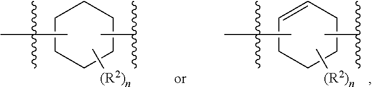

wherein each R² is independently selected from the group consisting of C₁-C₆ alkyl, C₁-C₆ alkoxy, halo, and CN; and n is 0, 1 or 2.

In another embodiment,

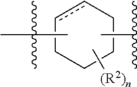

is

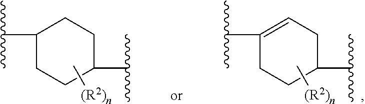

wherein each R² is independently selected from the group consisting of C₁-C₆ alkyl, C₁-C₆ alkoxy, halo, and CN; and n is 0, 1, or 2.

In another embodiment,

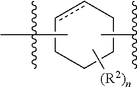

is

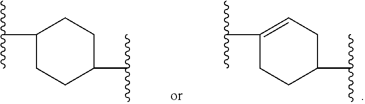 [SMILES: *C1CCC(*)CC1 ; *C1=CCC(*)CC1]

In one embodiment of a compound of formula I, or a single stereoisomer or mixture of stereoisomers thereof, or a pharmaceutically acceptable salt thereof, Y is C₁-C₆ alkylene optionally substituted with OH, NH₂, CN, halo, or C₁-C₆ alkoxy, wherein one methylene unit of the C₁-C₆ alkylene is optionally replaced by O, NH, N—(C₁-C₆ alkyl), N—(C₁-C₆ hydroxyalkyl), N—(C₁-C₆ haloalkyl), N—(C₁₋₆ alkylene-C₃₋₈ cycloalkyl), N—(C₃₋₈ cycloalkyl), NH(C═O), N—(C₁₋₆ alkyl)(C═O), or (C═O).

In another embodiment, Y is C₁-C₃ alkylene optionally substituted with OH, NH₂, halo, or C₁-C₆ alkoxy, and wherein one methylene unit of the C₁-C₃ alkylene is optionally replaced by O, NH, N—(C₁-C₆ alkyl), N—(C₁-C₆ hydroxyalkyl), N—(C₁-C₆ haloalkyl), N—(C₃₋₈ cycloalkyl), N—(C₁₋₆ alkylene-C₃₋₈ cycloalkyl), NH(C═O), N—(C₁₋₆ alkyl)(C═O), or (C═O).

In another embodiment, Y is C₁-C₃ alkylene, wherein one methylene unit of the C₁-C₃ alkylene is optionally replaced by NH, N—(C₁-C₆ haloalkyl), or N—(C₁-C₆ alkyl).

In another embodiment, Y is selected from the group consisting of —CH₂—, —CH₂NH—, —CH₂NMe-, —CH₂N(CH₂CH₂F)—, and —CH₂NEt-.

In another embodiment of a compound of formula I, or a single stereoisomer or mixture of stereoisomers thereof, or a pharmaceutically acceptable salt thereof, wherein

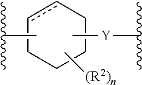

is selected from the group consisting of

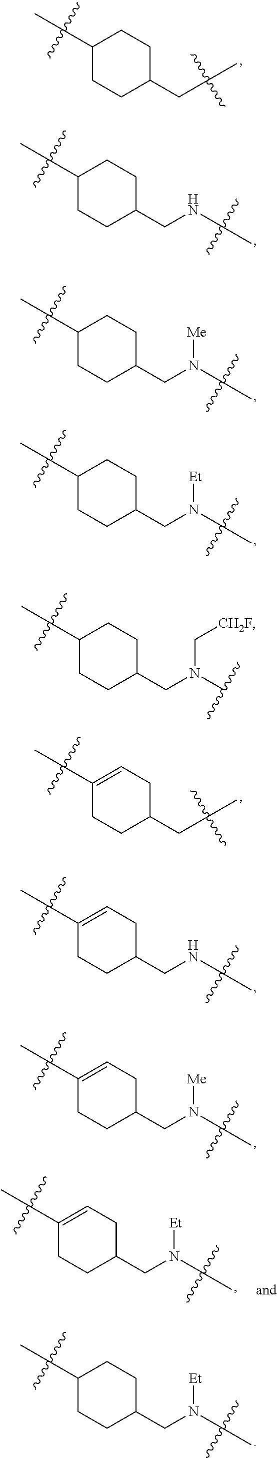

In another embodiment of a compound of formula I or a single stereoisomer or mixture of stereoisomers thereof, or a pharmaceutically acceptable salt thereof, ring B is a 3-8 membered monocyclic cycloalkylene, 3-8 membered monocyclic heterocycloalkylene, or a 6-12 membered bicyclic heterocycloalkylene, each of which is optionally substituted with up to three substituents independently selected from the group consisting of C₁-C₆ alkyl, C₁-C₆ alkoxy, halo, CN, C₁-C₆ haloalkyl, OH, COOH, COO(C₁-C₆ alkyl), CONH₂, CONH(C₁-C₆ alkyl), CON(C₁-C₆ alkyl)₂, and C₁-C₆ hydroxyalkyl.

In another embodiment, ring B is a 3-8 membered monocyclic cycloalkylene, 3-8 membered monocyclic heterocycloalkylene optionally substituted with C₁-C₆ alkyl, or a 6-12 membered bicyclic heterocycloalkylene.

In another embodiment, ring B is a 5-6 membered monocyclic cycloalkylene, 4-7 membered monocyclic heterocycloalkylene, or a 6-9 membered bicyclic heterocycloalkylene.

In another embodiment, ring B is a 3-8 membered monocyclic cycloalkylene optionally substituted with up to three substituents independently selected from the group consisting of C₁-C₆ alkyl, C₁-C₆ alkoxy, halo, CN, C₁-C₆ haloalkyl, OH, COOH, COO(C₁-C₆ alkyl), CONH₂, CONH(C₁-C₆ alkyl), CON(C₁-C₆ alkyl)₂, and C₁-C₆ hydroxyalkyl.

In another embodiment, ring B is a 4-6 membered monocyclic cycloalkylene optionally substituted with up to three substituents independently selected from the group consisting of C₁-C₆ alkyl, C₁-C₆ alkoxy, halo, CN, C₁-C₆ haloalkyl, OH, and C₁-C₆ hydroxyalkyl.

In another embodiment, ring B is a 5-6 membered monocyclic cycloalkylene optionally substituted with up to three substituents independently selected from the group consisting of C₁-C₆ alkyl, C₁-C₆ alkoxy, halo, CN, C₁-C₆ haloalkyl, OH, and C₁-C₆ hydroxyalkyl.

In another embodiment, ring B is a 4-6 membered monocyclic cycloalkylene. In another embodiment, ring B is cyclopentylene or cyclohexylene.

In another embodiment, ring B is a 3-8 membered monocyclic heterocycloalkylene optionally substituted with up to three substituents independently selected from the group consisting of C₁-C₆ alkyl, C₁-C₆ alkoxy, halo, CN, C₁-C₆ haloalkyl, OH, COOH, COO(C₁-C₆ alkyl), CONH₂, CONH(C₁-C₆ alkyl), CON(C₁-C₆ alkyl)₂, and C₁-C₆ hydroxyalkyl.

In another embodiment, ring B is a 4-7 membered monocyclic heterocycloalkylene optionally substituted with up to three substituents independently selected from the group consisting of C₁-C₆ alkyl, C₁-C₆ alkoxy, halo, CN, C₁-C₆ haloalkyl, OH, and C₁-C₆ hydroxyalkyl, and wherein ring B contains up to 2 nitrogen atoms.

In another embodiment, ring B is a 4-7 membered monocyclic heterocycloalkylene containing up to two heteroatoms selected from nitrogen and oxygen, wherein ring B is optionally substituted with C₁-C₆ alkyl. In another embodiment, ring B is a 4-7 membered monocyclic heterocycloalkylene containing one nitrogen atom, wherein ring B is optionally substituted with C₁-C₆ alkyl.

In another embodiment, ring B is a 6-10 membered bicyclic cycloalkylene optionally substituted with up to three substituents independently selected from the group consisting of C₁-C₆ alkyl, C₁-C₆ alkoxy, halo, C₁-C₆ haloalkyl, OH, and C₁-C₆ hydroxyalkyl.

In another embodiment, ring B is a 6-9 membered bicyclic cycloalkylene optionally substituted with up to three substituents independently selected from the group consisting of C₁-C₆ alkyl, C₁-C₆ alkoxy, halo, C₁-C₆ haloalkyl, OH, and C₁-C₆ hydroxyalkyl.

In another embodiment, ring B is a 6-10 membered fused, spiro, or bridged bicyclic cycloalkylene. In another embodiment, ring B is a 6-10 membered fused bicyclic cycloalkylene. In another embodiment, ring B is a 6-10 membered bridged bicyclic cycloalkylene. In another embodiment, ring B is a 6-10 membered spiro bicyclic cycloalkylene.

In another embodiment, ring B is a 6-12 membered bicyclic heterocycloalkylene optionally substituted with up to three substituents independently selected from the group consisting of C₁-C₆ alkyl, C₁-C₆ alkoxy, halo, C₁-C₆ haloalkyl, OH, and C₁-C₆ hydroxyalkyl.

In another embodiment, ring B is a 6-10 membered bicyclic heterocycloalkylene optionally substituted with up to three substituents independently selected from the group consisting of C₁-C₆ alkyl, C₁-C₆ alkoxy, halo, C₁-C₆ haloalkyl, OH, and C₁-C₆ hydroxyalkyl.

In another embodiment, ring B is a 6-9 membered bicyclic heterocycloalkylene optionally substituted with up to three substituents independently selected from the group consisting of C₁-C₆ alkyl, C₁-C₆ alkoxy, halo, C₁-C₆ haloalkyl, OH, and C₁-C₆ hydroxyalkyl.

In another embodiment, ring B is a 6-10 membered fused, spiro, or bridged bicyclic heterocycloalkylene containing up to 2 nitrogen atoms. In another embodiment, ring B is a 6-9 membered fused, spiro, or bridged bicyclic heterocycloalkylene containing up to 2 nitrogen atoms.

In another embodiment, ring B is a 6-10 membered fused, spiro, or bridged bicyclic heterocycloalkylene containing one nitrogen atom. In another embodiment, ring B is a 6-9 membered fused bicyclic heterocycloalkylene containing one nitrogen atom. In another embodiment, ring B is a 6-9 membered spiro bicyclic heterocycloalkylene containing one nitrogen atom. In another embodiment, ring B is a 6-9 membered bridged bicyclic heterocycloalkylene containing one nitrogen atom.

In another embodiment, ring B is selected from the group consisting of

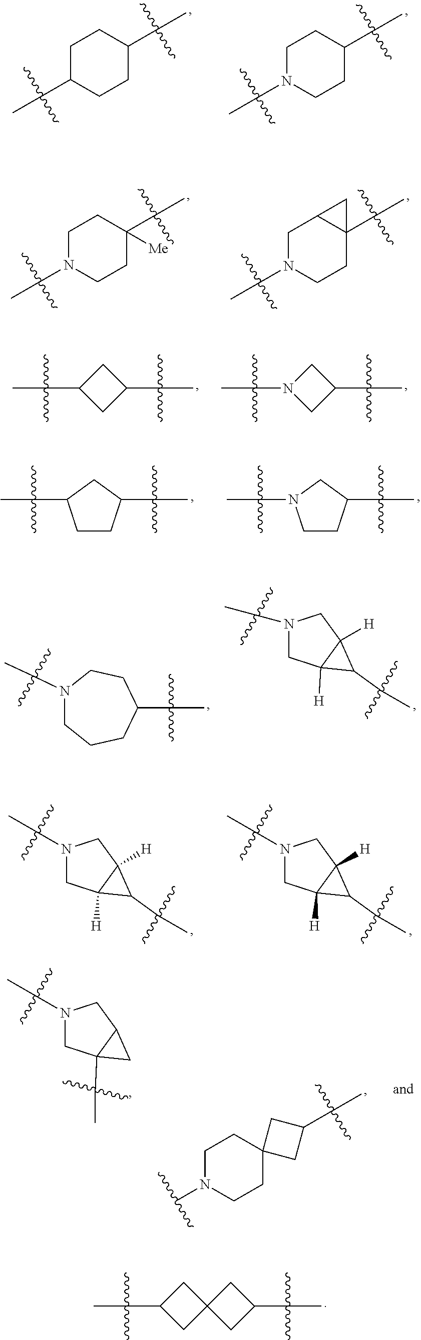

In another embodiment of a compound of formula I, or a single stereoisomer or mixture of stereoisomers thereof, or a pharmaceutically acceptable salt thereof, wherein L is a bond.

In another embodiment of a compound of formula I, or a single stereoisomer or mixture of stereoisomers thereof, or a pharmaceutically acceptable salt thereof, wherein L is C₁-C₆ alkylene, wherein up to two methylene units of the C₁-C₆ alkylene are optionally and independently replaced with O, NH, (C═O), NH(C═O), N—(C₁₋₆ alkyl)(C═O), (C═NH), NH(C═N), or N—(C₁₋₆ alkyl).

In another embodiment, L is C₁-C₃ alkylene, wherein one methylene unit of the C₁-C₆ alkylene is optionally and independently replaced with O, NH, (C═O), NH(C═O), N—(C₁₋₆ alkyl)(C═O), (C═NH), NH(C═N), or N—(C₁₋₆ alkyl).

In another embodiment, L is C₁-C₆ alkylene. In another embodiment, L is —CH₂— or —CH₂—CH₂—.

In another embodiment, L is a bond or C₁-C₃ alkylene. In another embodiment, L is a bond, —CH₂—, or —CH₂—CH₂—.

In another embodiment of a compound of formula I, or a single stereoisomer or mixture of stereoisomers thereof, or a pharmaceutically acceptable salt thereof, R_{x′} and R_{y′} are each independently H. In another embodiment, R_{x′} is H and R_{y′} is C₁-C₆ alkyl. In another embodiment, R_{x′} is H and R_{y′} is an amino protecting group. In another embodiment, R_{x′} is H and R_{y′} is a Boc group. In another embodiment, R_{x′} and R_{y′} are each independently C₁-C₆ alkyl.

In another embodiment of a compound of formula I or a pharmaceutically acceptable salt thereof, Y is C₁-C₃ alkylene, and wherein one methylene unit of the C₁-C₃ alkylene is optionally replaced by NH, N—(C₁-C₆ haloalkyl), or N—(C₁-C₆ alkyl); ring B is a 5-6 membered monocyclic cycloalkylene, 4-7 membered monocyclic heterocycloalkylene optionally substituted with C₁-C₆ alkyl, or 6-9 membered bicyclic heterocycloalkylene; L is a bond or C₁-C₃ alkylene; and R_{x′} and R_{y′} are each independently H or C₁-C₆ alkyl.

In another embodiment, Y is selected from the group consisting of —CH₂—, —CH₂NH—, —CH₂NMe-, —CH₂N(CH₂CH₂F)—, and —CH₂NEt-; ring B is selected from the group consisting of

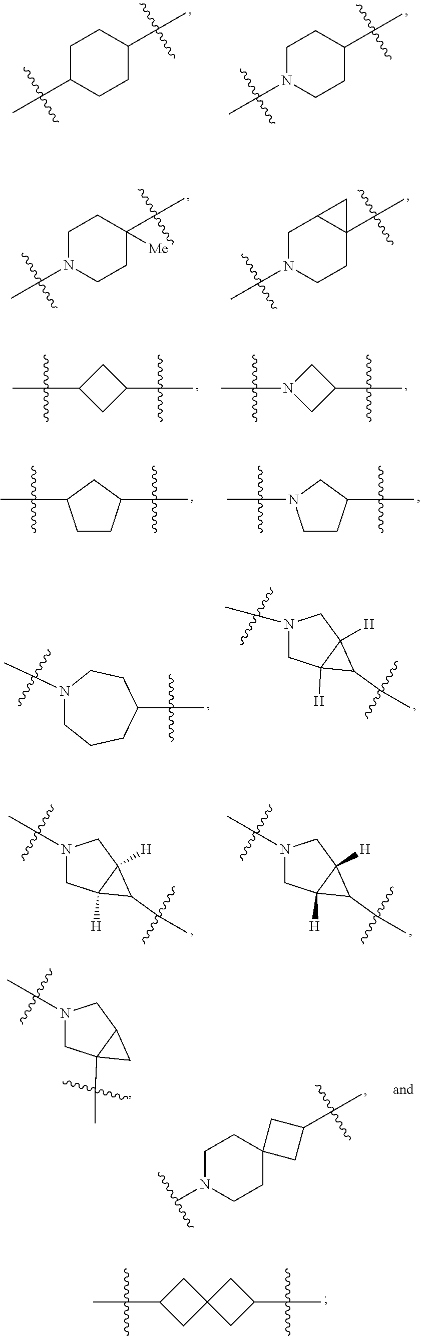

L is a bond, —CH₂—, or —CH₂—CH₂—; and R_{x′} and R_{y′} are each independently H.

In another embodiment of a compound of formula I or a single stereoisomer or mixture of stereoisomers thereof, or a pharmaceutically acceptable salt thereof,

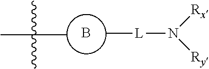

is selected from the group consisting of

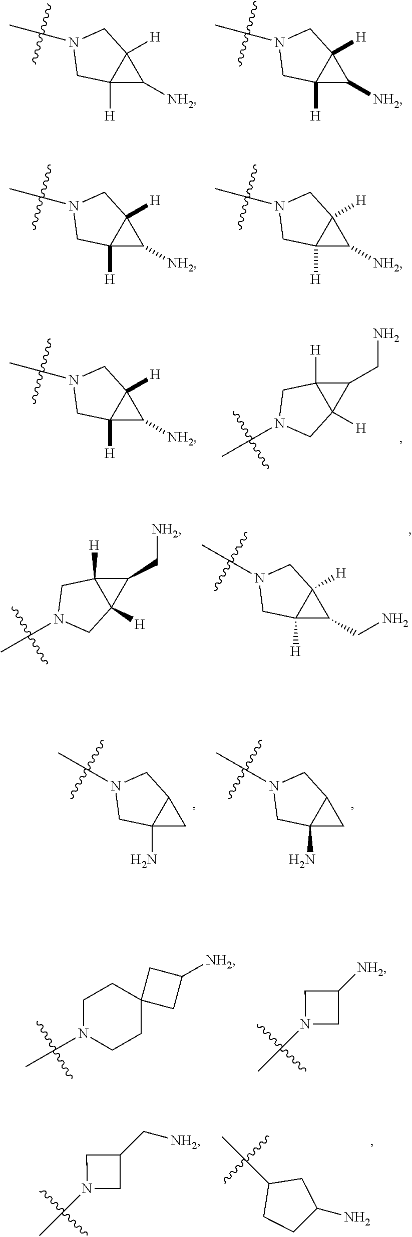

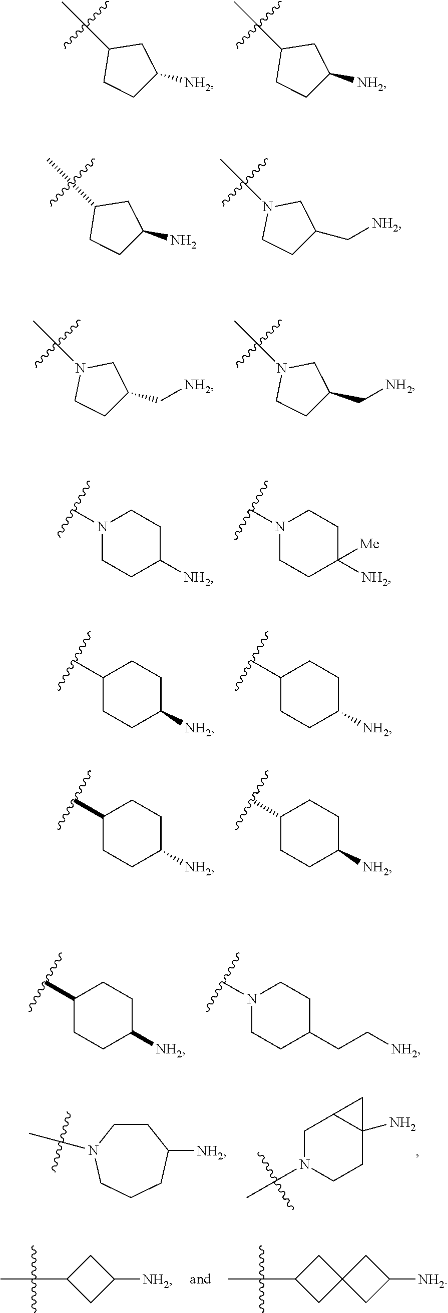

In another embodiment of a compound of formula I, or a single stereoisomer or mixture of stereoisomers thereof, or a pharmaceutically acceptable salt thereof, R¹ and R² are each independently selected from the group consisting of C₁-C₆ alkyl, halo, C₁-C₆ haloalkyl, oxo, and C₁-C₆ alkoxy, and m and n are each independently 0, 1, or 2. In another embodiment, R¹ and R² are each independently C₁-C₆ alkyl, halo, oxo, or C₁-C₆ haloalkyl, and m and n are each independently 0 or 1. In another embodiment, m and n are 0.

In another embodiment, the compound of formula I is a compound of formula IA:

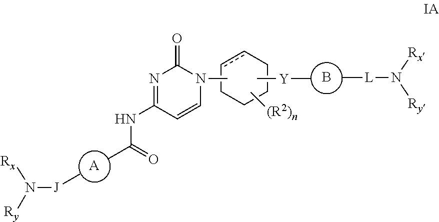

or a single stereoisomer or mixture of stereoisomers thereof, or a pharmaceutically acceptable salt thereof, wherein ring A, ring B, J, L, Y, R², Rₓ, R_{y}, R_{x′}, R_{y′}, and n are the same as defined herein.

In another embodiment, the compound of formula I or IA is a compound of formula IA-1:

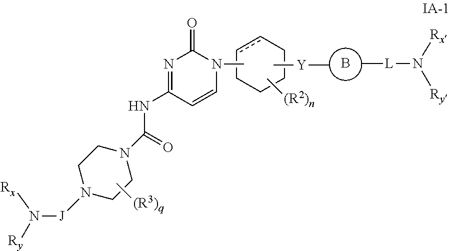

or a single stereoisomer or mixture of stereoisomers thereof, or a pharmaceutically acceptable salt thereof, wherein ring B, J, L, Y, R², Rₓ, R_{y}, R_{x′}, R_{y′}, and n are the same as defined herein; each R³ is independently selected from the group consisting of H, C₁-C₆ alkyl, C₁-C₆ alkoxy, halo, CN, C₁-C₆ haloalkyl, OH, NH₂, and oxo, and q is 0, 1, 2, or 3.

In another embodiment, the compound of formula I, IA, or IA-1 is a compound of formula IA-2:

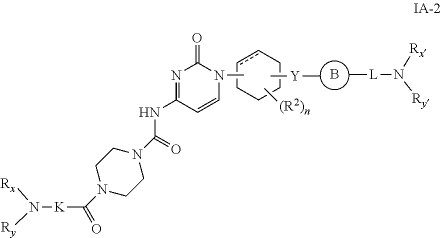

or a single stereoisomer or mixture of stereoisomers thereof, or a pharmaceutically acceptable salt thereof, wherein ring B, L, Y, R², Rₓ, R_{y}, R_{x′}, R_{y′}, and n are the same as defined herein; K is C₁-C₄ alkylene optionally substituted with halo, hydroxyl or C₁-C₆ alkoxy group.

In another embodiment, the compound of formula I, IA, IA-1, or IA-2 is a compound of formula IA-3:

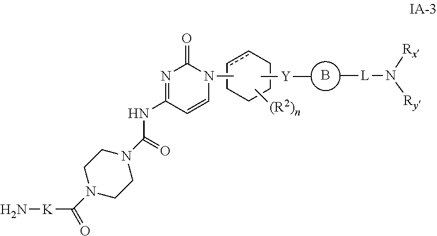

or a single stereoisomer or mixture of stereoisomers thereof, or a pharmaceutically acceptable salt thereof, wherein ring B, L, Y, R², R_{x′}, R_{y′}, and n are the same as defined herein; and K is C₁-C₄ alkylene.

In another embodiment, the compound of formula I, IA, IA-1, IA-2, or IA-3 is a compound of formula IA-4:

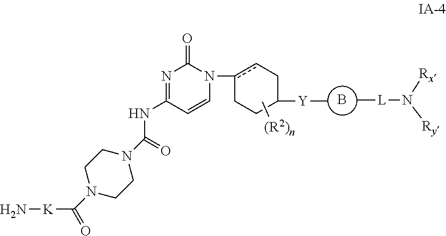

or a single stereoisomer or mixture of stereoisomers thereof, or a pharmaceutically acceptable salt thereof, wherein ring B, L, Y, R², R_{x′}, R_{y′}, and n are the same as defined herein; and K is C₁-C₃ alkylene.

In another embodiment, the compound of formula I, IA, IA-1, IA-2, IA-3, or IA-4 is a compound of formula IA-5:

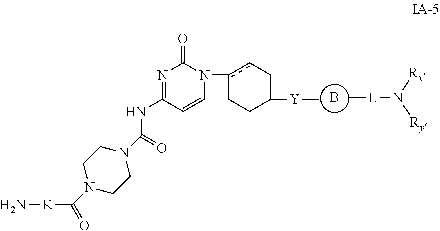

or a single stereoisomer or mixture of stereoisomers thereof, or a pharmaceutically acceptable salt thereof, wherein ring B, L, Y, K, R², R_{x′}, R_{y′}, and n are the same as defined herein.

In another embodiment, the compound of formula I, IA, IA-1, IA-2, IA-3, IA-4, or IA-5 is a compound of formula IA-6:

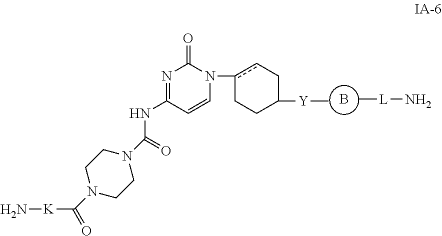

or a single stereoisomer or mixture of stereoisomers thereof, or a pharmaceutically acceptable salt thereof, wherein ring B, L, Y, and K are the same as defined herein.

In another embodiment, the compound of formula I, IA, IA-1, IA-2, IA-3, IA-4, IA-5, or IA-6 is a compound of formula IA-7:

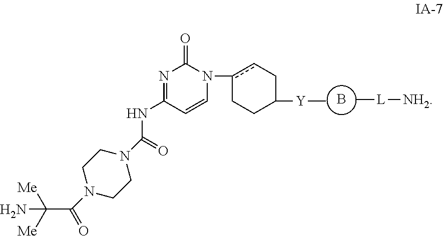

or a single stereoisomer or mixture of stereoisomers thereof, or a pharmaceutically acceptable salt thereof, wherein ring B, L, and Y are the same as defined herein.

In another embodiment, the compound of formula I, IA, IA-1, IA-2, IA-3, IA-4, IA-5, IA-6, or IA-7 is a compound of formula IA-7a, formula IA-7b, formula IA-7c, formula IA-7d, IA-7e, IA-7f, or IA-7g:

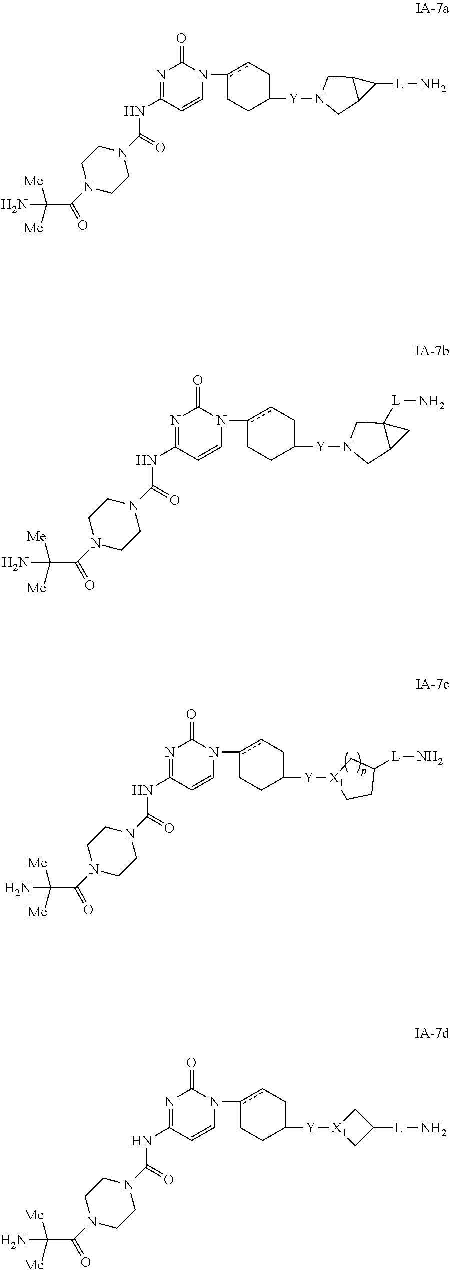

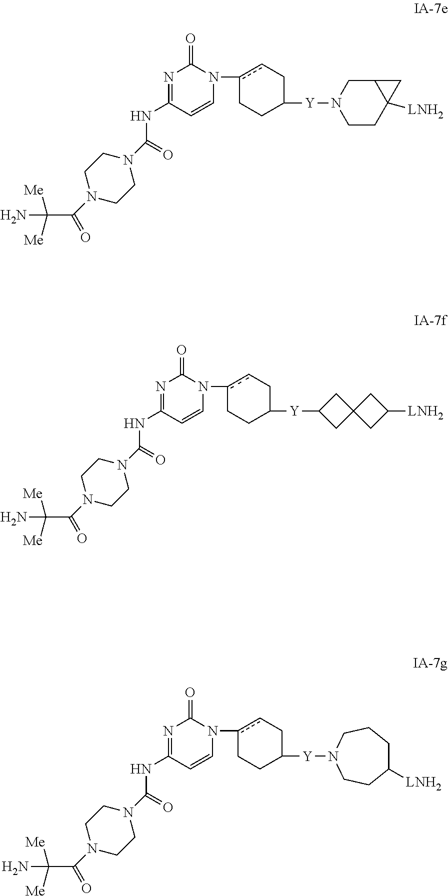

or a single stereoisomer or mixture of stereoisomers thereof, or a pharmaceutically acceptable salt thereof, wherein L and Y are the same as defined herein; each X₁ is independently CH or N; and p is 1, 2, or 3.

In another embodiment, Y is C₁-C₃ alkylene, wherein one methylene unit of the C₁-C₃ alkylene is optionally replaced by NH, N—(C₁-C₆ haloalkyl), or N—(C₁-C₆ alkyl); and L is a bond or C₁-C₃ alkylene.

In another embodiment, Y is C₁-C₃ alkylene, and wherein one methylene unit of the C₁-C₃ alkylene is optionally replaced by NH, N—(C₁-C₆ haloalkyl), or N—(C₁-C₆ alkyl); ring B is a 5-6 membered monocyclic cycloalkylene, 4-7 membered monocyclic heterocycloalkylene optionally substituted with C₁-C₆ alkyl, or a 6-9 membered bicyclic heterocycloalkylene; L is a bond or C₁-C₃ alkylene; and R_{x′} and R_{y′} are each independently H or C₁-C₆ alkyl.

In another embodiment, Y is C₁-C₃ alkylene, and wherein one methylene unit of the C₁-C₃ alkylene is optionally replaced by NH, N—(C₁-C₆ haloalkyl), or N—(C₁-C₆ alkyl); ring B is a 5-6 membered monocyclic cycloalkylene, 4-7 membered monocyclic heterocycloalkylene optionally substituted with C₁-C₆ alkyl, or a 6-9 membered bicyclic heterocycloalkylene; and L is a bond or C₁-C₃ alkylene.

In another embodiment, Y is selected from the group consisting of —CH₂—, —CH₂NH—, —CH₂NMe-, —CH₂N(CH₂CH₂F)—, CH₂NCH₂F—, and —CH₂NEt-; ring B is selected from the group consisting of

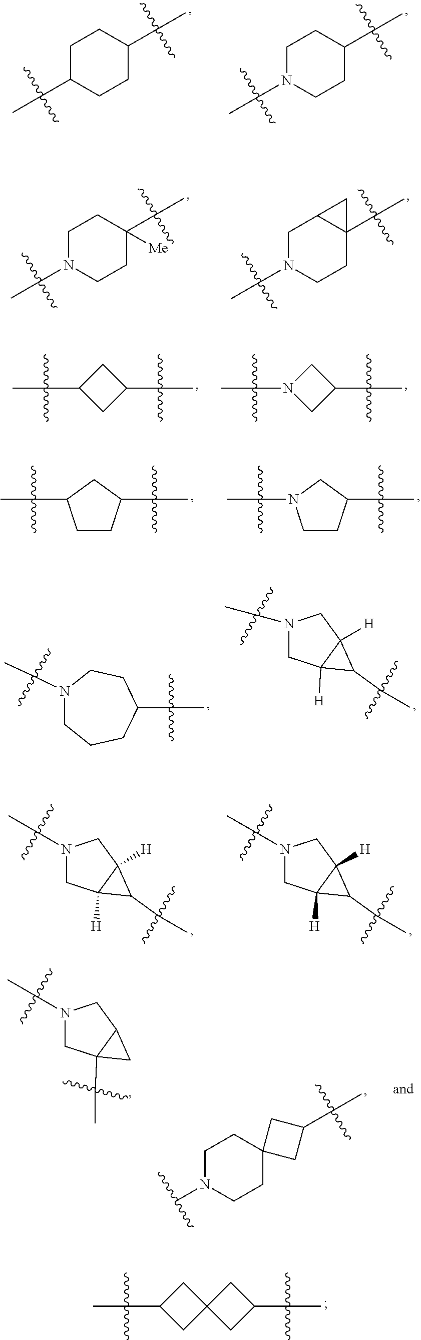

L is a bond, —CH₂—, or —CH₂—CH₂—; and R_{x′} and R_{y′} are each independently H.

In another aspect, the disclosure provides a compound, or a single stereoisomer or mixture of stereoisomers thereof, or a pharmaceutically acceptable salt thereof, which is depicted in Table 1. In Table 1, free base and salt structures of the compounds are depicted.

TABLE 1
Compounds of Formula I

| No. | Salt Structure |
|---|---|
| 1 | 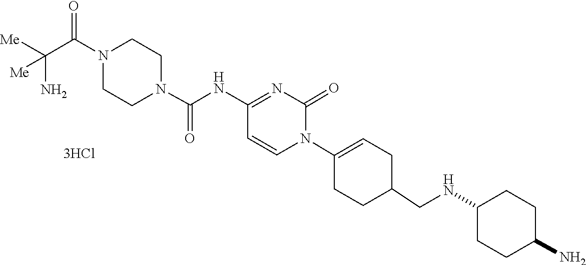 |
| 2 | 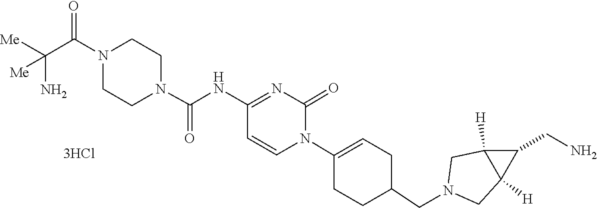 [SMILES: CC(C)(N)C(=O)N1CCN(C(=O)Nc2ccn(C3=CCC(CN4C[C@@H]5[C@@H](CN)[C@@H]5C4)CC3)c(=O)n2)CC1] |
| 3 | 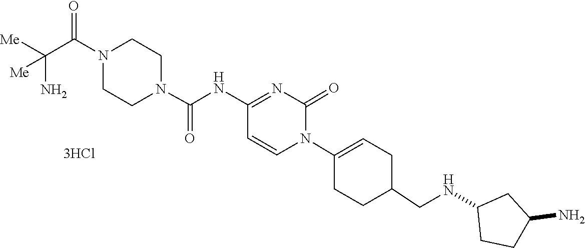 |
| 4 | 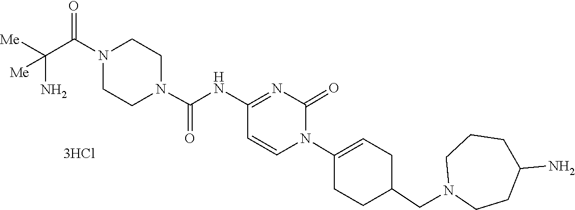 [SMILES: CC(C)(N)C(=O)N1CCN(C(=O)Nc2ccn(C3=CCC(CN4CCCC(N)CC4)CC3)c(=O)n2)CC1] |
| 5 | 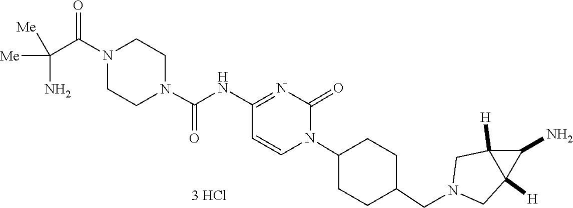 [SMILES: CC(C)(N)C(=O)N1CCN(C(=O)Nc2ccn(C3CCC(CN4C[C@@H]5[C@@H](N)[C@@H]5C4)CC3)c(=O)n2)CC1] |
| 6 | 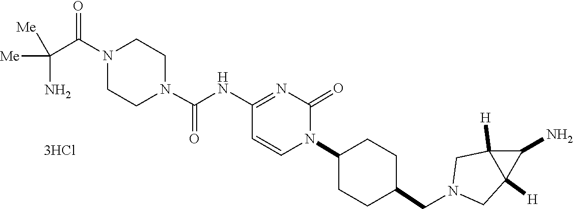 |
| 7 | 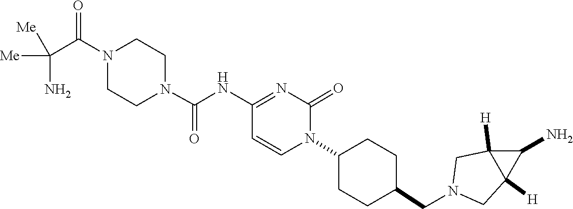 |
| 8 | 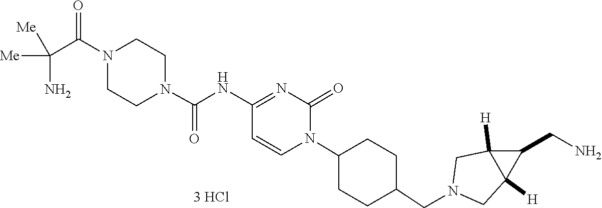 [SMILES: CC(C)(N)C(=O)N1CCN(C(=O)Nc2ccn(C3CCC(CN4C[C@@H]5[C@@H](CN)[C@@H]5C4)CC3)c(=O)n2)CC1] |
| 9 | 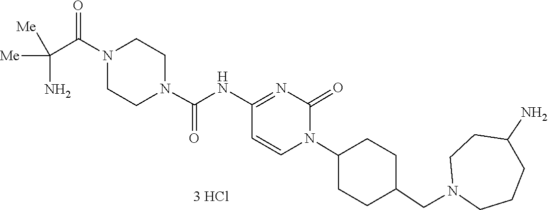 [SMILES: CC(C)(N)C(=O)N1CCN(C(=O)Nc2ccn(C3CCC(CN4CCCC(N)CC4)CC3)c(=O)n2)CC1] |
| 10 | 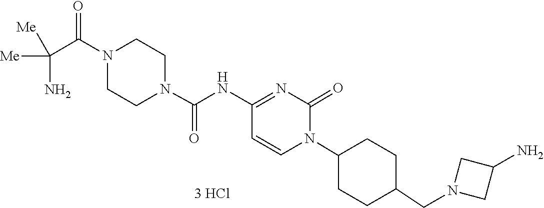 [SMILES: CC(C)(N)C(=O)N1CCN(C(=O)Nc2ccn(C3CCC(CN4CC(N)C4)CC3)c(=O)n2)CC1] |
| 11 | 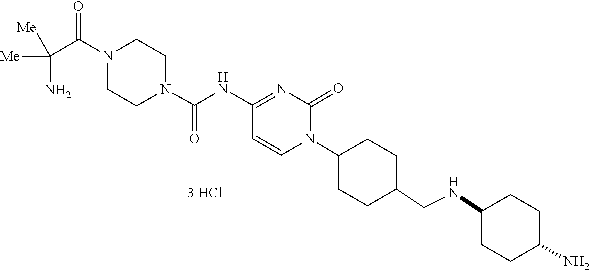 |
| 12 | 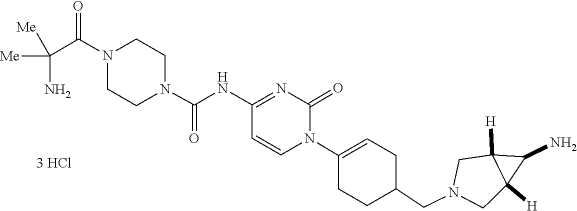 [SMILES: CC(C)(N)C(=O)N1CCN(C(=O)Nc2ccn(C3=CCC(CN4C[C@@H]5[C@@H](N)[C@@H]5C4)CC3)c(=O)n2)CC1] |

Compounds of Formula I

| No. | Free Base Structure |
|---|---|
| 1 | 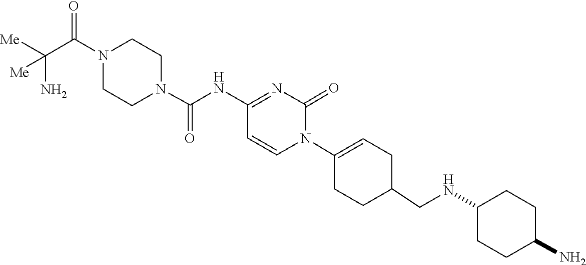 |
| 2 | 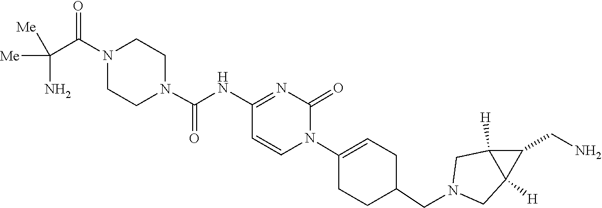 [SMILES: CC(C)(N)C(=O)N1CCN(C(=O)Nc2ccn(C3=CCC(CN4C[C@@H]5[C@@H](CN)[C@@H]5C4)CC3)c(=O)n2)CC1] |
| 3 | 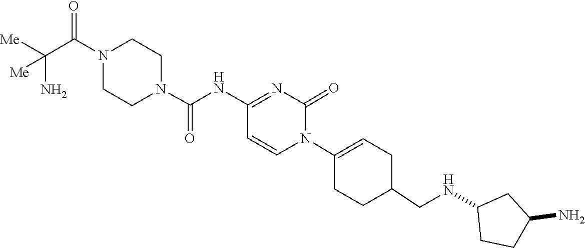 |
| 4 | 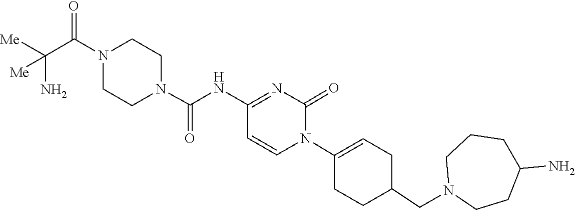 [SMILES: CC(C)(N)C(=O)N1CCN(C(=O)Nc2ccn(C3=CCC(CN4CCCC(N)CC4)CC3)c(=O)n2)CC1] |
| 5 | 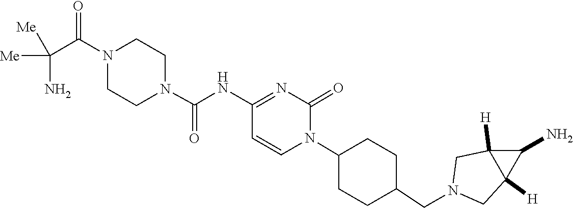 [SMILES: CC(C)(N)C(=O)N1CCN(C(=O)Nc2ccn(C3CCC(CN4C[C@@H]5[C@@H](N)[C@@H]5C4)CC3)c(=O)n2)CC1] |
| 6 | 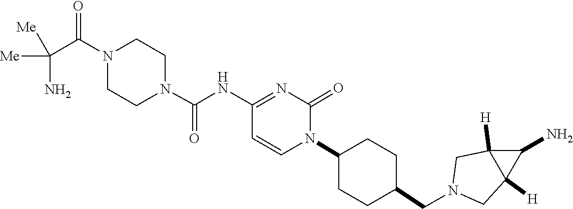 |
| 7 | 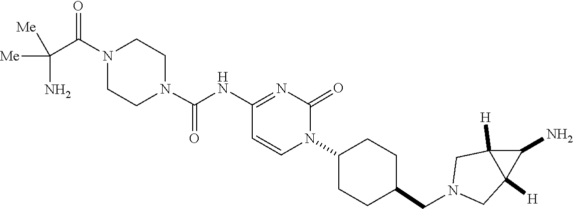 |
| 8 | 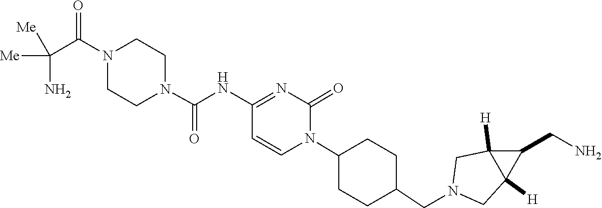 [SMILES: CC(C)(N)C(=O)N1CCN(C(=O)Nc2ccn(C3CCC(CN4C[C@@H]5[C@@H](CN)[C@@H]5C4)CC3)c(=O)n2)CC1] |
| 9 | 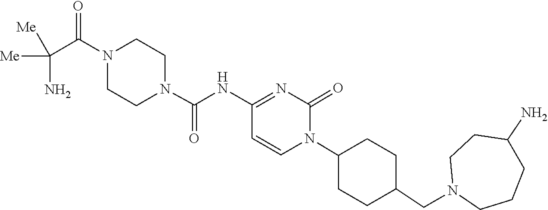 [SMILES: CC(C)(N)C(=O)N1CCN(C(=O)Nc2ccn(C3CCC(CN4CCCC(N)CC4)CC3)c(=O)n2)CC1] |
| 10 | 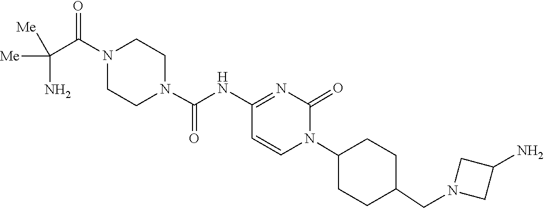 [SMILES: CC(C)(N)C(=O)N1CCN(C(=O)Nc2ccn(C3CCC(CN4CC(N)C4)CC3)c(=O)n2)CC1] |
| 11 | 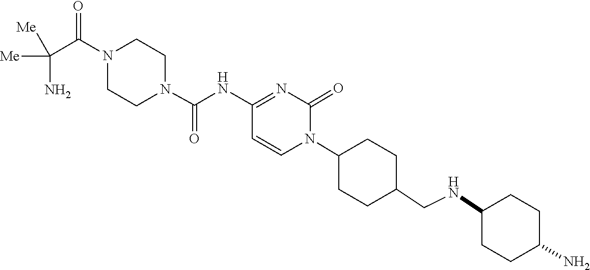 |
| 12 | 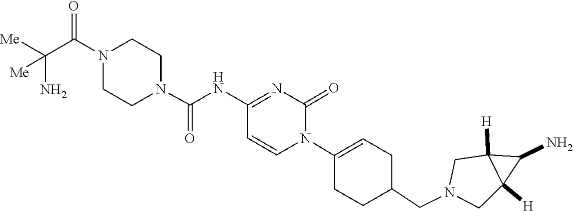 [SMILES: CC(C)(N)C(=O)N1CCN(C(=O)Nc2ccn(C3=CCC(CN4C[C@@H]5[C@@H](N)[C@@H]5C4)CC3)c(=O)n2)CC1] |

In another aspect, the disclosure provides a compound, or a single stereoisomer or mixture of stereoisomers thereof, or a pharmaceutically acceptable salt thereof which is depicted in Table 2. In Table 2, free base and salt structures of the compounds are depicted.

TABLE 2
Additional Compounds of Formula I

| No. | Salt Structure |
|---|---|
| 13 | 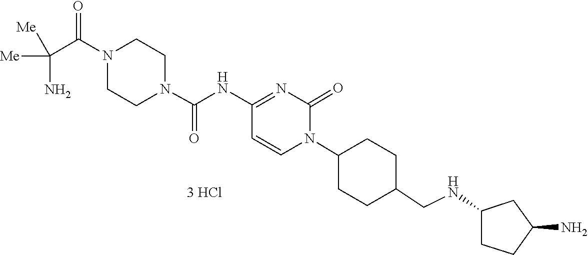 [SMILES: CC(C)(N)C(=O)N1CCN(C(=O)Nc2ccn(C3CCC(CN[C@H]4CC[C@H](N)C4)CC3)c(=O)n2)CC1] |
| 14 | 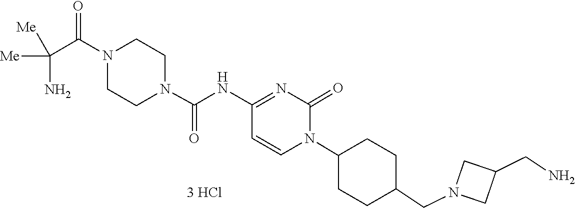 [SMILES: CC(C)(N)C(=O)N1CCN(C(=O)Nc2ccn(C3CCC(CN4CC(CN)C4)CC3)c(=O)n2)CC1] |
| 15 | 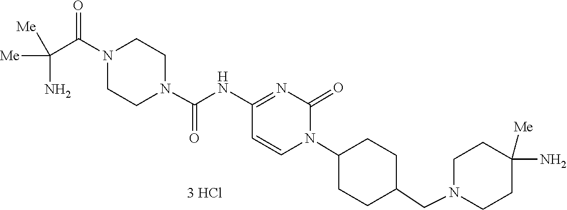 [SMILES: CC1(N)CCN(CC2CCC(n3ccc(NC(=O)N4CCN(C(=O)C(C)(C)N)CC4)nc3=O)CC2)CC1] |
| 16 | 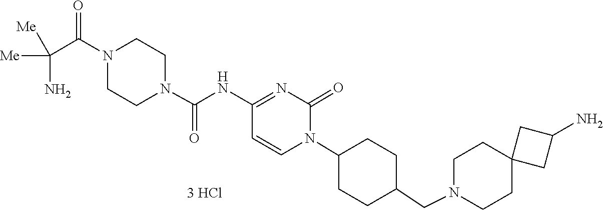 [SMILES: CC(C)(N)C(=O)N1CCN(C(=O)Nc2ccn(C3CCC(CN4CCC5(CC4)CC(N)C5)CC3)c(=O)n2)CC1] |
| 17 | 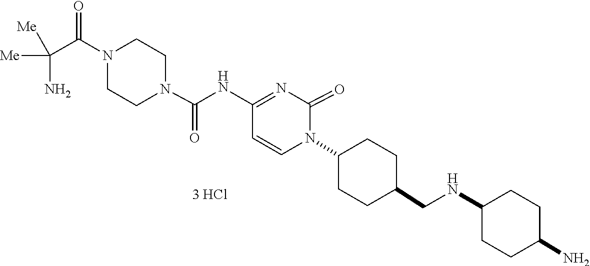 [SMILES: CC(C)(N)C(=O)N1CCN(C(=O)Nc2ccn([C@H]3CC[C@H](CN[C@H]4CC[C@@H](N)CC4)CC3)c(=O)n2)CC1] |
| 18 | 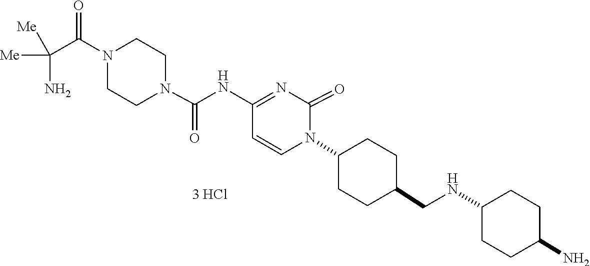 [SMILES: CC(C)(N)C(=O)N1CCN(C(=O)Nc2ccn([C@H]3CC[C@H](CN[C@H]4CC[C@H](N)CC4)CC3)c(=O)n2)CC1] |
| 19 | 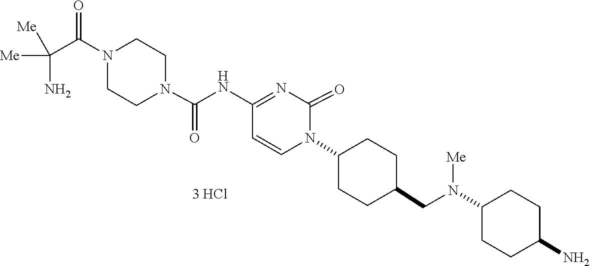 [SMILES: CN(C[C@H]1CC[C@H](n2ccc(NC(=O)N3CCN(C(=O)C(C)(C)N)CC3)nc2=O)CC1)[C@H]1CC[C@H](N)CC1] |
| 20 | 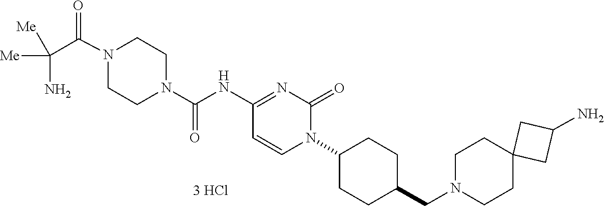 [SMILES: CC(C)(N)C(=O)N1CCN(C(=O)Nc2ccn([C@H]3CC[C@H](CN4CCC5(CC4)CC(N)C5)CC3)c(=O)n2)CC1] |
| 21 | 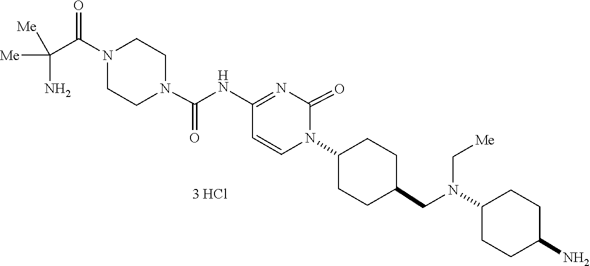 [SMILES: CCN(C[C@H]1CC[C@H](n2ccc(NC(=O)N3CCN(C(=O)C(C)(C)N)CC3)nc2=O)CC1)[C@H]1CC[C@H](N)CC1] |
| 22 | 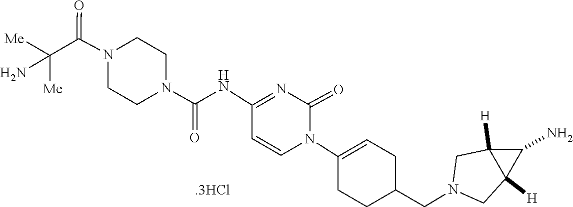 [SMILES: CC(C)(N)C(=O)N1CCN(C(=O)Nc2ccn(C3=CCC(CN4C[C@@H]5[C@H](N)[C@@H]5C4)CC3)c(=O)n2)CC1] |
| 23 | 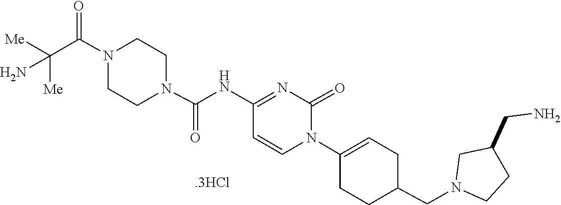 [SMILES: CC(C)(N)C(=O)N1CCN(C(=O)Nc2ccn(C3=CCC(CN4CC[C@H](CN)C4)CC3)c(=O)n2)CC1] |
| 24 | 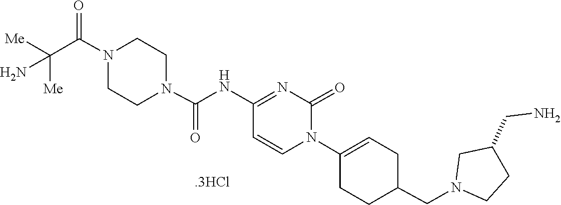 [SMILES: CC(C)(N)C(=O)N1CCN(C(=O)Nc2ccn(C3=CCC(CN4CC[C@@H](CN)C4)CC3)c(=O)n2)CC1] |
| 25 | 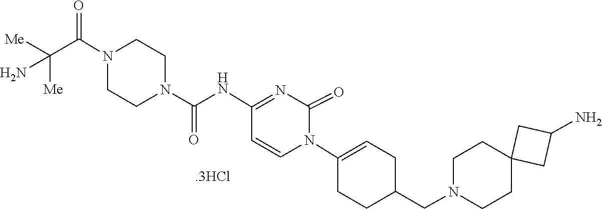 [SMILES: CC(C)(N)C(=O)N1CCN(C(=O)Nc2ccn(C3=CCC(CN4CCC5(CC4)CC(N)C5)CC3)c(=O)n2)CC1] |
| 26 | 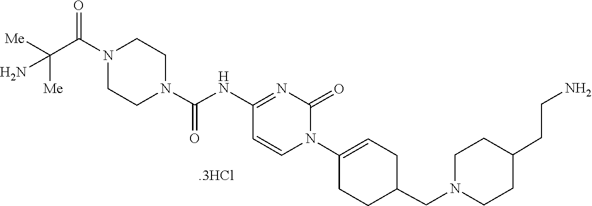 [SMILES: CC(C)(N)C(=O)N1CCN(C(=O)Nc2ccn(C3=CCC(CN4CCC(CCN)CC4)CC3)c(=O)n2)CC1] |
| 27 | 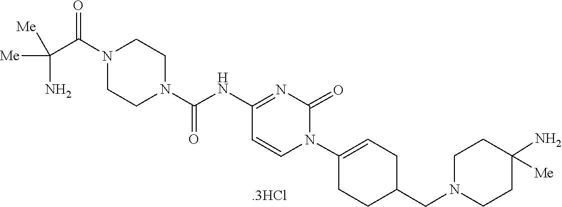 [SMILES: CC1(N)CCN(CC2CC=C(n3ccc(NC(=O)N4CCN(C(=O)C(C)(C)N)CC4)nc3=O)CC2)CC1] |
| 28 | 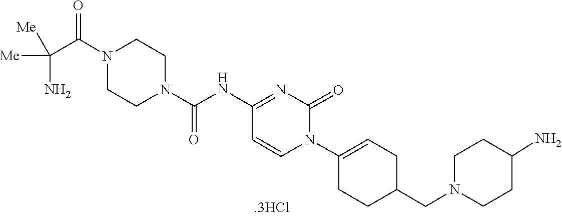 [SMILES: CC(C)(N)C(=O)N1CCN(C(=O)Nc2ccn(C3=CCC(CN4CCC(N)CC4)CC3)c(=O)n2)CC1] |
| 29 | 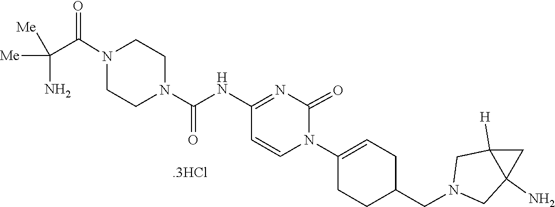 [SMILES: CC(C)(N)C(=O)N1CCN(C(=O)Nc2ccn(C3=CCC(CN4CC5CC5(N)C4)CC3)c(=O)n2)CC1] |
| 30 | 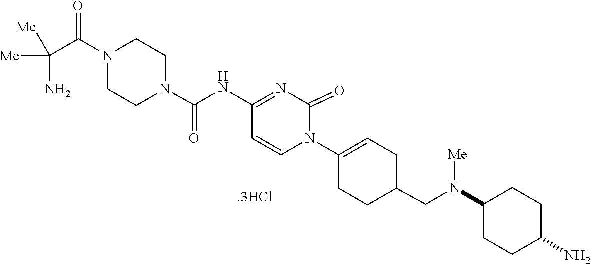 [SMILES: CN(CC1CC=C(n2ccc(NC(=O)N3CCN(C(=O)C(C)(C)N)CC3)nc2=O)CC1)[C@H]1CC[C@H](N)CC1] |
| 31 | 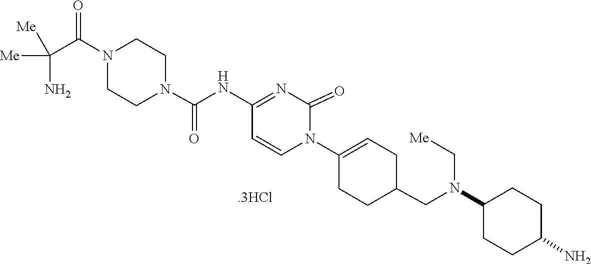 [SMILES: CCN(CC1CC=C(n2ccc(NC(=O)N3CCN(C(=O)C(C)(C)N)CC3)nc2=O)CC1)[C@H]1CC[C@H](N)CC1] |
| 32 | 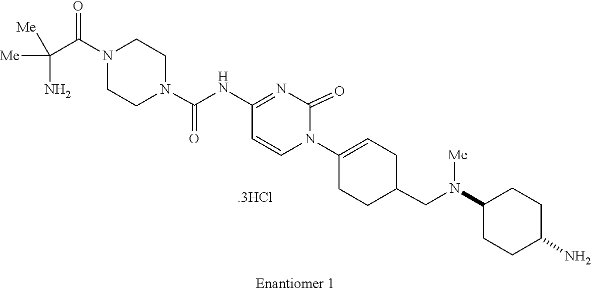 [SMILES: CN(CC1CC=C(n2ccc(NC(=O)N3CCN(C(=O)C(C)(C)N)CC3)nc2=O)CC1)[C@H]1CC[C@H](N)CC1] |
| 33 | 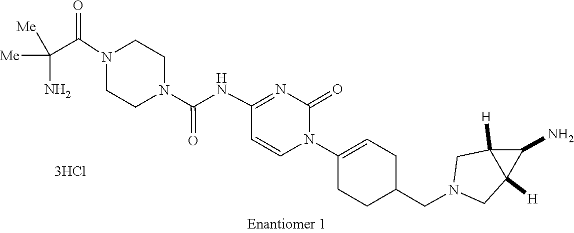 [SMILES: CC(C)(N)C(=O)N1CCN(C(=O)Nc2ccn(C3=CCC(CN4C[C@@H]5[C@@H](N)[C@@H]5C4)CC3)c(=O)n2)CC1] |
| 34 | 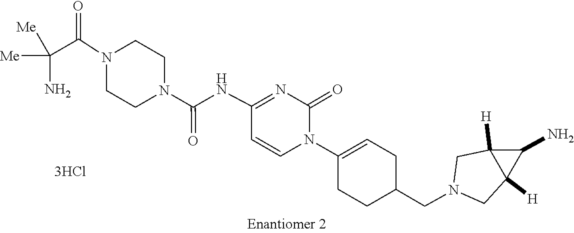 [SMILES: CC(C)(N)C(=O)N1CCN(C(=O)Nc2ccn(C3=CCC(CN4C[C@@H]5[C@@H](N)[C@@H]5C4)CC3)c(=O)n2)CC1] |
| 35 | 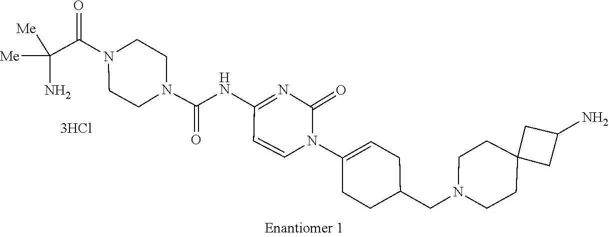 [SMILES: CC(C)(N)C(=O)N1CCN(C(=O)Nc2ccn(C3=CCC(CN4CCC5(CC4)CC(N)C5)CC3)c(=O)n2)CC1] |
| 36 | 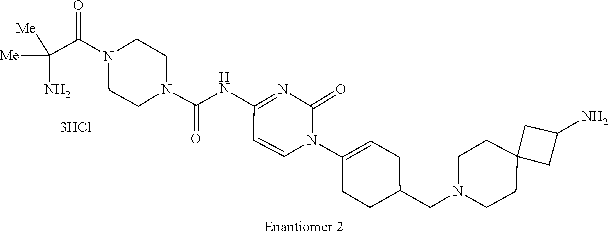 [SMILES: CC(C)(N)C(=O)N1CCN(C(=O)Nc2ccn(C3=CCC(CN4CCC5(CC4)CC(N)C5)CC3)c(=O)n2)CC1] |
| 37 | 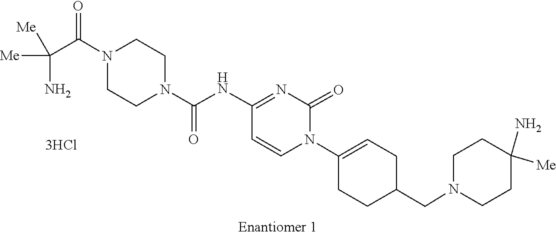 [SMILES: CC1(N)CCN(CC2CC=C(n3ccc(NC(=O)N4CCN(C(=O)C(C)(C)N)CC4)nc3=O)CC2)CC1] |
| 38 | 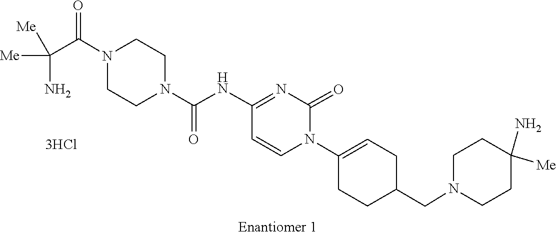 [SMILES: CC1(N)CCN(CC2CC=C(n3ccc(NC(=O)N4CCN(C(=O)C(C)(C)N)CC4)nc3=O)CC2)CC1] |
| 39 | 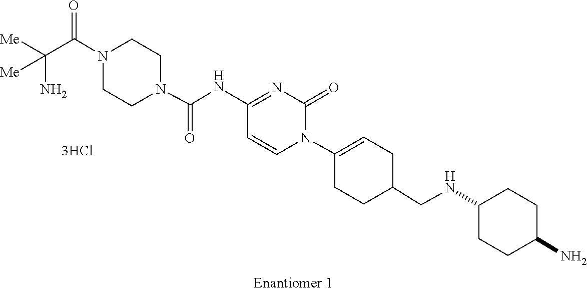 [SMILES: CC(C)(N)C(=O)N1CCN(C(=O)Nc2ccn(C3=CCC(CN[C@H]4CC[C@H](N)CC4)CC3)c(=O)n2)CC1] |
| 40 | 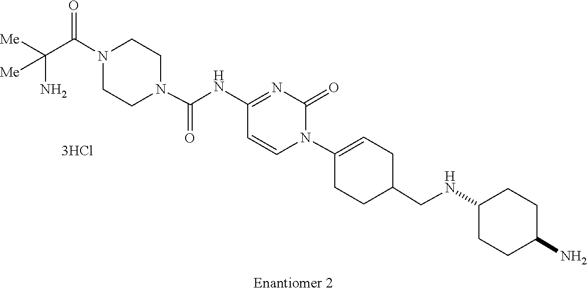 [SMILES: CC(C)(N)C(=O)N1CCN(C(=O)Nc2ccn(C3=CCC(CN[C@H]4CC[C@H](N)CC4)CC3)c(=O)n2)CC1] |
| 41 | 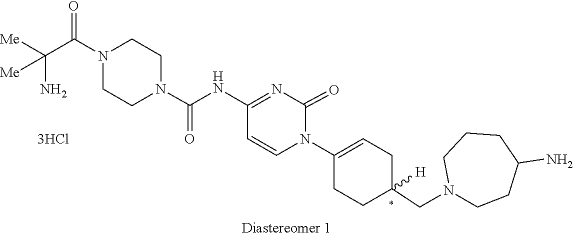 [SMILES: CC(C)(N)C(=O)N1CCN(C(=O)Nc2ccn(C3=CCC(CN4CCCC(N)CC4)CC3)c(=O)n2)CC1] |
| 42 | 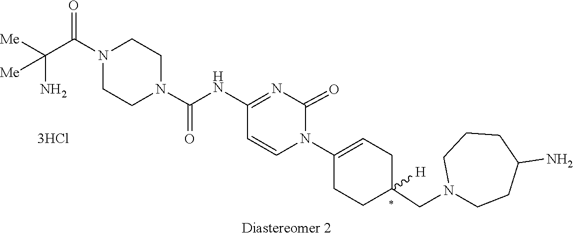 [SMILES: CC(C)(N)C(=O)N1CCN(C(=O)Nc2ccn(C3=CCC(CN4CCCC(N)CC4)CC3)c(=O)n2)CC1] |
| 43 | 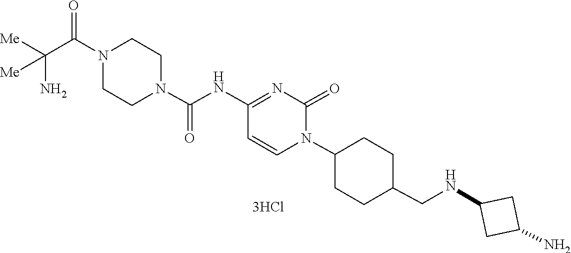 [SMILES: CC(C)(N)C(=O)N1CCN(C(=O)Nc2ccn(C3CCC(CN[C@H]4C[C@H](N)C4)CC3)c(=O)n2)CC1] |
| 44 | 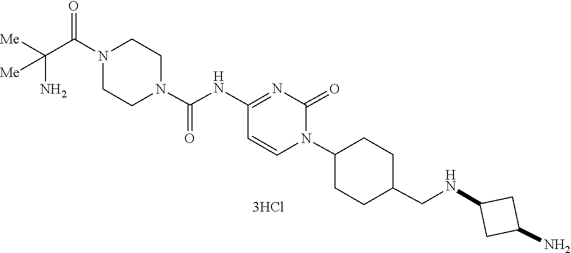 [SMILES: CC(C)(N)C(=O)N1CCN(C(=O)Nc2ccn(C3CCC(CN[C@H]4C[C@@H](N)C4)CC3)c(=O)n2)CC1] |
| 45 | 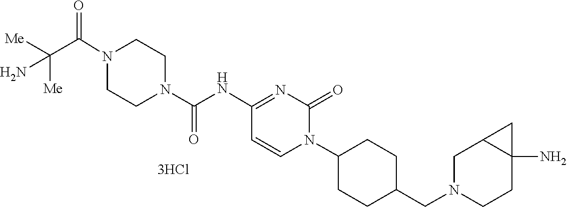 [SMILES: CC(C)(N)C(=O)N1CCN(C(=O)Nc2ccn(C3CCC(CN4CCC5(N)CC5C4)CC3)c(=O)n2)CC1] |
| 46 | 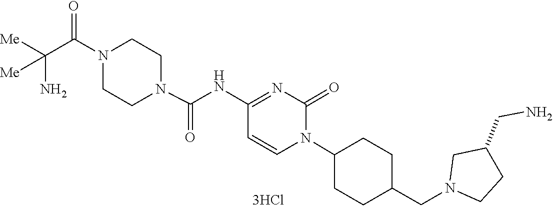 [SMILES: CC(C)(N)C(=O)N1CCN(C(=O)Nc2ccn(C3CCC(CN4CC[C@@H](CN)C4)CC3)c(=O)n2)CC1] |
| 47 | 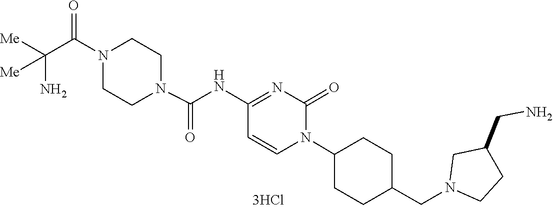 [SMILES: CC(C)(N)C(=O)N1CCN(C(=O)Nc2ccn(C3CCC(CN4CC[C@H](CN)C4)CC3)c(=O)n2)CC1] |
| 48 | 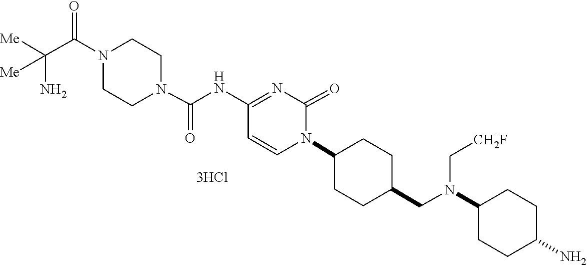 |
| 49 | 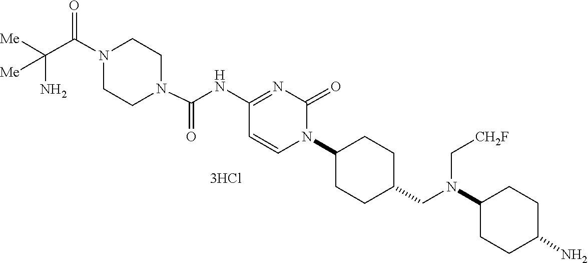 |
| 50 | 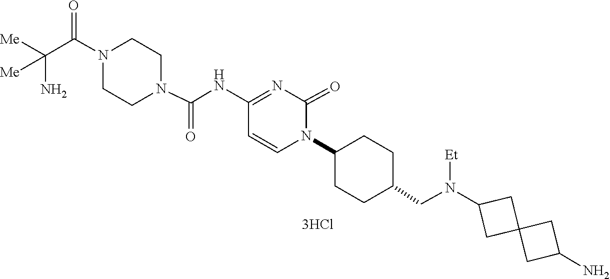 [SMILES: CCN(C[C@H]1CC[C@H](n2ccc(NC(=O)N3CCN(C(=O)C(C)(C)N)CC3)nc2=O)CC1)C1CC2(CC(N)C2)C1] |
| 51 | 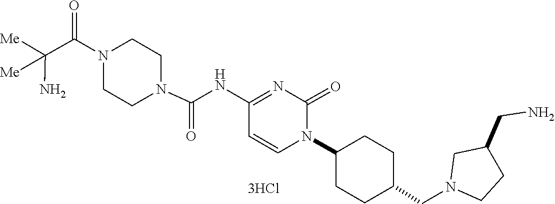 [SMILES: CC(C)(N)C(=O)N1CCN(C(=O)Nc2ccn([C@H]3CC[C@H](CN4CC[C@H](CN)C4)CC3)c(=O)n2)CC1] |
| 52 | 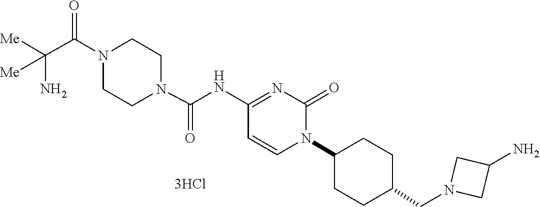 [SMILES: CC(C)(N)C(=O)N1CCN(C(=O)Nc2ccn([C@H]3CC[C@H](CN4CC(N)C4)CC3)c(=O)n2)CC1] |
| 53 | 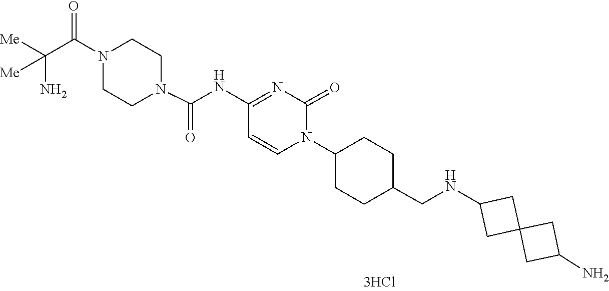 [SMILES: CC(C)(N)C(=O)N1CCN(C(=O)Nc2ccn(C3CCC(CNC4CC5(CC(N)C5)C4)CC3)c(=O)n2)CC1] |

Additional Compounds of Formula I

| No. | Free Base Structure |
|---|---|
| 13 | 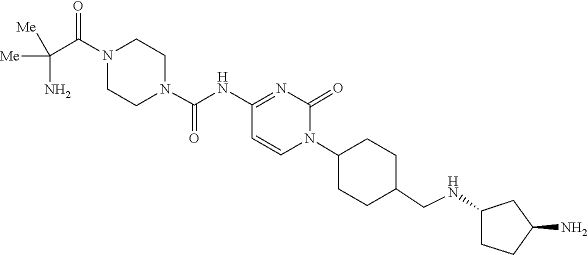 [SMILES: CC(C)(N)C(=O)N1CCN(C(=O)Nc2ccn(C3CCC(CN[C@H]4CC[C@H](N)C4)CC3)c(=O)n2)CC1] |
| 14 | 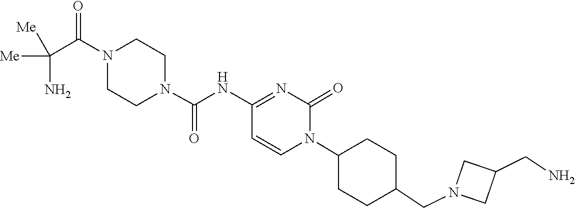 [SMILES: CC(C)(N)C(=O)N1CCN(C(=O)Nc2ccn(C3CCC(CN4CC(CN)C4)CC3)c(=O)n2)CC1] |
| 15 | 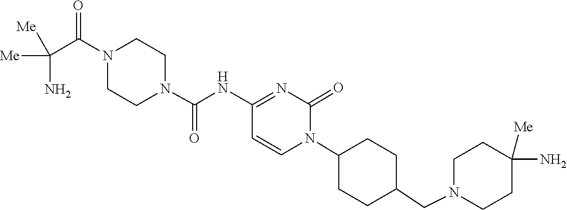 [SMILES: CC1(N)CCN(CC2CCC(n3ccc(NC(=O)N4CCN(C(=O)C(C)(C)N)CC4)nc3=O)CC2)CC1] |
| 16 | 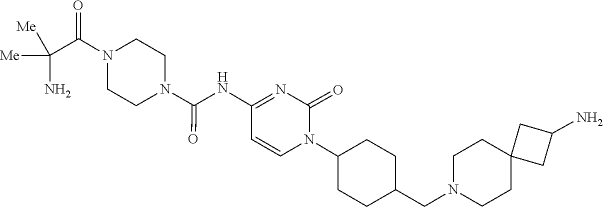 [SMILES: CC(C)(N)C(=O)N1CCN(C(=O)Nc2ccn(C3CCC(CN4CCC5(CC4)CC(N)C5)CC3)c(=O)n2)CC1] |
| 17 | 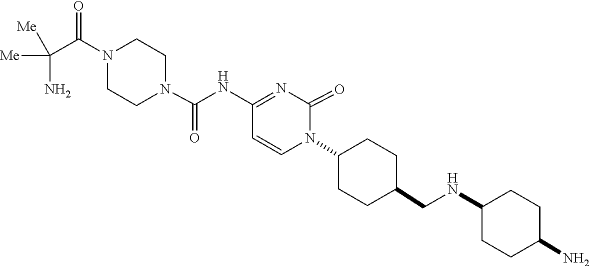 [SMILES: CC(C)(N)C(=O)N1CCN(C(=O)Nc2ccn([C@H]3CC[C@H](CN[C@H]4CC[C@@H](N)CC4)CC3)c(=O)n2)CC1] |
| 18 | 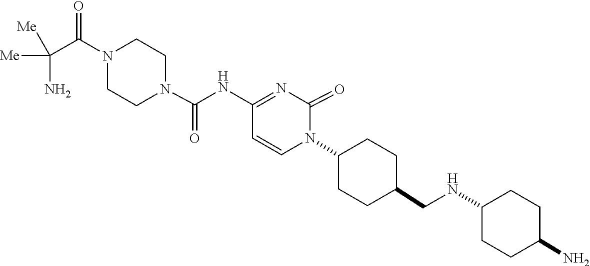 [SMILES: CC(C)(N)C(=O)N1CCN(C(=O)Nc2ccn([C@H]3CC[C@H](CN[C@H]4CC[C@H](N)CC4)CC3)c(=O)n2)CC1] |
| 19 | 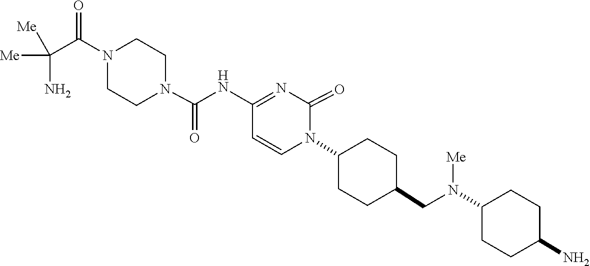 [SMILES: CN(C[C@H]1CC[C@H](n2ccc(NC(=O)N3CCN(C(=O)C(C)(C)N)CC3)nc2=O)CC1)[C@H]1CC[C@H](N)CC1] |
| 20 | 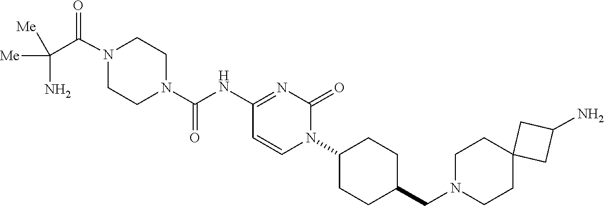 [SMILES: CC(C)(N)C(=O)N1CCN(C(=O)Nc2ccn([C@H]3CC[C@H](CN4CCC5(CC4)CC(N)C5)CC3)c(=O)n2)CC1] |
| 21 | 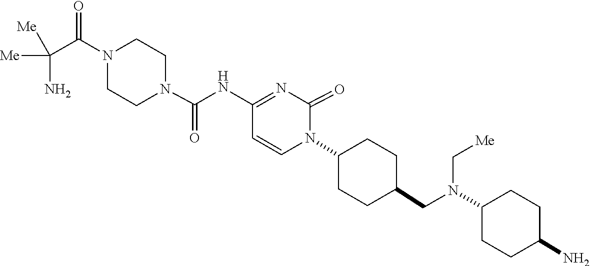 [SMILES: CCN(C[C@H]1CC[C@H](n2ccc(NC(=O)N3CCN(C(=O)C(C)(C)N)CC3)nc2=O)CC1)[C@H]1CC[C@H](N)CC1] |
| 22 | 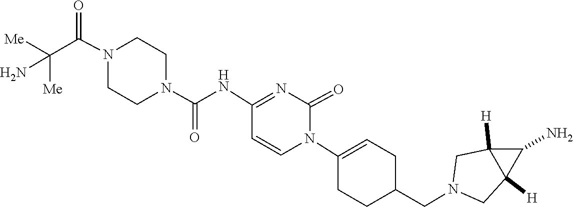 [SMILES: CC(C)(N)C(=O)N1CCN(C(=O)Nc2ccn(C3=CCC(CN4C[C@@H]5[C@H](N)[C@@H]5C4)CC3)c(=O)n2)CC1] |
| 23 | 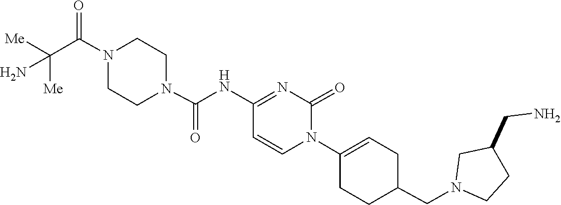 [SMILES: CC(C)(N)C(=O)N1CCN(C(=O)Nc2ccn(C3=CCC(CN4CC[C@H](CN)C4)CC3)c(=O)n2)CC1] |
| 24 | 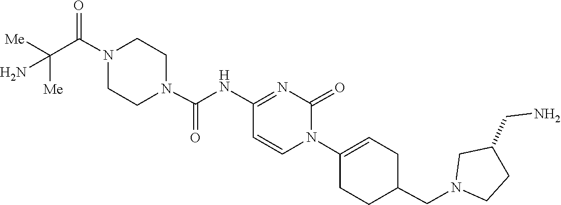 [SMILES: CC(C)(N)C(=O)N1CCN(C(=O)Nc2ccn(C3=CCC(CN4CC[C@@H](CN)C4)CC3)c(=O)n2)CC1] |
| 25 | 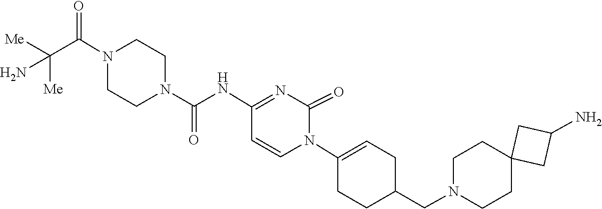 [SMILES: CC(C)(N)C(=O)N1CCN(C(=O)Nc2ccn(C3=CCC(CN4CCC5(CC4)CC(N)C5)CC3)c(=O)n2)CC1] |
| 26 | 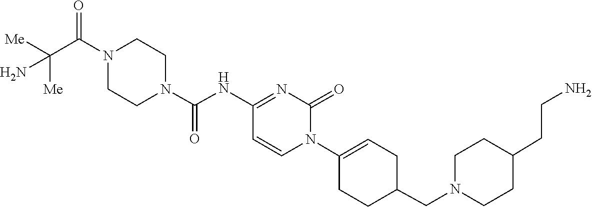 [SMILES: CC(C)(N)C(=O)N1CCN(C(=O)Nc2ccn(C3=CCC(CN4CCC(CCN)CC4)CC3)c(=O)n2)CC1] |
| 27 | 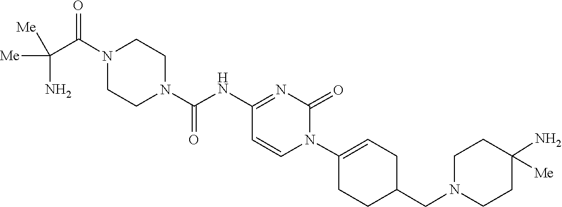 [SMILES: CC1(N)CCN(CC2CC=C(n3ccc(NC(=O)N4CCN(C(=O)C(C)(C)N)CC4)nc3=O)CC2)CC1] |
| 28 | 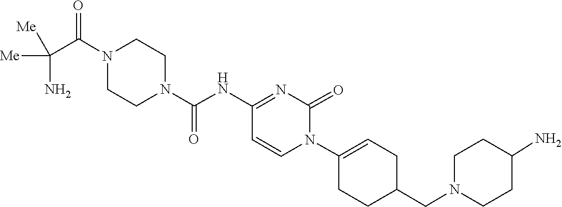 [SMILES: CC(C)(N)C(=O)N1CCN(C(=O)Nc2ccn(C3=CCC(CN4CCC(N)CC4)CC3)c(=O)n2)CC1] |
| 29 | 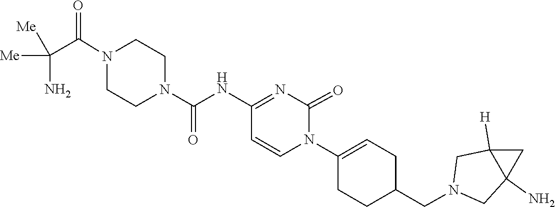 [SMILES: CC(C)(N)C(=O)N1CCN(C(=O)Nc2ccn(C3=CCC(CN4CC5CC5(N)C4)CC3)c(=O)n2)CC1] |
| 30 | 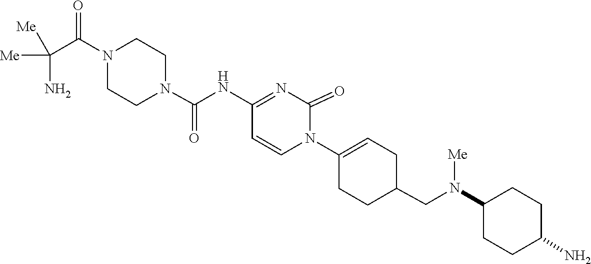 [SMILES: CN(CC1CC=C(n2ccc(NC(=O)N3CCN(C(=O)C(C)(C)N)CC3)nc2=O)CC1)[C@H]1CC[C@H](N)CC1] |
| 31 | 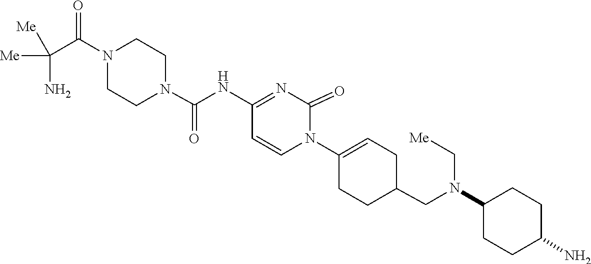 [SMILES: CCN(CC1CC=C(n2ccc(NC(=O)N3CCN(C(=O)C(C)(C)N)CC3)nc2=O)CC1)[C@H]1CC[C@H](N)CC1] |
| 32 | 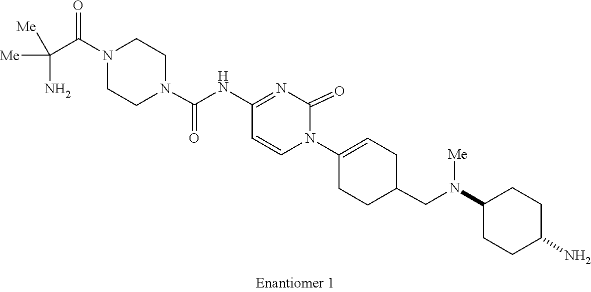 [SMILES: CN(CC1CC=C(n2ccc(NC(=O)N3CCN(C(=O)C(C)(C)N)CC3)nc2=O)CC1)[C@H]1CC[C@H](N)CC1] |
| 33 | 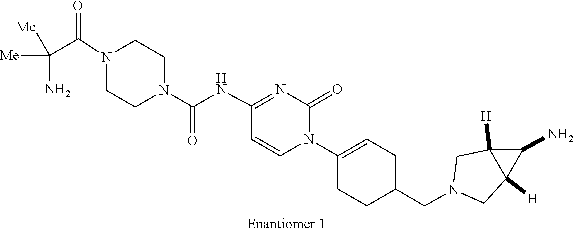 [SMILES: CC(C)(N)C(=O)N1CCN(C(=O)Nc2ccn(C3=CCC(CN4C[C@@H]5[C@@H](N)[C@@H]5C4)CC3)c(=O)n2)CC1] |
| 34 | 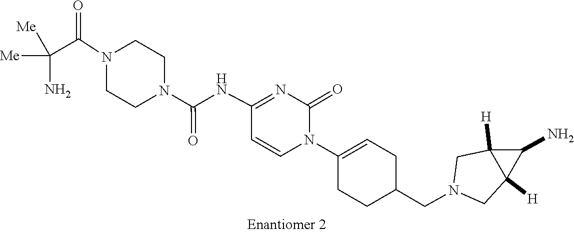 [SMILES: CC(C)(N)C(=O)N1CCN(C(=O)Nc2ccn(C3=CCC(CN4C[C@@H]5[C@@H](N)[C@@H]5C4)CC3)c(=O)n2)CC1] |
| 35 | 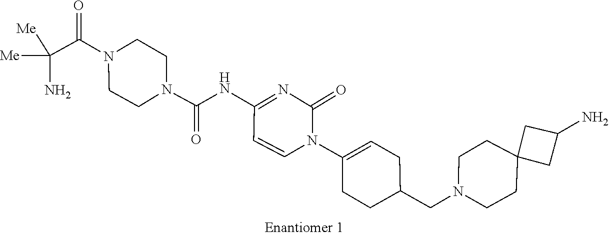 [SMILES: CC(C)(N)C(=O)N1CCN(C(=O)Nc2ccn(C3=CCC(CN4CCC5(CC4)CC(N)C5)CC3)c(=O)n2)CC1] |
| 36 | 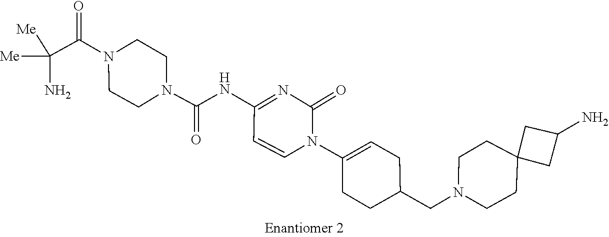 [SMILES: CC(C)(N)C(=O)N1CCN(C(=O)Nc2ccn(C3=CCC(CN4CCC5(CC4)CC(N)C5)CC3)c(=O)n2)CC1] |
| 37 | 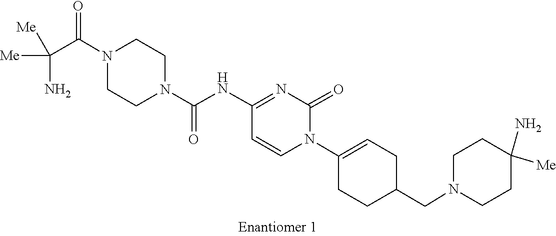 [SMILES: CC1(N)CCN(CC2CC=C(n3ccc(NC(=O)N4CCN(C(=O)C(C)(C)N)CC4)nc3=O)CC2)CC1] |
| 38 | 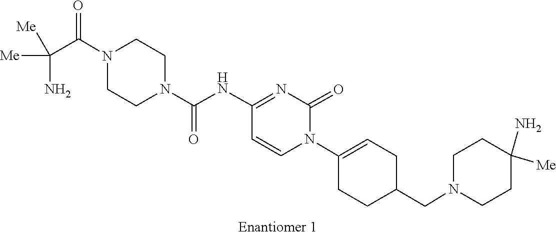 [SMILES: CC1(N)CCN(CC2CC=C(n3ccc(NC(=O)N4CCN(C(=O)C(C)(C)N)CC4)nc3=O)CC2)CC1] |
| 39 | 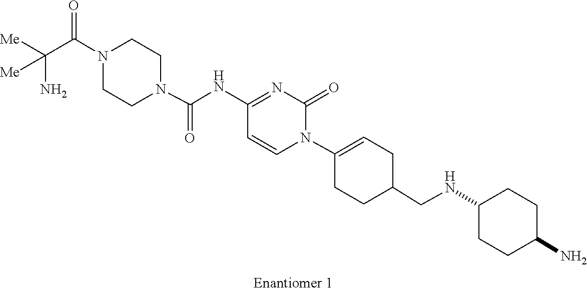 [SMILES: CC(C)(N)C(=O)N1CCN(C(=O)Nc2ccn(C3=CCC(CN[C@H]4CC[C@H](N)CC4)CC3)c(=O)n2)CC1] |
| 40 | 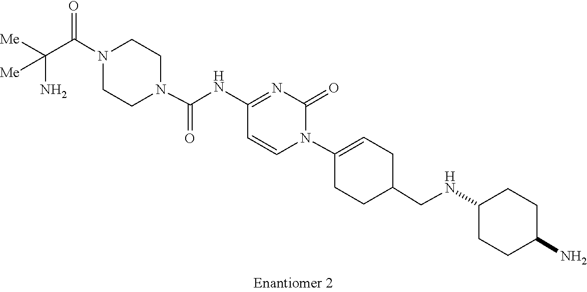 [SMILES: CC(C)(N)C(=O)N1CCN(C(=O)Nc2ccn(C3=CCC(CN[C@H]4CC[C@H](N)CC4)CC3)c(=O)n2)CC1] |
| 41 | 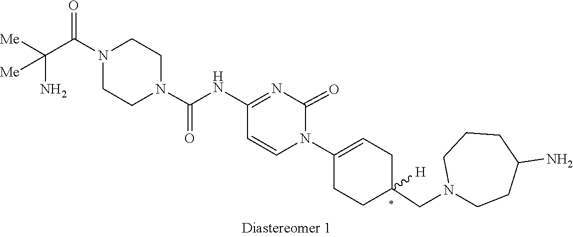 [SMILES: CC(C)(N)C(=O)N1CCN(C(=O)Nc2ccn(C3=CCC(CN4CCCC(N)CC4)CC3)c(=O)n2)CC1] |
| 42 | 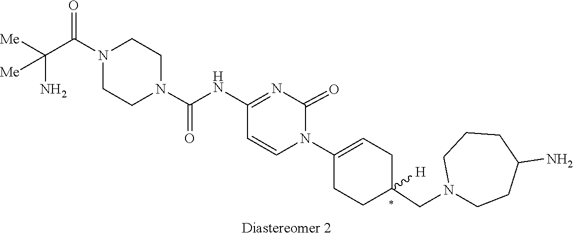 [SMILES: CC(C)(N)C(=O)N1CCN(C(=O)Nc2ccn(C3=CCC(CN4CCCC(N)CC4)CC3)c(=O)n2)CC1] |
| 43 | 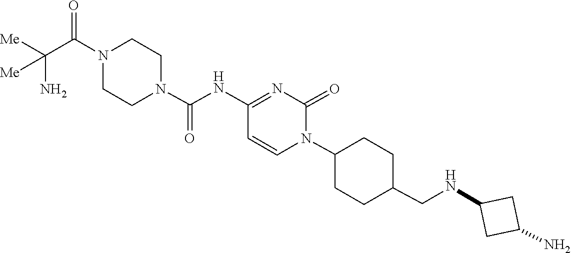 [SMILES: CC(C)(N)C(=O)N1CCN(C(=O)Nc2ccn(C3CCC(CN[C@H]4C[C@H](N)C4)CC3)c(=O)n2)CC1] |
| 44 | 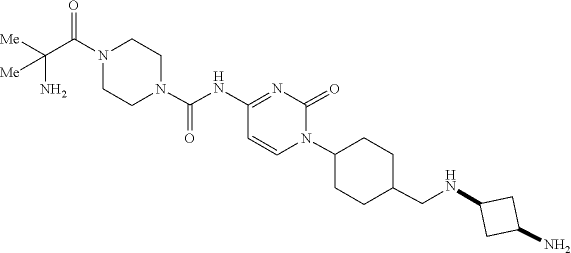 [SMILES: CC(C)(N)C(=O)N1CCN(C(=O)Nc2ccn(C3CCC(CN[C@H]4C[C@@H](N)C4)CC3)c(=O)n2)CC1] |
| 45 | 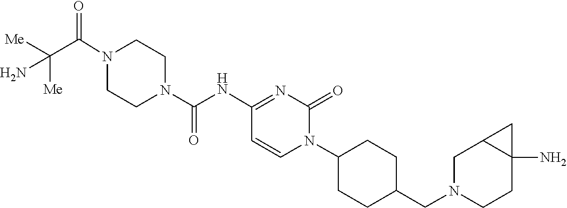 [SMILES: CC(C)(N)C(=O)N1CCN(C(=O)Nc2ccn(C3CCC(CN4CCC5(N)CC5C4)CC3)c(=O)n2)CC1] |
| 46 | 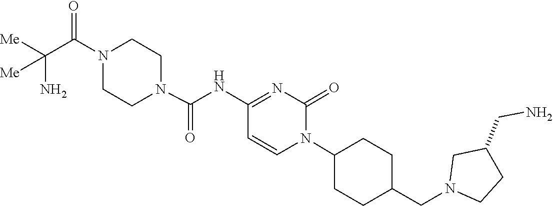 [SMILES: CC(C)(N)C(=O)N1CCN(C(=O)Nc2ccn(C3CCC(CN4CC[C@@H](CN)C4)CC3)c(=O)n2)CC1] |
| 47 | 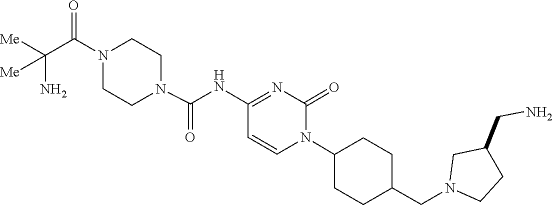 [SMILES: CC(C)(N)C(=O)N1CCN(C(=O)Nc2ccn(C3CCC(CN4CC[C@H](CN)C4)CC3)c(=O)n2)CC1] |
| 48 | 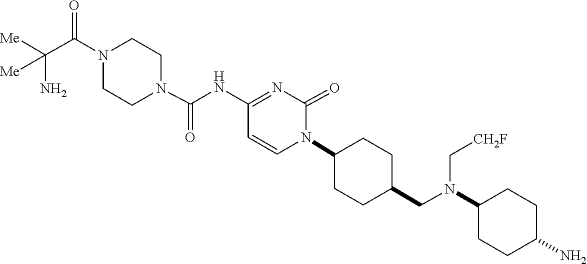 |
| 49 | 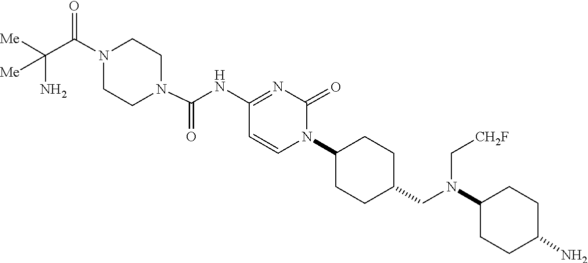 |
| 50 | 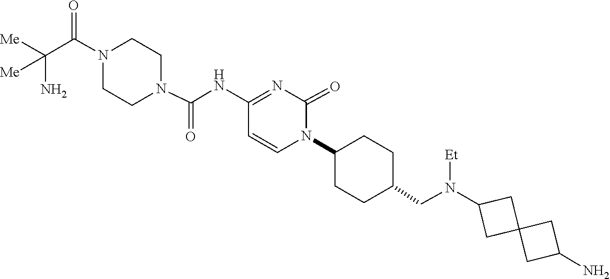 [SMILES: CCN(C[C@H]1CC[C@H](n2ccc(NC(=O)N3CCN(C(=O)C(C)(C)N)CC3)nc2=O)CC1)C1CC2(CC(N)C2)C1] |
| 51 | 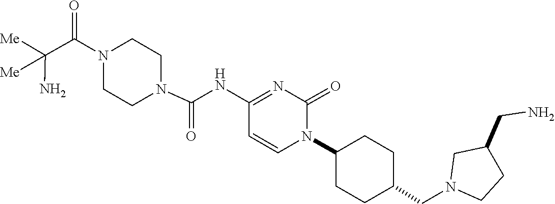 [SMILES: CC(C)(N)C(=O)N1CCN(C(=O)Nc2ccn([C@H]3CC[C@H](CN4CC[C@H](CN)C4)CC3)c(=O)n2)CC1] |
| 52 | 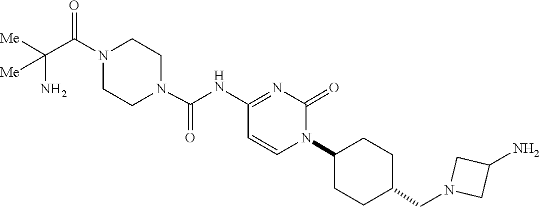 [SMILES: CC(C)(N)C(=O)N1CCN(C(=O)Nc2ccn([C@H]3CC[C@H](CN4CC(N)C4)CC3)c(=O)n2)CC1] |
| 53 | 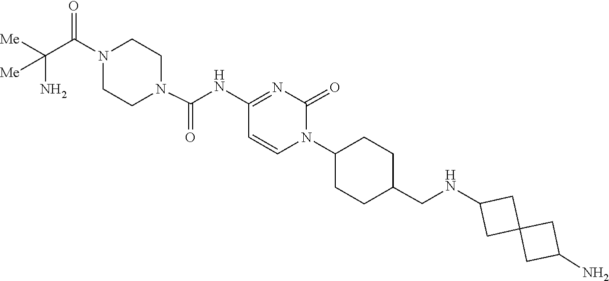 [SMILES: CC(C)(N)C(=O)N1CCN(C(=O)Nc2ccn(C3CCC(CNC4CC5(CC(N)C5)C4)CC3)c(=O)n2)CC1] |

What is also provided is a compound which is

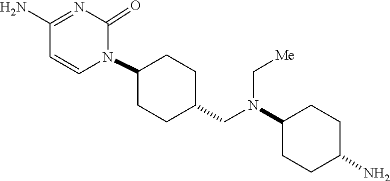

or a pharmaceutically acceptable salt thereof.

In another embodiment, the compound of formula I, or a single stereoisomer or mixture of stereoisomers thereof, or a pharmaceutically acceptable salt thereof is selected from the compounds listed in any one of Table 1 and Table 2.

In another aspect, the disclosure provides a compound of formula E:

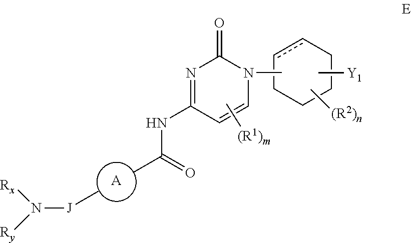

or a pharmaceutically acceptable salt thereof, wherein ring A, J, R¹, R², Rₓ, R_{y}, m, and n have the same definitions in the preceding paragraphs; and Y₁ is C₁-C₆ alkylene, wherein one methylene unit of the C₁-C₆ alkylene is replaced by (C═O).

In another embodiment of the compound of formula E or a pharmaceutically acceptable salt, Y₁ is a linear C₁-C₃ alkylene, wherein one methylene unit of the C₁-C₃ alkylene is replaced by (C═O).

In an embodiment, the compound of formula E or pharmaceutically acceptable salt thereof is selected from the group consisting of

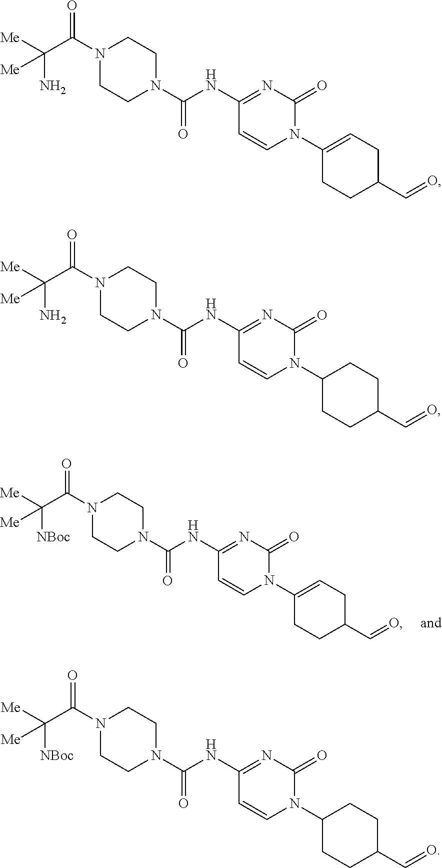

### Pharmaceutical Compositions and Administration

The present invention provides pharmaceutical compositions comprising a compound of the present invention and a pharmaceutically acceptable excipient. In certain embodiments, the compound of the present invention is provided in an effective amount in the pharmaceutical composition. In certain embodiments, the effective amount is a therapeutically effective amount. In certain embodiments, the effective amount is a prophylactically effective amount.

Pharmaceutically acceptable excipients include any and all solvents, diluents, or other liquid vehicles, dispersions, suspension aids, surface active agents, isotonic agents, thickening or emulsifying agents, preservatives, solid binders, lubricants and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing the compound of the present invention (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof: malic acid and salts and hydrates thereof: phosphoric acid and salts and hydrates thereof; and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, camauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the conjugates of this invention with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner Examples of embedding compositions which can be used include polymeric substances and waxes.

Dosage forms for topical and/or transdermal administration of a compound of this invention may include ointments, pastes, creams, lotions, gels, powders, solutions, sprays, inhalants and/or patches. Generally, the active ingredient is admixed under sterile conditions with a pharmaceutically acceptable carrier and/or any needed preservatives and/or buffers as can be required. Additionally, the present invention contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions can be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes can be used in the classical mantoux method of intradermal administration.

A pharmaceutical composition of the invention can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions of the invention formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A pharmaceutical composition of the invention can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

To practice the method of this invention, the above-described compound or its pharmaceutical composition can be administered orally, parenterally, by inhalation spray, topically, rectally, nasally, buccally, vaginally, rectally, or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. The compounds or compositions can be administered in combination with additional therapeutically active agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually. Additional therapeutically active agents include antibiotic agents, e.g., antibiotics useful for treating tuberculosis. Exemplary antibiotics include, but are not limited to, isoniazid, rifampin, pyrazinamide, ethambutol, and streptomycin.

Also encompassed by the invention are kits (e.g., pharmaceutical packs). The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one unit dosage form.

### Uses and Methods of Treatment

In another aspect, the invention provides a method of treating a bacterial infection in a patient in need of such treatment, comprising administering an effective amount of a compound of formula I or a pharmaceutically acceptable salt thereof or a composition comprising a compound of formula I or a pharmaceutically acceptable salt thereof. In certain embodiments, the effective amount is a therapeutically effective amount. In certain other embodiments, the effective amount is a prophylactically effective amount.

In some embodiments, the compounds of the invention can be active against a wide range of both Gram-positive and Gram-negative organisms. In these and other embodiments, the compounds of the invention can be used to treat infections and to inhibit microbial growth. Thus, the compounds of the invention can be used to treat humans and animals having a broad spectrum of bacterial infections such as impetigo, pneumonia, bronchitis, pharyngitis, endocarditis, urinary tract infections, diabetes foot ulcers, gastro-intestinal infections and bacteremia. These bacterial infections could be caused by any of the following bacteria—Staphylococcus aureus, coagulase negative staphylococci, methicillin-resistant Staphylococcus aureus, methicillin-resistant coagulase negative staphylococci, enterococci, beta-haemolytic streptococci, viridans group of streptococci, Bacillus mycobacterial infections due to multi-drug resistant M. tuberculosis and other atypical mycobacteria such as M. intracellulare and M. avium, as well as newly emerging Gram-negative pathogens such as Chryseobacterium meningosepticum, Chryseobacterium indologense and other Gram-negative pathogens such as E. coli, Klebsiella, Proteus, Serratia, Citrobacter, Pseudomonas, Burkholderia, Brucella, Yersinia, Francisella, Coxiella, Chlamydia, Salmonella, Rickettsia, Shigella and Campylobacter.

In one embodiment, the bacterial infection is tuberculosis. In certain embodiments, the tuberculosis infection is a Mycobacterium tuberculosis infection. In certain embodiments, the tuberculosis infection is multi-drug-resistant tuberculosis (MDR-TB) infection, e.g., resistant to first-line TB drugs rifampicin and/or isoniazid. In certain embodiments, the tuberculosis infection is extensively-drug-resistant tuberculosis (XDR-TB) infection, e.g., also resistant to three or more of the six classes of second-line drugs (see, e.g., Centers for Disease Control and Prevention (CDC) (2006). “Emergence of Mycobacterium tuberculosis with extensive resistance to second-line drugs worldwide, 2000-2004”. MMWR Morb Mortal Wkly Rep 55 (11): 301-5).

### Process

In some aspects, the compounds and intermediates of the present disclosure can be prepared according to General Synthetic Scheme G-1 below. In Scheme G-1, variables such as ring A, ring B, J, L, Y, R¹, R², Rₓ, R_{y}, R_{x′}, R_{y′}, m, and n have the same definitions in the preceding paragraphs; X is a leaving group such as halo, mesylate, tosylate, or triflate; Y₁ is C₁-C₆ alkylene, wherein one methylene unit of the C₁-C₆ alkylene is replaced by (C═O); Y₃ is C₁-C₆ alkylene, wherein one methylene unit of the C₁-C₆ alkylene is optionally replaced by (C═O); and P is C₁-C₆ alkyl or a hydroxyl protecting group. In some embodiments, Y₁ is (C₁-C₃ alkylene)-C(═O)H. In some embodiments, Y₁ is —CH₂—C(═O)H. In some embodiments, Y₃ is C₁-C₃ alkylene, wherein one methylene unit of the C₁-C₃ alkylene is optionally replaced by (C═O). In some embodiments, Y₃ is —CH₂— or —C═O—. In some embodiments, —Y₃—OP is C₁-C₆ alkylene-OTBS, (C₁-C₅ alkylene)-C(═O)O—(C₁-C₆ alkyl), or —COO(C₁-C₆ alkyl). In some embodiments, —Y₃—OP is —CH₂—OTBS or —COO(C₁-C₆ alkyl).

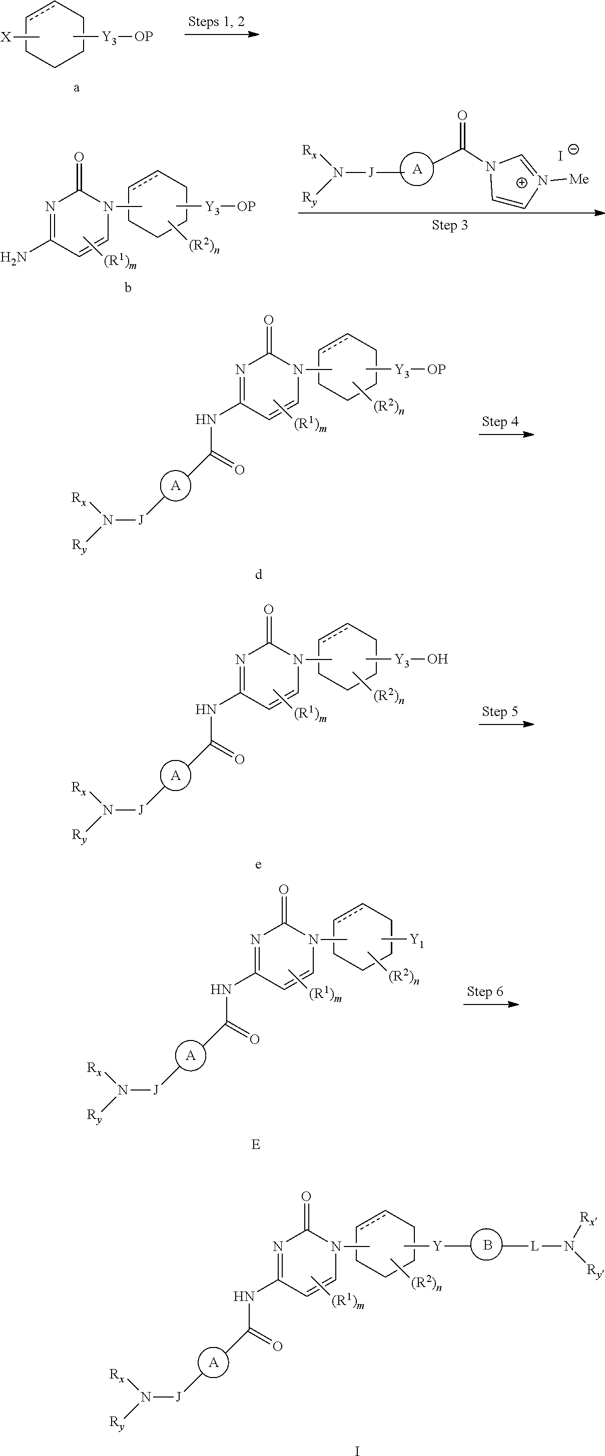

In step 1 of General Synthetic Scheme G-1, the compound of formula (a) is reacted with a boron reagent such as bis(pinacolato)diboron (B₂pin₂) to form a boronic ester of compound of formula (a) in the presence of a phosphine ligand such as [1,1′-Bis(diphenylphosphino)ferrocene]dichloropalladium (Pd(dppf)Cl₂), a base, and a suitable solvent. The base includes but is not limited to sodium bicarbonate, sodium carbonate, potassium carbonate, sodium acetate, potassium acetate, and cesium carbonate. The suitable solvent can be an aprotic solvent such as dioxane, dichloromethane, dimethylformamide, acetonitrile, and the like. In some processes, 1.0 molar equivalents of a compound of formula (a) are combined with about 1.0 to 2.0 molar equivalent of the boron reagent together with the base, the phosphine ligand in a suitable solvent such as dioxane. The mixture is then allowed to undergo reaction at a temperature of from about 0° C. to 150° C. for a sufficient time. For example, the temperature is from about 25° C. to 130° C. or from about 50° C. to 125° C. and the reaction time is from about 1 to 24 hours, from 2 to 24 hours, or from about 10 to 24 hours.

In step 2 of General Synthetic Scheme G-1, the boronic ester is cross-coupled with cytosine in the presence of a base such as a tertiary amine and a copper reagent such as a copper (II) reagent to afford the compound of formula (b).

Alternatively, to produce the compound of formula (b), in step 1 of General Synthetic Scheme G-1, N⁴-benzoyl cytosine can be used to react with the compound of formula (a) in the presence of 18-crown-6 and a base such as sodium bicarbonate, sodium carbonate, potassium carbonate, sodium acetate, potassium acetate, and cesium carbonate. The reaction can be carried out at a temperature of from about 0° C. to 150° C. for a sufficient time. For example, the temperature is from about 25° C. to 130° C. or from about 50° C. to 125° C., and the reaction time is from about 1 to 48 hours, or from 2 to 36 hours or from about 10 to 30 hours. In step 2, the benzoyl group is then removed under an acidic condition to afford the compound of formula (b).

In step 3 of General Synthetic Scheme G-1, the compound of formula (b) and the iodide (c) undergo an amide coupling to yield the intermediate (d). In some processes, 1 molar equivalents of the compound of formula (b) is combined with about 1.1 to 2.0 molar equivalent of the iodide (c) in a suitable solvent, such as a polar aprotic solvent. Polar aprotic solvents include solvents such as dichloromethane, dimethylformamide, acetonitrile, and the like. The mixture in the polar aprotic solvent are then allowed to undergo reaction at a temperature of from about 0° C. to 100° C. for a sufficient time. For example, the temperature is from about 25° C. to 95° C. or from about 50° C. to 95° C., and the reaction time is from about 1 to 24 hours, or from 2 to 20 hours, or from about 5 to 18 hours.

In steps 4 and 5 of General Synthetic Scheme G-1, the compound of formula (d) is deprotected to yield a free alcohol (e) and then oxidized to a ketone or aldehyde, a compound of formula E. In the case that —Y₃—OP is (C₁-C₅ alkylene)-C(═O)O—(C₁-C₆ alkyl), the compound of formula (d) is reduced to a free alcohol (e) and then oxidized to a ketone or aldehyde, the compound of formula (E).

In steps 6 of General Synthetic Scheme G-1, the compound of formula E is reacted with an amine under a reductive amination condition to afford the compound of formula I. The reductive amination can be performed in the presence of a reducing agent and a suitable solvent. A suitable solvent includes protic solvents or aprotic solvents. Protic solvents include but is not limited to water and alcohols such as methanol, ethanol, propanol, and the like. Aprotic solvents include but is not limited to solvents such as dichloromethane, dimethylformamide, acetonitrile, and the like. The suitable solvent may also be a combination of two or three solvents. The reducing agent includes but is not limited to a borohydride reagent or a metal hydride reagent. Non-limiting examples are lithium borohydride, sodium borohydride, sodium cyanoborohydride and sodium triacetoxyborohydride.

In one embodiment, the disclosure provides a process for preparing a compound of formula I:

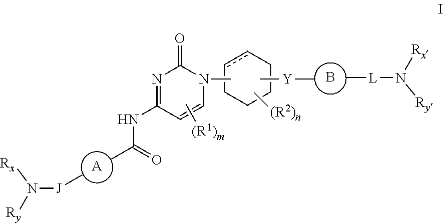

or a pharmaceutically acceptable salt thereof, the process comprising:
combining a compound of formula E:

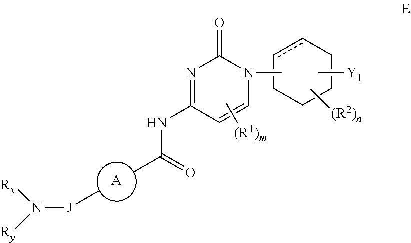

with a compound of:
formula D

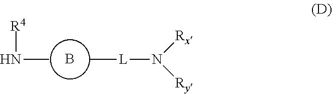

or formula D′

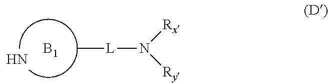

under a reductive amination condition to provide the compound of formula I,
wherein ring A, ring B, J, L, R¹, R², Rₓ, R_{y}, R_{x′}, R_{y′}, m, and n have the same definitions in the preceding paragraphs;
ring B₁ is a nitrogen containing 3-8 membered monocyclic heterocycloalkylene or a nitrogen containing 6-12 membered bicyclic heterocycloalkylene, each of which is optionally substituted with up to three substituents independently selected from the group consisting of C₁-C₆ alkyl, C₁-C₆ alkoxy, halo, CN, C₁-C₆ haloalkyl, OH, COOH, COO(C₁-C₆ alkyl), CONH₂, CONH(C₁-C₆ alkyl), CON(C₁-C₆ alkyl)₂, and C₁-C₆ hydroxyalkyl;
Y is C₁-C₆ alkylene, and wherein one methylene unit of the C₁-C₆ alkylene is optionally replaced by NH, N—(C₁-C₆ haloalkyl), or N—(C₁-C₆ alkyl);
Y₁ is C₁-C₆ alkylene, wherein one methylene unit of the C₁-C₆ alkylene is replaced by (C═O); and
R⁴ is H or C₁-C₆ alkyl.

Processes and conditions for performing the reductive amination of the compound of formula E are as in the General Synthetic Scheme Step 6.

In one embodiment, the process further comprises the step of removing the amino protecting group when any of Rₓ, R_{y}, R_{x′}, and R_{y′} is an amino protecting group.

In some embodiments, ring B₁ is a nitrogen containing 4-7 membered monocyclic heterocycloalkylene or a nitrogen containing 6-9 membered bicyclic heterocycloalkylene, each of which is optionally substituted with up to three substituents independently selected from the group consisting of C₁-C₆ alkyl, C₁-C₆ alkoxy, halo, CN, C₁-C₆ haloalkyl, OH, COOH, COO(C₁-C₆ alkyl), CONH₂, CONH(C₁-C₆ alkyl), CON(C₁-C₆ alkyl)₂, and C₁-C₆ hydroxyalkyl.

In some embodiments, ring B₁ is a nitrogen containing 4-7 membered monocyclic heterocycloalkylene or a nitrogen containing 6-9 membered bicyclic heterocycloalkylene, wherein the monocyclic and bicyclic heterocycloalkylenes are optionally substituted with C₁-C₆ alkyl. In some embodiments, ring B₁ is a nitrogen containing 4-7 membered monocyclic heterocycloalkylene or a nitrogen containing 6-9 membered bicyclic heterocycloalkylene.

In some embodiments, ring B in formula D is

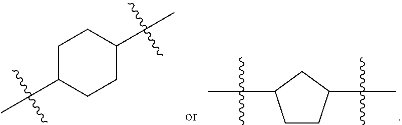 [SMILES: *C1CCC(*)CC1 ; *C1CCC(*)C1]

In some embodiments, ring B₁ is selected from the group consisting of,

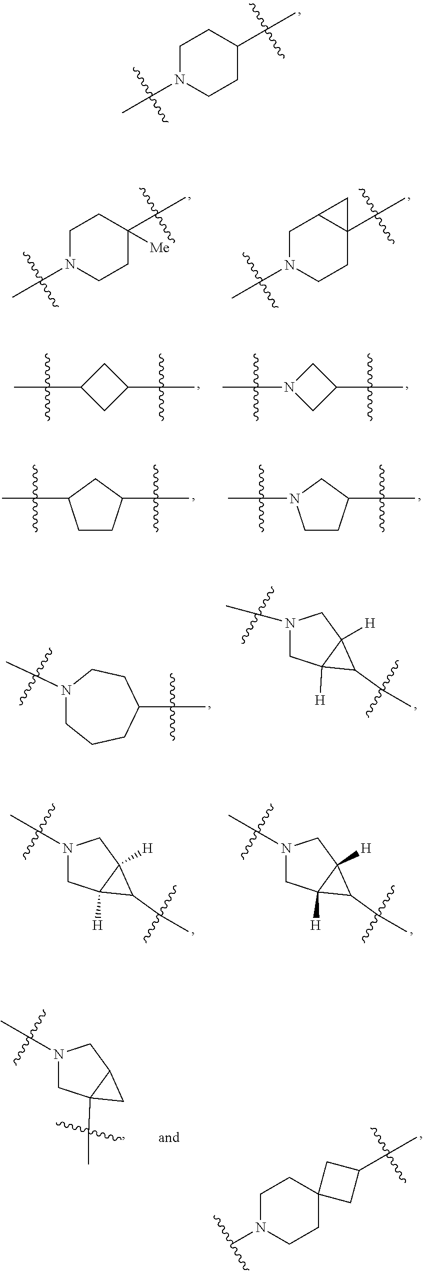

In some embodiments, L is a bond, —CH₂—, or —CH₂—CH₂—; and R_{x′} and R_{y′} are each independently H or Boc.

In another embodiment, the compound of formula e is selected from the group consisting of:

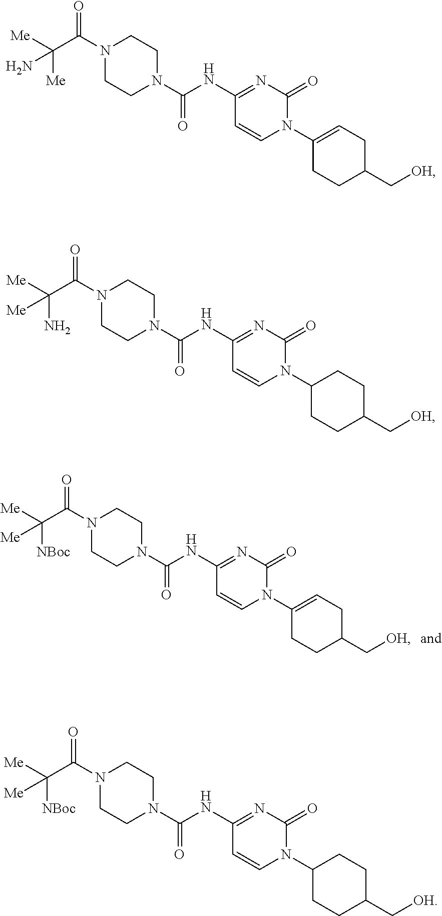

In another embodiment, the compound of formula E is selected from the group consisting of:

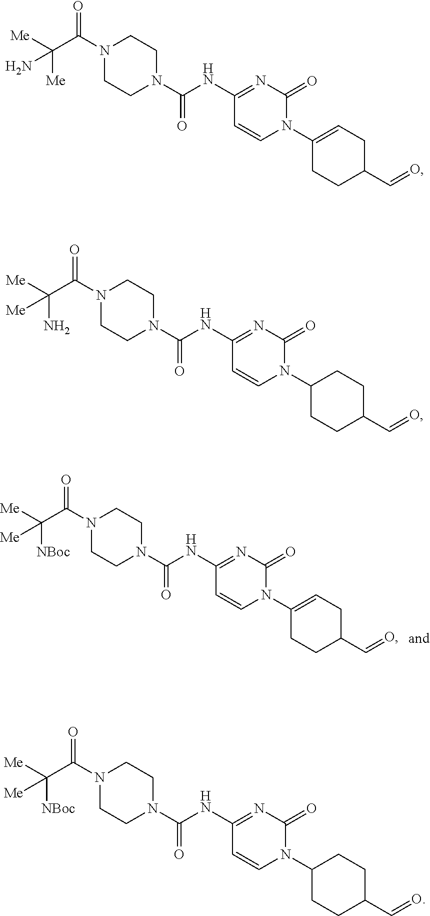

### Compound Preparation

The preparation of starting materials that are commercially available, described in the literature, or readily obtainable by those skilled in the art is not described. It will be appreciated by the skilled person that where it is stated that compounds were prepared analogously to earlier examples or intermediates, the reaction time, number of equivalents of reagents, and temperature, can be modified for each specific reaction and that it may be necessary or desirable to employ different work-up or purification techniques. Where reactions are carried out using microwave irradiation, the microwave oven used was either a Biotage Initiator or in CEM Discover System Model 908005. The actual power supplied varies during the reaction in order to maintain a constant temperature.

### General Methods

Reactions requiring anhydrous conditions were conducted in oven-dried glassware under a positive pressure of either nitrogen or argon. Commercially available reagents were used as received; otherwise, materials were purified according to Purification of Laboratory Chemicals. Dichloromethane (CH₂Cl₂), N,N′-dimethylformamide (DMF), toluene and tetrahydrofuran (THF) were degassed with nitrogen and passed through a solvent purification system (Innovative Technologies Pure Solv). Dry 1,4-dioxane was purchased from Acros Organics in a Acros Seal™ bottle. Triethylamine (Et₃N) and N,N-diisopropylethylamine (DIPEA) were stored over 4 Å molecular sieves or distilled over 4 Å molecular sieves prior to usage. Microwave reactions were done in CEM Discover System Model 908005. Reactions were monitored by TLC or LCMS and visualized by a dual short wave/long wave UV lamp and/or stained with ethanolic solutions of either KMnO₄, 12-phosphomolybdic acid or other commonly used stains. Flash chromatography was performed on Merck silica gel Kieselgel 60 (230-400 mesh) from EM Science with the indicated HPLC grade solvent or an automated medium pressure column chromatography system (Teledyne ISCO CombiFlash RF75 or CombiFlash Rf+). Reverse phase HPLC was conducted on a Waters HPLC Semi Prep 150B system with Sunfire C18 Prep Column or Atlantis T3 Prep Column with isocratic or gradient conditions with H₂O (0.1% TFA) and 10% H₂O:90 CH₃CN (0.1% TFA) as eluents

Melting points were determined using Mel-Temp® Capillary Melting Point Apparatus. Infrared spectra were obtained using Nicolet 380-FT IR spectrometer fitted with a Smart Orbit sample system. Optical rotations were obtained at ambient temperature on a Perkin Elmer Model 343 polarimeter (Na D line) using a microcell with a 1 decimeter path length. Mass spectra determined by LCMS were collected on Thermo Scientific™ UltiMate™ 3000 UHPLC with electrochemical detector with a fluorescence detector monitored at either 214 or 254 nm, or a Waters Aquity UPLC H-Class Series with photodiode array detector and QDa mass detector. ¹H NMR spectra were recorded at 500 MHz, 400 MHz, and 300 MHz, and ¹³C at 125 MHz. Proton resonances were reported relative to the deuterated solvent peak: 7.27 ppm for CDCl₃, 3.31 ppm (center line signal) for CD₃OD, 2.50 for D6-DMSO and 4.79 for D₂O using the following format: chemical shift (6) [multiplicity (s=singlet, br s=broad singlet, d=doublet, t=triplet, q=quartet, m=multiplet)]. Carbon resonances were reported as chemical shifts (6) in parts per million, relative to the center line signal of the respective solvent peak: 77.23 ppm for CDCl₃ and 49.15 ppm for CD₃OD. Commercially available chemicals are purchased from multiple vendors including Sigma-Aldrich, Acros, Enamine, TCI America, Combi-Blocks, Alfa-Aesar, Angene, Ark Pharma, PharmaBlock, Strem Chemicals, Frontier Scientific, and AstaTech, Inc.

### Liquid Chromatography-Mass Spectrometry Methods

### Liquid Chromatography-Mass Spectrometry Method A

Total ion current (TIC) and DAD UV chromatographic traces together with MS and UV spectra associated with the peaks were taken on a UPLC/MS Acquity™ system equipped with PDA detector and coupled to a Waters single quadrupole mass spectrometer operating in alternated positive and negative electrospray ionization mode. [LC/MS-ES (+/−): analyses performed using an Acquity UPLC™ CSH, C18 column (50×2.1 mm, 1.7 μm particle size), column temperature 40° C., mobile phase: A—water+0.1% HCOOH/B—CH₃CN+0.1% HCOOH, flow rate: 1.0 mL/min, runtime=2.0 min, gradient: t=0 min 3% B, t=1.5 min 99.9% B, t=1.9 min 99.9% B, t=2.0 min 3% B, stop time 2.0 min. Positive ES 100-1000, Negative ES 100-1000, UV detection DAD 210-350 nm.

### Liquid Chromatography-Mass Spectrometry Method B

Total ion current (TIC) and DAD UV chromatographic traces together with MS and UV spectra associated with the peaks were taken on a UPLC/MS Acquity™ system equipped with PDA detector and coupled to a Waters single quadrupole mass spectrometer operating in alternated positive and negative electrospray ionization mode. [LC/MS-ES (+/−): analyses performed using an Acquity UPLC™ BEH, C18 column (50×2.1 mm, 1.7 μm particle size), column temperature 40° C., mobile phase: A—0.1% v/v aqueous (aq) ammonia solution pH 10/B—CH₃CN, flow rate: 1.0 mL/min, runtime=2.0 min, gradient: t=0 min 3% B, t=1.5 min 99.9% B, t=1.9 min 99.9% B, t=2.0 min 3% B, stop time 2.0 min. Positive ES 100-1000, Negative ES 100-1000, UV detection DAD 210-350 nm.

### Liquid Chromatography-Mass Spectrometry Method C

LC/MS-ES (+/−): analyses performed using an AQUITY with PDA detector and QDA Performance, C18 column (50×2.1 mm, 1.6 μm particle size), column temperature 35° C., mobile phase: A—0.1% Formic acid in Milli Q water (pH=2.70)/B—0.1% Formic acid in water:Acetonitrile (10:90), flow rate: 0.8-1.0 mL/min, runtime=4.0 min, gradient: t=0 min 3% B, t=2.7 min 98% B, t=3.0 min 100% B, t=3.51 min 3% B, stop time 4.0 min.

### Liquid Chromatography-Mass Spectrometry Method D

LC/MS-ES (+/−): analyses performed using AQUITY H-Class with PDA detector and QDA, C18 column (50×2.1 mm, 1.6 μm particle size), column temperature 35° C., mobile phase: A—0.1% Formic acid in Milli Q water (pH=2.70)/B—0.1% Formic acid in water:Acetonitrile (10:90), flow rate: 0.8-1.0 mL/min, runtime=4.0 min, gradient: t=0 min 3% B, t=2.7 min 98% B, t=3.0 min 100% B, t=3.51 min 3% B, stop time 4.0 min.

### Liquid Chromatography-Mass Spectrometry Method E

LC/MS-ES (+/−): analyses performed using AQUITY H-Class with PDA detector and QDA, C18 column (50×2.1 mm, 1.6 μm particle size), column temperature 35° C., mobile phase: A 0.1% Formic acid in water (pH=2.70)/B 0.1% Formic acid in water:Acetonitrile (10:90), runtime=9.0 min, gradient: t=0 min 1% B, t=2.5 min 50% B, t=4.5 min 97.5% B, t=6.5 min 1% B, stop time 9.0 min.

### Liquid Chromatography-Mass Spectrometry Method F

LC/MS-ES (+/−): analyses performed using Agilent Infinity II G6125C LCMS, C18 column (50×4.6 mm, 3.5 μm particle size), column temperature 35° C., mobile phase: A 5 mM Ammonium Bicarbonate in Milli-Q water (pH=7.35)/B—Methanol, runtime=7.0 min, gradient: t=0 min 8% B, t=3.0 min 70% B, t=3.7 min 95% B, t=4.2 min 100% B, t=5.21 min 8% B, stop time 7.0 min.

### Liquid Chromatography-Mass Spectrometry Method G

LC/MS-ES (+/−): analyses performed using Waters Alliance 2690 and 996 PDA detector with Micromass ZQ, C18 column (150×4.6 mm, 3.5 μm particle size), column temperature 35° C., mobile phase: A—5 mM Ammonium Acetate+0.1% FA in Water/B—Methanol, runtime=17.0 min, gradient: t=0 min 10% B, t=7.0 min 90% B, t=9.0 min 100% B, t=14.01 min 10% B, stop time 17.0 min.

### Liquid Chromatography-Mass Spectrometry Method H

LC/MS-ES (+/−): analyses performed using AQUITY with PDA detector and QDA Performance, C18 column (50×2.1 mm, 1.6 μm particle size), column temperature 35° C., mobile phase: A—0.1% Formic acid in Milli Q water (pH=2.70)/B—0.1% Formic acid in water:Acetonitrile (10:90), flow rate: 0.9 mL/min, runtime=3.0 min, gradient: t=0 min 5% B, t=1.8 min 98% B, t=2.0 min 100% B, t=2.51 min 5% B, stop time 17.0 min.

### Analytical Methods

¹H Nuclear magnetic resonance (NMR) spectroscopy was carried out using one of the following instruments: a Bruker Avance 400 instrument equipped with probe DUAL 400 MHz S1, a Bruker Avance 400 instrument equipped with probe 6 S1 400 MHz 5 mm ¹H-¹³C ID, a Bruker Avance III 400 instrument with nanobay equipped with probe Broadband BBFO 5 mm direct, a 400 MHz Agilent Direct Drive instrument with ID AUTO-X PFG probe, all operating at 400 MHz, or an Agilent VNMRS500 Direct Drive instrument equipped with a 5 mm Triple Resonance ¹H{¹³C/¹⁵N}cryoprobe operating at 500 MHz. The spectra were acquired in the stated solvent at around room temperature unless otherwise stated. In all cases, NMR data were consistent with the proposed structures. Characteristic chemical shifts (6 (ppm)) are given in parts-per-million using conventional abbreviations for designation of major peaks: e.g. s, singlet; d, doublet; t, triplet; q, quartet; dd, doublet of doublets; dt, doublet of triplets; br, broad.

Thin layer chromatography (TLC) refers to silica gel TLC using silica gel F254 (Merck) plates. Column chromatography was performed using an automatic column chromatography (Biotage SP1 or Isolera) system over Biotage silica gel cartridges (KP-Sil or KP-NH) or in the case of reverse phase chromatography over Biotage C18 cartridges (KP-C 18).

Prep HPLC were performed on Shimadzu LC-20AP, Waters 2545 and Agilent 1260 infinity. Purity was determined on Waters Alliance e2695-PDA detector 2998 and Agilent 1260 Infinity-II. (Mobile phase: 0.05% HCl in Water/Methanol in gradient elution method).

TABLE 3
Abbreviations and Names of Reagents

| Abbreviations/ | |
|---|---|
| Acronyms | Full Name/Description |
| AcOH | Acetic acid |
| aq. | Aqueous |
| CH₃CN | Acetonitrile |
| B₂pin₂ | Bis(pinacolato)diboron |
| Boc₂O | Di-tert-butyl dicarbonate |
| BH₃·SMe₂ | Borane dimethyl sulfide complex |
| i-BuMgBr | Isobutyl magnesium bromide |
| n-BuLi | n-Butyllithium |
| B(O-iPr)₃. | Triisopropyl borate |
| CBzCl | Benzyl chloroformate |
| CDI | 1,1'-Carbonyldiimidazole |
| DAST | Diethylaminosulfur trifluoride |
| DCE | 1,2-Dichloethane |
| DCM | Dichloromethane |
| DIAD | Diisopropyl azodicarboxylate |
| DIBAL | Diisobutylaluminium hydride |
| DIPEA | N,N-Diisopropylethylamine |
| DMAP | N,N-dimethylaminopyridine |
| DMF | N,N'-dimethylformamide |
| DMP | Dess-Martin periodinane |
| DMSO | Dimethylsulfoxide |
| EDCI | 1-Ethyl-3-(3- |
| | dimethylaminopropyl)carbodiimide |
| DPPA | Diphenylphosphoryl azide |
| Et₂O | Diethyl ether |
| Et₃N | Triethylamine |
| EtOAc | Ethyl acetate |
| EtOH | Ethanol |
| EtMgBr | Ethylmagnesium bromide |
| HATU | Hexafluorophosphate |
| | azabenzotriazole tetramethyl |
| | uronium |
| HOBt | Hydroxybenzotriazole |
| HPLC | High performance liquid chromatography |
| KHMDS | Potassium bis(trimethylsilyl)amide |
| LCMS | Liquid chromatography mass spectrometry |
| LDA | Lithium diisopropylamide |
| Li(ALH)₄ | Lithium aluminum hydride |
| LiAlH(Ot-Bu)₃ | Lithium tri-tert-butoxyaluminum hydride |
| LHMDS | Lithium bis(trimethylsilyl)amide |
| MeOH | Methanol |
| Mel | Methyl Iodide |
| min. | minutes |
| NMR | Nuclear magnetic resonance |
| rt | Room temperature |
| NaBH₄ | Sodium borohydride |
| NaBH(OAc)₃ | Sodium triacetoxy borohydride |
| NaOAc | Sodium acetate |
| NaBH₃CN | Sodium cyanoborohydride |
| PCC | Pyridinium chlorochromate |
| Pd(dba)₂ | Bis(dibenzylideneacetone)palladium |
| Pd(dppf)Cl₂ | [1,1′- |
| | Bis(diphenylphosphino)ferrocene]di- |
| | chloropalladium |
| PPh₃ | Triphenylphosphine |
| i-PrBr | 2-Bromopropane |
| Sat. | Saturated |
| TBAF | Tetrabutlyammonium fluoride |
| TBSCl/TBDMSCl | t-butyldimethylsilyl chloride |
| Ti(O-iPr)₄ | Titanium isopropoxide |
| TEA | Triethylamine |
| TFA | Trifluoroacetic acid |
| TFAA | Trifluoroacetic anhydride |
| THF | Tetrahydrofuran |
| TMEDA | N,N,N′,N′-Tetramethylethylenediamine |
| TMSCN | Trimethylsilyl cyanide |
| Tf₂O | Trifluoromethanesulfonic anhydride |
| TsOH | p-Toluenesulfonic acid |

### Intermediate Synthesis

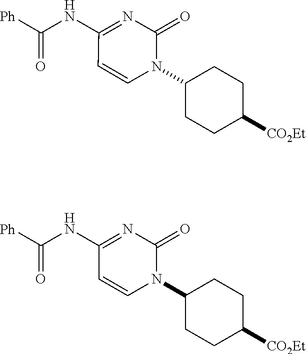 [SMILES: CCOC(=O)[C@H]1CC[C@H](n2ccc(NC(=O)c3ccccc3)nc2=O)CC1 ; CCOC(=O)[C@H]1CC[C@@H](n2ccc(NC(=O)c3ccccc3)nc2=O)CC1]

### Ethyl trans-4-(4-benzamido-2-oxopyrimidin-1(2H)-yl)cyclohexane-1-carboxylate and ethyl cis-4-(4-benzamido-2-oxopyrimidin-1(2H)-yl)cyclohexane-1-carboxylate

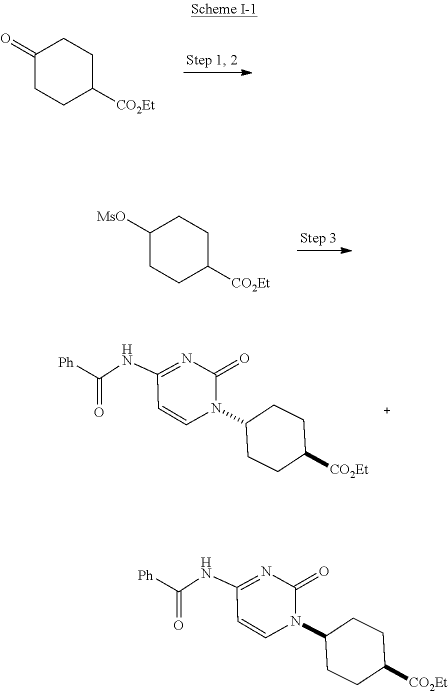 [SMILES: CCOC(=O)C1CCC(=O)CC1 ; CCOC(=O)C1CCC(OS(C)(=O)=O)CC1 ; CCOC(=O)[C@H]1CC[C@H](n2ccc(NC(=O)c3ccccc3)nc2=O)CC1 ; CCOC(=O)[C@H]1CC[C@@H](n2ccc(NC(=O)c3ccccc3)nc2=O)CC1]

Reagents: 1) NaBH₄, MeOH, 0° C. to rt, 16 h 2) MsCl, Et₃N, DCM, rt, 1.5 h 3) N⁴-benzoyl cytosine, 18-crown-6, K₂CO₃, DMF, 100° C., 24 h.

Step 1: ethyl 4-hydroxycyclohexane-1-carboxylate. Sodium borohydride (4.56 g, 121 mmol) was added portionwise to a solution of ethyl 4-oxocyclohexane-1-carboxylate (10 g, 58.8 mmol) in methanol (300 mL) at 0° C., and the mixture was warmed to rt while stirring for 16 h. The reaction mixture was concentrated, EtOAc (1 L) added and washed with sat. aq. NaHCO₃ (2×500 mL) and brine (1×500 mL), dried over Na₂SO₄), filtered, and concentrated to dryness to give the title compound (8.06 g).

Step 2: ethyl 4-((methylsulfonyl)oxy)cyclohexane-1-carboxylate. Methanesulfonyl chloride (4.4 mL, 56 mmol) was added dropwise to a solution of ethyl 4-hydroxycyclohexane-1-carboxylate (8.06 g, 46.8 mmol) and Et₃N (9.8 mL, 70 mmol) in DCM (225 mL) and the mixture was stirred at rt for 1.5 h. The mixture was poured into sat. aq. NaHCO₃ (500 mL) and extracted with DCM (2×500 mL). The extracts were dried over Na₂SO₄, filtered, and concentrated to give the title compound (11.7 g).

Step 3: ethyl trans-4-(4-benzamido-2-oxopyrimidin-1(2H)-yl)cyclohexane-1-carboxylate and ethyl cis-4-(4-benzamido-2-oxopyrimidin-1(2H)-yl)cyclohexane-1-carboxylate. A mixture of N⁴-benzoyl cytosine (201 mg, 0.93 mmol), ethyl 4-((methylsulfonyl)oxy)cyclohexane-1-carboxylate (280 mg, 1.12 mmol), 18-crown-6 (47 mg, 0.18 mmol), and K₂CO₃ (267 mg, 1.93 mmol) in DMF (5.0 mL) was stirred at 75° C. for 18 h then at 100° C. for another 24 h. The mixture was cooled, diluted with EtOAc (75 mL), washed with sat. aq. NaHCO₃ (2×50 mL) and brine (2×50 mL), dried over Na₂SO₄, filtered through Celite®, and concentrated. The residue was purified by flash chromatography (EtOAc/Hex) to afford the title compounds:

ethyl trans-4-(4-benzamido-2-oxopyrimidin-1(2H)-yl)cyclohexane-1-carboxylate (8.0 mg). ¹H NMR (500 MHz, CDCl₃) δ 8.98-8.53 (m, 1H), 7.90 (d, 2H), 7.68 (d, 1H), 7.61 (t, 1H), 7.56-7.44 (m, 3H), 4.70 (tt, 1H), 4.15 (q, 2H), 2.32 (tt, 1H), 2.24-2.16 (m, 2H), 2.15-2.08 (m, 2H), 1.77-1.63 (m, 2H), 1.61-1.49 (m, 2H), 1.29-1.23 (m, 3H). LCMS[M+H] 370.3.

ethyl cis-4-(4-benzamido-2-oxopyrimidin-1(2H)-yl)cyclohexane-1-carboxylate (35 mg). ¹H NMR (500 MHz, CDCl₃) δ 8.84-8.54 (m, 1H), 7.88 (d, 2H), 7.67 (d, 1H), 7.59 (t, 1H), 7.53-7.42 (m, 3H), 4.78-4.67 (m, 1H), 4.19 (q, 2H), 2.74-2.68 (m, 1H), 2.35-2.25 (m, 2H), 1.95-1.86 (m, 2H), 1.78-1.63 (m, 4H), 1.28 (t, 3H). LCMS[M+H] 370.3.

Alternatively, ethyl trans-4-(4-benzamido-2-oxopyrimidin-1(2H)-yl)cyclohexane-1-carboxylate may be prepared as depicted in Scheme I-2.

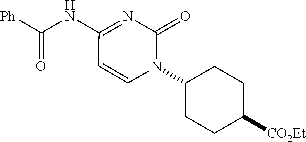 [SMILES: CCOC(=O)[C@H]1CC[C@H](n2ccc(NC(=O)c3ccccc3)nc2=O)CC1]

### Ethyl trans-4-(4-benzamido-2-oxopyrimidin-1(2H)-yl)cyclohexane-1-carboxylate

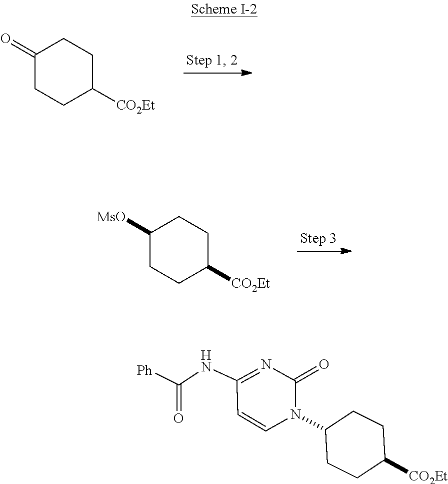 [SMILES: CCOC(=O)C1CCC(=O)CC1 ; CCOC(=O)[C@H]1CC[C@@H](OS(C)(=O)=O)CC1 ; CCOC(=O)[C@H]1CC[C@H](n2ccc(NC(=O)c3ccccc3)nc2=O)CC1]

Reagents: 1) L-Selectride, THF, −78° C., 2 h 2) MsCl, Et₃N, DCM, rt, 22 h 3) N⁴-benzoyl cytosine, 18-crown-6, K₂CO₃, DMF, 100° C., 4 d.

Step 1: ethyl cis-4-hydroxycyclohexane-1-carboxylate. L-Selectride 1M in THF (100 mL) was added dropwise to a solution of ethyl 4-oxocyclohexane-1-carboxylate (14.3 g, 84 mmol) in THF (500 mL) at −78° C., and the mixture was stirred for 2 h. Sat. aq. NaHCO₃ (100 mL) was added by dropwise addition, the mixture was warmed to 0° C., 30% H₂O₂ (25 mL) was added, and the mixture was stirred at rt overnight. EtOAc (2 L) was added, the layers were separated and washed with sat. aq. NaHCO₃ (1 L) and brine (1 L), dried over Na₂SO₄, filtered, and concentrated. The residue was purified by column chromatography (EtOAc/Hexanes) to give the title compound (11.96 g). ¹H NMR (500 MHz, CDCl₃) δ 4.14 (q, 2H), 3.93-3.85 (m, 1H), 2.37 (tt, 1H), 2.02-1.90 (m, 2H), 1.74-1.60 (m, 6H), 1.26 (t, 3H). The minor diastereomer, ethyl trans-4-hydroxycyclohexane-1-carboxylate (1.06 g) was also recovered.

Step 2: ethyl cis-4-((methylsulfonyl)oxy)cyclohexane-1-carboxylate. Methanesulfonyl chloride (6.5 mL, 83 mmol) was added dropwise to a solution of ethyl cis-4-hydroxycyclohexane-1-carboxylate (11.9 g, 69 mmol) and Et₃N (14.5 mL, 104 mmol) in DCM (350 mL) at 0° C. The mixture was warmed to rt and stirred for 3 h. Another portion of MsCl (1.3 mL, 17 mmol) was added and the mixture was stirred for another 19 h. The crude material was purified by column chromatography (Hex/DCM/CH₃CN) to give the title compound (14.6 g).

Step 3: ethyl trans-4-(4-benzamido-2-oxopyrimidin-1(2H)-yl)cyclohexane-1-carboxylate. A mixture of N⁴-benzoyl cytosine (15.1 g, 70 mmol), ethyl cis-4-((methylsulfonyl)oxy)cyclohexane-1-carboxylate (14.6 g, 58 mmol), 18-crown-6 (3.1 g, 11.7 mmol), and K₂CO₃ (16.1 g, 116 mmol) in DMF (285 mL) was stirred at 100° C. for 4 d. The mixture was cooled, poured into water (2.5 L), and the precipitate was collected by vacuum filtration. The product was purified by column chromatography (Hex/EtOAc/MeOH) to give the title compound (1.40 g). ¹H NMR (500 MHz, CDCl₃) δ 7.94 (d, 2H), 7.70 (d, 1H), 7.62 (t, 1H), 7.58-7.47 (m, 3H), 4.69 (tt, 1H), 4.15 (q, 2H), 2.33 (tt, 1H), 2.25-2.17 (m, 2H), 2.16-2.08 (m, 2H), 1.70 (qd, 2H), 1.56 (qd, 2H), 1.27 (t, 3H). LCMS[M+H] 370.3.

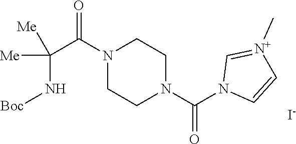 [SMILES: C[n+]1ccn(C(=O)N2CCN(C(=O)C(C)(C)NC(=O)OC(C)(C)C)CC2)c1 ; [I-]]

### 1-(4-(2-((tert-Butoxycarbonyl)amino)-2-methylpropanoyl)piperazine-1-carbonyl)-3-methyl-1H-imidazol-3-ium iodide

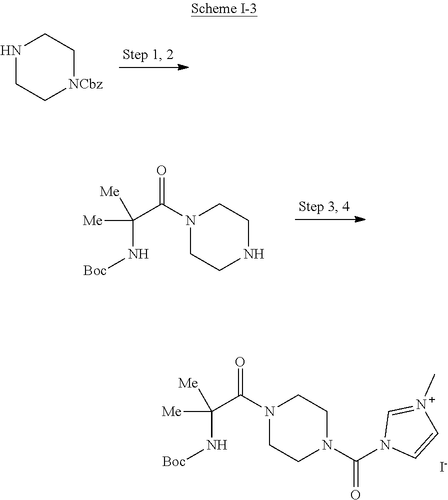 [SMILES: O=C(OCc1ccccc1)N1CCNCC1 ; CC(C)(C)OC(=O)NC(C)(C)C(=O)N1CCNCC1 ; C[n+]1ccn(C(=O)N2CCN(C(=O)C(C)(C)NC(=O)OC(C)(C)C)CC2)c1 ; [I-]]

Reagents: 1) 2-(Boc-amino)isobutyric acid, EDC, HOBt, DIPEA, DMF, rt, 44 h 2) H₂, 10% Pd/C, EtOH, rt, 24 h 3) CDI, DCM, rt, 22 h 4) CH₃I, CH₃CN, rt, 2 d.

Step 1: benzyl 4-(2-((tert-butoxycarbonyl)amino)-2-methylpropanoyl)piperazine-1-carboxylate. Benzyl piperazine-1-carboxylate (19.5 mL, 101 mmol) was added to a mixture of 2-(boc-amino)isobutyric acid (22.7 g, 111.5 mmol), EDC (23.3 g, 122 mmol), HOBt (19.4 g, 85%, 128 mmol), and DIPEA (35 mL, 200 mmol) in DMF (200 mL), and the mixture was stirred at rt for 44 h. Water (1 L) was added, a gummy precipitate formed, and the liquid was poured off EtOAc (1 L) was added and the solution was washed with sat. aq. NaHCO₃ (2×500 mL) and brine (1×500 mL), dried over Na₂SO₄, filtered, and concentrated to afford the title compound (30.7 g) as a white foam.

Step 2: tert-butyl (2-methyl-1-oxo-1-(piperazin-1-yl)propan-2-yl)carbamate. A mixture of benzyl 4-(2-((tert-butoxycarbonyl)amino)-2-methylpropanoyl)piperazine-1-carboxylate (30.7 g, 76 mmol) and 10 wt %, Pd/C (ca. 50% H₂O, 4.58 g) in EtOH (350 mL) was stirred under H₂ (atm) for 24 h. The mixture was filtered through a pad of Celite® and the filtrate was concentrated to dryness and dried from toluene then Et₂O to afford the title compound (20.4 g).

Step 3: tert-butyl (1-(4-(1H-imidazole-1-carbonyl)piperazin-1-yl)-2-methyl-1-oxopropan-2-yl)carbamate. A mixture of tert-butyl (2-methyl-1-oxo-1-(piperazin-1-yl)propan-2-yl)carbamate (21.85 g, 77 mmol) and CDI (16.7 g, 93 mmol) in DCM (300 mL) was stirred at rt for 22 h. The mixture was diluted with DCM (1 L), washed with sat. aq. NaHCO₃ (2×500 mL) and brine (1×500 mL), dried over Na₂SO₄, filtered, and concentrated to dryness to afford the title compound (29.3 g).

Step 4: 1-(4-(2-((tert-butoxycarbonyl)amino)-2-methylpropanoyl)piperazine-1-carbonyl)-3-methyl-1H-imidazol-3-ium iodide. Methyl iodide (25 mL, 403 mmol) was added to a suspension of tert-butyl (1-(4-(1H-imidazole-1-carbonyl)piperazin-1-yl)-2-methyl-1-oxopropan-2-yl)carbamate (29.3 g, 80 mmol) in CH₃CN (375 mL), and the mixture was stirred at rt under N₂ for 2 d. The reactions mixture was concentrated to 100 mL total volume and Et₂O (350 mL) was added. The precipitate was collected by vacuum filtration, rinsing with additional Et₂O, to afford the title compound (38.8 g). ¹H NMR (500 MHz, DMSO-d₆) δ 9.54 (s, 1H), 8.01 (s, 1H), 7.87-7.82 (m, 1H), 7.39-7.31 (m, 1H), 3.91 (s, 3H), 3.86-3.57 (m, 4H), 3.55-3.43 (m, 4H), 1.39 (s, 9H), 1.31 (s, 6H).

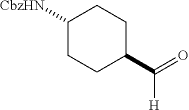 [SMILES: O=C[C@H]1CC[C@H](NC(=O)OCc2ccccc2)CC1]

### Benzyl (trans-4-formylcyclohexyl)carbamate

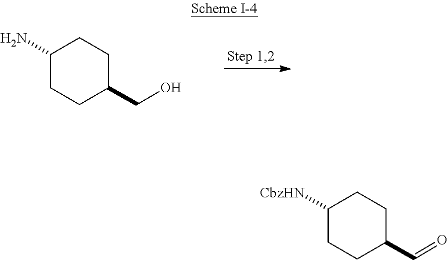 [SMILES: N[C@H]1CC[C@H](CO)CC1 ; O=C[C@H]1CC[C@H](NC(=O)OCc2ccccc2)CC1]

Reagents: 1) Cbz-Cl, 1M NaOH, dioxane, rt, 16 h 2) SO₃ pyridine, DIPEA, DMSO, 0° C. to rt, 19 h.

Step 1: benzyl (trans-4-(hydroxymethyl)cyclohexyl)carbamate. A mixture of (trans-4-aminocyclohexyl)methanol (25 g, 190 mmol), 1M NaOH (580 mL, 580 mmol), and Cbz-Cl (33 mL, 230 mmol) in dioxane (300 mL) was stirred at rt for 16 h. The mixture was neutralized with 1M HCl (600 mL) to approximately pH 4-7 and the precipitate was collected by vacuum filtration to give the title compound (46 g). ¹H NMR (500 MHz, CDCl₃) δ 7.37-7.28 (m, 5H), 5.08 (s, 2H), 4.68 (s, 1H), 3.50-3.39 (m, 3H), 2.09-1.98 (m, 2H), 1.86-1.71 (m, 3H), 1.48-1.38 (m, 1H), 1.18-0.98 (m, 4H).

Step 2: benzyl (trans-4-formylcyclohexyl)carbamate. A solution of SO₃ pyridine (21 g, 132 mmol) in DMSO (75 mL, 1.1 mol) was added dropwise to a vigorously stirred mixture of benzyl (trans-4-(hydroxymethyl)cyclohexyl)carbamate (11.6 g, 44 mmol) and DIPEA (75 mL, 430 mmol) in DMSO (75 mL) at 0° C., and the mixture was warmed to rt and stirred for 19 h. 10% citric acid (500 mL) was added dropwise. Water (750 mL) was added and the mixture was stirred for 1 h. The precipitate was collected by vacuum filtration. The product was added to Et₂O (1 L), washed with 5% citric acid (600 mL), sat. aq. NaHCO₃ (600 mL), brine (600 mL), dried over (Na₂SO₄), filtered, and concentrated in vacuo to give the title compound (12.0 g). ¹H NMR (500 MHz, CDCl₃) δ 9.62 (s, 1H), 7.41-7.27 (m, 5H), 5.09 (s, 2H), 4.68-4.57 (m, 1H), 3.55-3.41 (m, 1H), 2.22-2.09 (m, 3H), 2.08-1.98 (m, 2H), 1.47-1.33 (m, 2H), 1.23-1.12 (m, 2H).

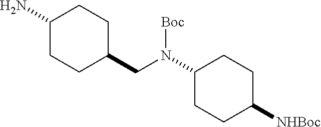 [SMILES: CC(C)(C)OC(=O)N[C@H]1CC[C@H](N(C[C@H]2CC[C@H](N)CC2)C(=O)OC(C)(C)C)CC1]

### tert-Butyl ((trans-4-aminocyclohexyl)methyl)(trans-4-((tert-butoxycarbonyl)amino)cyclohexyl)carbamate

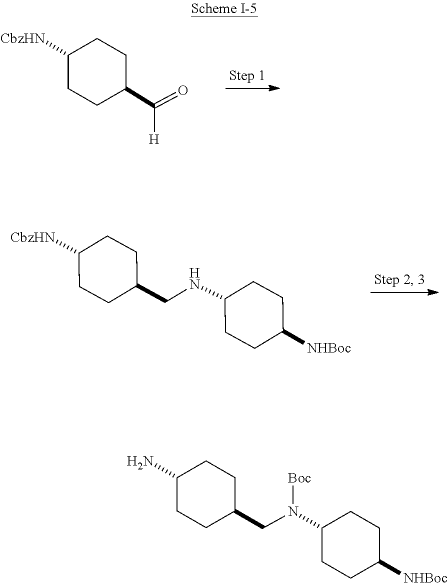 [SMILES: O=C[C@H]1CC[C@H](NC(=O)OCc2ccccc2)CC1 ; CC(C)(C)OC(=O)N[C@H]1CC[C@H](NC[C@H]2CC[C@H](NC(=O)OCc3ccccc3)CC2)CC1 ; CC(C)(C)OC(=O)N[C@H]1CC[C@H](N(C[C@H]2CC[C@H](N)CC2)C(=O)OC(C)(C)C)CC1]

Reagents: 1) tert-butyl (trans-4-aminocyclohexyl)carbamate, NaBH(OAc)₃, DCE, rt, 18 h 2) Boc₂O, 1M NaOH, dioxane, rt, 24 h 3) H₂, 10% Pd/C, MeOH, rt, 20 h.

Step 1: benzyl (trans-4-(((trans-4-((tert-butoxycarbonyl)amino)cyclohexyl)amino)methyl)cyclohexyl)carbamate. Sodium triacetoxyborohydride (9.7 g, 46 mmol) was added to a mixture of benzyl (trans-4-formylcyclohexyl)carbamate (5.96 g, 23 mmol) and tert-butyl (trans-4-aminocyclohexyl)carbamate (5.86 g, 27 mmol) in dichloroethane (DCE, 250 mL), and the mixture was stirred at rt for 18 h. 1M K₂CO₃ (50 mL) was added and the reaction mixture stirred vigorously for 2 h. The mixture was poured into sat. aq. NaHCO₃ (500 mL) and extracted with DCM (2×250 mL). The extracts were dried over Na₂SO₄, filtered, and concentrated to give the title compound (10.7 g).

Step 2: tert-butyl ((trans-4-(((benzyloxy)carbonyl)amino)cyclohexyl)methyl)(trans-4-((tert-butoxycarbonyl)amino)cyclohexyl)carbamate. A mixture of benzyl (trans-4-(((trans-4-((tert-butoxycarbonyl)amino)cyclohexyl)amino)methyl)cyclohexyl)carbamate (10.5 g, 22.8 mmol), Boc₂O (6.8 mL, 30 mmol), and 1M NaOH (69 mL, 69 mmol) in dioxane (100 mL) was stirred at rt for 24 h. Another portion each of 1M NaOH (35 mL, 35 mmol) and Boc₂O (3.4 mL, 15 mmol) were added and the reaction mixture stirred for an additional 20 h. Water was added (350 mL) and the mixture was brought to neutral pH with 1M HCl. The mixture was poured into EtOAc (500 mL), and the organic layer was washed with brine (1×500 mL), dried over Na₂SO₄, filtered, and concentrated. The residue was purified by column chromatography (EtOAc/Hexanes) to give the title compound (7.12 g) as a cream colored foam. ¹H NMR (500 MHz, MeOD) δ 7.39-7.24 (m, 5H), 5.05 (s, 2H), 3.39-3.20 (m, 3H), 3.02 (d, 2H), 2.00-1.89 (m, 4H), 1.81-1.64 (m, 6H), 1.58-1.48 (m, 1H), 1.45 (s, 9H), 1.43 (s, 9H), 1.32-1.13 (m, 4H), 1.10-0.97 (m, 2H).

Step 3: tert-butyl ((trans-4-aminocyclohexyl)methyl)(trans-4-((tert-butoxycarbonyl)amino)cyclohexyl)carbamate. A mixture of tert-butyl ((trans-4-(((benzyloxy)carbonyl)amino)cyclohexyl)methyl)(trans-4-((tert-butoxycarbonyl)amino)cyclohexyl)carbamate (7.12 g, 12.7 mmol) and 10% Pd/C (ca 50% water, 720 mg) in MeOH (200 mL) was stirred under an atmosphere of H₂ for 20 h. The mixture was filtered through a pad of Celite® and the filtrate was concentrated in vacuo then concentrated from toluene to give the title compound (5.24 g).

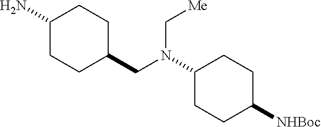 [SMILES: CCN(C[C@H]1CC[C@H](N)CC1)[C@H]1CC[C@H](NC(=O)OC(C)(C)C)CC1]

### tert-Butyl (trans-4-(((trans-4-aminocyclohexyl)methyl)(ethyl)amino)cyclohexyl)carbamate

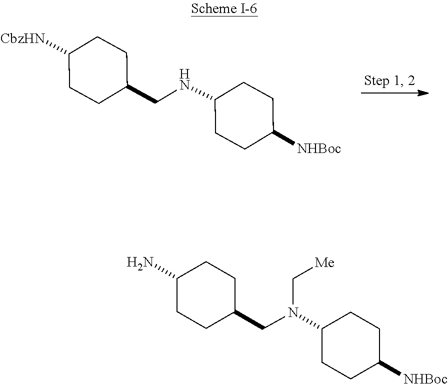 [SMILES: CC(C)(C)OC(=O)N[C@H]1CC[C@H](NC[C@H]2CC[C@H](NC(=O)OCc3ccccc3)CC2)CC1 ; CCN(C[C@H]1CC[C@H](N)CC1)[C@H]1CC[C@H](NC(=O)OC(C)(C)C)CC1]

Reagents: 1) acetaldehyde, NaBH(OAc)₃, AcOH, DCE, rt, 22 h 2) H₂, 10% Pd/C, 7M NH₃ in MeOH, MeOH, rt, 20 h.

Step 1: benzyl (trans-4-(((trans-4-((tert-butoxycarbonyl)amino)cyclohexyl)(ethyl)amino)methyl)cyclohexyl)carbamate. NaBH(OAc)₃ (1.9 g, 9.0 mmol) was added to a mixture of benzyl (trans-4-(((trans-4-((tert-butoxycarbonyl)amino)cyclohexyl)amino)methyl)cyclohexyl)carbamate (2.05 g, 4.46 mmol), acetaldehyde (2.5 mL, 45 mmol), and acetic acid (0.41 mL, 7.2 mmol) in DCE (50 mL), and the mixture was stirred at rt for 22 h. The mixture was poured into sat. aq. NaHCO₃ (125 mL) and extracted with DCM (2×125 mL). The extracts were dried (Na₂SO₄), filtered, and concentrated to dryness to give the title compound (2.07 g). ¹H NMR (500 MHz, DMSO-d₆) δ 7.40-7.27 (m, 5H), 7.13 (d, 1H), 6.63 (d, 1H), 4.98 (s, 2H), 3.26-3.16 (m, 1H), 3.16-3.05 (m, 1H), 2.39 (q, 2H), 2.35-2.25 (m, 1H), 2.13 (d, 2H), 1.85-1.71 (m, 6H), 1.66-1.57 (m, 2H), 1.36 (s, 9H), 1.27-1.04 (m, 7H), 0.91 (t, 3H), 0.86-0.73 (m, 2H). LCMS[M+H] 488.3.

Step 2: tert-butyl (trans-4-(((trans-4-aminocyclohexyl)methyl)(ethyl)amino)cyclohexyl)carbamate. A mixture of benzyl (trans-4-(((trans-4-((tert-butoxycarbonyl)amino)cyclohexyl)(ethyl)amino)methyl)cyclohexyl) carbamate (1.96 g, 4.02 mmol) and 10% Pd/C (ca 50% water, 193 mg) in MeOH (80 mL) and 7M NH₃ in MeOH (4 mL) was stirred under an atmosphere of H₂ for 20 h. The mixture was filtered through a pad of Celite® and the filtrate was concentrated in vacuo then concentrated from toluene to give the title compound (1.42 g). ¹H NMR (500 MHz, MeOD) δ 3.26-3.15 (m, 1H), 2.59 (tt, 1H), 2.51 (q, 2H), 2.48-2.41 (m, 1H), 2.22 (d, 2H), 1.98-1.82 (m, 6H), 1.81-1.72 (m, 2H), 1.43 (s, 9H), 1.38-1.25 (m, 3H), 1.24-1.06 (m, 4H), 1.00 (t, 3H), 0.95-0.83 (m, 2H).

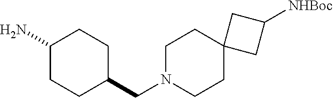 [SMILES: CC(C)(C)OC(=O)NC1CC2(CCN(C[C@H]3CC[C@H](N)CC3)CC2)C1]

### tert-Butyl (7-((trans-4-aminocyclohexyl)methyl)-7-azaspiro[3.5]nonan-2-yl)carbamate

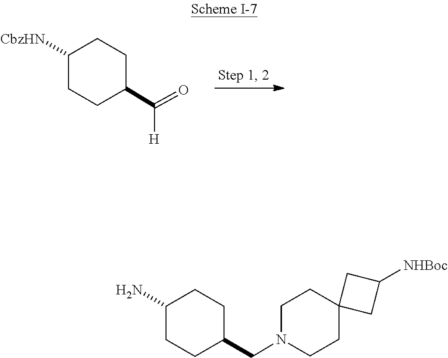 [SMILES: O=C[C@H]1CC[C@H](NC(=O)OCc2ccccc2)CC1 ; CC(C)(C)OC(=O)NC1CC2(CCN(C[C@H]3CC[C@H](N)CC3)CC2)C1]

Reagents: 1) tert-butyl (7-azaspiro[3.5]nonan-2-yl)carbamate, NaBH(OAc)₃, DCE, rt, 3 d 2) H₂, 5% Pd/C, 7M NH₃ in MeOH, MeOH, rt, 3 h.

Step 1: benzyl (trans-4-((2-((tert-butoxycarbonyl)amino)-7-azaspiro[3.5]nonan-7-yl)methyl)cyclohexyl)carbamate. NaBH(OAc)₃ (2.35 g, 11.1 mmol) was added to a mixture of benzyl (trans-4-formylcyclohexyl)carbamate (1.43 g, 5.47 mmol) and tert-butyl (7-azaspiro[3.5]nonan-2-yl)carbamate (1.2 g, 5.0 mmol) in DCE (50 mL), and the mixture was stirred at rt for 3 d. The reaction was quenched with 1M K₂CO₃ (35 mL) and that was stirred vigorously for 1 h. The mixture was then poured into sat. aq. NaHCO₃ (75 mL) and extracted with DCM (2×75 mL). The extracts were dried (Na₂SO₄), decanted and concentrated. The residue was purified by column chromatography (Hex/EtOAc/MeOH/NH₄OH) to give the title compound). ¹H NMR (500 MHz, MeOD) δ 7.38-7.24 (m, 5H), 5.05 (s, 2H), 4.00-3.91 (m, 1H), 3.39-3.32 (m, 1H), 2.48-2.21 (m, 4H), 2.21-2.14 (m, 2H), 2.12 (d, 2H), 1.98-1.89 (m, 2H), 1.88-1.79 (m, 2H), 1.68-1.53 (m, 6H), 1.53-1.37 (m, 10H), 1.27-1.15 (m, 2H), 1.06-0.92 (m, 2H).

Step 2: tert-butyl (7-((trans-4-aminocyclohexyl)methyl)-7-azaspiro[3.5]nonan-2-yl)carbamate. A mixture of benzyl (trans-4-((2-((tert-butoxycarbonyl)amino)-7-azaspiro[3.5]nonan-7-yl)methyl)cyclohexyl)carbamate (2.31 g, 4.76 mmol) and 5% Pd/C (323 mg) in MeOH (60 mL) and 7M NH₃ in MeOH (4 mL) was stirred under an atmosphere of H₂ for 3 h. The mixture was filtered through a pad of Celite®, and the filtrate was concentrated in vacuo then concentrated from toluene/DCM to give the title compound (1.67 g).

### Compound Synthesis

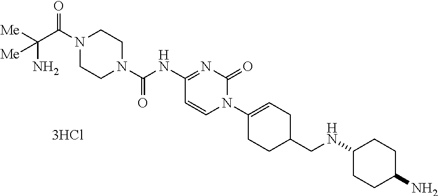 [SMILES: CC(C)(N)C(=O)N1CCN(C(=O)Nc2ccn(C3=CCC(CN[C@H]4CC[C@H](N)CC4)CC3)c(=O)n2)CC1]

### 4-(2-Amino-2-methylpropanoyl)-N-(1-(4-(((trans-4-aminocyclohexyl)amino)methyl)cyclohex-1-en-1-yl)-2-oxo-1,2-dihydropyrimidin-4-yl)piperazine-1-carboxamide hydrochloride salt (Compound 1)

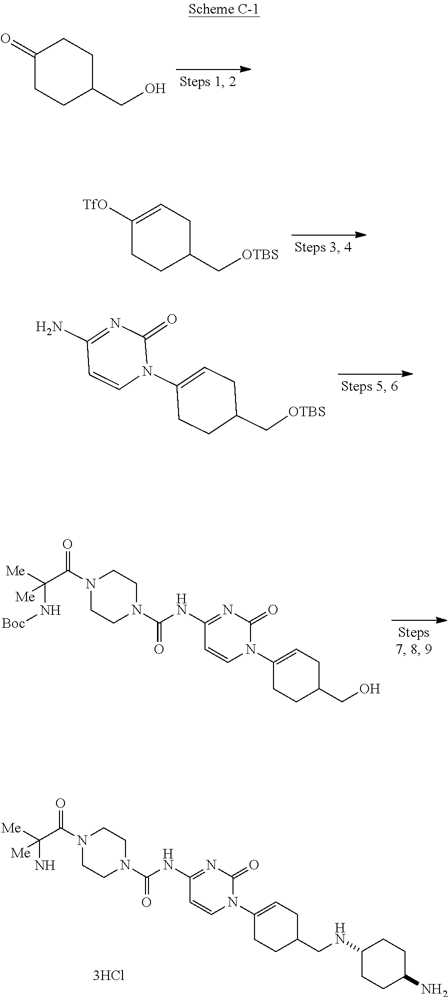

Reagents: 1) TBSCl, Imidazole, DMF, 16 h, rt 2) 2,6-di-tert-butyl-4-methylpyridine, Tf₂O, CH₂Cl₂, 0° C. to rt. 8 h, 3) Pd(dppf)Cl₂·CH₂Cl₂, KOAc, B₂pin₂, 1,4-dioxane, 105° C., 16 h 4) cytosine, Cu(OAc)₂·H₂O, TMEDA, MeOH:H₂O (4:1), rt, 48 h 5) 1-(4-(2-((tert-butoxycarbonyl)amino)-2-methylpropanoyl)piperazine-1-carbonyl)-3-methyl-1H-imidazol-3-ium iodide, MeCN, 85° C., 16 h 6) MeOH, TsOH, rt, 16 h 7) DMP, CH₂Cl₂, rt 3 h 8) tert-butyl (trans-4-aminocyclohexyl)carbamate, Na(OAc)₃BH, DCE, rt, 16 h 9) HCl/MeOH, rt, 4 h.

Step 1: 4-(((tert-butyldimethylsilyl)oxy)methyl)cyclohexan-1-one. To a solution of 4-(hydroxymethyl)cyclohexan-1-one (1.5 g, 12 mmol) in DMF (25 mL) was added TBSCl (2.6 g, 18 mmol) and imidazole (1.6 g, 23 mmol). The reaction was stirred for 16 h at rt. The reaction mixture was diluted with EtOAc (150 mL) and washed with sat. aq. LiCl (3×150 mL). The organic layer was concentrated under reduced pressure and purified by column chromatography (Hex:EtOAc) to afford the desired product (56%).

Step 2: 4-(((tert-butyldimethylsilyl)oxy)methyl)cyclohex-1-en-1-yl trifluoromethanesulfonate. To a solution of 4-(((tert-butyldimethylsilyl)oxy)methyl)cyclohexan-1-one (1.6 g, 6.6 mmol) and 2,6-di-tert-butyl-4-methylpyridine (1.76 g, 8.6 mmol) at 0° C. in CH₂Cl₂ (40 mL) was added Tf₂O (1.34 mL, 7.9 mmol) dropwise over 10 min. The reaction was warmed to rt and stirred for 8 h. Sat. aq.NaHCO₃ (40 mL) The reaction was quenched upon addition of was added, the organic layer was separated, concentrated under reduced pressure and purified by column chromatography (Hex:EtOAc) to afford the desired product as an amber oil.

Step 3: tert-butyldimethyl((4-(4,4,5,5-tetramethyl-1,3,2-dioxaborolan-2-yl)cyclohex-3-en-1-yl)methoxy)silane. A flask containing 4-(((tert-butyldimethylsilyl)oxy)methyl)cyclohex-1-en-1-yl trifluoromethanesulfonate (2.25 g, 6.0 mmol), Pd(dppf)Cl₂·CH₂Cl₂ (245 mg, 0.3 mmol), KOAc (1.77 g, 18 mmol) and B₂pin₂ (1.68 g, 6.6 mmol) was evacuated and flushed with N₂. 1,4-Dioxane (100 mL) was added, and the reaction mixture was degassed and heated to 105° C. for 16 h. The reaction mixture was cooled to rt, diluted with EtOAc (250 mL) and filtered through a pad of Celite®. The crude reaction mixture was purified by flash chromatography (EtOAc:Hex) to afford the desired product as a brown oil. ¹H NMR (500 MHz, CDCl₃) δ 6.56 (s, 1H), 3.47 (d, 2H), 2.29-2.04 (m, 2H), 1.88-1.66 (m, 3H), 1.54 (s, 1H), 1.26 (s, 12H), 1.23-1.16 (m, 1H), 0.89 (s, 9H) 0.04 (s, 6H).

Step 4: 4-amino-1-(4-(((tert-butyldimethylsilyl)oxy)methyl)cyclohex-1-en-1-yl)pyrimidin-2(1H)-one. A suspension of tert-butyldimethyl((4-(4,4,5,5-tetramethyl-1,3,2-dioxaborolan-2-yl)cyclohex-3-en-1-yl)methoxy)silane (1.00 g, 2.84 mmol) and cytosine (315 mg, 2.84 mmol) in MeOH:H₂O (4:1, 125 mL) was stirred at rt for 30 min. Cu(OAc)₂·H₂O (567 mg, 2.8 mmol) and TMEDA (0.51 mL, 3.4 mmol) were added and the reaction was stirred at rt for 48 h. The reaction mixture was concentrated under reduced pressure and H₂O was added (150 mL). The solids were filtered and washed with Et₂O (25 mL) and cold H₂O (25 mL) to afford the desired product. LCMS[M+H] 336.3.

Step 5: tert-butyl (1-(4-((1-(4-(((tert-butyldimethylsilyl)oxy)methyl)cyclohex-1-en-1-yl)-2-oxo-1,2-dihydropyrimidin-4-yl)carbamoyl)piperazin-1-yl)-2-methyl-1-oxopropan-2-yl)carbamate. To a solution of 4-amino-1-(4-(((tert-butyldimethylsilyl)oxy)methyl)cyclohex-1-en-1-yl)pyrimidin-2(1H)-one (200 mg, 0.6 mmol) in MeCN (50 mL), was added 1-(4-(2-((tert-butoxycarbonyl)amino)-2-methylpropanoyl)piperazine-1-carbonyl)-3-methyl-1H-imidazol-3-ium iodide (363 mg, 0.7 mmol). The reaction mixture was heated to 85° C. for 16 h. The volatiles were removed under reduced pressure and the crude solid was dissolved in CH₂Cl₂ (100 mL) and the organic layer was washed with H₂O (1×100 mL) and concentrated under reduced pressure. The crude solid was purified by column chromatography (CH₂Cl₂:MeOH) to afford the desired product. LCMS[M+H] 633.4.

Step 6: tert-butyl (1-(4-((1-(4-(hydroxymethyl)cyclohex-1-en-1-yl)-2-oxo-1,2-dihydropyrimidin-4-yl)carbamoyl)piperazin-1-yl)-2-methyl-1-oxopropan-2-yl)carbamate. To a stirred solution of tert-butyl (1-(4-((1-(4-(((tert-butyldimethylsilyl)oxy)methyl)cyclohex-1-en-1-yl)-2-oxo-1,2-dihydropyrimidin-4-yl)carbamoyl)piperazin-1-yl)-2-methyl-1-oxopropan-2-yl)carbamate (290 mg, 0.46 mmol) in MeOH (10 mL) at rt, was added TsOH (174 mg, 0.92 mmol). The reaction was maintained at rt for 2 h. The reaction mixture was concentrated under reduced pressure and the crude solid was dissolved in CH₂Cl₂ (25 mL). The organic layer was washed with sat. aq. NaHCO₃ (1×25 mL), dried over Na₂SO₄ and purified by column chromatography (CH₂Cl₂:MeOH) to afford the desired product. LCMS[M+H] 519.3.

Step 7: tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl)carbamate. To a stirred solution of tert-butyl (1-(4-((1-(4-(hydroxymethyl)cyclohex-1-en-1-yl)-2-oxo-1,2-dihydropyrimidin-4-yl)carbamoyl)piperazin-1-yl)-2-methyl-1-oxopropan-2-yl)carbamate (50 mg, 0.096 mmol) in DCM (25 mL) was added Dess-Martin periodinane (82 mg, 0.2 mmol). The solution was stirred at rt for 2 h. The crude reaction mixture was dissolved in CH₂Cl₂ (20 mL) and washed with aq. NaHCO₃/Na₂S₂O₃ (1×20 mL). The aq. layer was extracted with CH₂Cl₂ (1×10 mL). The combined organic layers were dried over Na₂SO₄ and concentrated under reduced pressure to afford the desired product. LCMS[M+H]517.3.

Step 8: tert-butyl (1-(4-((1-(4-(((trans-4-((tert-butoxycarbonyl)amino)cyclohexyl)amino)methyl)cyclohex-1-en-1-yl)-2-oxo-1,2-dihydropyrimidin-4-yl)carbamoyl)piperazin-1-yl)-2-methyl-1-oxopropan-2-yl)carbamate. To a stirred solution tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl)carbamate (25 mg, 0.05 mmol) in 1,2-dichloroethane (5 mL), was added tert-butyl (trans-4-aminocyclohexyl)carbamate (10 mg 0.05 mmol) and Na(OAc)₃BH (15 mg, 0.07 mmol). The reaction was stirred at rt for 16 h. The reaction mixture was diluted with CH₂Cl₂ and washed with saturated NaHCO₃ (1×15 mL). The aqueous layer was extracted with CH₂Cl₂ (2×20 mL). The combined organics were dried over Na₂SO₄ and concentrated under reduced pressure to afford the desired product.

Step 9: 4-(2-amino-2-methylpropanoyl)-N-(1-(4-(((trans-4-aminocyclohexyl)amino)methyl)cyclohex-1-en-1-yl)-2-oxo-1,2-dihydropyrimidin-4-yl)piperazine-1-carboxamide hydrochloride salt. tert-butyl (1-(4-((1-(4-(((trans-4-((tert-butoxycarbonyl)amino)cyclohexyl)amino)methyl)cyclohex-1-en-1-yl)-2-oxo-1,2-dihydropyrimidin-4-yl)carbamoyl)piperazin-1-yl)-2-methyl-1-oxopropan-2-yl)carbamate was dissolved in a solution of HCl/MeOH (5 mL) and stirred for 4 h. The reaction mixture was concentrated under reduced pressure and the crude solid was purified by reverse phase HPLC (H₂O:CH₃CN:TFA). Concentration of desired fractions under reduced pressure, addition of HCl/MeOH (3×15 mL) and evaporation under reduced pressure afforded the desired product. ¹H NMR (400 MHz, D₂O) δ 7.94 (d, 1H), 6.75 (d, 1H), 5.99 (s, 1H), 3.78 (br. s, 3H), 3.72 (br. s, 5H), 3.32-3.20 (m, 2H), 3.13 (d, 2H), 2.51-2.36 (m, 3H), 2.36-1.97 (m, 7H), 1.74 (s, 6H), 1.67-1.48 (m, 5H). LCMS[(M+2H)/2]258.2.

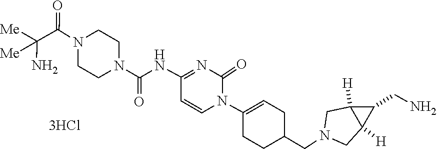 [SMILES: CC(C)(N)C(=O)N1CCN(C(=O)Nc2ccn(C3=CCC(CN4C[C@@H]5[C@@H](CN)[C@@H]5C4)CC3)c(=O)n2)CC1]

### 4-(2-Amino-2-methylpropanoyl)-N-(1-(4-((exo-6-(aminomethyl)-3-azabicyclo[3.1.0]hexan-3-yl)methyl)cyclohex-1-en-1-yl)-2-oxo-1,2-dihydropyrimidin-4-yl)piperazine-1-carboxamide hydrochloride salt (Compound 2)

Prepared as in Scheme C-1 from tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl)carbamate and tert-butyl ((exo-3-azabicyclo[3.1.0]hexan-6-yl)methyl)carbamate ¹H NMR (400 MHz, D₂O) δ 7.84 (d, 1H), 6.77 (d, 1H), 5.95 (s, 1H), 3.88 (d, 2H), 3.78 (br. s, 3H), 3.72 (br s, 5H), 3.53 (d, 2H), 3.27 (d, 2H), 3.01-2.93 (m, 2H), 2.53-2.28 (m, 3H), 2.26-1.93 (m, 5H), 1.74 (s, 6H), 1.69-1.40 (m, 2H). LCMS[M+H] 513.3.

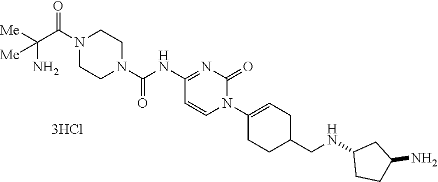 [SMILES: CC(C)(N)C(=O)N1CCN(C(=O)Nc2ccn(C3=CCC(CN[C@H]4CC[C@H](N)C4)CC3)c(=O)n2)CC1]

### 4-(2-Amino-2-methylpropanoyl)-N-(1-(4-((((1S,3S)-3-aminocyclopentyl)amino)methyl)cyclohex-1-en-1-yl)-2-oxo-1,2-dihydropyrimidin-4-yl)piperazine-1-carboxamide hydrochloride salt (Compound 3)

Prepared as in Scheme C-1 from tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl)carbamate and tert-butyl ((1S,3S)-3-aminocyclopentyl)carbamate. ¹H NMR (500 MHz, D₂O) δ 7.91 (d, 1H), 6.79 (d, 1H), 6.00 (s, 1H), 3.99-3.87 (m, 2H), 3.80 (s, 3H), 3.74 (s, 5H), 3.20-3.09 (m, 2H), 2.52-2.28 (m, 7H), 2.22-2.01 (m, 3H), 1.91-1.79 (m, 2H), 1.77 (s, 6H), 1.68-1.59 (m, 1H). LCMS[M+H] 501.3.

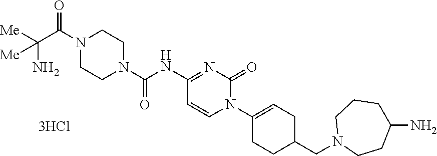 [SMILES: CC(C)(N)C(=O)N1CCN(C(=O)Nc2ccn(C3=CCC(CN4CCCC(N)CC4)CC3)c(=O)n2)CC1]

### 4-(2-Amino-2-methylpropanoyl)-N-(1-(4-((4-aminoazepan-1-yl)methyl)cyclohex-1-en-1-yl)-2-oxo-1,2-dihydropyrimidin-4-yl)piperazine-1-carboxamide hydrochloride salt (Compound 4)

Prepared as in Scheme C-1 from tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl)carbamate and tert-butyl azepan-4-ylcarbamate. ¹H NMR (500 MHz, D₂O) δ 7.96 (d, 1H), 6.77 (d, 1H), 6.03 (d, 1H), 3.81 (s, 3H), 3.75 (s, 5H), 3.66-3.53 (m, 3H), 3.47-3.38 (m, 1H), 3.34-3.16 (m, 3H), 2.55-2.22 (m, 7H), 2.22-1.99 (m, 4H), 1.97-1.86 (m, 1H), 1.77 (s, 6H), 1.69-1.59 (m, 1H). LCMS[(M+2H)/2]258.3.

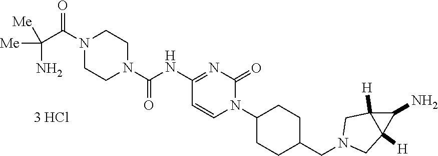 [SMILES: CC(C)(N)C(=O)N1CCN(C(=O)Nc2ccn(C3CCC(CN4C[C@@H]5[C@@H](N)[C@@H]5C4)CC3)c(=O)n2)CC1]

### 4-(2-Amino-2-methylpropanoyl)-N-(1-(4-((exo-6-amino-3-azabicyclo[3.1.0]hexan-3-yl)methyl)cyclohexyl)-2-oxo-1,2-dihydropyrimidin-4-yl)piperazine-1-carboxamide hydrochloride salt (Compound 5)

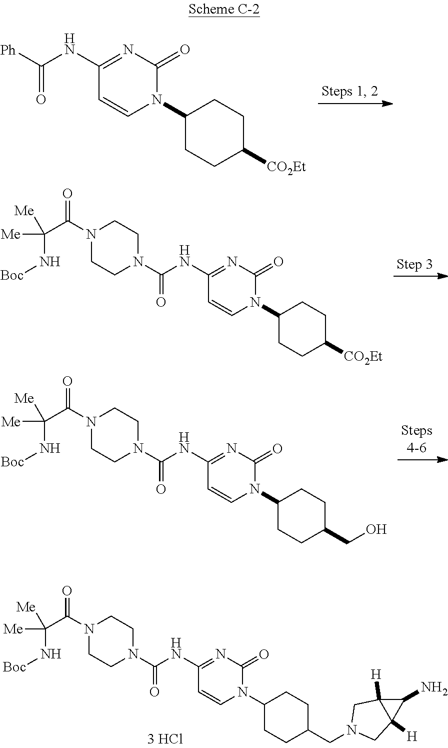

Reagents: 1) 1M HCl in EtOH, 75° C., 16 h 2) 1-(4-(2-((tert-butoxycarbonyl)amino)-2-methylpropanoyl)piperazine-1-carbonyl)-3-methyl-1H-imidazol-3-ium iodide, Et₃N, CH₃CN, heat, 17 h 3) DIBAL (25 wt % in toluene), THF, 0° C., 4 h 4) DMP, DCM, rt, 2 h 5) tert-butyl (exo-3-azabicyclo[3.1.0]hexan-6-yl)carbamate, NaBH(OAc)₃, DIPEA, DCE, CH₃CN, rt, 20 h 6) 2 M HCl in MeOH, rt, 18 h.

Step 1: ethyl cis-4-(4-amino-2-oxopyrimidin-1(2H)-yl)cyclohexane-1-carboxylate. A mixture of ethyl cis-4-(4-benzamido-2-oxopyrimidin-1(2H)-yl)cyclohexane-1-carboxylate (984 mg, 2.66 mmol) and 1M HCl in EtOH (25 mL, 25 mmol) was stirred at 7° C. for 16 h. The mixture was concentrated to dryness to afford the title compound (1.03 g). LCMS[M+H] 266.2.

Step 2: ethyl cis-4-(4-(4-(2-((tert-butoxycarbonyl)amino)-2-methylpropanoyl)piperazine-1-carboxamido)-2-oxopyrimidin-1(2H)-yl)cyclohexane-1-carboxylate. A mixture of ethyl cis-4-(4-amino-2-oxopyrimidin-1(2H)-yl)cyclohexane-1-carboxylate (984 mg, 2.66 mmol), Et₃N (0.52 mL, 3.7 mmol) and 1-(4-(2-((tert-butoxycarbonyl)amino)-2-methylpropanoyl)piperazine-1-carbonyl)-3-methyl-1H-imidazol-3-ium iodide (1.88 g, 3.7 mmol) in acetonitrile (25 mL) was stirred at reflux for 17 h. The mixture was cooled, Et₃N (0.52 mL) was added and the mixture was concentrated in vacuo. The residue was purified by flash chromatography (MeOH/EtOAc/Hexanes) to give the title compound (1.25 g) as a light yellow oil. ¹H NMR (500 MHz, CDCl₃) δ 13.08-12.57 (m, 1H), 7.24-7.09 (m, 1H), 5.90-5.66 (m, 1H), 4.95-4.69 (m, 1H), 4.58-4.40 (m, 1H), 4.17 (q, 2H), 3.91-3.64 (m, 6H), 3.63-3.49 (m, 2H), 2.73-2.65 (m, 1H), 2.34-2.21 (m, 2H), 1.84-1.75 (m, 2H), 1.74-1.60 (m, 4H), 1.49 (s, 6H), 1.41 (s, 9H), 1.30-1.26 (m, 3H). LCMS[M+H] 563.4.

Step 3: tert-butyl (1-(4-((1-(cis-4-(hydroxymethyl)cyclohexyl)-2-oxo-1,2-dihydropyrimidin-4-yl)carbamoyl)piperazin-1-yl)-2-methyl-1-oxopropan-2-yl)carbamate. DIBAL (25 wt % in toluene, 3.1 mL, 4.6 mmol) was added dropwise to a solution of ethyl cis-4-(4-(4-(2-((tert-butoxycarbonyl)amino)-2-methylpropanoyl)piperazine-1-carboxamido)-2-oxopyrimidin-1(2H)-yl)cyclohexane-1-carboxylate (513 mg, 0.912 mmol) in THF (15 mL) at 0° C. under N₂, and the mixture was stirred at ° C. for 4 h. The mixture was quenched with aq. potassium sodium tartrate (15 mL), stirred vigorously for 30 min, poured into additional aq. potassium sodium tartrate (35 mL), and extracted with DCM (3×50 mL). The extracts were washed with brine (35 mL), dried over Na₂SO₄, filtered, and concentrated in vacuo to afford the title compound (419 mg, 0.81 mmol). ¹H NMR (500 MHz, CDCl₃) δ 12.90-12.73 (m, 1H), 7.17 (d, 1H), 5.71 (d, 1H), 4.92-4.81 (m, 1H), 4.48-4.37 (m, 1H), 3.89-3.53 (m, 10H), 1.99-1.87 (m, 3H), 1.78-1.55 (m, 6H), 1.50 (s, 6H), 1.42 (s, 9H). LCMS[M+H] 521.3.

Step 4: tert-butyl (1-(4-((1-(cis-4-formylcyclohexyl)-2-oxo-1,2-dihydropyrimidin-4-yl)carbamoyl)piperazin-1-yl)-2-methyl-1-oxopropan-2-yl)carbamate. A mixture of tert-butyl (1-(4-((1-(cis-4-(hydroxymethyl)cyclohexyl)-2-oxo-1,2-dihydropyrimidin-4-yl)carbamoyl)piperazin-1-yl)-2-methyl-1-oxopropan-2-yl)carbamate (134 mg, 0.26 mmol) and DMP (327 mg, 0.77 mmol) in DCM (6 mL) was stirred at rt for 2 h. The mixture was poured into 1:1 aq. Na₂S₂O₃:sat. aq. NaHCO₃ (20 mL) and extracted with DCM (3×20 mL). The extracts were dried over Na₂SO₄, filtered, and concentrated to give the crude title compound as an off-white foam.

Step 5: tert-butyl (1-(4-((1-(4-((exo-6-((tert-butoxycarbonyl)amino)-3-azabicyclo[3.1.0]hexan-3-yl)methyl)cyclohexyl)-2-oxo-1,2-dihydropyrimidin-4-yl)carbamoyl)piperazin-1-yl)-2-methyl-1-oxopropan-2-yl)carbamate. Sodium triacetoxyborohydride (166 mg, 0.78 mmol) was added to a mixture of tert-butyl (1-(4-((1-(cis-4-formylcyclohexyl)-2-oxo-1,2-dihydropyrimidin-4-yl)carbamoyl)piperazin-1-yl)-2-methyl-1-oxopropan-2-yl)carbamate (133 mg, 0.26 mmol), tert-butyl (exo-3-azabicyclo[3.1.0]hexan-6-yl)carbamate (64 mg, 0.32 mmol), and DIPEA (90 μL, 0.52 mmol) in DCE (4 mL) and CH₃CN (2 mL), and the mixture was stirred at rt for 20 h. The mixture was poured into sat. aq. NaHCO₃ (20 mL) and extracted with DCM (3×15 mL). The extracts were dried over Na₂SO₄, decanted, and concentrated. The residue was purified by flash chromatography twice (MeOH/EtOAc/Hexanes then DCM/MeOH/NH4OH) to afford the title compound (61 mg, 0.09 mmol). LCMS[M+H] 701.6.

Step 6: 4-(2-amino-2-methylpropanoyl)-N-(1-(4-((exo-6-amino-3-azabicyclo[3.1.0]hexan-3-yl)methyl)cyclohexyl)-2-oxo-1,2-dihydropyrimidin-4-yl)piperazine-1-carboxamide hydrochloride salt. A mixture of tert-butyl (1-(4-((1-(4-((exo-6-((tert-butoxycarbonyl)amino)-3-azabicyclo[3.1.0]hexan-3-yl)methyl)cyclohexyl)-2-oxo-1,2-dihydropyrimidin-4-yl)carbamoyl)piperazin-1-yl)-2-methyl-1-oxopropan-2-yl)carbamate (61 mg, 0.09 mmol) and 2M HCl in MeOH (3 mL) was stirred at rt for 18 h. The mixture was concentrated, Et₂O (5 mL) added, the mixture sonicated, and the precipitate was collected by vacuum filtration to afford the title compound (44 mg, 0.07 mmol). ¹H NMR (500 MHz, D₂O) δ 8.14 (d, 1H), 6.75 (d, 1H), 4.52-4.43 (m, 1H), 4.30-4.19 (m, 1H), 4.02-3.92 (m, 1H), 3.88-3.69 (m, 8H), 3.67-3.57 (m, 1H), 3.24-3.16 (m, 1H), 3.15-2.92 (m, 3H), 2.56-2.48 (m, 1H), 2.48-2.41 (m, 1H), 2.10-1.95 (m, 3H), 1.92-1.82 (m, 2H), 1.80-1.68 (m, 8H), 1.38-1.26 (m, 2H). LCMS[M+H] 501.3.

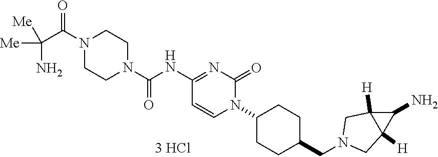

### 4-(2-Amino-2-methylpropanoyl)-N-(1-(trans-4-((exo-6-amino-3-azabicyclo[3.1.0]hexan-3-yl)methyl)cyclohexyl)-2-oxo-1,2-dihydropyrimidin-4-yl)piperazine-1-carboxamide hydrochloride salt (Compound 7)

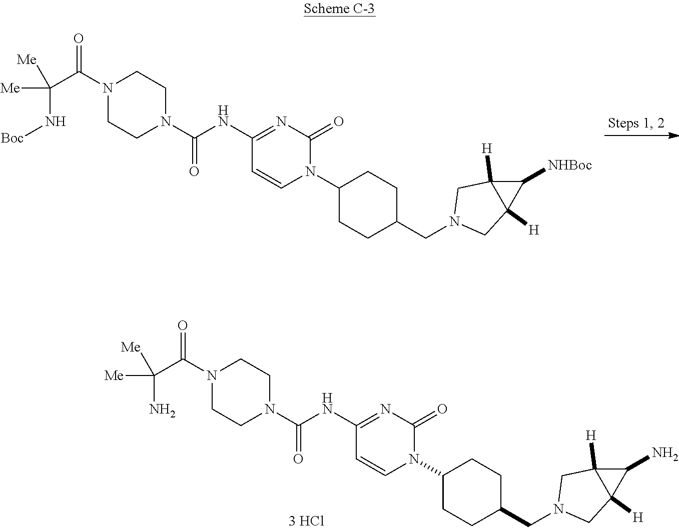

Reagents: 1) column chromatography 2) 2M HCl in MeOH, rt, 18 h.

Step 1: tert-butyl (1-(4-((1-(trans-4-((exo-6-((tert-butoxycarbonyl)amino)-3-azabicyclo[3.1.0]hexan-3-yl)methyl)cyclohexyl)-2-oxo-1,2-dihydropyrimidin-4-yl)carbamoyl)piperazin-1-yl)-2-methyl-1-oxopropan-2-yl)carbamate. A mixture of cis and trans isomers of tert-butyl (1-(4-((1-(4-((exo-6-((tert-butoxycarbonyl)amino)-3-azabicyclo[3.1.0]hexan-3-yl)methyl)cyclohexyl)-2-oxo-1,2-dihydropyrimidin-4-yl)carbamoyl)piperazin-1-yl)-2-methyl-1-oxopropan-2-yl)carbamate was purified by column chromatography (DCM/MeOH/NH₄OH then DCM/MeOH) to provide the title compound (94.0 mg). ¹H NMR (500 MHz, DMSO-d₆) δ 7.95-7.77 (m, 1H), 7.39 (s, 1H), 6.94-6.86 (m, 1H), 4.36-4.24 (m, 1H), 3.80-3.40 (m, 8H), 2.92 (d, 2H), 2.62-2.56 (m, 1H), 2.21 (d, 2H), 2.16 (d, 2H), 1.83 (d, 2H), 1.77-1.69 (m, 2H), 1.65-1.53 (m, 2H), 1.43-1.33 (m, 18H), 1.29 (s, 9H), 1.00-0.88 (m, 2H). LCMS[M+H] 701.7.

Step 2: 4-(2-amino-2-methylpropanoyl)-N-(1-(trans-4-((exo-6-amino-3-azabicyclo[3.1.0]hexan-3-yl)methyl)cyclohexyl)-2-oxo-1,2-dihydropyrimidin-4-yl)piperazine-1-carboxamide hydrochloride salt. tert-Butyl (1-(4-((1-(trans-4-((exo-6-((tert-butoxycarbonyl)amino)-3-azabicyclo[3.1.0]hexan-3-yl)methyl)cyclohexyl)-2-oxo-1,2-dihydropyrimidin-4-yl)carbamoyl)piperazin-1-yl)-2-methyl-1-oxopropan-2-yl)carbamate (94 mg, 134 μmol) and 2M HCl in MeOH (3.3 mL, 6.7 mmol) was stirred at rt for 18 h. IPA (3 mL) was added and the precipitate was collected by vacuum filtration, and washed with IPA and Et₂O. The filtrate was concentrated and purified by reverse phase prep HPLC (CH₃CN/H₂O w/0.1% TFA). Product fractions were collected, concentrated and 2M HCl in MeOH added. The solvent was removed under reduced pressure and the solid was combined with the original precipitate to afford the title compound (71 mg). ¹H NMR (500 MHz, D₂O) δ 8.18 (d, 1H), 6.74 (d, 1H), 4.53-4.43 (m, 1H), 4.29-4.20 (m, 1H), 4.00-3.93 (m, 1H), 3.87-3.70 (m, 8H), 3.66-3.56 (m, 1H), 3.20 (d, 1H), 3.16-3.01 (m, 2H), 2.99-2.92 (m, 1H), 2.56-2.48 (m, 1H), 2.48-2.41 (m, 1H), 2.11-1.95 (m, 4H), 1.95-1.81 (m, 2H), 1.80-1.68 (m, 7H), 1.38-1.25 (m, 2H). LCMS[M+H] 501.4.

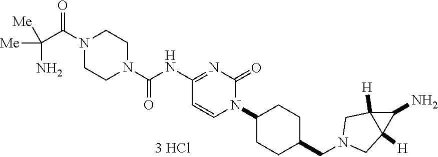

### 4-(2-Amino-2-methylpropanoyl)-N-(1-(cis-4-((exo-6-amino-3-azabicyclo[3.1.0]hexan-3-yl)methyl)cyclohexyl)-2-oxo-1,2-dihydropyrimidin-4-yl)piperazine-1-carboxamide hydrochloride salt (Compound 6)

Prepared as in Scheme C-3 from a mixture of cis and trans isomers of tert-butyl (1-(4-((1-(4-((exo-6-((tert-butoxycarbonyl)amino)-3-azabicyclo[3.1.0]hexan-3-yl)methyl)cyclohexyl)-2-oxo-1,2-dihydropyrimidin-4-yl)carbamoyl)piperazin-1-yl)-2-methyl-1-oxopropan-2-yl)carbamate. ¹H NMR (500 MHz, D₂O) δ 8.14 (d, 1H), 6.75 (d, 1H), 4.47 (t, 1H), 4.30-4.18 (m, 1H), 3.96 (d, 1H), 3.89-3.67 (m, 8H), 3.66-3.57 (m, 1H), 3.24-3.15 (m, 1H), 3.14-2.99 (m, 2H), 2.98-2.90 (m, 1H), 2.57-2.49 (m, 1H), 2.48-2.40 (m, 1H), 2.10-1.95 (m, 3H), 1.94-1.80 (m, 2H), 1.80-1.68 (m, 8H), 1.38-1.24 (m, 2H). LCMS[M+H] 501.4.

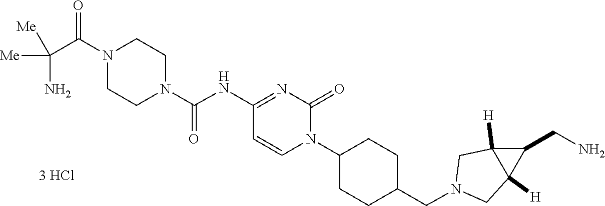 [SMILES: CC(C)(N)C(=O)N1CCN(C(=O)Nc2ccn(C3CCC(CN4C[C@@H]5[C@@H](CN)[C@@H]5C4)CC3)c(=O)n2)CC1]

### 4-(2-Amino-2-methylpropanoyl)-N-(1-(4-((exo-6-(aminomethyl)-3-azabicyclo[3.1.0]hexan-3-yl)methyl)cyclohexyl)-2-oxo-1,2-dihydropyrimidin-4-yl)piperazine-1-carboxamide hydrochloride salt (Compound 8)

Prepared in a similar fashion to Scheme C-2 from tert-butyl (1-(4-((1-(cis-4-formylcyclohexyl)-2-oxo-1,2-dihydropyrimidin-4-yl)carbamoyl)piperazin-1-yl)-2-methyl-1-oxopropan-2-yl)carbamate and tert-butyl ((exo-3-azabicyclo[3.1.0]hexan-6-yl)methyl)carbamate. ¹H NMR (500 MHz, D₂O) δ 8.15 (d, 1H), 6.75 (d, 1H), 4.52-4.43 (m, 1H), 4.20-4.11 (m, 1H), 3.93-3.84 (m, 1H), 3.83-3.68 (m, 8H), 3.57-3.47 (m, 2H), 3.45-3.40 (m, 1H), 3.18 (d, 1H), 3.04-2.93 (m, 2H), 2.16-2.09 (m, 1H), 2.09-1.95 (m, 4H), 1.94-1.80 (m, 3H), 1.80-1.67 (m, 7H), 1.52-1.41 (m, 1H), 1.37-1.25 (m, 2H). LCMS[M+H] 515.3.

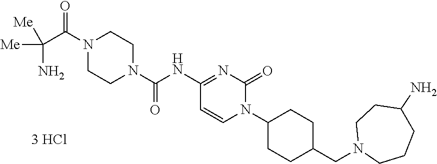 [SMILES: CC(C)(N)C(=O)N1CCN(C(=O)Nc2ccn(C3CCC(CN4CCCC(N)CC4)CC3)c(=O)n2)CC1]

### 4-(2-Amino-2-methylpropanoyl)-N-(1-(4-((4-aminoazepan-1-yl)methyl)cyclohexyl)-2-oxo-1,2-dihydropyrimidin-4-yl)piperazine-1-carboxamide hydrochloride salt (Compound 9)

Prepared in a similar fashion to Scheme C-2 from tert-butyl (1-(4-((1-(cis-4-formylcyclohexyl)-2-oxo-1,2-dihydropyrimidin-4-yl)carbamoyl)piperazin-1-yl)-2-methyl-1-oxopropan-2-yl)carbamate and tert-butyl azepan-4-ylcarbamate. ¹H NMR (500 MHz, D₂O) δ 8.05 (dd, 1H), 6.78 (d, 1H), 4.52-4.42 (m, 1H), 3.91-3.67 (m, 8H), 3.65-3.49 (m, 3H), 3.44-3.34 (m, 1H), 3.28-3.11 (m, 3H), 2.42-2.28 (m, 2H), 2.28-2.20 (m, 1H), 2.20-2.08 (m, 2H), 2.08-1.94 (m, 4H), 1.93-1.84 (m, 2H), 1.82-1.68 (m, 8H), 1.41-1.27 (m, 2H). LCMS[M+H] 517.4.

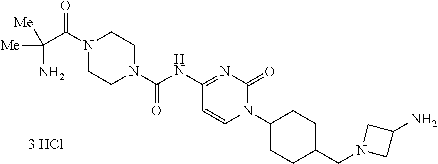 [SMILES: CC(C)(N)C(=O)N1CCN(C(=O)Nc2ccn(C3CCC(CN4CC(N)C4)CC3)c(=O)n2)CC1]

### 4-(2-Amino-2-methylpropanoyl)-N-(1-(4-((3-aminoazetidin-1-yl)methyl)cyclohexyl)-2-oxo-1,2-dihydropyrimidin-4-yl)piperazine-1-carboxamide hydrochloride salt (Compound 10)

Prepared in a similar fashion to Scheme C-2 from tert-butyl (1-(4-((1-(cis-4-formylcyclohexyl)-2-oxo-1,2-dihydropyrimidin-4-yl)carbamoyl)piperazin-1-yl)-2-methyl-1-oxopropan-2-yl)carbamate and tert-butyl azetidin-3-ylcarbamate. ¹H NMR (500 MHz, D₂O) δ 8.14 (d, 1H), 6.75 (d, 1H), 4.63-4.36 (m, 5H), 3.88-3.68 (m, 8H), 3.37-3.31 (m, 1H), 2.11-2.02 (m, 2H), 1.99-1.92 (m, 2H), 1.92-1.80 (m, 2H), 1.79-1.68 (m, 7H), 1.40-1.29 (m, 2H). LCMS[M+H] 475.3.

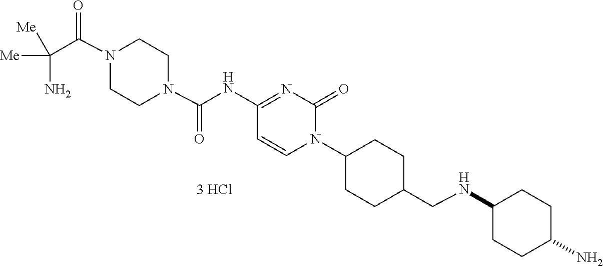 [SMILES: CC(C)(N)C(=O)N1CCN(C(=O)Nc2ccn(C3CCC(CN[C@H]4CC[C@H](N)CC4)CC3)c(=O)n2)CC1]

### 4-(2-Amino-2-methylpropanoyl)-N-(1-(4-(((trans-4-aminocyclohexyl)amino)methyl)cyclohexyl)-2-oxo-1,2-dihydropyrimidin-4-yl)piperazine-1-carboxamide hydrochloride salt (Compound 11)

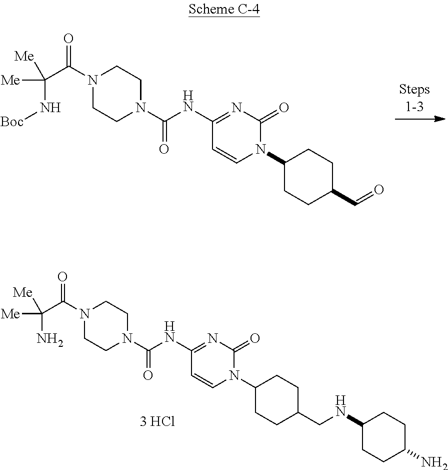

Reagents: 1) tert-butyl (trans-4-aminocyclohexyl)carbamate, NaBH₃CN, MeOH, rt, 20 h 2) Boc₂O, sat. NaHCO₃, THF, rt, 19 h 3) 2M HCl in MeOH, rt, 19 h.

Step 1: tert-butyl (1-(4-((1-(-4-(((trans-4-((tert-butoxycarbonyl)amino)cyclohexyl)amino)methyl)cyclohexyl)-2-oxo-1,2-dihydropyrimidin-4-yl)carbamoyl)piperazin-1-yl)-2-methyl-1-oxopropan-2-yl)carbamate. Sodium cyanoborohydride (25 mg, 0.4 mmol) was added to a mixture of tert-butyl (1-(4-((1-(cis-4-formylcyclohexyl)-2-oxo-1,2-dihydropyrimidin-4-yl)carbamoyl)piperazin-1-yl)-2-methyl-1-oxopropan-2-yl)carbamate (61 mg, 0.12 mmol) and tert-butyl (trans-4-aminocyclohexyl)carbamate (32 mg, 0.15 mmol) in MeOH (2 mL) and the mixture was stirred at rt for 20 h. The reaction mixture was purified by flash chromatography (DCM/MeOH/NH₄OH) to give the title compound (50 mg), as a mixture of cis and trans diastereomers. LCMS[M+H] 717.6.

Step 2: tert-butyl ((4-(4-(4-(2-((tert-butoxycarbonyl)amino)-2-methylpropanoyl)piperazine-1-carboxamido)-2-oxopyrimidin-1(2H)-yl)cyclohexyl)methyl)(trans-4-((tert-butoxycarbonyl)amino)cyclohexyl)carbamate. A mixture of tert-butyl (1-(4-((1-(4-(((trans-4-((tert-butoxycarbonyl)amino)cyclohexyl)amino)methyl)cyclohexyl)-2-oxo-1,2-dihydropyrimidin-4-yl)carbamoyl)piperazin-1-yl)-2-methyl-1-oxopropan-2-yl)carbamate (50 mg, 0.07 mmol) and di-tert-butyl dicarbonate (30 mg, 0.14 mmol) in THF (0.75 mL) and sat. sodium bicarbonate (0.75 mL) was stirred at rt for 19 h. The mixture was diluted with EtOAc (10 mL), washed with sat. NaHCO₃ (2×10 mL) and brine (1×10 mL), dried over Na₂SO₄, decanted, and concentrated. The residue was purified by flash chromatography (MeOH/EtOAc/Hexanes) to afford the title compound (31 mg) as a mixture of cis and trans diastereomers. LCMS[M+H] 817.7.

Step 3: 4-(2-amino-2-methylpropanoyl)-N-(1-(4-(((trans-4-aminocyclohexyl)amino)methyl)cyclohexyl)-2-oxo-1,2-dihydropyrimidin-4-yl)piperazine-1-carboxamide hydrochloride salt. A mixture of tert-butyl ((4-(4-(4-(2-((tert-butoxycarbonyl)amino)-2-methylpropanoyl)piperazine-1-carboxamido)-2-oxopyrimidin-1(2H)-yl)cyclohexyl)methyl)(trans-4-((tert-butoxycarbonyl)amino)cyclohexyl)carbamate (31 mg, 0.04 mmol) and 2M HCl in MeOH (1.5 mL) was stirred at rt for 19 h. The mixture was concentrated in vacuo and dried from EtOH and Et₂O to afford the title compound (24 mg). ¹H NMR (500 MHz, D₂O) δ 8.26-8.15 (m, 1H), 6.79-6.70 (m, 1H), 4.54-4.44 (m, 1H), 3.93-3.66 (m, 8H), 3.33-3.19 (m, 3H), 3.09-3.01 (m, 1H), 2.39-2.27 (m, 2H), 2.26-2.18 (m, 2H), 2.11-1.98 (m, 2H), 1.94-1.81 (m, 4H), 1.80-1.69 (m, 7H), 1.66-1.51 (m, 4H), 1.39-1.22 (m, 2H). LCMS[M+H] 517.4.

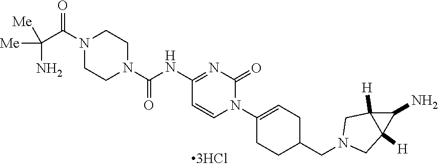 [SMILES: CC(C)(N)C(=O)N1CCN(C(=O)Nc2ccn(C3=CCC(CN4C[C@@H]5[C@@H](N)[C@@H]5C4)CC3)c(=O)n2)CC1]

### 4-(2-Amino-2-methylpropanoyl)-N-(1-(4-(((1R,5S,6s)-6-amino-3-azabicyclo[3.1.0]hexan-3-yl)methyl)cyclohex-1-en-1-yl)-2-oxo-1,2-dihydropyrimidin-4-yl)piperazine-1-carboxamide hydrochloride salt (Compound 12)

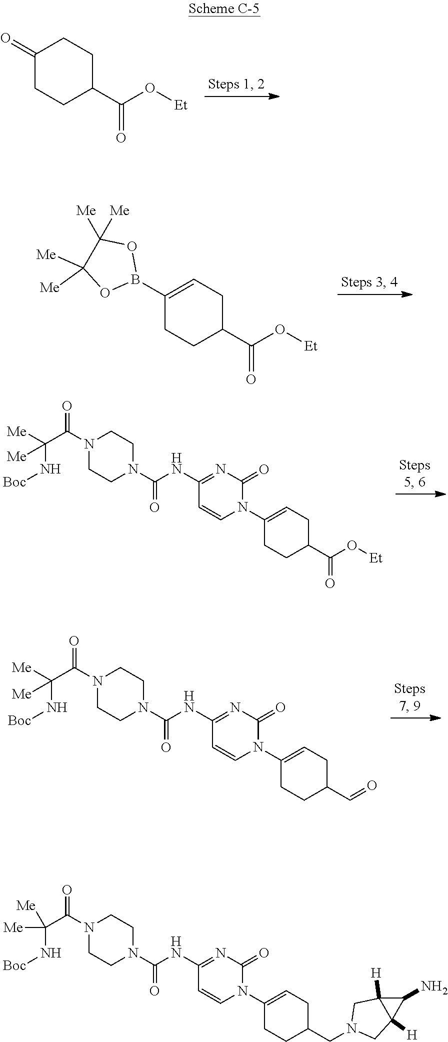 [SMILES: CCOC(=O)C1CCC(=O)CC1 ; CCOC(=O)C1CC=C(B2OC(C)(C)C(C)(C)O2)CC1 ; CCOC(=O)C1CC=C(n2ccc(NC(=O)N3CCN(C(=O)C(C)(C)NC(=O)OC(C)(C)C)CC3)nc2=O)CC1 ; CC(C)(C)OC(=O)NC(C)(C)C(=O)N1CCN(C(=O)Nc2ccn(C3=CCC(C=O)CC3)c(=O)n2)CC1 ; CC(C)(C)OC(=O)NC(C)(C)C(=O)N1CCN(C(=O)Nc2ccn(C3=CCC(CN4C[C@@H]5[C@@H](N)[C@@H]5C4)CC3)c(=O)n2)CC1]

Reagents: Step 1) 2,6-di-tert-butyl-4-methylpyridine, Tf₂O, DCM, rt, 8 h 2) PdCl₂(dppf).DCM, KOAc, B₂pin₂, dioxane, 100° C., 2 h 3) cytosine, Cu(OAc)₂·H₂O, TMEDA, MeOH, water, rt, 72 h 4) ACN, 80° C., 16 h 5) LiBH₄, THF, rt, 18 h 6) DMP, DCM, rt, 2 h 7) Exo-tert-butyl (3-azabicyclo[3.1.0]hexan-6-yl)carbamate, NaBH(OAc)₃, DCM, rt, 2 h 8) 4M HCl in Dioxane, DCM, rt, 2 h.

Step 1: ethyl 4-(((trifluoromethyl)sulfonyl)oxy)cyclohex-3-ene-1-carboxylate. To a stirred solution of ethyl 4-oxocyclohexane-1-carboxylate (1.0 g, 5.8 mmol) and 2,6-di-tert-butyl-4-methylpyridine (1.5 g, 7.6 mmol) in DCM (25 mL) was dropwise added Tf₂O (1.14 mL, 7.0 mmol) at 0° C. The resulting reaction mixture was stirred at rt for 8 h. The reaction mixture was poured into NaHCO₃ solution (100 mL) and extracted with DCM (2×100 mL). The combined organic phases were dried over Na₂SO₄, concentrated under reduced pressure. The crude material was purified by column chromatography (5% EtOAc in Hexane) to afford the title compound (1.2 g) as a colorless liquid. ¹H NMR (400 MHz, CDCl₃): δ 5.78 (s, 1H), 4.20-4.14 (m, 2H), 2.63-2.57 (m, 1H), 2.47-2.45 (m, 2H), 2.43-2.42 (m, 2H), 2.18-2.11 (m, 1H), 1.98-1.90 (m, 1H), 1.29 (t, 3H).

Step 2: ethyl 4-(4,4,5,5-tetramethyl-1,3,2-dioxaborolan-2-yl)cyclohex-3-ene-1-carboxy late. To a stirred solution of ethyl 4-(((trifluoromethyl)sulfonyl)oxy)cyclohex-3-ene-1-carboxylate (0.75 g, 2.4 mmol) in dioxane (10 mL) was added KOAc (0.73 g, 7.4 mmol) and B₂Pin₂ (0.69 g, 2.7 mmol) then reaction mixture was purged with N₂ for 30 min. PdCl₂(dppf).DCM (0.1 g, 0.12 mmol) was added at rt. The reaction mixture was heated at 100° C. for 2 h. The reaction mixture was poured into water (100 mL) and extracted with EtOAc (2×100 mL). The combined organic phase was dried over Na₂SO₄, concentrated under reduced pressure. The crude material was purified by column chromatography (EtOAc/Hexane) to afford the title compound (1.5 g) as a pale yellow liquid.

Step 3: ethyl 4-(4-amino-2-oxopyrimidin-1(2H)-yl)cyclohex-3-ene-1-carboxylate. To a stirred solution of ethyl 4-(4,4,5,5-tetramethyl-1,3,2-dioxaborolan-2-yl)cyclohex-3-ene-1-carboxylate (1.5 g, 5.3 mmol) in methanol:H₂O (40 mL:10 mL) was added cytosine (0.59 g, 5.3 mmol) at rt in open air. After 30 min, Cu(OAc)₂·H₂O (1.06 g, 5.3 mmol) and TMEDA (1.0 mL, 6.4 mmol) were added. The resulting reaction mixture was stirred at rt for 72 h. The reaction mixture was concentrated under reduced pressure and diluted with H₂O (50 mL). The solids that precipitated were filtered and washed with H₂O (3×50 mL) and hexanes (20 mL) to afford the title compound (0.26 g). ¹H NMR (400 MHz, DMSO-d₆): δ 7.34 (d, 1H), 7.13 (s, 1H), 7.04 (s, 1H), 5.62 (t, 2H), 4.11-4.06 (m, 2H), 2.57 (m, 1H), 2.32-2.30 (m, 4H), 2.00-1.96 (m, 1H), 1.71-1.65 (m, 1H), 1.21 (t, 3H). LCMS [M+H] 264.1.

Step 4: ethyl 4-(4-(4-(2-((tert-butoxycarbonyl)amino)-2-methylpropanoyl)piperazine-1-carboxamido)-2-oxopyrimidin-1(2H)-yl)cyclohex-3-ene-1-carboxylate. To a stirred solution of ethyl 4-(4-amino-2-oxopyrimidin-1(2H)-yl)cyclohex-3-ene-1-carboxylate (0.25 g, 0.95 mmol) and 1-(4-(2-((tert-butoxycarbonyl)amino)-2-methylpropanoyl)piperazine-1-carbonyl)-3-methyl-1H-imidazol-3-ium iodide (0.73 g, 1.42 mmol) in ACN (15 mL). The reaction mixture was heated to 80° C. for 16 h. The progress of reaction was monitored by TLC. After completion of reaction, the reaction mixture was concentrated under reduced pressure to get crude material which was purified by column chromatography (7% MeOH:MDC) to afford the title compound (0.36 g, 67%) as a pale yellow. LCMS[M+H] 561.31.

Step 5: tert-butyl (1-(4-((1-(4-(hydroxymethyl)cyclohex-1-en-1-yl)-2-oxo-1,2-dihydro pyrimidin-4-yl)carbamoyl)piperazin-1-yl)-2-methyl-1-oxopropan-2-yl)carbamate. To a stirred solution of ethyl 4-(4-(4-(2-((tert-butoxycarbonyl)amino)-2-methylpropanoyl) piperazine-1-carboxamido)-2-oxopyrimidin-1(2H)-yl)cyclohex-3-ene-1-carboxylate (0.35 g, 0.62 mmol) in THF (10 mL) was added LiBH₄ (0.07 g, 3.12 mmol) at rt. The resulting reaction mixture was stirred at rt for 18 h. The progress of reaction was monitored by TLC. After completion of reaction, the reaction mixture was poured into saturated NH₄Cl solution (50 mL) and extracted with MDC:IPA (7:3) (2×50 mL) and the combined organic phase was dried over Na₂SO₄, concentrated under reduced pressure to get crude material which was purified by column chromatography (15% MeOH:MDC) to afford the title compound (0.23 g, 70%). LCMS [M+H] 519.37.

Step 6: tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl)carbamate. To a stirred solution of tert-butyl (1-(4-((1-(4-(hydroxymethyl)cyclohex-1-en-1-yl)-2-oxo-1,2-dihydropyrimidin-4-yl)carbamoyl)piperazin-1-yl)-2-methyl-1-oxopropan-2-yl)carbamate (0.23 g, 0.44 mmol) in DCM (10 mL) was added DMP (0.38 g, 0.88 mmol) at rt. The resulting reaction mixture was stirred at rt for 2 h. The progress of reaction was monitored by TLC. After completion of reaction, the reaction mixture was diluted with DCM (100 mL) and washed with saturated NaHCO₃ solution (2×50 mL). The organic phase was dried over Na₂SO₄, filtered and concentrated under reduced pressure to get crude material which was purified by trituration (diethyl ether) to afford the title compound (0.2 g, 80%). LCMS[M+H] 517.22.

Step 7: Exo-tert-butyl (1-(4-((1-(4-((6-((tert-butoxycarbonyl)amino)-3-azabicyclo [3.1.0]hexan-3-yl)methyl)cyclohex-1-en-1-yl)-2-oxo-1,2-dihydropyrimidin-4-yl)carbamoyl) piperazin-1-yl)-2-methyl-1-oxopropan-2-yl)carbamate. To a stirred solution of tert-butyl (1-(4-((1-(4-formylcyclohex-1-en-1-yl)-2-oxo-1,2-dihydropyrimidin-4-yl)carbamoyl) piperazin-1-yl)-2-methyl-1-oxopropan-2-yl)carbamate (0.2 g, 0.38 mmol) and exo-tert-butyl (3-azabicyclo[3.1.0]hexan-6-yl)carbamate (0.08 g, 0.38 mmol) in DCE (5 mL) was added NaBH(OAc)₃ (0.12 g, 0.58 mmol) after 15 min. The reaction mixture was stirred at rt for 2 h. The progress of reaction was monitored by TLC. After completion of reaction, the resulting reaction mixture was poured into saturated NaHCO₃ solution (50 mL) and extracted with DCM (2×50 mL). The combined organic phase was dried over Na₂SO₄, filtered and concentrated under reduced pressure to get crude material which was purified by column chromatography (8% MeOH:MDC) to afford the title compound (0.12 g, 25%). LCMS[M+H] 699.4.

Step 8: exo-4-(2-Amino-2-methylpropanoyl)-N-(1-(4-((6-amino-3-azabicyclo[3.1.0]hexan-3-yl)methyl)cyclohex-1-en-1-yl)-2-oxo-1,2-dihydropyrimidin-4-yl)piperazine-1-carboxamide. To a stirred solution exo-tert-butyl (1-(4-((1-(4-((6-((tert-butoxy carbonyl)amino)-3-azabicyclo[3.1.0]hexan-3-yl)methyl)cyclohex-1-en-1-yl)-2-oxo-1,2-dihydropyrimidin-4-yl)carbamoyl)piperazin-1-yl)-2-methyl-1-oxopropan-2-yl)carbamate (0.12 g, 0.11 mmol) in DCM (20 mL) was added 4N HCl in dioxane (0.5 mL) at rt. The reaction mixture was stirred at rt for 2 h. The reaction mixture was concentrated under reduced pressure and purified by prep HPLC (0.01% HCl in H₂O:MeOH) to afford the title compound (0.06 g, 50%). ¹H NMR (400 MHz, D₂O): δ 7.75 (d, 1H), 6.61 (d, 1H), 5.82 (s, 1H), 4.10 (bs, 1H), 3.84-3.81 (m, 2H), 3.36-3.57 (m, 8H), 3.49-3.41 (m, 1H), 3.15-3.05 (m, 2H), 2.93-2.81 (m, 2H), 2.29-2.18 (m, 4H), 2.10-2.03 (m, 1H), 1.94-1.85 (m, 2H), 1.58 (s, 6H), 1.43-1.35 (m, 1H). LCMS [M+H] 499.4.

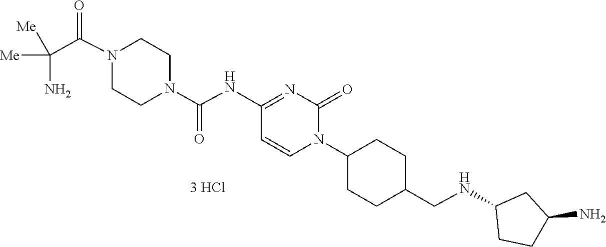 [SMILES: CC(C)(N)C(=O)N1CCN(C(=O)Nc2ccn(C3CCC(CN[C@H]4CC[C@H](N)C4)CC3)c(=O)n2)CC1]

### 4-(2-Amino-2-methylpropanoyl)-N-(1-(4-((((1S,3S)-3-aminocyclopentyl)amino)methyl)cyclohexyl)-2-oxo-1,2-dihydropyrimidin-4-yl)piperazine-1-carboxamide hydrochloride salt (Compound 13)

Prepared in a similar fashion to Scheme C-5 from tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and tert-butyl ((1S,3S)-3-aminocyclopentyl)carbamate. ¹H NMR (500 MHz, D₂O) δ 8.21-8.14 (m, 1H), 6.78-6.72 (m, 1H), 4.54-4.44 (m, 1H), 3.97-3.85 (m, 2H), 3.85-3.69 (m, 8H), 3.30-3.21 (m, 1H), 3.08-2.99 (m, 1H), 2.49-2.35 (m, 2H), 2.35-2.26 (m, 2H), 2.26-2.19 (m, 1H), 2.10-1.98 (m, 2H), 1.93-1.78 (m, 6H), 1.76 (s, 6H), 1.40-1.28 (m, 2H). LCMS[M+H] 503.3.

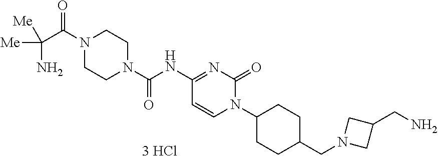 [SMILES: CC(C)(N)C(=O)N1CCN(C(=O)Nc2ccn(C3CCC(CN4CC(CN)C4)CC3)c(=O)n2)CC1]

### 4-(2-Amino-2-methylpropanoyl)-N-(1-(4-((3-(aminomethyl)azetidin-1-yl)methyl)cyclohexyl)-2-oxo-1,2-dihydropyrimidin-4-yl)piperazine-1-carboxamide hydrochloride salt (Compound 14)

Prepared in a similar fashion to Scheme C-2 from tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and tert-butyl (azetidin-3-ylmethyl)carbamate as a mixture of diastereomers. ¹H NMR (500 MHz, D₂O) δ 8.14-8.07 (m, 1H), 6.80-6.73 (m, 1H), 4.55-4.40 (m, 2H), 4.37-4.25 (m, 2H), 4.13-4.03 (m, 1H), 3.88-3.66 (m, 8H), 3.56-3.42 (m, 2H), 3.40-3.32 (m, 2H), 3.31-3.19 (m, 1H), 2.19-2.00 (m, 2H), 1.98-1.79 (m, 4H), 1.79-1.67 (m, 7H), 1.40-1.26 (m, 2H). LCMS[M+H] 489.2.

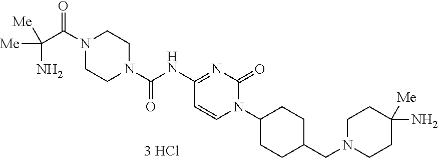 [SMILES: CC1(N)CCN(CC2CCC(n3ccc(NC(=O)N4CCN(C(=O)C(C)(C)N)CC4)nc3=O)CC2)CC1]

### 4-(2-amino-2-methylpropanoyl)-N-(1-(4-((4-amino-4-methylpiperidin-1-yl)methyl)cyclohexyl)-2-oxo-1,2-dihydropyrimidin-4-yl)piperazine-1-carboxamide hydrochloride salt (Compound 15)

Prepared in a similar fashion to Scheme C-2 from tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and tert-butyl (4-methylpiperidin-4-yl)carbamate. ¹H NMR (500 MHz, D₂O) δ 8.19-8.11 (m, 1H), 6.79-6.73 (m, 1H), 4.54-4.44 (m, 1H), 3.91-3.62 (m, 9H), 3.45-3.36 (m, 2H), 3.36-3.22 (m, 2H), 3.22-3.12 (m, 1H), 2.43-2.35 (m, 1H), 2.33-2.12 (m, 4H), 2.11-1.97 (m, 2H), 1.96-1.85 (m, 3H), 1.84-1.70 (m, 7H), 1.62-1.52 (m, 3H), 1.42-1.29 (m, 2H). LCMS[M+H] 517.3.

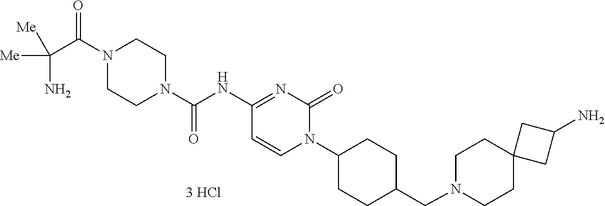 [SMILES: CC(C)(N)C(=O)N1CCN(C(=O)Nc2ccn(C3CCC(CN4CCC5(CC4)CC(N)C5)CC3)c(=O)n2)CC1]

### 4-(2-Amino-2-methylpropanoyl)-N-(1-(4-((2-amino-7-azaspiro[3.5]nonan-7-yl)methyl)cyclohexyl)-2-oxo-1,2-dihydropyrimidin-4-yl)piperazine-1-carboxamide hydrochloride salt (Compound 16)

Prepared in a similar fashion to Scheme C-2 from tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and tert-butyl (7-azaspiro[3.5]nonan-2-yl)carbamat. ¹H NMR (500 MHz, D₂O) δ 8.21-8.13 (m, 1H), 6.78-6.72 (m, 1H), 4.54-4.43 (m, 1H), 3.92 (p, 1H), 3.86-3.68 (m, 8H), 3.64-3.47 (m, 2H), 3.30 (d, 1H), 3.12-2.94 (m, 3H), 2.58-2.48 (m, 1H), 2.41-2.31 (m, 2H), 2.18-2.03 (m, 4H), 2.03-1.84 (m, 7H), 1.83-1.69 (m, 8H), 1.40-1.27 (m, 1H). LCMS[M+H] 543.5.

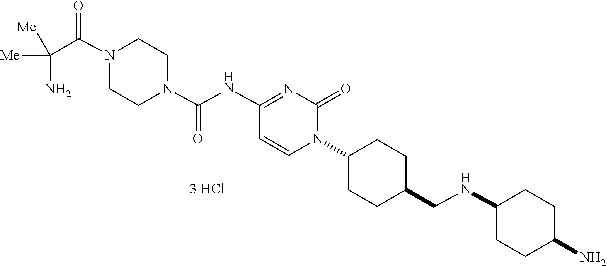 [SMILES: CC(C)(N)C(=O)N1CCN(C(=O)Nc2ccn([C@H]3CC[C@H](CN[C@H]4CC[C@@H](N)CC4)CC3)c(=O)n2)CC1]

### 4-(2-Amino-2-methylpropanoyl)-N-(1-(trans-4-(((cis-4-aminocyclohexyl)amino)methyl)cyclohexyl)-2-oxo-1,2-dihydropyrimidin-4-yl)piperazine-1-carboxamide hydrochloride salt (Compound 17)

Prepared in a similar fashion to Schemes C-3 from tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and tert-butyl (cis-4-aminocyclohexyl)carbamate. ¹H NMR (400 MHz, D₂O) δ 8.04 (d, 1H), 6.60 (d, 1H), 4.42-4.30 (m, 1H), 3.80-3.53 (m, 8H), 3.51-3.40 (m, 1H), 3.29-3.16 (m, 1H), 2.93 (d, 2H), 2.03-1.78 (m, 10H), 1.78-1.53 (m, 11H), 1.29-1.11 (m, 2H). LCMS[M+H] 517.9.

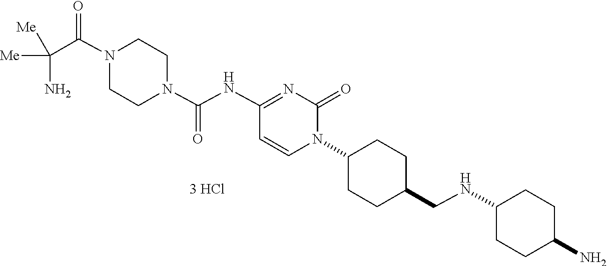 [SMILES: CC(C)(N)C(=O)N1CCN(C(=O)Nc2ccn([C@H]3CC[C@H](CN[C@H]4CC[C@H](N)CC4)CC3)c(=O)n2)CC1]

### 4-(2-Amino-2-methylpropanoyl)-N-(1-(trans-4-(((trans-4-aminocyclohexyl)amino)methyl)cyclohexyl)-2-oxo-1,2-dihydropyrimidin-4-yl)piperazine-1-carboxamide hydrochloride salt (Compound 18)

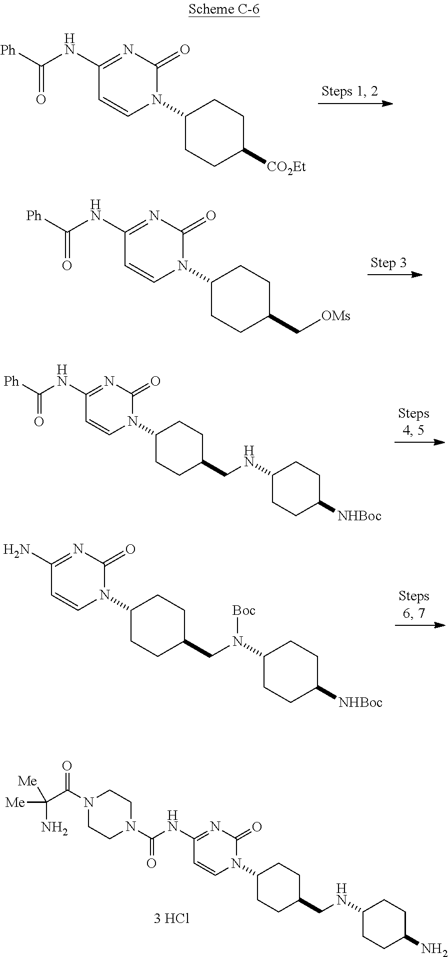

Reagents: 1) DIBAL, THF, 0° C., 2 h 2) MsCl, Et₃N, DCM, rt, 2.5 h 3) tert-butyl (trans-4-aminocyclohexyl)carbamate, K₂CO₃, NaI, CH₃CN, 92° C., 42 h 4) Boc₂O, dioxane, sat. aq. NaHCO₃, rt, 16 h 5) NH₄OH, MeOH, 50° C., 2.5 h 6) 1-(4-(2-((tert-butoxycarbonyl)amino)-2-methylpropanoyl)piperazine-1-carbonyl)-3-methyl-1H-imidazol-3-ium iodide, CH₃CN, Q, 22 h 7) 2M HCl in MeOH, rt, 17 h.

Step 1: N-(1-(trans-4-(hydroxymethyl)cyclohexyl)-2-oxo-1,2-dihydropyrimidin-4-yl)benzamide. DIBAL (25 wt % in toluene, 8.3 mL) was added dropwise to a solution of ethyl trans-4-(4-benzamido-2-oxopyrimidin-1(2H)-yl)cyclohexane-1-carboxylate (1.13 g, 3.06 mmol) in THF (30 mL) at 0° C., and the mixture was stirred at 0° C. for 2 h. 1M HCl (50 mL) was added the mixture extracted with EtOAc (3×50 mL). The extracts were washed with brine (75 mL). The biphasic mixture was filtered. The filtrate was separated and the aqueous layer was extracted with EtOAc (75 mL). The combined organic layers were dried (Na₂SO₄), filtered, and concentrated. The precipitate and the EtOAc extracts were combined to give the title compound (1.0 g). LCMS[M+H] 328.1.

Step 2: (trans-4-(4-benzamido-2-oxopyrimidin-1(2H)-yl)cyclohexyl)methyl methanesulfonate. MsCl (0.36 mL, 4.6 mmol) was added dropwise to a mixture of N-(1-(trans-4-(hydroxymethyl)cyclohexyl)-2-oxo-1,2-dihydropyrimidin-4-yl)benzamide (1.0 g, 3.0 mmol) and Et₃N (0.85 mL, 6.1 mmol) in DCM (60 mL), and the mixture was stirred at rt for 2.5 h. The mixture was purified by column chromatography (Hex/EtOAc/MeOH then MeOH/DCM) to give the title compound (1.57 g). LCMS[M+H] 406.2.

Step 3: tert-butyl (trans-4-(((trans-4-(4-benzamido-2-oxopyrimidin-1(2H)-yl)cyclohexyl)methyl)amino)cyclohexyl)carbamate. A mixture of (trans-4-(4-benzamido-2-oxopyrimidin-1(2H)-yl)cyclohexyl)methyl methanesulfonate (943 mg, 1.86 mmol), tert-butyl (trans-4-aminocyclohexyl)carbamate (797 mg, 3.72 mmol), K₂CO₃ (514 mg, 3.72 mmol), and NaI (55.8 mg, 372 μmol) in CH₃CN (25 mL) was stirred at 92° C. for 42 h. The mixture was cooled, diluted with MeOH (50 mL), concentrated and purified by column chromatography (DCM/MeOH/NH₄OH) to give the title compound (409 mg). LCMS[M+H] 524.3.

Step 4: tert-butyl ((trans-4-(4-benzamido-2-oxopyrimidin-1(2H)-yl)cyclohexyl)methyl)(trans-4-((tert-butoxycarbonyl)amino)cyclohexyl)carbamate. A mixture of tert-butyl (trans-4-(((trans-4-(4-benzamido-2-oxopyrimidin-1(2H)-yl)cyclohexyl)methyl)amino)cyclohexyl)carbamate (306 mg, 584 μmol) and di-tert-butyl dicarbonate (0.32 mL, 1.4 mmol) in dioxane (12 mL) and sat. aq. NaHCO₃ (6.0 mL) was stirred at rt for 16 h. It was diluted with EtOAc (65 mL), washed with sat. aq. NaHCO₃ (1×50 mL) and brine (1×50 mL), dried (Na₂SO₄), decanted, and concentrated. The residue was purified by column chromatography (Hex/EtOAc/MeOH) to give the title compound (129 mg). ¹H NMR (500 MHz, CDCl₃) δ 7.89 (d, 2H), 7.69 (d, 1H), 7.61 (t, 1H), 7.55-7.42 (m, 3H), 4.73-4.61 (m, 1H), 4.42-4.30 (m, 2H), 3.45-3.31 (m, 2H), 3.02 (d, 2H), 2.12-1.96 (m, 6H), 1.89 (d, 2H), 1.82-1.73 (m, 1H), 1.70-1.55 (m, 2H), 1.50-1.38 (m, 18H), 1.28-1.12 (m, 6H). LCMS[M+H] 624.4.

Step 5: tert-butyl ((trans-4-(4-amino-2-oxopyrimidin-1(2H)-yl)cyclohexyl)methyl)(trans-4-((tert-butoxycarbonyl)amino)cyclohexyl)carbamate. A mixture of tert-butyl ((trans-4-(4-benzamido-2-oxopyrimidin-1(2H)-yl)cyclohexyl)methyl)(trans-4-((tert-butoxycarbonyl)amino)cyclohexyl)carbamate (129 mg, 207 μmol) and 29 wt % NH₄OH (0.71 mL) in MeOH (3 mL) was stirred at 50° C. for 2.5 h. The mixture was concentrated in vacuo, then concentrated from MeOH then CH₃CN to give the title compound. LCMS[M+H] 520.3.

Step 6: tert-butyl ((trans-4-(4-(4-(2-((tert-butoxycarbonyl)amino)-2-methylpropanoyl)piperazine-1-carboxamido)-2-oxopyrimidin-1(2H)-yl)cyclohexyl)methyl)(trans-4-((tert-butoxycarbonyl)amino)cyclohexyl)carbamate. A mixture of tert-butyl ((trans-4-(4-amino-2-oxopyrimidin-1(2H)-yl)cyclohexyl)methyl)(trans-4-((tert-butoxycarbonyl)amino)cyclohexyl)carbamate (207 μmol) and 1-(4-(2-((tert-butoxycarbonyl)amino)-2-methylpropanoyl)piperazine-1-carbonyl)-3-methyl-1H-imidazol-3-ium iodide (191 mg, 376 μmol) in CH₃CN (5.0 mL) was stirred at reflux for 22 h. The mixture was cooled, Et₃N (0.1 mL) was added, and the mixture was concentrated. The residue was purified by column chromatography (Hex/EtOAc/MeOH) to give the title compound (116 mg). LCMS[M+H] 817.6.

Step 7: 4-(2-amino-2-methylpropanoyl)-N-(1-(trans-4-(((trans-4-aminocyclohexyl)amino)methyl)cyclohexyl)-2-oxo-1,2-dihydropyrimidin-4-yl)piperazine-1-carboxamide hydrochloride salt. A mixture of tert-butyl ((trans-4-(4-(4-(2-((tert-butoxycarbonyl)amino)-2-methylpropanoyl)piperazine-1-carboxamido)-2-oxopyrimidin-1(2H)-yl)cyclohexyl)methyl)(trans-4-((tert-butoxycarbonyl)amino)cyclohexyl)carbamate (116 mg, 142 μmol) and 2M HCl in MeOH (2.8 mL, 5.5 mmol) was stirred at rt for 17 h. IPA (3 mL) was added to the mixture and the precipitate was collected by vacuum filtration, rinsed with additional IPA and with Et₂O, to afford the title compound (88.3 mg). ¹H NMR (500 MHz, D₂O) δ 8.14 (d, 1H), 6.75 (d, 1H), 4.53-4.44 (m, 1H), 3.93-3.65 (m, 8H), 3.32-3.18 (m, 2H), 3.05 (d, 2H), 2.35-2.17 (m, 4H), 2.10-1.96 (m, 4H), 1.91-1.80 (m, 1H), 1.79-1.67 (m, 8H), 1.64-1.50 (m, 4H), 1.39-1.26 (m, 2H). LCMS[M+H] 517.3.

Alternatively, Compound 18 may be prepared according to Scheme C-7.

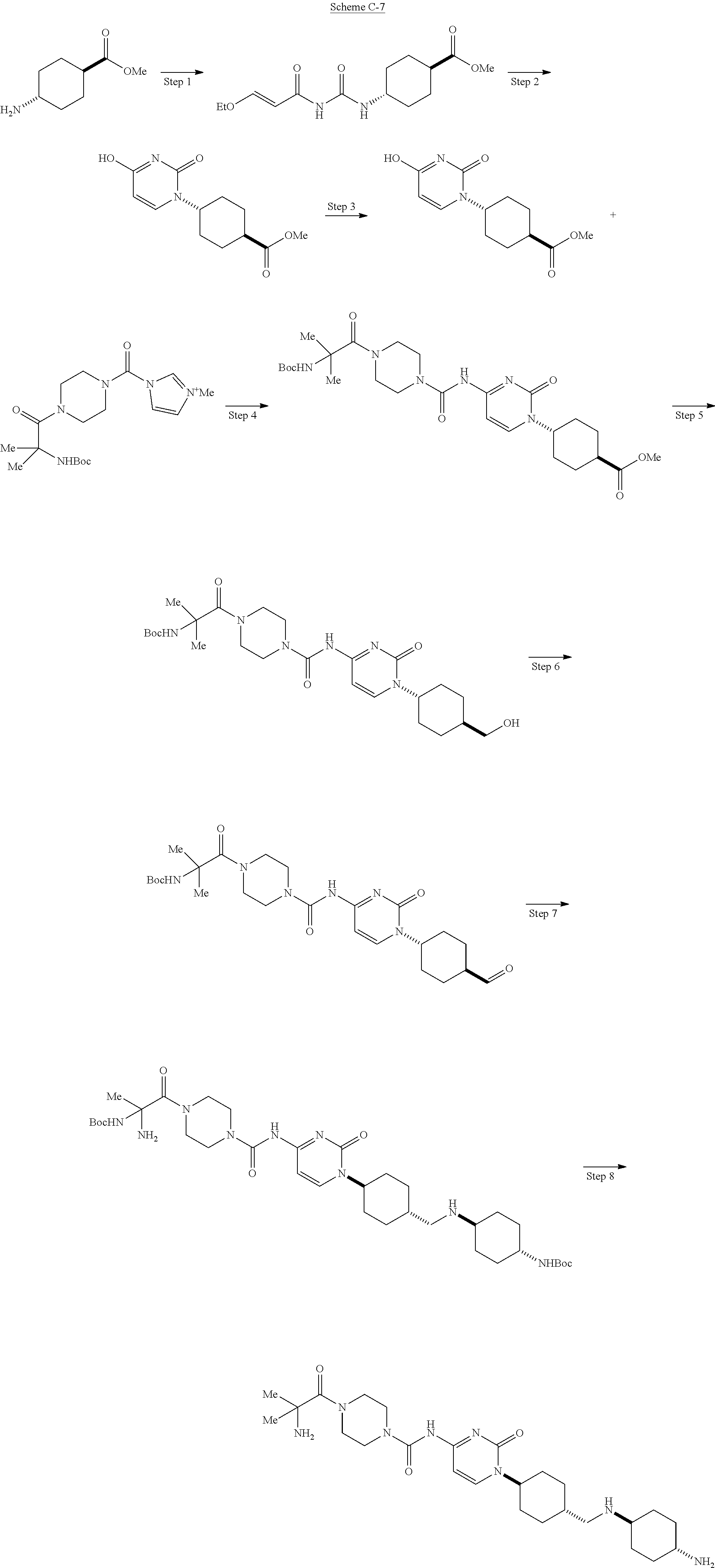

Reagents: Step 1) 3-ethoxyacryloyl chloride, silver cyanate, toluene, THF, rt, 16 h 2) 2M HCl, MeOH, 80° C., 12 h 3) 1,2,4 triazole, POCl₃, TEA, ACN, 20° C., 6 h, 28% NH₄OH solution, 50° C., 16 h 4) ACN, 80° C., 16 h 5) LiBH4, THF, rt, 16 h 6) DMP, DCM, rt, 2 h 7) tert-butyl (4-aminocyclohexyl) carbamate, NaBH(OAc)₃, DCE, rt, 2 h 8) 4M HCl in dioxane, DCM, rt, 2 h.

Step 1: trans-methyl (E)-4-(3-(3-ethoxyacryloyl)ureido) cyclohexane-1-carboxylate. To a stirred solution of silver cyanate (2.3 g, 15.48 mmol) in toluene (30 mL) was dropwise added 3-ethoxy acryloyl chloride (2.08 g, 15.46 mmol) at rt. The resulting reaction mixture was stirred at 80° C. for 30 min and cooled to rt, Trans-methyl-4-aminocyclohexane-1-carboxylate HCl salt (1.0 g, 5.16 mmol) in THF (40 mL) was added at −30° C. The reaction mixture was stirred at for 16 h. The reaction mixture was filtered through Celite® and washed with ethyl acetate (100 mL). The organic layer was concentrated under reduced pressure and the crude material was purified by column chromatography (40% EtOAc in Hexane) to afford the title compound (3.7 g). LCMS [M+H] 298.7.

Step 2: trans-methyl 4-(4-hydroxy-2-oxopyrimidin-1(2H)-yl)cyclohexane-1-carboxylate. To a stirred solution of trans-methyl (E)-4-(3-(3-ethoxyacryloyl)ureido) cyclohexane-1-carboxylate (0.5 g, 1.84 mmol) in MeOH (10 mL) was added 2M HCl solution (4.5 mL, 9.0 mmol). The resulting reaction mixture was heated at 80° C. for 12 h. The reaction mixture was poured into NaHCO₃ solution (200 mL) and extracted with DCM (2×200 mL). The combined organic phases were dried over Na₂SO₄ and concentrated under reduced pressure to afford the title compound (1.3 g, 30% over two step). LCMS [M+H] 252.8.

Step 3: trans-methyl 4-(4-amino-2-oxopyrimidin-1(2H)-yl)cyclohexane-1-carboxylate. POCl₃ (0.9 mL, 9.7 mmol) was added dropwise to a mixture of 1,2,4 triazole (4.8 g, 69 mmol) and TEA (11.2 mL, 77.7 mmol) in ACN (5 mL) at 0° C. The reaction mixture was stirred at 0° C. for 20 min. Trans-methyl 4-(4-hydroxy-2-oxopyrimidin-1(2H)-yl)cyclohexane-1-carboxylate (0.7 g, 2.8 mmol) in ACN (10 mL) was added and then reaction mixture was warmed to 20° C. and stirred for 6 h. Ammonium hydroxide solution (19 mL) was added to the reaction mixture and stirred at 50° C. for 16 h. The reaction mixture was concentrated under reduced pressure and purified by reverse phase chromatography (40% MeOH in H₂O) to afford the title compound (0.35 g, 65%). ¹H NMR (400 MHz, D₂O): δ 7.60 (d, 1H), 7.00-6.91 (m, 2H), 5.66 (d, 1H), 4.36-4.30 (m, 1H), 3.58 (s, 3H), 2.35-2.29 (m, 1H), 2.02-1.99 (m, 2H), 1.74-1.70 (m, 2H), 1.63-1.56 (m, 2H), 1.54-1.47 (m, 2H). LCMS [M+1]251.9.

Step 4: trans-methyl 4-(4-(4-(2-((tert-butoxycarbonyl)amino)-2-methylpropanoyl) piperazine-1-carboxamido)-2-oxopyrimidin-1(2H)-yl)cyclohexane-1-carboxylate. To a stirred solution of trans-methyl 4-(4-amino-2-oxopyrimidin-1(2H)-yl)cyclohexane-1-carboxylate (0.45 g, 1.8 mmol) and 1-(4-(2-((tert-butoxycarbonyl)amino)-2-methylpropanoyl)piperazine-1-carbonyl)-3-methyl-1H-imidazol-3-ium iodide (1.36 g, 2.68 mmol) in ACN (20 mL). The reaction mixture was heated at 80° C. for 16 h. The reaction mixture was concentrated under reduced pressure and purified by flash chromatography (9% MeOH:DCM) to afford the title compound (0.9 g, 90%). LCMS[M+H] 549.1.

Step 5: trans-tert-butyl (1-(4-((1-(4-(hydroxymethyl)cyclohexyl)-2-oxo-1,2-dihydropyrimidin-4-yl)carbamoyl)piperazin-1-yl)-2-methyl-1-oxopropan-2-yl)carbamate. To a stirred solution of trans-methyl 4-(4-(4-(2-((tert-butoxycarbonyl)amino)-2-methylpropanoyl) piperazine-1-carboxamido)-2-oxopyrimidin-1(2H)-yl)cyclohexane-1-carboxylate (0.9 g, 1.64 mmol) in THF (30 mL) was added LiBH₄ (0.36 g, 16.4 mmol) at rt. The reaction mixture was poured into ice water (100 mL) and extracted with DCM (2×100 mL) and the combined organic phases were dried over Na₂SO₄, filtered and concentrated under reduced pressure. The crude material was purified by reverse phase chromatography (25% MeOH in H₂O) to afford the title compound (0.55 g, 75%). LCMS [M+H] 521.3.

Step 6: trans-tert-butyl (1-(4-((1-(4-formylcyclohexyl)-2-oxo-1,2-dihydropyrimidin-4-yl)carbamoyl) piperazin-1-yl)-2-methyl-1-oxopropan-2-yl)carbamate. To a stirred solution of trans-tert-butyl (1-(4-((1-(4-(hydroxymethyl)cyclohexyl)-2-oxo-1,2-dihydropyrimidin-4-yl)carbamoyl)piperazin-1-yl)-2-methyl-1-oxopropan-2-yl)carbamate (0.55 g, 1.05 mmol) in DCM (20 mL) was added DMP (0.89 g, 2.1 mmol) at rt. The resulting reaction mixture was stirred at rt for 2 h. The reaction mixture was diluted with DCM (100 mL) and washed with saturated NaHCO₃ solution (3×100 mL). The organic layer was dried over Na₂SO₄, filtered and concentrated under reduced pressure. The crude material was purified by trituration with diethyl ether (10 mL) to afford the title compound (0.5 g, 80%).

Step 7: trans-trans-tert-butyl (1-(4-((1-(4-((4-((tert-butoxycarbonyl)amino)cyclohexylamino) methyl) cyclohexyl)-2-oxo-1,2-dihydropyrimidin-4-yl)carbamoyl)piperazin-1-yl)-2-methyl-1-oxopropan-2-yl)carbamate. To a stirred solution of trans-tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate (0.5 g, 0.96 mmol) and trans-tert-butyl (4-aminocyclohexyl) carbamate (0.3 g, 1.4 mmol) in DCE (15 mL) was added NaBH(OAc)₃ (1.0 g, 4.8 mmol) at rt. The reaction mixture was poured into saturated NaHCO₃ solution (50 mL) and extracted with DCM (2×100 mL). The organic layer was washed with NH₄Cl solution (3×50 mL), dried over Na₂SO₄, filtered and concentrated under reduced pressure. The crude material was purified by reverse phase chromatography (80% MeOH in H₂O) to afford the title compound (0.13 g, 17%). LCMS[M+H] 717.5.

Step 8: trans-trans-4-(2-amino-2-methylpropanoyl)-N-(1-(4-((4-aminocyclohexylamino) methyl) cyclohexyl)-2-oxo-1,2-dihydropyrimidin-4-yl)piperazine-1-carboxamide hydrochloride. To a stirred solution of trans-trans-tert-butyl (1-(4-((1-(4-((4-((tert-butoxycarbonyl)amino)cyclohexyl amino) methyl) cyclohexyl)-2-oxo-1,2-dihydropyrimidin-4-yl)carbamoyl)piperazin-1-yl)-2-methyl-1-oxopropan-2-yl)carbamate (0.12 g, 0.16 mmol) in DCM (20 mL) was added 4N HCl in dioxane (1.0 mL) at rt. The reaction mixture was stirred at rt for 2 h. The reaction mixture was concentrated under reduced pressure and purified by prep HPLC purification (0.01% HCl in H₂O/MeOH) to afford the title compound (0.035 g, 35%). 1H NMR (400 MHz, D₂O): δ 7.98 (d, 1H), 6.62 (d, 1H), 4.34 (t, 1H), 3.65-3.59 (m, 8H), 3.23-3.02 (m, 2H), 2.90 (d, 2H), 2.61 (s, 1H), 2.14-2.09 (m, 4H), 1.89 (t, 4H), 1.78-1.69 (m, 1H), 1.61 (s, 7H), 1.57-1.43 (m, 4H), 1.23-1.17 (m, 2H). LCMS [M+1]517.8.

Alternatively, Compound 18 may be prepared according to Scheme C-8.

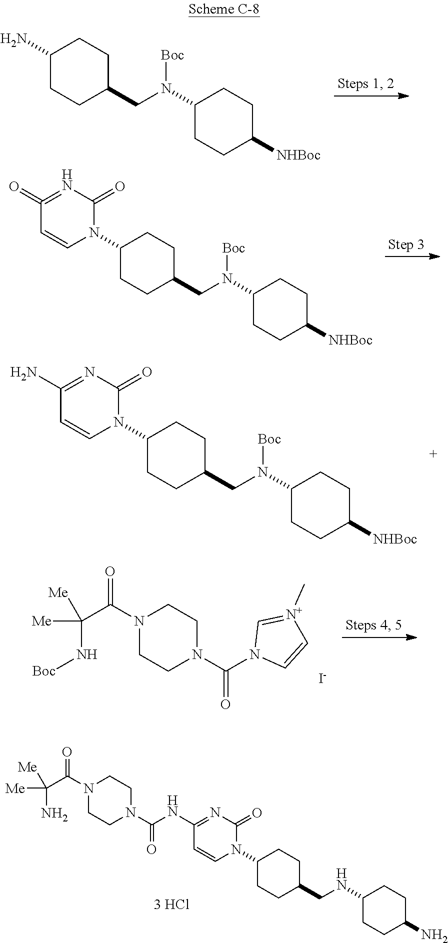

Reagents: 1) i) 3-ethoxyacryloyl chloride, AgOCN, toluene, Q, 4 h ii) DCM, 0° C. to rt, 18 h 2) i) TFA, rt, 3 h ii) Boc₂O, sat. aq. NaHCO₃, dioxane, 4 h 3) POCl₃, 1,2,4-triazole, Et₃N, CH₃CN, rt, 3 h, then NH₄OH, 50° C., 18 h 4) CH₃CN, heat, 17 h 5) 2M HCl/MeOH, rt, 18 h.

Step 1: tert-butyl (trans-4-((tert-butoxycarbonyl)amino)cyclohexyl) ((trans-4-(3-(3-ethoxyacryloyl)ureido)cyclohexyl) methyl)carbamate. A solution of 3-ethoxyacryloyl chloride (3.15 g, 23 mmol) in toluene (67 mL) was added dropwise to a suspension of silver cyanate (3.87 g, 26 mmol) in toluene (50 mL) and the mixture was stirred at reflux for 4 h. The reaction mixture was cooled to rt and allowed to settle. A portion of the solution formed (82 mL, 0, 16.4 mmol) was transferred by cannula into an addition funnel and added dropwise into a cooled (−78° C.) solution of tert-butyl ((trans-4-aminocyclohexyl)methyl)(trans 4-((tert-butoxycarbonyl)amino)cyclohexyl)carbamate (5 g, 12 mmol) in dry DCM (100 mL) under N₂. The mixture was warmed to rt and stirred for 18 h. MeOH (50 mL) was added and the mixture was concentrated to dryness. The residue was purified by column chromatography (EtOAc/Hexanes) to give the title compound (5.90 g) as a light yellow foam. ¹H NMR (500 MHz, MeOD) δ 7.64 (d, 1H), 5.41 (d, 1H), 3.97 (q, 2H), 3.70-3.51 (m, 2H), 3.30-3.21 (m, 1H), 3.04 (d, 2H), 2.08-1.99 (m, 2H), 1.99-1.91 (m, 2H), 1.85-1.65 (m, 6H), 1.63-1.51 (m, 1H), 1.46 (s, 9H), 1.43 (s, 9H), 1.33 (t, 3H), 1.30-1.17 (m, 4H), 1.14-1.02 (m, 2H). LCMS[M-2(t-Bu)+H] 455.2.

Step 2: tert-butyl (trans-4-((tert-butoxycarbonyl)amino)cyclohexyl) ((trans-4-(2,4-dioxo-3,4-dihydropyrimidin-1(2H)-yl)cyclohexyl)methyl)carbamate. A mixture of tert-butyl (trans-4-((tert-butoxycarbonyl)amino)cyclohexyl)((trans-4-(3-(3-ethoxyacryloyl)ureido)cyclohexyl)methyl)carbamate (5.9 g, 10.4 mmol) and TFA (40 mL, 520 mmol) was stirred at rt for 3 h, then concentrated to dryness. Dioxane (100 mL) and sat. aq. NaHCO₃ (100 mL) were added. 2M NaOH (20 mL) was added until the pH was 8. Di-tert-butyl dicarbonate (4.8 mL, 21 mmol) was added and the mixture was stirred vigorously for 3 d. Additional di-tert-butyl dicarbonate (2.4 mL, 11 mmol) was added and the reaction mixture stirred for 1 d. The mixture was diluted with EtOAc (500 mL), washed with sat. aq. NaHCO₃ (2×500 mL) and brine (1×500 mL), dried over Na₂SO₄, filtered, and concentrated. The residue was purified by column chromatography (EtOAc/Hex) to give the title compound (5.30 g) as a white foam. ¹H NMR (500 MHz, MeOD) δ 7.69 (d, 1H), 5.66 (d, 1H), 4.36 (tt, 1H), 3.34-3.22 (m, 2H), 3.07 (d, 2H), 2.00-1.93 (m, 2H), 1.93-1.84 (m, 4H), 1.84-1.69 (m, 4H), 1.69-1.58 (m, 3H), 1.47 (s, 9H), 1.43 (s, 9H), 1.34-1.22 (m, 2H), 1.22-1.10 (m, 2H). LCMS[M-2(t-Bu)+H] 409.1.

Step 3: tert-butyl ((trans-4-(4-amino-2-oxopyrimidin-1(2H)-yl)cyclohexyl)methyl)(trans-4-((tert-butoxycarbonyl)amino)cyclohexyl)carbamate. POCl₃ (2.85 mL, 30.5 mmol) was added dropwise to a mixture of 1,2,4-triazole (17 g, 244 mmol) and Et₃N (34.1 mL, 244 mmol) in CH₃CN (50 mL) at 0° C., and the mixture was stirred at 0° C. for 15 min. A solution of tert-butyl (trans-4-((tert-butoxycarbonyl)amino)cyclohexyl)((trans-4-(2,4-dioxo-3,4-dihydropyrimidin-1(2H)-yl)cyclohexyl)methyl)carbamate (5.3 g, 10 mmol) in CH₃CN (50 mL) was added dropwise, and the mixture was warmed to rt and stirred for 3 h. 28 wt % NH₄OH (72 mL, 509 mmol) was added and the mixture was stirred at 50° C. for 18 h. Water and ice were added (500 mL) and the mixture was stirred for 30 min. The precipitate was collected by vacuum filtration, CH₃CN (200 mL) added, concentrated under reduced pressure, and dried under vacuum to give the title compound (4.66 g). ¹H NMR (500 MHz, MeOD) δ 7.66 (d, 1H), 5.87 (d, 1H), 4.45 (tt, 1H), 3.73-3.59 (m, 1H), 3.31-3.23 (m, 1H), 3.08 (d, 2H), 2.00-1.92 (m, 2H), 1.92-1.83 (m, 4H), 1.83-1.68 (m, 4H), 1.68-1.53 (m, 3H), 1.47 (s, 9H), 1.43 (s, 9H), 1.34-1.11 (m, 4H). LCMS[M+H] 520.2.

Step 4: tert-butyl ((trans-4-(4-(4-(2-((tert-butoxycarbonyl)amino)-2-methylpropanoyl)piperazine-1-carboxamido)-2-oxopyrimidin-1(2H)-yl)cyclohexyl)methyl)(trans-4-((tert-butoxycarbonyl)amino)cyclohexyl)carbamate. A mixture of tert-butyl ((trans-4-(4-amino-2-oxopyrimidin-1(2H)-yl)cyclohexyl)methyl)(trans-4-((tert-butoxycarbonyl)amino)cyclohexyl)carbamate (4.66 g, 8.97 mmol) and 1-(4-(2-((tert-butoxycarbonyl)amino)-2-methylpropanoyl)piperazine-1-carbonyl)-3-methyl-1H-imidazol-3-ium iodide (8.24 g, 16 mmol) in CH₃CN (90 mL) was stirred at reflux for 17 h. The mixture was cooled, diluted with EtOAc (500 mL), washed with sat. aq. NaHCO₃ (2×500 mL) and brine (500 mL), dried over Na₂SO₄, decanted, and concentrated. The residue was purified by column chromatography (Hex/EtOAc/MeOH) to give the title compound (5.91 g). LCMS[M+H] 817.6.

Step 5: 4-(2-amino-2-methylpropanoyl)-N-(1-(trans-4-(((trans-4-aminocyclohexyl)amino)methyl)cyclohexyl)-2-oxo-1,2-dihydropyrimidin-4-yl)piperazine-1-carboxamide hydrochloride salt. A mixture of tert-butyl ((trans-4-(4-(4-(2-((tert-butoxycarbonyl)amino)-2-methylpropanoyl)piperazine-1-carboxamido)-2-oxopyrimidin-1(2H)-yl)cyclohexyl)methyl)(trans-4-((tert-butoxycarbonyl)amino)cyclohexyl)carbamate (5.9 g, 7.2 mmol) and 2M HCl in MeOH (150 mL, 300 mmol) was stirred at rt for 18 h. IPA (150 mL) was added and the mixture was stirred for 1 h, the precipitate was collected by vacuum filtration and washed with IPA and Et₂O. The solid was lyophilized from water to give the title compound (4.50 g). ¹H NMR (500 MHz, D₂O) δ 8.20 (d, 1H), 6.74 (d, 1H), 4.50 (tt, 1H), 3.94-3.66 (m, 8H), 3.32-3.18 (m, 2H), 3.05 (d, 2H), 2.35-2.18 (m, 4H), 2.11-1.97 (m, 4H), 1.91-1.80 (m, 1H), 1.80-1.68 (m, 8H), 1.64-1.50 (m, 4H), 1.40-1.27 (m, 2H). LCMS[M+H] 517.3.

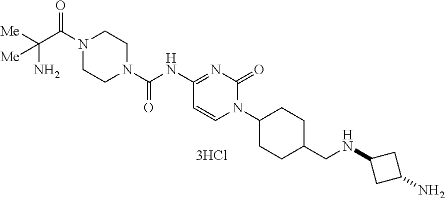 [SMILES: CC(C)(N)C(=O)N1CCN(C(=O)Nc2ccn(C3CCC(CN[C@H]4C[C@H](N)C4)CC3)c(=O)n2)CC1]

### trans-4-(2-Amino-2-methylpropanoyl)-N-(1-(4-(((3-aminocyclobutyl)amino)methyl)cyclohexyl)-2-oxo-1,2-dihydropyrimidin-4-yl)piperazine-1-carboxamide hydrochloride salt (Compound 43)

Prepared in a similar fashion to Scheme C-7 from trans-tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and trans-tert-butyl (3-aminocyclobutyl)carbamate. LCMS [M+H] 489.6.

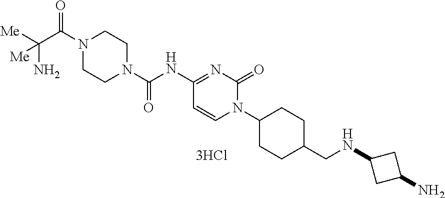 [SMILES: CC(C)(N)C(=O)N1CCN(C(=O)Nc2ccn(C3CCC(CN[C@H]4C[C@@H](N)C4)CC3)c(=O)n2)CC1]

### cis-4-(2-Amino-2-methylpropanoyl)-N-(1-(4-(((3-aminocyclobutyl)amino)methyl)cyclohexyl)-2-oxo-1,2-dihydropyrimidin-4-yl)piperazine-1-carboxamide hydrochloride salt (Compound 44)

Prepared in a similar fashion to Scheme C-7 from trans-tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and cis-tert-butyl (3-aminocyclobutyl)carbamate. LCMS [M+H] 489.6.

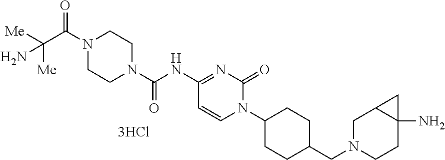 [SMILES: CC(C)(N)C(=O)N1CCN(C(=O)Nc2ccn(C3CCC(CN4CCC5(N)CC5C4)CC3)c(=O)n2)CC1]

### 4-(2-Amino-2-methylpropanoyl)-N-(1-(4-((6-amino-3-azabicyclo[4.1.0]heptan-3-yl)methyl)cyclohexyl)-2-oxo-1,2-dihydropyrimidin-4-yl)piperazine-1-carboxamide hydrochloride salt (Compound 45)

Prepared in a similar fashion to Scheme C-7 from trans-tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and tert-butyl (3-azabicyclo[4.1.0]heptan-6-yl)carbamate. LCMS [M+H] 515.8.

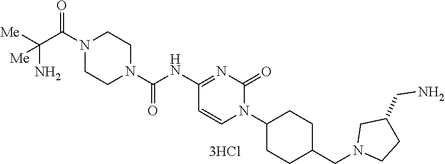 [SMILES: CC(C)(N)C(=O)N1CCN(C(=O)Nc2ccn(C3CCC(CN4CC[C@@H](CN)C4)CC3)c(=O)n2)CC1]

### (S)-4-(2-Amino-2-methylpropanoyl)-N-(1-(4-((3-(aminomethyl)pyrrolidin-1-yl)methyl)cyclohexyl)-2-oxo-1,2-dihydropyrimidin-4-yl)piperazine-1-carboxamide hydrochloride salt (Compound 46)

Prepared in a similar fashion to Scheme C-7 from trans-tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and (S)-tert-butyl (pyrrolidin-3-ylmethyl)carbamate. ¹H NMR: (400 MHz, D₂O): δ 7.92 (d, 1H), 6.63 (d, 1H), 4.32 (t, 2H), 3.84-3.81 (m, 1H), 3.64-3.57 (m, 8H), 3.39 (d, 1H), 3.12-3.02 (m, 6H), 2.84-2.81 (m, 1H), 2.70-2.60 (m, 1H), 2.91-2.80 (m, 1H), 2.78-2.68 (m, 1H), 1.91-1.86 (m, 4H), 1.85-1.76 (m, 1H), 1.60 (s, 6H), 1.24-1.18 (m, 2H). LCMS [M+H] 503.7.

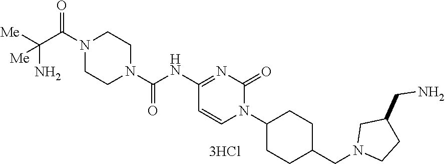 [SMILES: CC(C)(N)C(=O)N1CCN(C(=O)Nc2ccn(C3CCC(CN4CC[C@H](CN)C4)CC3)c(=O)n2)CC1]

### (R)-4-(2-Amino-2-methylpropanoyl)-N-(1-(4-((3-(aminomethyl)pyrrolidin-1-yl)methyl)cyclohexyl)-2-oxo-1,2-dihydropyrimidin-4-yl)piperazine-1-carboxamide Hydrochloride (Compound 47)

Prepared in a similar fashion to Scheme C-7 using trans-tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and (R)-tert-butyl (pyrrolidin-3-ylmethyl)carbamate. LCMS[M+H] 503.4.

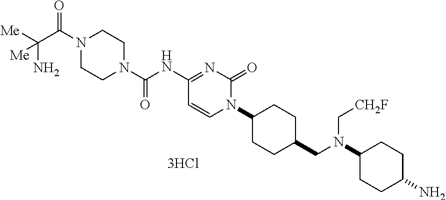

### trans-cis-4-(2-Amino-2-methylpropanoyl)-N-(1-(4-(((4-aminocyclohexyl)(2-fluoroethyl)amino)methyl)cyclohexyl)-2-oxo-1,2-dihydropyrimidin-4-yl)piperazine-1-carboxamide hydrochloride salt (Compound 48)

Prepared in a similar fashion to Scheme C-7 from trans-tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and -tert-butyl ((trans)-4-((2-fluoroethyl)amino)cyclohexyl) carbamate. A cis/trans mixture was observed by TLC and ¹H NMR of tert-butyl (1-(4-((1-((trans)-4-((((trans)-4-((tert-butoxycarbonyl)amino)cyclohexyl)(2-fluoroethyl)amino)methyl)cyclohexyl)-2-oxo-1,2-dihydropyrimidin-4-yl)carbamoyl)piperazin-1-yl)-2-methyl-1-oxopropan-2-yl)carbamate. Purification of individual isomers was accomplished by Waters SFC 350 Column Chiralpack® IA 250*50 mm, 5 micron, 2489 UV detector, Liq.CO₂ mobile phase with 0.1% triethylamine in isopropyl alcohol:tert-butylmethylether:acetonitrile (50:25:25), with an Isocratic Method (55% A, 45% B), Flow rate 180 ml/min, Sample loading 20 mg, 32 min. run. Deprotection as in Scheme C-7 afforded the desired product. First Fraction: ¹H NMR: (400 MHz, D2O): δ 7.93 (d, 1H), 6.65 (d, 1H), 4.62-4.59 (m, 1H), 4.39-4.28 (m, 1H), 3.72-3.59 (m, 8H), 3.49-3.43 (m, 2H), 3.42-3.37 (m, 1H), 3.24-3.17 (m, 3H), 2.23-2.08 (m, 5H), 1.77-1.72 (m, 6H), 1.69-1.66 (m, 4H), 1.62. (s, 6H), 1.54-1.45 (m, 3H). LCMS[M+H] 563.6.

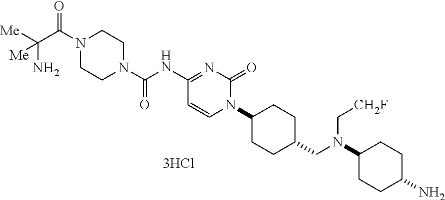

### trans-trans-4-(2-amino-2-methylpropanoyl)-N-(1-(4-(((4-aminocyclohexyl)(2-fluoroethyl)amino)methyl)cyclohexyl)-2-oxo-1,2-dihydropyrimidin-4-yl)piperazine-1-carboxamide hydrochloride salt (Compound 49)

Prepared in a similar fashion to trans-cis-4-(2-Amino-2-methylpropanoyl)-N-(1-(4-(((4-aminocyclohexyl)(2-fluoroethyl)amino)methyl)cyclohexyl)-2-oxo-1,2-dihydropyrimidin-4-yl)piperazine-1-carboxamide hydrochloride salt. 2^{nd} Fraction. ¹H NMR: (400 MHz, D₂O): δ 7.92 (d, 1H), 6.64 (d, 1H), 4.89-4.84 (m, 1H), 4.46-4.32 (m, 1H), 3.83-3.59 (m, 8H), 3.50-3.39 (m, 2H), 3.30-3.04 (m, 4H), 2.25-2.04 (m, 4H), 2.02-1.89 (m, 4H), 1.56 (s, 13H), 1.24-1.13 (m, 2H). LCMS[M+H] 563.6.

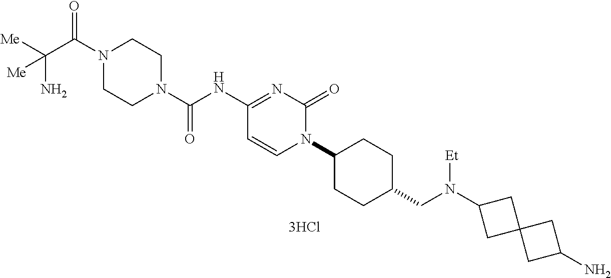

### trans-4-(2-Amino-2-methylpropanoyl)-N-(1-(4-(((6-aminospiro[3.3]heptan-2-yl)(ethyl)amino)methyl)cyclohexyl)-2-oxo-1,2-dihydropyrimidin-4-yl)piperazine-1-carboxamide hydrochloride salt (Compound 50)

Prepared in a similar fashion to Scheme C-7 using trans-tert-butyl (6-(((4-(4-(4-(2-((tert-butoxycarbonyl)amino)-2-methylpropanoyl)piperazine-1-carboxamido)-2-oxopyrimidin-1(2H)-yl) cyclohexyl)methyl)amino)spiro[3.3]heptan-2-yl)carbamate and acetaldehyde. A cis/trans mixture was observed by TLC and ¹H NMR of tert-butyl (1-(4-((1-((trans)-4-(((6-((tert-butoxycarbonyl)amino)spiro[3.3]heptan-2-yl)(ethyl)amino)methyl)cyclohexyl)-2-oxo-1,2-dihydropyrimidin-4-yl)carbamoyl)piperazin-1-yl)-2-methyl-1-oxopropan-2-yl)carbamate Purification of the major isomer was accomplished by Waters SFC 350 with a Chromegachiral™ column 250*20 mm, 5 micro, a 2489 UV detector, Liq.CO₂ Mobile Phase (A) with (B) 0.1% methanolic ammonia in methanol:acetonitrile (50:50), 90 ml/min, 6 min run time and 10 mg sample loading 10 mg. Isolation of peak and deprotection as in Scheme C-7 afforded the desired product. ¹H NMR: (400 MHz, D₂O): δ 7.97 (d, 1H), 6.63 (d, 1H), 4.48-4.28 (m, 1H), 3.78-3.74 (m, 3H), 3.72-3.60 (m, 8H), 3.12-3.05 (m, 2H), 2.89-2.81 (m, 2H), 2.48-2.46 (m, 2H), 2.35-2.26 (m, 3H), 2.24-2.15 (m, 3H), 1.93-1.84 (m, 4H), 1.81-1.72 (m, 1H), 1.66-1.58 (m, 9H), 1.23-1.33 (m, 3H). LCMS[M+H] 557.4.

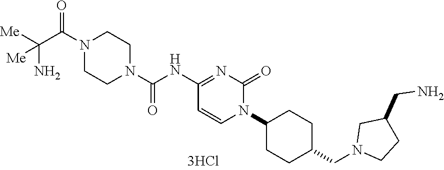

### (R)-Trans-4-(2-amino-2-methylpropanoyl)-N-(1-(4-((3-(aminomethyl)pyrrolidin-1-yl)methyl)cyclohexyl)-2-oxo-1,2-dihydropyrimidin-4-yl)piperazine-1-carboxamide Hydrochloride (Compound 51)

Prepared in a similar fashion to Scheme C-7 using trans-tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and (R)-tert-butyl (pyrrolidin-3-ylmethyl)carbamate. A cis/trans mixture was observed by TLC and ¹H NMR of tert-butyl (2-methyl-1-(4-((1-((trans)-4-(((R)-3-((methylamino)methyl)pyrrolidin-1-yl)methyl)cyclohexyl)-2-oxo-1,2-dihydropyrimidin-4-yl)carbamoyl)piperazin-1-yl)-1-oxopropan-2-yl)carbamate. Purification of the major isomer was accomplished by Waters SFC 350 with a Chromegachiral™ column 250*20 mm, 5 micro, a 2489 UV detector, Liq.CO₂ Mobile Phase (A) with (B) 0.1% methanolic ammonia in methanol:acetonitrile (50:50), 90 ml/min, 7 min run time and 15 mg sample loading. Isolation of peak and deprotection as in Scheme C-7 afforded the desired product. ¹H NMR: (400 MHz, D₂O): δ 7.92 (d, 1H), 6.65 (d, 1H), 4.34 (t, 1H), 3.87-3.82 (m, 1H), 3.79-3.51 (m, 8H), 3.40 (d, 1H), 3.22-3.20 (m, 1H), 3.13-3.01 (m, 4H), 2.87-2.82 (m, 1H), 2.71-2.60 (m, 1H), 2.42-2.31 (m, 1H), 2.30-2.18 (m, 1H), 1.97-1.83 (m, 4H), 1.81-1.75 (m, 2H), 1.62 (s, 6H), 1.59-1.54 (m, 1H), 1.25-1.16 (m, 2H). LCMS[M+H] 503.4.

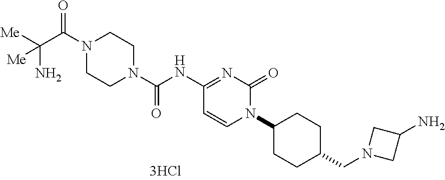

### trans-4-(2-Amino-2-methylpropanoyl)-N-(1-(4-((3-aminoazetidin-1-yl)methyl)cyclohexyl)-2-oxo-1,2-dihydropyrimidin-4-yl)piperazine-1-carboxamide hydrochloride salt (Compound 52)

Prepared in a similar fashion to Scheme C-7 using trans-tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and tert-butyl azetidin-3-ylcarbamate. Purification of individual isomer on tert-butyl (1-(4-((1-((cis/trans)-4-((3-((tert-butoxycarbonyl)amino)azetidin-1-yl)methyl)cyclohexyl)-2-oxo-1,2-dihydropyrimidin-4-yl)carbamoyl)piperazin-1-yl)-2-methyl-1-oxopropan-2-yl)carbamate was accomplished by Waters SFC 350 Column Chiralpack® IA 250*50 mm, 5 micron, 2489 UV detector, Liq.CO₂ mobile phase with 0.1% methanolic ammonia in isopropyl alcohol:acetonitrile (70:30), with an Isocratic method (55% A, 45% B), Flow rate 150 ml/min, Sample loading 10 mg, 12 min. run. Deprotection as in Scheme C-7 afforded the major product. ¹H NMR: (400 MHz, D₂O): δ 7.99 (d, 1H), 6.62 (d, 1H), 4.78-4.75 (m, 2H), 4.62-4.55 (m, 1H), 4.45-4.32 (m, 2H), 4.31-4.19 (m, 1H), 3.65-3.53 (m, 8H), 3.30-3.17 (m, 2H), 3.15-2.97 (m, 1H), 1.93-1.79 (m, 4H), 1.72-1.66 (m, 1H), 1.62 (s, 6H), 1.58-1.54 (m, 1H), 1.25-1.19 (m, 2H). LCMS[M+H] 475.6.

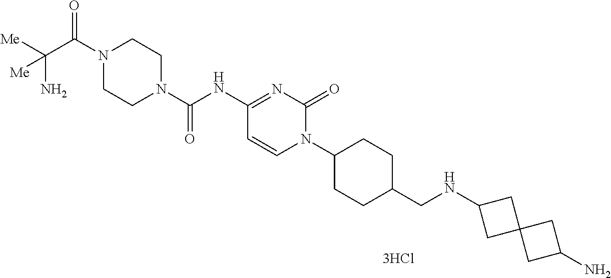 [SMILES: CC(C)(N)C(=O)N1CCN(C(=O)Nc2ccn(C3CCC(CNC4CC5(CC(N)C5)C4)CC3)c(=O)n2)CC1]

### 4-(2-Amino-2-methylpropanoyl)-N-(1-(4-(((6-aminospiro[3.3]heptan-2-yl)amino)methyl)cyclohexyl)-2-oxo-1,2-dihydropyrimidin-4-yl)piperazine-1-carboxamide hydrochloride salt (Compound 53)

Prepared in a similar fashion to from trans-tert-butyl (1-(4-((1-(4-formylcyclohexyl)-2-oxo-1,2-dihydropyrimidin-4-yl)carbamoyl)piperazin-1-yl)-2-methyl-1-oxopropan-2-yl)carbamate and tert-butyl (6-aminospiro[3.3]heptan-2-yl)carbamate. LCMM+H] 529.6.

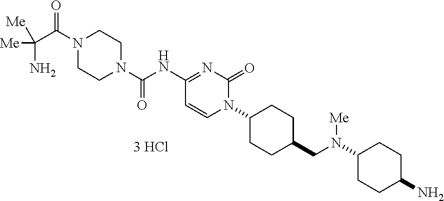

### 4-(2-Amino-2-methylpropanoyl)-N-(1-(trans-4-(((trans-4-aminocyclohexyl)(methyl)amino)methyl)cyclohexyl)-2-oxo-1,2-dihydropyrimidin-4-yl)piperazine-1-carboxamide hydrochloride salt (Compound 19)

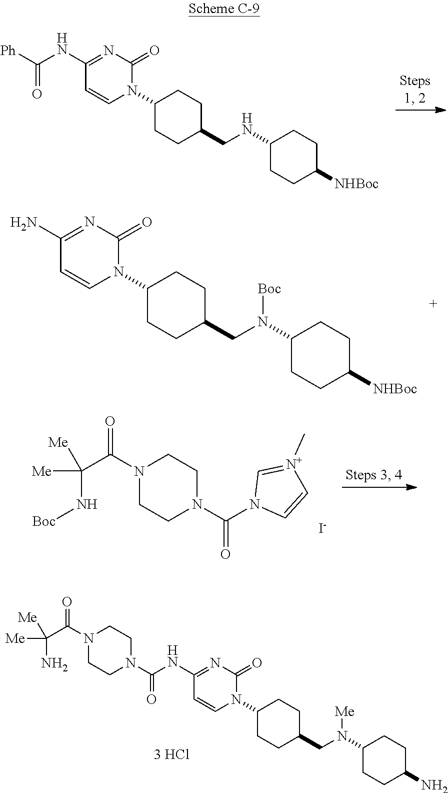

Reagents: 1) formaldehyde, NaBH₃CN, MeOH, rt, 24 h 2) NH₄OH, MeOH, 50° C., 3 h 3) CH₃CN, heat, 21 h 4) 2M HCl in MeOH, rt, 16 h.

Step 1: tert-butyl (trans-4-(((trans-4-(4-benzamido-2-oxopyrimidin-1(2H)-yl)cyclohexyl)methyl)(methyl)amino)cyclohexyl)carbamate. NaBH₃CN (19 mg, 307 μmol) was added to a mixture of tert-butyl (trans-4-(((trans-4-(4-benzamido-2-oxopyrimidin-1(2H)-yl)cyclohexyl)methyl)amino)cyclohexyl)carbamate (51 mg, 97 μmol) and 37 wt % formaldehyde (40 μL, 0.54 mmol) in MeOH (1 mL), and the mixture was stirred at rt for 24 h. The crude material was purified by column chromatography (DCM/MeOH/NH₄OH) to give the title compound (6.7 mg). LCMS[M+H] 538.4.

Step 2: tert-butyl (trans-4-(((trans-4-(4-amino-2-oxopyrimidin-1(2H)-yl)cyclohexyl)methyl)(methyl)amino)cyclohexyl)carbamate. A mixture of tert-butyl (trans-4-(((trans-4-(4-benzamido-2-oxopyrimidin-1(2H)-yl)cyclohexyl)methyl)(methyl)amino)cyclohexyl)carbamate (6.7 mg, 12 μmol) and 29 wt % NH₄OH (0.050 mL, 0.36 mmol) in MeOH (1 mL) was stirred at 50° C. for 3 h then concentrated to dryness to give the title compound. LCMS[M+H] 434.3.

Step 3: tert-butyl (1-(4-((1-(trans-4-(((trans-4-((tert-butoxycarbonyl)amino)cyclohexyl)(methyl)amino)methyl)cyclohexyl)-2-oxo-1,2-dihydropyrimidin-4-yl)carbamoyl)piperazin-1-yl)-2-methyl-1-oxopropan-2-yl)carbamate. A mixture of tert-butyl (trans-4-(((trans-4-(4-amino-2-oxopyrimidin-1(2H)-yl)cyclohexyl)methyl)(methyl)amino)cyclohexyl)carbamate (5.2 mg, 12 μmol) and 1-(4-(2-((tert-butoxycarbonyl)amino)-2-methylpropanoyl)piperazine-1-carbonyl)-3-methyl-1H-imidazol-3-ium iodide (12.1 mg, 24 μmol) in CH₃CN (1 mL) was stirred at 85° C. for 21 h. The mixture was cooled, Et₃N (2 drops) was added, and the mixture was concentrated. The residue was purified by column chromatography (DCM/MeOH/NH₄OH) to give the title compound (5.6 mg). LCMS[M+H] 731.7.

Step 4: 4-(2-amino-2-methylpropanoyl)-N-(1-(trans-4-(((trans-4-aminocyclohexyl)(methyl)amino)methyl)cyclohexyl)-2-oxo-1,2-dihydropyrimidin-4-yl)piperazine-1-carboxamide hydrochloride salt. A mixture of tert-butyl (1-(4-((1-(trans-4-(((trans-4-((tert-butoxycarbonyl)amino)cyclohexyl)(methyl)amino)methyl)cyclohexyl)-2-oxo-1,2-dihydropyrimidin-4-yl)carbamoyl)piperazin-1-yl)-2-methyl-1-oxopropan-2-yl)carbamate (5.6 mg, 7.7 μmol) and 2M HCl in MeOH (0.50 mL, 1.0 mmol) was stirred at rt for 16 h. The reaction mixture was concentrated to afford the title compound (5.0 mg). ¹H NMR (500 MHz, D₂O) δ 8.14 (d, 1H), 6.75 (d, 1H), 4.54-4.45 (m, 1H), 3.88-3.68 (m, 8H), 3.44-3.36 (m, 1H), 3.32-3.17 (m, 2H), 3.10-3.03 (m, 1H), 2.88 (s, 3H), 2.32-2.15 (m, 4H), 2.12-2.03 (m, 3H), 2.03-1.89 (m, 2H), 1.83-1.69 (m, 9H), 1.67-1.55 (m, 2H), 1.43-1.27 (m, 3H). LCMS[M+H] 531.3.

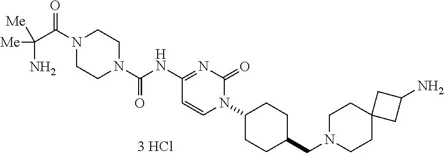

### 4-(2-Amino-2-methylpropanoyl)-N-(1-(trans-4-((2-amino-7-azaspiro[3.5]nonan-7-yl)methyl)cyclohexyl)-2-oxo-1,2-dihydropyrimidin-4-yl)piperazine-1-carboxamide hydrochloride salt (Compound 20)

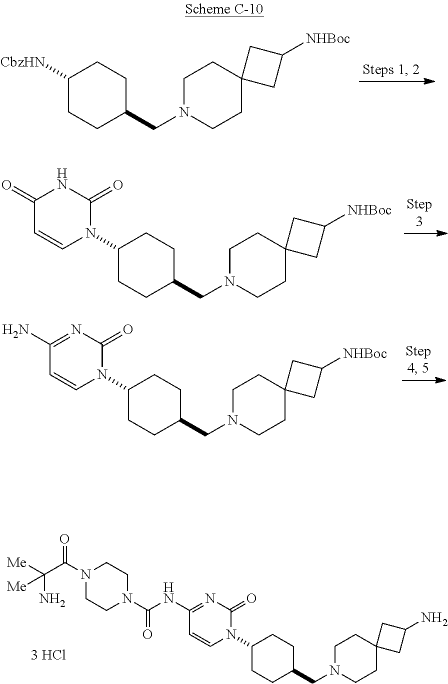

Reagents: 1) i) 3-ethoxyacryloyl chloride, AgOCN, toluene, Q, 3 h ii) amine, DCM, −78° C. to rt, 20 h 2) i) TFA, rt, 2.5 h ii) Boc₂O, 2M NaOH, THF, rt, 20 h 3) i) POCl₃, 1,2,4-triazole, Et₃N, CH₃CN, rt, 17 h ii) NH₄OH, CH₃CN, 50° C., 48 h 4) CH₃CN, heat, 17 h 5) 2 M HCl/MeOH, rt, 22 h.

Step 1: tert-butyl (7-((trans-4-(3-(3-ethoxyacryloyl)ureido)cyclohexyl)methyl)-7-azaspiro[3.5]nonan-2-yl)carbamate. A solution of 3-ethoxyacryloyl chloride (2.0 g, 15 mmol) in toluene (36 mL) was added dropwise to a suspension of silver cyanate (2.5 g, 16.7 mmol) in toluene (40 mL), and the mixture was stirred at reflux for 3 h, cooled and allowed to settle. A portion of the solution (44.4 mL, 8.9 mmol) was filtered through a syringe filter and added dropwise into a solution of tert-butyl (7-((trans-4-aminocyclohexyl)methyl)-7-azaspiro[3.5]nonan-2-yl)carbamate (2.6 g, 7.4 mmol) in dry DCM (74 mL) under N₂ cooled to −78° C. The mixture was warmed to rt and stirred for 20 h. MeOH (50 mL) was added, the solution concentrated, Et₂O (350 mL) added and extracted with 5% citric acid (2×325 mL). The aqueous extracts were brought to pH 12 with NaOH (20 g) then extracted with DCM (2×350 mL). The extracts were dried over Na₂SO₄, filtered, and concentrated to dryness to give the title compound (3.39 g). LCMS[M+H] 493.3.

Step 2: tert-butyl (7-((trans-4-(2,4-dioxo-3,4-dihydropyrimidin-1(2H)-yl)cyclohexyl)methyl)-7-azaspiro[3.5]nonan-2-yl)carbamate. A mixture of tert-butyl (7-((trans-4-(3-(3-ethoxyacryloyl)ureido)cyclohexyl)methyl)-7-azaspiro[3.5]nonan-2-yl)carbamate (3.39 g, 6.88 mmol) and TFA (26.5 mL, 344 mmol) was stirred at rt for 2.5 h. The mixture was concentrated to dryness. The residue was taken up in THF (70 mL), and 2M NaOH (51.6 mL, 103 mmol). Di-tert-butyl dicarbonate (3.16 mL, 13.8 mmol) was added and the mixture was stirred vigorously at rt for 20 h. The mixture was poured into sat. aq. NaHCO₃ (350 mL) and extracted with EtOAc (2×350 mL). The extracts were washed with brine (350 mL), dried (Na₂SO₄), decanted, and concentrated. The residue was purified by column chromatography (DCM/MeOH/NH₄OH) to give the title compound (2.60 g). ¹H NMR (500 MHz, MeOD) δ 7.67 (d, 1H), 5.66 (d, 1H), 4.36 (tt, 1H), 4.03-3.90 (m, 1H), 2.50-2.23 (m, 4H), 2.23-2.11 (m, 4H), 2.03-1.93 (m, 2H), 1.93-1.81 (m, 3H), 1.72-1.54 (m, 8H), 1.42 (s, 9H), 1.20-1.06 (m, 2H). LCMS[M+H] 447.3.

Step 3: tert-butyl (7-((trans-4-(4-amino-2-oxopyrimidin-1(2H)-yl)cyclohexyl)methyl)-7-azaspiro[3.5]nonan-2-yl)carbamate. POCl₃ (0.63 mL, 6.8 mmol) was added dropwise to a mixture of 1,2,4-triazole (3.7 g, 54 mmol) and Et₃N (7.5 mL, 54 mmol) in CH₃CN (25 mL) at 0° C., and the mixture was stirred at 0° C. for 10 min. Meanwhile, CH₃CN (25 mL) was added to tert-butyl (7-((trans-4-(2,4-dioxo-3,4-dihydropyrimidin-1(2H)-yl)cyclohexyl)methyl)-7-azaspiro[3.5]nonan-2-yl)carbamate (1.01 g, 2.26 mmol). The POCl₃ mixture was then poured into that suspension, and the mixture was stirred at rt for 17 h. Water (150 mL) was added and the precipitate was collected by vacuum filtration to give the intermediate (821 mg, 1.65 mmol). LCMS[M+H] 498.3. CH₃CN (20 mL) and 28 wt % NH₄OH (11 mL, 80 mmol) were added and the reaction mixture was stirred at 50° C. for 48 h. The mixture was cooled, diluted with water (50 mL), and the precipitate was collected by vacuum filtration and dried under vacuum to give the title compound (681 mg). ¹H NMR (500 MHz, MeOD) δ 7.64 (d, 1H), 5.87 (d, 1H), 4.44 (tt, 1H), 4.02-3.90 (m, 1H), 2.50-2.23 (m, 4H), 2.23-2.10 (m, 4H), 2.01-1.92 (m, 2H), 1.92-1.82 (m, 2H), 1.70-1.52 (m, 9H), 1.42 (s, 9H), 1.20-1.06 (m, 2H). LCMS[M+H] 446.3.

Step 4: tert-butyl (1-(4-((1-(trans-4-((2-((tert-butoxycarbonyl)amino)-7-azaspiro[3.5]nonan-7-yl)methyl)cyclohexyl)-2-oxo-1,2-dihydropyrimidin-4-yl)carbamoyl)piperazin-1-yl)-2-methyl-1-oxopropan-2-yl)carbamate. A mixture of tert-butyl (7-((trans-4-(4-amino-2-oxopyrimidin-1(2H)-yl)cyclohexyl)methyl)-7-azaspiro[3.5]nonan-2-yl)carbamate (593 mg, 1.33 mmol) and 1-(4-(2-((tert-butoxycarbonyl)amino)-2-methylpropanoyl)piperazine-1-carbonyl)-3-methyl-1H-imidazol-3-ium iodide (1.17 g, 2.3 mmol) in CH₃CN (12 mL) was stirred at reflux for 17 h. The mixture was cooled, diluted with EtOAc (150 mL), washed with sat. aq. NaHCO₃ (2×125 mL) and brine (125 mL), dried over Na₂SO₄, filtered and concentrated. The residue was purified by column chromatography (DCM/MeOH/NH₄OH) to give the title compound (691 mg). LCMS[M+H] 743.6.

Step 5: 4-(2-amino-2-methylpropanoyl)-N-(1-(trans-4-((2-amino-7-azaspiro[3.5]nonan-7-yl)methyl)cyclohexyl)-2-oxo-1,2-dihydropyrimidin-4-yl)piperazine-1-carboxamide hydrochloride salt. A mixture of tert-butyl (1-(4-((1-(trans-4-((2-((tert-butoxycarbonyl)amino)-7-azaspiro[3.5]nonan-7-yl)methyl)cyclohexyl)-2-oxo-1,2-dihydropyrimidin-4-yl)carbamoyl)piperazin-1-yl)-2-methyl-1-oxopropan-2-yl)carbamate (691 mg, 0.93 mmol) and 2M HCl in MeOH (20 mL, 40 mmol) was stirred at rt for 22 h. IPA was added and the precipitate was collected by vacuum filtration, washed with IPA and Et₂O, and dried under vacuum to give the title compound (573 mg). ¹H NMR (500 MHz, D₂O) δ 8.06 (d, 1H), 6.77 (d, 1H), 4.52-4.42 (m, 1H), 3.91 (p, 1H), 3.87-3.65 (m, 8H), 3.62-3.46 (m, 2H), 3.12-2.92 (m, 4H), 2.58-2.47 (m, 1H), 2.40-2.30 (m, 1H), 2.19-1.86 (m, 11H), 1.82-1.66 (m, 8H), 1.40-1.26 (m, 2H). LCMS[M+H] 543.4.

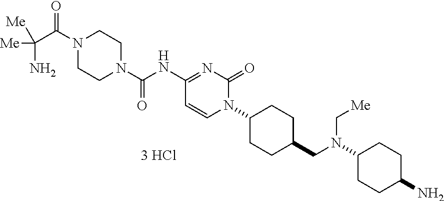

### 4-(2-Amino-2-methylpropanoyl)-N-(1-(trans-4-(((trans-4-aminocyclohexyl)(ethyl)amino)methyl)cyclohexyl)-2-oxo-1,2-dihydropyrimidin-4-yl)piperazine-1-carboxamide hydrochloride salt (Compound 21)

Prepared in a similar fashion to Scheme C-10 from tert-butyl ((trans)-4-((((trans)-4-(4-amino-2-oxopyrimidin-1(2H)-yl)cyclohexyl)methyl) (ethyl)amino)cyclohexyl)carbamate. ¹H NMR (500 MHz, D₂O) δ 8.12 (d, 1H), 6.76 (d, 1H), 4.49 (tt, 1H), 3.93-3.65 (m, 8H), 3.45 (tt, 1H), 3.39-3.23 (m, 3H), 3.20 (dd, 1H), 3.06 (dd, 1H), 2.32-2.14 (m, 4H), 2.12-1.98 (m, 4H), 1.96-1.85 (m, 1H), 1.85-1.68 (m, 10H), 1.68-1.55 (m, 2H), 1.44-1.29 (m, 5H). LCMS[M+H] 545.4.

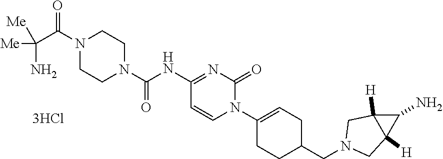 [SMILES: CC(C)(N)C(=O)N1CCN(C(=O)Nc2ccn(C3=CCC(CN4C[C@@H]5[C@H](N)[C@@H]5C4)CC3)c(=O)n2)CC1]

### endo-4-(2-Amino-2-methylpropanoyl)-N-(1-(4-((6-amino-3-azabicyclo[3.1.0]hexan-3-yl)methyl)cyclohex-1-en-1-yl)-2-oxo-1,2-dihydropyrimidin-4-yl)piperazine-1-carboxamide hydrochloride salt (Compound 22)

Prepared in a similar fashion to Scheme C-5 from tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl) carbamate and endo-tert-butyl (3-azabicyclo[3.1.0]hexan-6-yl)carbamate. ¹H NMR: (400 MHz, D₂O): δ 7.73 (d, 1H), 6.61 (d, 1H), 5.81 (s, 1H), 4.21-4.02 (m, 1H), 3.82 (d, 1H), 3.62-3.56 (m, 8H), 3.49-3.47 (m, 2H), 3.51-3.13 (m, 1H), 3.11-3.02 (m, 1H), 2.93-2.85 (m, 1H), 2.85-2.75 (m, 1H), 2.35-2.22 (m, 4H), 2.11-2.01 (m, 1H), 1.94-1.80 (m, 2H), 1.59 (s, 6H), 1.49-1.35 (m, 1H). LCMS [M+H] 499.4.

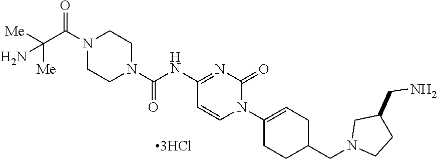 [SMILES: CC(C)(N)C(=O)N1CCN(C(=O)Nc2ccn(C3=CCC(CN4CC[C@H](CN)C4)CC3)c(=O)n2)CC1]

### (S)-4-(2-Amino-2-methylpropanoyl)-N-(1-(4-((3-(aminomethyl)pyrrolidin-1-yl)methyl)cyclohex-1-en-1-yl)-2-oxo-1,2-dihydropyrimidin-4-yl)piperazine-1-carboxamide hydrochloride salt (Compound 23)

Prepared in a similar fashion to Scheme C-5 from tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl) carbamate and (S)-tert-butyl (pyrrolidin-3-ylmethyl)carbamate. ¹H NMR: (400 MHz, D₂O): δ 7.79 (d, 1H), 6.60 (d, 1H), 5.85 (s, 1H), 3.87-3.83 (m, 1H), 3.76-3.58 (m, 8H), 3.41 (d, 1H), 3.21-3.16 (m, 3H), 3.11-3.00 (m, 2H), 2.85-2.81 (m, 1H), 2.66-2.62 (m, 1H), 2.33-2.20 (m, 4H), 2.09 (bs, 1H), 1.94-1.89 (m, 2H), 1.69-1.66 (m, 1H), 1.59 (s, 6H), 1.47-1.45 (m, 1H). LCMS [M+H] 501.5.

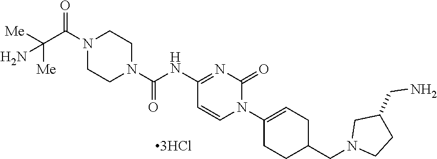 [SMILES: CC(C)(N)C(=O)N1CCN(C(=O)Nc2ccn(C3=CCC(CN4CC[C@@H](CN)C4)CC3)c(=O)n2)CC1]

### (R)-4-(2-Amino-2-methylpropanoyl)-N-(1-(4-((3-(aminomethyl)pyrrolidin-1-yl)methyl)cyclohex-1-en-1-yl)-2-oxo-1,2-dihydropyrimidin-4-yl)piperazine-1-carboxamide hydrochloride salt (Compound 24)

Prepared in a similar fashion to Scheme C-5 from tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl) carbamate and (R)-tert-butyl (pyrrolidin-3-ylmethyl)carbamate. ¹H NMR: (400 MHz, D₂O): δ 7.71 (d, 1H), 6.63 (d, 1H), 5.82 (s, 1H), 3.85-3.83 (m, 1H), 3.81-3.55 (m, 8H), 3.43 (d, 1H), 3.21-3.16 (m, 3H), 3.11-3.00 (m, 2H), 2.85-2.83 (m, 1H), 2.71-2.62 (m, 1H), 2.33-2.23 (m, 4H), 2.09 (bs, 1H), 1.94-1.88 (m, 2H), 1.75-1.66 (m, 1H), 1.59 (s, 6H), 1.47-1.44 (m, 1H). LCMS [M+H] 501.6.

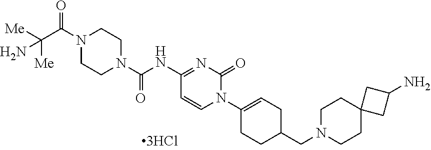 [SMILES: CC(C)(N)C(=O)N1CCN(C(=O)Nc2ccn(C3=CCC(CN4CCC5(CC4)CC(N)C5)CC3)c(=O)n2)CC1]

### 4-(2-Amino-2-methylpropanoyl)-N-(1-(4-((2-amino-7-azaspiro[3.5]nonan-7-yl)methyl)cyclohexan-1-en-1-yl)-2-oxo-1,2-dihydropyrimidin-4-yl)piperazine-1-carboxamide hydrochloride salt (Compound 25)

Prepared in a similar fashion to Scheme C-5 from tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl) carbamate and tert-butyl (7-azaspiro[3.5]nonan-2-yl)carbamate. ¹H NMR: (400 MHz, D₂O): δ 7.74 (d, 1H), 6.63 (d, 1H), 5.84 (s, 1H), 3.77 (t, 1H), 3.74-3.58 (m, 8H), 3.46-3.39 (m, 2H), 3.02 (d, 2H), 2.96-2.90 (m, 2H), 2.49-2.39 (m, 1H), 2.32-2.21 (m, 5H), 2.01-1.77 (m, 8H), 1.61 (s, 6H), 1.47-1.45 (m, 1H). LCMS [M+H] 541.5.

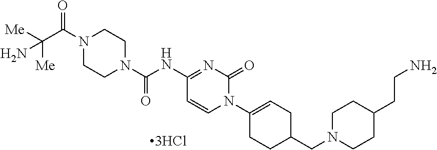 [SMILES: CC(C)(N)C(=O)N1CCN(C(=O)Nc2ccn(C3=CCC(CN4CCC(CCN)CC4)CC3)c(=O)n2)CC1]

### 4-(2-Amino-2-methylpropanoyl)-N-(1-(4-((4-(2-aminoethyl)piperidin-1-yl)methyl)cyclohex-1-en-1-yl)-2-oxo-1,2-dihydropyrimidin-4-yl)piperazine-1-carboxamide hydrochloride salt (Compound 26)

Prepared in a similar fashion to Scheme C-5 from tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl) carbamate and tert-butyl (2-(piperidin-4-yl)ethyl)carbamate. ¹H NMR: (400 MHz, D₂O): δ 7.75-7.73 (m, 1H), 6.62-6.60 (m, 1H), 5.83 (s, 1H), 4.64-4.61 (m, 1H), 3.63-3.51 (m, 9H), 3.25-3.11 (m, 1H), 3.01-3.00 (m, 2H), 2.92-2.83 (m, 4H), 2.30-2.23 (m, 3H), 2.21-2.09 (m, 1H), 1.90-1.87 (m, 4H), 1.59-1.54 (m, 8H), 1.45-1.35 (m, 3H). LCMS [M+H] 529.6.

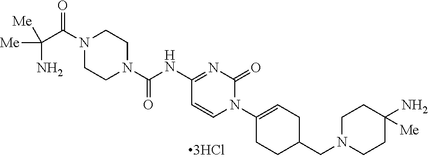 [SMILES: CC1(N)CCN(CC2CC=C(n3ccc(NC(=O)N4CCN(C(=O)C(C)(C)N)CC4)nc3=O)CC2)CC1]

### 4-(2-Amino-2-methylpropanoyl)-N-(1-(4-((4-amino-4-methylpiperidin-1-yl)methyl)cyclohex-1-en-1-yl)-2-oxo-1,2-dihydropyrimidin-4-yl)piperazine-1-carboxamide hydrochloride salt (Compound 27)

Prepared in a similar fashion to Scheme C-5 from tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl) carbamate and tert-butyl (4-methylpiperidin-4-yl)carbamate. ¹H NMR: (400 MHz, D₂O): δ 7.71 (d, 1H), 6.64 (d, 1H), 5.83 (s, 1H), 3.62-3.58 (m, 10H), 3.14-3.12 (m, 4H), 2.30-2.20 (m, 4H), 2.19-2.06 (m, 4H), 1.90-1.85 (m, 2H), 1.60 (s, 6H), 1.44-1.40 (m, 4H). LCMS [M+H] 515.6.

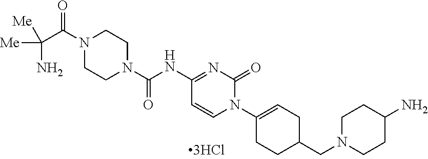 [SMILES: CC(C)(N)C(=O)N1CCN(C(=O)Nc2ccn(C3=CCC(CN4CCC(N)CC4)CC3)c(=O)n2)CC1]

### 4-(2-Amino-2-methylpropanoyl)-N-(1-(4-((4-aminopiperidin-1-yl)methyl)cyclohex-1-en-1-yl)-2-oxo-1,2-dihydropyrimidin-4-yl)piperazine-1-carboxamide hydrochloride salt (Compound 28)

Prepared in a similar fashion to Scheme C-5 from tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl) carbamate and tert-butyl piperidin-4-ylcarbamate. ¹H NMR: (400 MHz, D₂O): δ 7.69 (d, 1H), 6.62 (d, 1H), 5.81 (s, 1H), 3.63-3.55 (m, 8H), 3.44-3.35 (m, 2H), 3.07-2.98 (m, 4H), 2.41-2.30 (m, 3H), 2.22-2.19 (m, 4H), 1.88-1.70 (m, 4H), 1.58 (s, 6H), 1.51-1.45 (m, 1H). LCMS[M/2+H] 251.2.

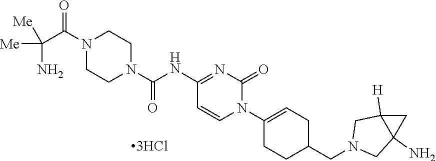 [SMILES: CC(C)(N)C(=O)N1CCN(C(=O)Nc2ccn(C3=CCC(CN4CC5CC5(N)C4)CC3)c(=O)n2)CC1]

### 4-(2-Amino-2-methylpropanoyl)-N-(1-(4-((1-amino-3-azabicyclo [3.1.0]hexan-3-yl) methyl) cyclohex-1-en-1-yl)-2-oxo-1,2-dihydropyrimidin-4-yl)piperazine-1-carboxamide hydrochloride salt (Compound 29)

Prepared in a similar fashion to Scheme C-5 from tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl) carbamate and tert-butyl (3-azabicyclo[3.1.0]hexan-1-yl)carbamate. ¹H NMR: (400 MHz, D₂O): δ 7.78-7.76 (d, 1H), 6.63-6.61 (d, 1H), 5.84 (s, 1H), 4.04 (bs, 1H), 3.64-3.58 (m, 9H), 3.24 (m, 2H), 2.29-2.24 (m, 4H), 2.07 (m, 3H), 1.95-1.89 (m, 6H), 1.60 (s, 2H), 1.44-1.30 (m, 3H). LCMS [M+H] 499.4.

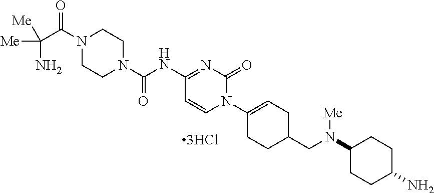 [SMILES: CN(CC1CC=C(n2ccc(NC(=O)N3CCN(C(=O)C(C)(C)N)CC3)nc2=O)CC1)[C@H]1CC[C@H](N)CC1]

### trans-4-(2-Amino-2-methylpropanoyl)-N-(1-(4-(((4-aminocyclohexyl)(methyl)amino)methyl)cyclohex-1-en-1-yl)-2-oxo-1,2-dihydropyrimidin-4-yl)piperazine-1-carboxamide hydrochloride salt (Compound 30)

Prepared in a similar fashion to Scheme C-5 from trans-tert-butyl (1-(4-((1-(4-(((4-((tert-butoxycarbonyl)amino)cyclohexyl)amino)methyl)cyclohex-1-en-1-yl)-2-oxo-1,2-dihydro pyrimidin-4-yl)carbamoyl)piperazin-1-yl)-2-methyl-1-oxopropan-2-yl)carbamate and formaldehyde. ¹H NMR: (400 MHz, D₂O): δ 7.67 (d, 1H), 6.62 (d, 1H), 5.80 (s, 1H), 3.61-3.55 (m, 8H), 3.26-3.24 (m, 1H), 3.17-3.07 (m, 2H), 3.02-2.91 (m, 1H), 2.73 (s, 3H), 2.33-2.18 (m, 3H), 2.08-2.02 (m, 5H), 1.91-1.80 (m, 3H), 1.57 (s, 6H), 1.48-1.42 (m, 4H). LCMS [M+H] 529.6.

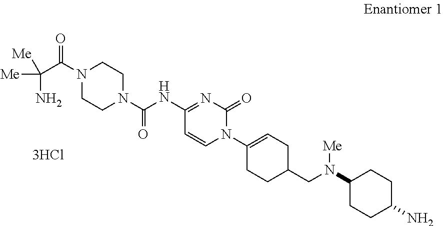 [SMILES: CN(CC1CC=C(n2ccc(NC(=O)N3CCN(C(=O)C(C)(C)N)CC3)nc2=O)CC1)[C@H]1CC[C@H](N)CC1]

### trans-4-(2-Amino-2-methylpropanoyl)-N-(1-(4-(((4-aminocyclohexyl)(methyl)amino)methyl)cyclohex-1-en-1-yl)-2-oxo-1,2-dihydropyrimidin-4-yl)piperazine-1-carboxamide hydrochloride salt (Enantiomer 1) (Compound 32)

Prepared by chiral chromatography of trans-4-(2-amino-2-methylpropanoyl)-N-(1-(4-(((4-aminocyclohexyl)(methyl)amino)methyl)cyclohex-1-en-1-yl)-2-oxo-1,2-dihydropyrimidin-4-yl)piperazine-1-carboxamide according to the following parameters: Instrument: Agilent 1260 infinity; Column: Chiralpack® IA 250*50 mm, 5 micron; DAD Detector; Mobile phases (A) 0.1% TEA in EtOH (B) ACN; Method: isocratic (97% A, 3% B); Flow rate 20 ml/min.; Run time 45 min.; Sample loading 5 mg. ¹H NMR: (400 MHz, D₂O): δ 7.69 (d, 1H), 6.68 (d, 1H), 5.85 (s, 1H), 3.73-3.53 (m, 8H), 3.42-3.25 (m, 2H), 3.22-3.12 (m, 2H), 3.07-2.99 (m, 1H), 2.79 (d, 3H), 2.34-2.22 (m, 3H), 2.16-2.11 (m, 5H), 1.99-1.86 (m, 2H), 1.67 (s, 6H), 1.53-1.38 (m, 4H). LCMS[M+H] 529.6.

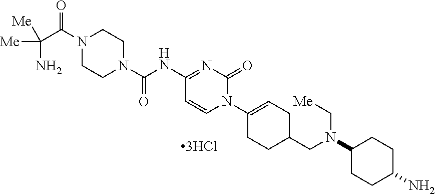 [SMILES: CCN(CC1CC=C(n2ccc(NC(=O)N3CCN(C(=O)C(C)(C)N)CC3)nc2=O)CC1)[C@H]1CC[C@H](N)CC1]

### trans-4-(2-Amino-2-methylpropanoyl)-N-(1-(4-(((4-aminocyclohexyl)(ethyl)amino)methyl)cyclohex-1-en-1-yl)-2-oxo-1,2-dihydropyrimidin-4-yl)piperazine-1-carboxamide hydrochloride salt (Compound 31)

Prepared in a similar fashion to Scheme C-5 from trans-tert-butyl (1-(4-((1-(4-(((4-((tert-butoxycarbonyl)amino)cyclohexyl)amino)methyl)cyclohex-1-en-1-yl)-2-oxo-1,2-dihydro pyrimidin-4-yl)carbamoyl)piperazin-1-yl)-2-methyl-1-oxopropan-2-yl)carbamate and acetaldehyde. ¹H NMR: (400 MHz, D₂O): δ 7.32 (d, 1H), 6.35-6.22 (m, 1H), 5.69 (s, 1H), 3.60-3.47 (m, 8H), 3.19 (s, 2H), 2.71 (d, 4H), 2.55 (bs, 2H), 2.25-1.15 (m, 3H), 1.88-1.75 (m, 6H), 1.35-1.33 (m, 2H), 1.27 (s, 6H), 1.76-1.11 (m, 2H), 0.99 (t, 3H). LCMS [M+H] 543.0.

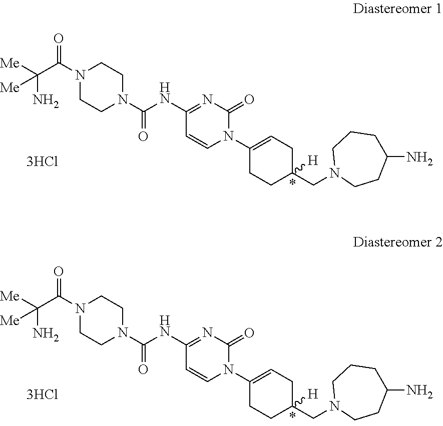 [SMILES: CC(C)(N)C(=O)N1CCN(C(=O)Nc2ccn(C3=CCC(CN4CCCC(N)CC4)CC3)c(=O)n2)CC1 ; CC(C)(N)C(=O)N1CCN(C(=O)Nc2ccn(C3=CCC(CN4CCCC(N)CC4)CC3)c(=O)n2)CC1]

### 4-(2-Amino-2-methylpropanoyl)-N-(1-(4-((4-aminoazepan-1-yl)methyl)cyclohex-1-en-1-yl)-2-oxo-1,2-dihydropyrimidin-4-yl)piperazine-1-carboxamide hydrochloride salt (Enantiomer 1) (Compound 41)

### 4-(2-Amino-2-methylpropanoyl)-N-(1-(4-((4-aminoazepan-1-yl)methyl)cyclohex-1-en-1-yl)-2-oxo-1,2-dihydropyrimidin-4-yl)piperazine-1-carboxamide hydrochloride salt (Enantiomer 2) (Compound 42)

Prepared in a similar fashion to Scheme C-5 from tert-butyl (1-(4-((1-(4-formylcyclohex-1-en-1-yl)-2-oxo-1,2-dihydropyrimidin-4-yl)carbamoyl)piperazin-1-yl)-2-methyl-1-oxopropan-2-yl) carbamate and tert-butyl azepan-4-ylcarbamate. Chiral chromatography of 4-(2-amino-2-methylpropanoyl)-N-(1-(4-((4-aminoazepan-1-yl)methyl)cyclohex-1-en-1-yl)-2-oxo-1,2-dihydropyrimidin-4-yl)piperazine-1-carboxamide was achieved according to the following parameters: Instrument: Agilent 1260 infinity; Column: Daicel Chiralpack®-AD 250*30 mm, 10 micron; DAD Detector; Mobile Phase (A) 0.1% Triethylamine in Acetonitrile: (B) 0.1% Triethylamine in Ethanol; Isocratic Method (40% A, 60% B): Flow rate 25 ml/min.; Run time 48 min.; Sample loading 25 mg. Fraction 1 ¹H NMR: (400 MHz, D₂O): δ 7.67 (d, 1H), 6.70 (d, 1H), 5.85 (s, 1H), 3.67-3.59 (m, 8H), 3.56-3.45 (m, 3H), 3.28-3.26 (m, 1H), 3.19-3.13 (m, 3H), 2.35-2.31 (m, 3H), 2.27-2.23 (m, 3H), 2.19-2.15 (m, 1H), 2.05-1.99 (m, 3H), 1.96-1.88 (m, 2H), 1.64 (s, 6H), 1.54-1.45 (m, 1H). LCMS [M+H] 515.6. Fraction 2 ¹H NMR: (400 MHz, D₂O): δ 7.67 (d, 1H), 6.70 (d, 1H), 5.84 (s, 1H), 3.67-3.59 (m, 8H), 3.54-3.45 (m, 3H), 3.33-3.28 (m, 1H), 3.18-3.08 (m, 3H), 2.35-2.31 (m, 2H), 2.28-2.23 (m, 3H), 2.21-2.11 (m, 2H), 2.09-1.98 (m, 3H), 1.97-1.87 (m, 2H), 1.64 (s, 6H), 1.53-1.48 (m, 1H). LCMS [M+H] 515.6.

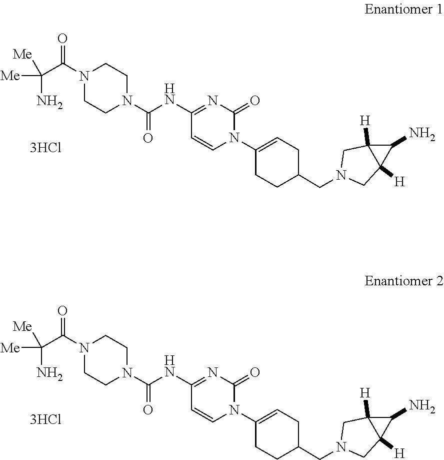 [SMILES: CC(C)(N)C(=O)N1CCN(C(=O)Nc2ccn(C3=CCC(CN4C[C@@H]5[C@@H](N)[C@@H]5C4)CC3)c(=O)n2)CC1 ; CC(C)(N)C(=O)N1CCN(C(=O)Nc2ccn(C3=CCC(CN4C[C@@H]5[C@@H](N)[C@@H]5C4)CC3)c(=O)n2)CC1]

### 4-(2-Amino-2-methylpropanoyl)-N-(1-(4-(((exo)-6-amino-3-azabicyclo[3.1.0]hexan-3-yl)methyl)cyclohex-1-en-1-yl)-2-oxo-1,2-dihydropyrimidin-4-yl)piperazine-1-carboxamide hydrochloride salt (Enantiomer 1) (Compound 33)

### 4-(2-Amino-2-methylpropanoyl)-N-(1-(4-(((exo)-6-amino-3-azabicyclo[3.1.0]hexan-3-yl)methyl)cyclohex-1-en-1-yl)-2-oxo-1,2-dihydropyrimidin-4-yl)piperazine-1-carboxamide hydrochloride salt (Enantiomer 2) (Compound 34)

Prepared by chiral chromatography of exo-tert-butyl (1-(4-((1-(4-((6-((tert-butoxycarbonyl)amino)-3-azabicyclo [3.1.0]hexan-3-yl)methyl)cyclohex-1-en-1-yl)-2-oxo-1,2-dihydropyrimidin-4-yl)carbamoyl) piperazin-1-yl)-2-methyl-1-oxopropan-2-yl)carbamate according to the following parameters and HCl deprotection of the Boc groups of the individual isomers as in as in Scheme C-5. Chiral Chromatography parameters: Instrument: Agilent 1260 infinity; Column: Chiralpack® IA 250*50 mm, 5 micron; Detector: DAD; Mobile Phases: (A) 0.1% TEA in EtOH (B) ACN; Method: isocratic (97% A, 3% B); Flow rate: 20 ml/min.; Run time: 45 min.; Sample loading: 5 mg. ¹H NMR (400 MHz, D₂O): δ 7.81 (d, 1H), 6.64 (d, 1H), 5.87 (s, 1H), 4.22-4.11 (m, 1H), 3.87 (d, 1H), 3.78-3.62 (m, 8H), 3.54-3.51 (m, 1H), 3.22-3.15 (m, 1H), 3.13-3.04 (m, 1H), 3.02-2.89 (m, 1H), 2.88-2.82 (m, 1H), 2.51-2.31 (m, 4H), 2.30-2.18 (m, 1H), 2.17-2.07 (m, 1H), 1.97-1.85 (m, 2H), 1.63 (s, 6H), 1.49-1.47 (m, 1H). LCMS [M+1]499.6.

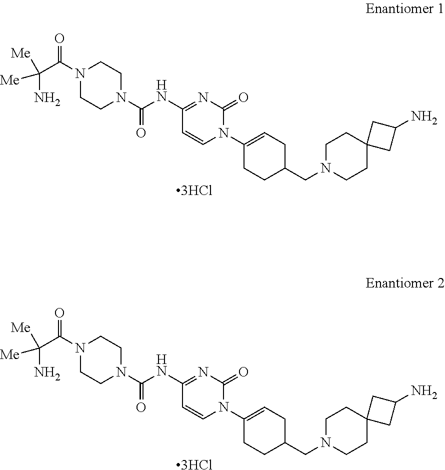 [SMILES: CC(C)(N)C(=O)N1CCN(C(=O)Nc2ccn(C3=CCC(CN4CCC5(CC4)CC(N)C5)CC3)c(=O)n2)CC1 ; "·3HCl" ; CC(C)(N)C(=O)N1CCN(C(=O)Nc2ccn(C3=CCC(CN4CCC5(CC4)CC(N)C5)CC3)c(=O)n2)CC1 ; "·3HCl"]

### 4-(2-Amino-2-methylpropanoyl)-N-(1-(4-((2-amino-7-azaspiro[3.5]nonan-7-yl)methyl)cyclohexan-1-yl)-2-oxo-1,2-dihydropyrimidin-4-yl)piperazine-1-carboxamide (Enantiomer 1) (Compound 35)

### 4-(2-Amino-2-methylpropanoyl)-N-(1-(4-((2-amino-7-azaspiro[3.5]nonan-7-yl)methyl)cyclohexan-1-yl)-2-oxo-1,2-dihydropyrimidin-4-yl)piperazine-1-carboxamide (Enantiomer 2) (Compound 36)

Prepared by chiral chromatography of tert-butyl (1-(4-((1-(4-((2-((tert-butoxycarbonyl)amino)-7-azaspiro[3.5]nonan-7-yl)methyl)cyclohexan-1-yl)-2-oxo-1,2-dihydropyrimidin-4-yl)carbamoyl)piperazin-1-yl)-2-methyl-1-oxopropan-2-yl)carbamate according to the following parameters and HCl deprotection of the Boc groups of the individual isomers as in Scheme C-1. Chiral Chromatography parameters: Instrument: Waters SFC 350; Column: Chiralpack® IA 250*50 mm, 5 micron; Detector: 2489 UV detector; Mobile Phase: (A) Liq.CO₂, (B) 0.1% TEA in IPA:tert-butylmethylether:CAN (50:25:25); Method: Isocratic (55% A, 45% B); Flow rate: 180 ml/min. Run time: 32 min.; Sample loading: 20 mg. ¹H NMR: (400 MHz, D₂O): δ 7.75 (d, 1H), 6.66 (d, 1H), 5.86 (s, 1H), 3.81-3.77 (m, 1H), 3.75-3.61 (m, 8H), 3.60-3.47 (m, 3H), 3.03 (d, 2H), 2.98-2.85 (m, 2H), 2.44-2.20 (m, 6H), 2.03-1.79 (m, 7H), 1.63 (s, 6H), 1.51-1.47 (m, 1H). LCMS [M+H] 541.7.

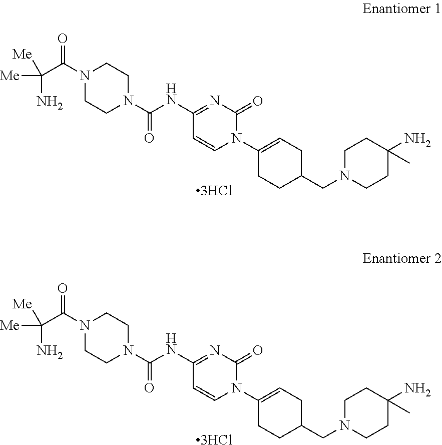 [SMILES: CC1(N)CCN(CC2CC=C(n3ccc(NC(=O)N4CCN(C(=O)C(C)(C)N)CC4)nc3=O)CC2)CC1 ; "·3HCl" ; CC1(N)CCN(CC2CC=C(n3ccc(NC(=O)N4CCN(C(=O)C(C)(C)N)CC4)nc3=O)CC2)CC1 ; "·3HCl"]

### 4-(2-Amino-2-methylpropanoyl)-N-(1-(4-((4-amino-4-methylpiperidin-1-yl)methyl)cyclohex-1-en-1-yl)-2-oxo-1,2-dihydropyrimidin-4-yl)piperazine-1-carboxamide hydrochloride salt (Enantiomer 1) (Compound 37)

### 4-(2-Amino-2-methylpropanoyl)-N-(1-(4-((4-amino-4-methylpiperidin-1-yl)methyl)cyclohex-1-en-1-yl)-2-oxo-1,2-dihydropyrimidin-4-yl)piperazine-1-carboxamide hydrochloride salt (Enantiomer 2) (Compound 38)

Prepared by chiral chromatography of tert-butyl (1-(4-((1-(4-((4-((tert-butoxycarbonyl)amino)-4-methylpiperidin-1-yl)methyl)cyclohex-1-en-1-yl)-2-oxo-1,2-dihydropyrimidin-4-yl)carbamoyl)piperazin-1-yl)-2-methyl-1-oxopropan-2-yl)carbamate according to the following parameters and HCl deprotection of the Boc groups of the individual isomers as in Scheme C-1. Chiral Chromatography parameters: Instrument: Agilent 1260 infinity; Column: Daicel Chiralpack®-AD 250*30 mm, 10 micron; Detector: DAD detector; Mobile phases: (A) 0.1% triethylamine in n-heptane, (B) IPA:MeOH (96:04); Method: Isocratic (75% A, 25% B); Flow rate: 35 ml/min.; Run time: 50 min.; Sample loading: 20 mg. ¹H NMR: (400 MHz, D₂O): δ 7.77 (d, 1H), 6.62 (d, 1H), 5.85 (s, 1H), 3.71-3.51 (m, 11H), 3.17-3.10 (m, 4H), 2.33-2.30 (m, 2H), 2.25-2.15 (m, 2H), 2.13-2.02 (m, 4H), 1.98-1.85 (m, 2H), 1.60 (s, 6H), 1.48-1.41 (m, 3H). LCMS[M+H] 515.6.

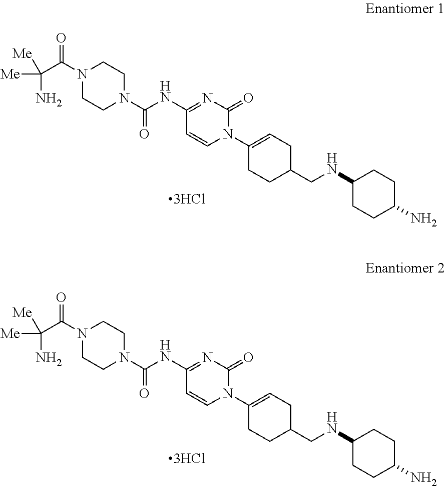 [SMILES: CC(C)(N)C(=O)N1CCN(C(=O)Nc2ccn(C3=CCC(CN[C@H]4CC[C@H](N)CC4)CC3)c(=O)n2)CC1 ; CC(C)(N)C(=O)N1CCN(C(=O)Nc2ccn(C3=CCC(CN[C@H]4CC[C@H](N)CC4)CC3)c(=O)n2)CC1]

### trans-4-(2-Amino-2-methylpropanoyl)-N-(1-(4-(((4-aminocyclohexyl)amino)methyl)cyclohex-1-en-1-yl)-2-oxo-1,2-dihydropyrimidin-4-yl)piperazine-1-carboxamide hydrochloride salt (Enantiomer 1) (Compound 39)

### trans-4-(2-Amino-2-methylpropanoyl)-N-(1-(4-(((4-aminocyclohexyl)amino)methyl)cyclohex-1-en-1-yl)-2-oxo-1,2-dihydropyrimidin-4-yl)piperazine-1-carboxamide hydrochloride salt (Enantiomer 2) (Compound 40)

Prepared by chiral chromatography of benzyl ((4-(4-(4-(2-((tert-butoxycarbonyl)amino)-2-methylpropanoyl)piperazine-1-carboxamido)-2-oxopyrimidin-1(2H)-yl)cyclohex-3-en-1-yl)methyl)((trans)-4-((tert-butoxycarbonyl)amino)cyclohexyl)carbamate followed by deprotection of the Boc/Cbz groups of the individual isomers in a similar fashion as in Scheme C-1. Chiral Chromatography parameters: Instrument: Agilent 1260 infinity; Column: Chiralpack® IA 250*50 mm, 5 μM; Detector: DAD detector; Mobile Phases: (A) 0.1% TEA in EtOH (B) ACN; Method: isocratic (97% A, 3% B); Flow rate: 20 ml/min.; Run time: 45 min.; Sample loading: 5 mg. ¹H NMR: (400 MHz, D₂O): δ 7.63 (d, 1H), 6.66 (d, 1H), 5.80 (s, 1H), 3.71-3.51 (m, 8H), 3.11-3.10 (m, 2H), 2.99 (d, 2H), 2.31-2.23 (m, 3H), 2.16-2.14 (m, 2H), 2.11-2.05 (m, 2H), 1.92-1.86 (m, 2H), 1.60 (s, 6H), 1.51-1.45 (m, 6H). LCMS[M+H] 515.6.

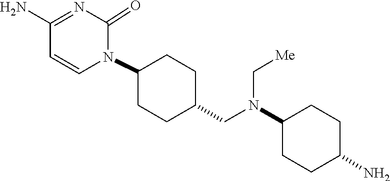

### 4-amino-1-((trans)-4-((((trans)-4-aminocyclohexyl)(ethyl)amino)methyl)cyclohexyl)pyrimidin-2(1H)-one

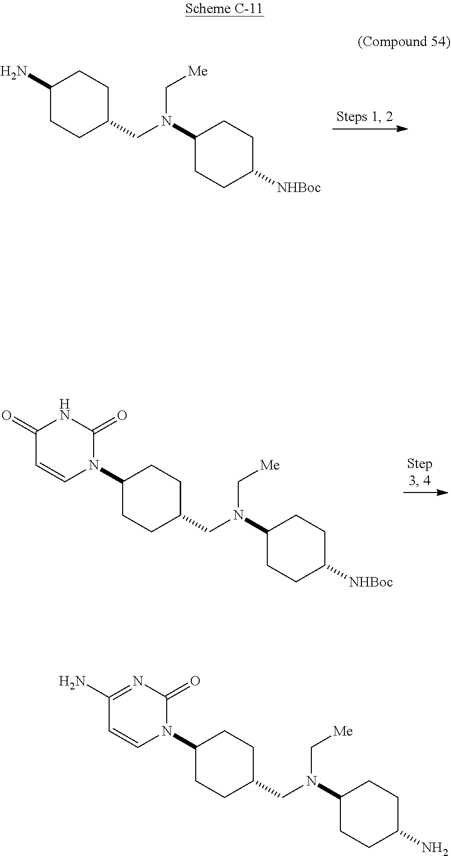

Reagents: 1) (E)-3-ethoxyacryloyl chloride, AgOCN, toluene, [symbol] , 3.5 h; DCM, rt, 16 h 2) TFA, rt, 2 h 3) POCl₃, Et₃N, ACN, 0° C. to rt, 4 h; NH₄OH, 50° C., 18 h 10) 2M HCl, MeOH, rt, 18 h.

Step 1: tert-butyl ((trans)-4-((((trans)-4-(3-((E)-3-ethoxyacryloyl)ureido)cyclohexyl)methyl) (ethyl)amino)cyclohexyl)carbamate. A solution of (E)-3-ethoxyacryloyl chloride (934 mg, 6.9 mmol) in toluene (6 mL) was added dropwise to a suspension of silver cyanate (1.2 g, 8.1 mmol) in toluene (6 mL), and the mixture was stirred at 110° C. for 3.5 hour, then cooled without stirring. A solution of tert-butyl ((trans)-4-((((trans)-4-aminocyclohexyl)methyl)(ethyl)amino)cyclohexyl)carbamate (818 mg, 2.3 mmol) in dry DCM (10 mL) under N₂ was cooled to 0° C. 10 mL of the clear acyl isocyanate solution formed was drawn into a syringe and added dropwise into the cooled mixture. The reaction was warm to rt and stirred for 4 h. The mixture was quenched with MeOH (5 mL), concentrated, taken up in Et₂O (100 mL) and extracted with 5% citric acid (2×100 mL). The aqueous layer was brought to ca. pH 12 with NaOH then extracted with DCM (2×100 mL). The extracts were dried (Na₂SO₄), filtered, and concentrated to give the desired product (1.21 g, quantitative). ¹H NMR (500 MHz, MeOD) δ 7.63 (d, 1H), 5.41 (d, 1H), 3.97 (q, 2H), 3.55 (tt, 1H), 3.27-3.17 (m, 1H), 2.51 (q, 2H), 2.48-2.41 (m, 1H), 2.24 (d, 2H), 2.05-1.98 (m, 2H), 1.97-1.85 (m, 4H), 1.82-1.73 (m, 2H), 1.43 (s, 9H), 1.38-1.28 (m, 6H), 1.26-1.14 (m, 4H), 1.05-0.89 (m, 5H). LCMS[M+H] 495.2.

Step 2: tert-butyl ((trans)-4-((((trans)-4-(3-((E)-3-ethoxyacryloyl)ureido)cyclohexyl)methyl)(ethyl)amino)cyclohexyl)carbamate. A mixture of tert-butyl ((trans)-4-((((trans)-4-(3-((E)-3-ethoxyacryloyl)ureido)cyclohexyl)methyl) (ethyl)amino)cyclohexyl)carbamate (0.8 g, 1.6 mmol) and TFA (6 mL) was stirred at 20° C. for 2 h. The residue was taken up in 1,4-dioxane (16 mL), and 1M sat. NaHCO₃ (16 mL, 16 mmol) and di-tert-butyl dicarbonate (743 μL, 3.23 mmol) were added. The mixture was stirred vigorously for 18 h. The mixture was cooled, diluted with EtOAc (100 mL), washed with sat. NaHCO₃ (×75 mL) and brine (1×75 mL). The aqueous layers were extracted with DCM (100 mL) and combined with the EtOAc layer, dried with Na₂SO₄, decanted, and concentrated. The residue was purified by flash chromatography (0-40% [80/20/2 DCM/MeOH/NH₄OH]/DCM to give the desired product (469 mg, 64.6%) as a foam. ¹H NMR (500 MHz, MeOD) δ 7.67 (d, 1H), 5.66 (d, 1H), 4.36 (tt, 1H), 3.28-3.17 (m, 1H), 2.58-2.44 (m, 3H), 2.28 (d, 2H), 2.05-1.98 (m, 2H), 1.98-1.91 (m, 2H), 1.91-1.83 (m, 2H), 1.83-1.74 (m, 2H), 1.69-1.58 (m, 2H), 1.48-1.27 (m, 12H), 1.26-1.13 (m, 2H), 1.11-0.98 (m, 5H). LCMS[M+H] 449.3.

Step 3: tert-butyl ((trans)-4-((((trans)-4-(4-amino-2-oxopyrimidin-1(2H)-yl)cyclohexyl)methyl)(ethyl)amino)cyclohexyl)carbamate. POCl₃ (332 μL, 3.56 mmol) was added dropwise to a mixture of 1,2,4-triazole (1.97 g, 28.5 mmol) and triethylamine (3.97 mL, 28.5 mmol) in CH₃CN (6 mL) at 0° C., and the mixture was stirred at 0° C. for 15 minutes. A solution of tert-butyl ((trans)-4-((((trans)-4-(2,4-dioxo-3,4-dihydropyrimidin-1(2H)-yl)cyclohexyl)methyl)(ethyl)amino)cyclohexyl)carbamate (533 mg, 1.2 mmol) in CH₃CN (6 mL) was then added dropwise, and the mixture was warmed to rt and stirred for 4 h. NH₄OH (3.6 mL, 89 mmol) was added and the mixture was stirred at 50° C. for 18 h. The mixture was concentrated until a fine precipitate formed that was collected by vacuum filtration to give the desired product (0.25 g, 47.0%). ¹H NMR (500 MHz, MeOD) δ 7.65 (d, 1H), 5.87 (d, 1H), 4.44 (tt, 1H), 3.28-3.17 (m, 1H), 2.59-2.42 (m, 3H), 2.28 (d, 2H), 2.05-1.97 (m, 2H), 1.97-1.91 (m, 2H), 1.91-1.83 (m, 2H), 1.83-1.75 (m, 2H), 1.63-1.52 (m, 2H), 1.51-1.26 (m, 12H), 1.26-1.13 (m, 2H), 1.13-0.98 (m, 5H). LCMS[M+H] 448.3.

Step 4: 4-amino-1-((trans)-4-((((trans)-4-aminocyclohexyl)(ethyl)amino)methyl)cyclohexyl)pyrimidin-2(1H)-one. A mixture of tert-butyl ((trans)-4-((((trans)-4-(4-amino-2-oxopyrimidin-1(2H)-yl)cyclohexyl)methyl) (ethyl)amino)cyclohexyl)carbamate (200 mg, 0.45 mmol) and 2M HCl in MeOH (7.6 mL) were stirred at rt for 18 h. The precipitate was collected by vacuum filtration to give the desired product (131 mg, 84.9%). ¹H NMR (500 MHz, D₂O) δ 7.65 (d, 1H), 6.05-5.91 (m, 1H), 4.35-4.16 (m, 1H), 3.38-3.25 (m, 1H), 3.25-2.98 (m, 3H), 2.98-2.84 (m, 1H), 2.20-1.79 (m, 8H), 1.79-1.38 (m, 4H), 1.29-1.10 (m, 4H). LCMS[M+H] 348.3.

### Biological Examples

### Standard Microbiological Activity:

A certified BSL-2 laboratory was used for testing. Compounds were evaluated using the broth microdilution minimum inhibitory concentration (MIC) and minimum bactericidal concentration (MBC) assays defined by Clinical and Laboratory Standards Institute (CLSI) in the M26-A guideline against S. aureus (Sa), E. coli (Ec), K. pneumoniae (Kp), A. baumannii (Ab), E. faecalis (Ef) and P. aeruginosa (Pa).

E. coli S30 extract: Inhibition of bacterial protein synthesis was determined using the E. coli S30 Extract System for Circular DNA (Promega catalog #L-2010) and Luciferase Assay Reagent (Promega catalog #E1500) with slight modifications to a published protocol. Fyfe, C., Sutcliffe, J. A. and Grossman, T. H. (2012) “Development and characterization of a Pseudomonas aeruginosa in vitro coupled transcription-translation assay system for evaluation of translation inhibitors” J. Microbiol. Methods 90(3), 256-261.

Compounds were serial diluted in 0.5 mL microcentrifuge tubes by mixing and transferring 50 μL from the highest concentration to 50 μL of water, mixing and transferring 50 μL of this 2-fold dilution to 50 μL of water. This mixing and transferring was repeated so that there are a total of 8 tubes with serial dilutions of compound at 10× the desired screening concentration that are ultimately diluted to 1× by the addition of S30 luciferase synthesis mixture. Serial dilutions of compounds were added (2 μL) to wells in a black round bottom 96-well plate. Water (2 μL) was used as a “no inhibitor” control in 4 wells/plate. No DNA control reaction mixture (20 μL; see below) was used as a control in 4 wells/plate for background luminescence. S30 luciferase synthesis mixture (18 μL; see below) was added to wells with compounds or water mixture and incubated at 37° C. for 1 hour. Reactions were stopped by transferring to 4° C. refrigerator for 5 minutes then 25 μL of luciferase activity mix was added. Luminescence was measured using a BioTek Synergy HTX plate reader. % Inhibition was determined relative to no inhibitor controls.

### S30 Luciferase Synthesis Mixture:

445 μL S30 extract, circular
712 μL S30 Premix without amino acids
4.45 μL pBESTluc™ DNA (1 μg/μL)
78 μL complete amino acid mixture
267 μL water

### No DNA Control:

20 μL S30 extract, circular
32 μL S30 Premix without amino acids
7 μL complete amino acid mixture
21 μL water

### Rabbit Reticulocyte Lysate

Inhibition of eukaryotic protein synthesis was determined using the Rabbit Reticulocyte Lysate System, Nuclease-Treated from Promega (catalog #L-4960) with slight modifications to the manufacturer's protocol. Compounds were serial diluted in 0.5 mL microcentrifuge tubes by mixing and transferring 50 μL from the highest concentration to 50 μL of water, mixing and transferring 50 μL of this 2-fold dilution to 50 μL of water. This mixing and transferring was repeated so that there are a total of 8 tubes with serial dilutions of compound at 10× the desired screening concentration that are ultimately diluted to 1× by the addition of rabbit reticulocyte luciferase synthesis mixture. Serial dilutions of compounds were added (2.5 L) to wells in a black round bottom 96-well plate. Water (2.5 μL) was used as a “no inhibitor” control in 4 wells/plate. No RNA control reaction mixture (2 μL; see below) was used as a control in 4 wells/plate for background luminescence. Rabbit reticulocyte luciferase synthesis mixture (22.5 μL; see below) was added to wells with compounds or water mixture and incubated at 30° C. for 90 minutes. Luciferase assay reagent (25 μL) was added with luminescence measured using a BioTek Synergy HTX plate reader. % Inhibition was determined relative to no inhibitor controls.

### Rabbit Reticulocyte Luciferase Synthesis Mixture:

1,000 μL rabbit reticulocyte lysate
5.7 μL Luciferase Control RNA (1 μg/μL)
26 μL complete amino acid mixture
395 μL water

### No RNA Control

70 μL rabbit reticulocyte lysate
2 μL complete amino acid mixture
28 μL water

### Minimum Inhibitory Concentration (MIC)

MICs were determined using the Clinical Laboratory and Standards Institute (CLSI) Broth Microdilution Method with slight modification. Clinical and Laboratory Standards Institute (2012). “Methods for dilution antimicrobial susceptibility tests for bacteria that grow aerobically; approved standard, 9th ed. M07-A9. Clinical and Laboratory Standards Institute, Wayne, PA.” Serial two-fold dilutions of compounds are prepared in sterile clear round-bottom 96-well plates.

To prepare microdilution trays, two-fold dilutions of antimicrobial agent are prepared in growth medium: Cation-Adjusted Mueller-Hinton Broth (CAMHB), or CAMHB supplemented with sodium bicarbonate (25 mM final concentration prepared from a 1.0 M stock solution) or CAMHB supplemented with heat inactivated human serum (Fisher Cat. #BP2657100) 0-50% by adding 200 μL of the highest concentration to be tested (64 μg/mL, for example) in row A, mixing and transferring 100 μL from row A to 100 μL growth medium in row B, then repeating the mixing and transferring through row H of the 96-well plate, discarding the excess 100 μL remaining. This slight modification to the CLSI protocol enables evaluation of MICs for 3 compounds per plate in triplicate, albeit with only 8 compound dilutions (CLSI protocol enables 2 compounds in triplicate with 10 dilutions). Bacterial suspensions are added to a final concentration of 5×10⁴ CFU/well by adding 5 μL of a 1:10 dilution of a 0.5 McFarland suspension (1×10⁸ CFU/mL) for each bacterium evaluated. Bacterial suspensions were prepared using the growth method described by CLSI. Well-isolated colonies (3-5 from an agar plate) were selected using a sterile loop and used to inoculate a tube containing 4 mL of CAMHB. The cultures are incubated at 35±2° C. until it achieves or exceeds the turbidity of the 0.5 McFarland standard, determined by measuring A₆₀₀ₙₘ (usually two to six hours). When growth exceeds a 0.5 McFarland standard, the turbidity is adjusted with broth to be equivalent to a 0.5 McFarland standard.

Data for compounds is provided in Tables 4 and 5. An IC₅₀ value (μM) that is 1 μM or greater (% inhibition is ≤50% @1 μM) is designated by a “+”. An IC₅₀ value that is 0.5 μM or greater and less than 1 μM (% inhibition is >50% and ≤90% @1 μM) is designated by a “++”. An IC₅₀ value that is less than 0.5 μM (% inhibition is >90% @1 μM) is designated by “+++”. An MIC value (μg/mL) that is 32 μg/mL or greater is designated by a “+”. An MIC value (μg/mL) that is 8 μg/mL or greater and less than 32 μg/mL is designated by a “++”. An MIC value (μg/mL) that is less than 8 μg/mL is designated by “+++”. “NA” means not available.

TABLE 4
Biological Activity of Compounds of Formula I or a pharmaceutically acceptable salt thereof

| | | | Kp | | | | | | |
|---|---|---|---|---|---|---|---|---|---|
| | Sa + | E. coli + | (1705) + | Kp (060) + | Pa + | Ab + | Ef + | | |
| Cmpd. | bicarb | bicarb | bicarb | bicarb | bicarb | bicarb | bicarb | S30 | Retic. |
| No. | 25 | 25 | 25 | 25 | 25 | 25 | 25 | IC₅₀ | IC₅₀ |
| 1 | +++ | +++ | ++ | +++ | + | + | ++ | +++ | + |
| 2 | +++ | +++ | ++ | +++ | + | + | +++ | +++ | + |
| 3 | +++ | +++ | +++ | +++ | + | + | + | +++ | + |
| 4 | +++ | +++ | ++ | +++ | + | + | +++ | +++ | + |
| 5 | +++ | +++ | +++ | +++ | + | ++ | +++ | +++ | + |
| 6 | +++ | +++ | ++ | + | + | + | ++ | + | + |
| 7 | +++ | +++ | +++ | +++ | + | ++ | +++ | +++ | + |
| 8 | +++ | +++ | | +++ | ++ | + | +++ | +++ | + |
| 9 | +++ | +++ | +++ | +++ | + | ++ | +++ | +++ | + |
| 10 | NA | NA | NA | NA | NA | NA | NA | + | + |
| 11 | +++ | +++ | +++ | +++ | + | + | +++ | +++ | + |
| 12 | +++ | +++ | +++ | ++ | + | ++ | +++ | +++ | + |

TABLE 5
Biological Activity of Additional Compounds of Formula
I or a pharmaceutically acceptable salt thereof

| | | | Kp | | | | | | |
|---|---|---|---|---|---|---|---|---|---|
| | Sa + | E. coli + | (1705) + | Kp (060) + | Pa + | Ab + | Ef + | | |
| | bicarb | bicarb | bicarb | bicarb | bicarb | bicarb | bicarb | S30 | Retic. |
| No. | 25 | 25 | 25 | 25 | 25 | 25 | 25 | IC₅₀ | IC₅₀ |
| 13 | +++ | +++ | +++ | +++ | + | + | ++ | +++ | + |
| 14 | +++ | +++ | +++ | +++ | + | + | ++ | +++ | + |
| 15 | NA | NA | NA | NA | NA | + | + | +++ | + |
| 16 | +++ | +++ | +++ | ++++ | ++ | +++ | +++ | +++ | + |
| 17 | +++ | +++ | +++ | +++ | + | + | +++ | +++ | + |
| 18 | +++ | +++ | +++ | +++ | + | + | ++ | +++ | + |
| 19 | +++ | +++ | +++ | +++ | + | ++ | +++ | +++ | + |
| 20 | +++ | +++ | +++ | +++ | ++ | +++ | +++ | +++ | + |
| 21 | +++ | +++ | +++ | +++ | ++ | ++ | +++ | +++ | + |
| 22 | +++ | ++++ | +++ | +++ | + | +++ | ++ | +++ | + |
| 23 | +++ | +++ | ++ | ++ | + | + | ++ | +++ | + |
| 24 | +++ | +++ | ++ | + | + | + | + | +++ | + |
| 25 | +++ | +++ | +++ | +++ | ++ | +++ | +++ | +++ | + |
| 26 | +++ | +++ | +++ | +++ | + | + | +++ | +++ | + |
| 27 | +++ | ++++ | +++ | ++ | + | ++ | +++ | +++ | + |
| 28 | NA | NA | NA | NA | NA | + | + | ++ | + |
| 29 | NA | NA | NA | NA | NA | NA | NA | ++ | - |
| 30 | +++ | +++ | +++ | +++ | + | + | ++ | +++ | + |
| 31 | +++ | +++ | ++ | +++ | + | | +++ | +++ | + |
| 32 | +++ | +++ | ++ | ++ | + | + | ++ | +++ | + |
| 33 | +++ | +++ | +++ | ++ | + | ++ | +++ | +++ | + |
| 34 | +++ | +++ | +++ | ++ | + | ++ | +++ | +++ | + |
| 35 | +++ | +++ | +++ | +++ | + | ++ | +++ | ++ | + |
| 36 | +++ | +++ | +++ | +++ | ++ | ++ | +++ | +++ | + |
| 37 | +++ | +++ | +++ | ++ | + | + | +++ | +++ | + |
| 38 | +++ | +++ | +++ | ++ | + | + | +++ | +++ | + |
| 39 | +++ | +++ | +++ | +++ | + | + | +++ | +++ | + |
| 40 | +++ | +++ | +++ | +++ | + | + | +++ | +++ | + |
| 41 | +++ | +++ | +++ | ++ | + | + | +++ | +++ | + |
| 42 | +++ | +++ | +++ | +++ | + | + | +++ | +++ | + |
| 43 | +++ | +++ | ++ | ++ | + | + | ++ | +++ | + |
| 44 | +++ | +++ | ++ | ++ | + | + | ++ | +++ | + |
| 45 | +++ | +++ | ++ | + | + | + | +++ | +++ | + |
| 46 | +++ | +++ | ++ | ++ | + | + | + | +++ | + |
| 47 | +++ | +++ | ++ | ++ | + | + | + | +++ | + |
| 48 | ++ | ++ | + | + | + | + | ++ | +++ | + |
| 49 | +++ | +++ | + | + | | + | ++ | +++ | + |
| 50 | +++ | +++ | +++ | +++ | + | ++ | +++ | +++ | + |
| 51 | +++ | +++ | ++ | +++ | + | + | ++ | +++ | + |
| 52 | ++ | +++ | + | + | + | + | + | ++ | + |
| 53 | +++ | ++ | +++ | +++ | + | + | ++ | +++ | + |
| 54 | + | + | + | + | + | + | + | + | + |

### Other Embodiments

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A compound of formula I: 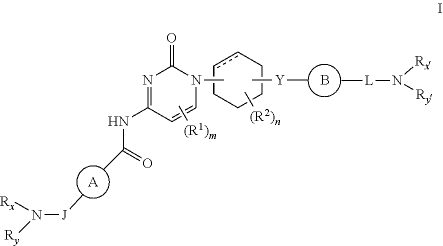
or a single stereoisomer or mixture of stereoisomers thereof, or a pharmaceutically acceptable salt thereof, wherein:
ring A is a 3-8 membered monocyclic heterocycloalkylene optionally substituted with up to three substituents independently selected from the group consisting of C₁-C₆ alkyl, C₁-C₆ alkoxy, halo, CN, C₁-C₆ haloalkyl, phenyl, OH, NH₂, NH(C₁-C₆ alkyl), N(C₁-C₆ alkyl)₂, COOH, COO(C₁-C₆ alkyl), CONH₂, CONH(C₁-C₆ alkyl), CON(C₁-C₆ alkyl)₂, and oxo;
J is C₁-C₆ alkylene or C₃-C₈ cycloalkylene, either of which is optionally substituted with halo, OH, or C₁-C₆ alkoxy, wherein up to two methylene units of the C₁-C₆ alkylene are optionally and independently replaced with O, S, SO, SO₂, or C═O;
Y is a bond or C₁-C₆ alkylene optionally substituted with OH, NH₂, CN, halo, or C₁-C₆ alkoxy, wherein up to two methylene units of the C₁-C₆ alkylene are optionally and independently replaced by O, NH, N—(C₁-C₆ alkyl), N—(C₁-C₆ hydroxyalkyl), N—(C₁-C₆ haloalkyl), N—(C₁₋₆ alkylene-C₃₋₈ cycloalkyl), N—(C₃₋₈ cycloalkyl), NH(C═O), N—(C₁₋₆ alkyl)(C═O), or (C═O);
ring B is a 3-8 membered monocyclic cycloalkylene, 3-8 membered monocyclic heterocycloalkylene, 6-12 membered bicyclic cycloalkylene, or a 6-12 membered bicyclic heterocycloalkylene, each of which is optionally substituted with up to three substituents independently selected from the group consisting of C₁-C₆ alkyl, C₁-C₆ alkoxy, halo, CN, C₁-C₆ haloalkyl, OH, COOH, COO(C₁-C₆ alkyl), CONH₂, CONH(C₁-C₆ alkyl), CON(C₁-C₆ alkyl)₂, and C₁-C₆ hydroxyalkyl;
L is a bond or C₁-C₆ alkylene, wherein up to two methylene units of the C₁-C₆ alkylene may independently be replaced with 0, NH, (C═O), NH(C═O), N—(C₁₋₆ alkyl)(C═O), (C═NH), NH(C═N), or N—(C₁₋₆ alkyl);
R¹ and R² are each independently selected from the group consisting of C₁-C₆ alkyl, halo, CN, OH, NH₂, O(C₁-C₆ haloalkyl), NH(C₁-C₆ alkyl), N(C₁-C₆ alkyl)₂, —COO(C₁-C₆ alkyl), CONH₂, C₁-C₆ haloalkyl, C₁-C₆ alkoxy, and C₁-C₆ haloalkoxy;
Rₓ, R_{y}, R_{x′}, and R_{y′} are each independently H, C₁-C₆ alkyl, or an amino protecting group;
m and n are each independently 0, 1, 2, or 3; and [symbol]
represents a single bond or a double bond.

2. The compound of claim 1, or a single stereoisomer or mixture of stereoisomers thereof, or a pharmaceutically acceptable salt thereof, wherein ring A is a 5-6 membered monocyclic heterocycloalkylene optionally substituted with up to three substituents independently selected from the group consisting of C₁-C₆ alkyl, C₁-C₆ alkoxy, halo, CN, C₁-C₆ haloalkyl, phenyl, OH, NH₂, and oxo.

3. The compound of claim 1 or 2, or a single stereoisomer or mixture of stereoisomers thereof, or a pharmaceutically acceptable salt thereof, wherein ring A is 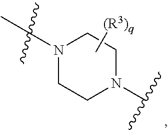
wherein each R³ is independently selected from the group consisting of C₁-C₆ alkyl, C₁-C₆ alkoxy, halo, CN, C₁-C₆ haloalkyl, phenyl, OH, NH₂, and oxo, wherein q is 0, 1, or 2.

4. The compound of any one of claims 1-3, or a single stereoisomer or mixture of stereoisomers thereof, or a pharmaceutically acceptable salt thereof, wherein ring A is

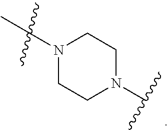 [SMILES: *N1CCN(*)CC1]

5. The compound of any one of claims 1-4, or a single stereoisomer or mixture of stereoisomers thereof, or a pharmaceutically acceptable salt thereof, wherein J is C₁-C₆ alkylene optionally substituted with halo, OH, or C₁-C₆ alkoxy, wherein up to two methylene units of the C₁-C₆ alkylene are optionally and independently replaced with O, S, SO, SO₂, or C═O.

6. The compound of any one of claims 1-5, or a single stereoisomer or mixture of stereoisomers thereof, or a pharmaceutically acceptable salt thereof, wherein J is C₁-C₆ alkylene, wherein one methylene unit of the C₁-C₆ alkylene is replaced with C═O.

7. The compound of any one of claims 1-6, or a single stereoisomer or mixture of stereoisomers thereof, or a pharmaceutically acceptable salt thereof, wherein J is

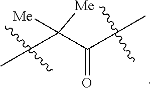 [SMILES: *C(=O)C(*)(C)C]

8. The compound of any one of claims 1-7, or a single stereoisomer or mixture of stereoisomers thereof, or a pharmaceutically acceptable salt thereof, wherein Rₓ and R_{y} are each independently H.

9. The compound of any one of claims 1-8, or a single stereoisomer or mixture of stereoisomers thereof, or a pharmaceutically acceptable salt thereof, wherein 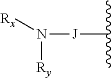
is

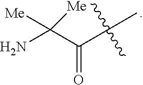 [SMILES: *C(=O)C(C)(C)N]

10. The compound of any one of claims 1-9, or a single stereoisomer or mixture of stereoisomers thereof, or a pharmaceutically acceptable salt thereof, wherein 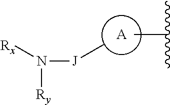
is

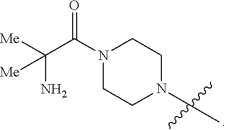 [SMILES: *N1CCN(C(=O)C(C)(C)N)CC1]

11. The compound of any one of claims 1-10, or a single stereoisomer or mixture of stereoisomers thereof, or a pharmaceutically acceptable salt thereof, wherein 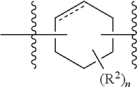
is 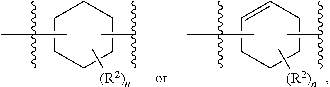
wherein each R² is independently selected from the group consisting of C₁-C₆ alkyl, C₁-C₆ alkoxy, halo, and CN; and n is 0, 1 or 2.

12. The compound of any one of claims 1-11, or a single stereoisomer or mixture of stereoisomers thereof, or a pharmaceutically acceptable salt thereof, wherein 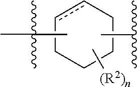
is 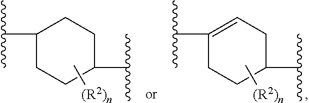
wherein each R² is independently selected from the group consisting of C₁-C₆ alkyl, C₁-C₆ alkoxy, halo, and CN; and n is 0, 1 or 2.

13. The compound of any one of claims 1-12, or a single stereoisomer or mixture of stereoisomers thereof, or a pharmaceutically acceptable salt thereof, wherein 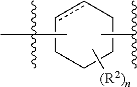
is

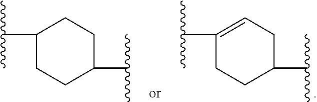 [SMILES: *C1CCC(*)CC1 ; *C1=CCC(*)CC1]

14. The compound of any one of claims 1-13, or a single stereoisomer or mixture of stereoisomers thereof, or a pharmaceutically acceptable salt thereof, wherein Y is C₁-C₃ alkylene optionally substituted with OH, NH₂, halo, or C₁-C₆ alkoxy, and wherein one methylene unit of the C₁-C₃ alkylene is optionally replaced by O, NH, N—(C₁-C₆ alkyl), N—(C₁-C₆ hydroxyalkyl), N—(C₁-C₆ haloalkyl), N—(C₃₋₈ cycloalkyl), N—(C₁₋₆ alkylene-C₃₋₈ cycloalkyl), NH(C═O), N—(C₁₋₆ alkyl)(C═O), or (C═O).

15. The compound of any one of claims 1-14, or a single stereoisomer or mixture of stereoisomers thereof, or a pharmaceutically acceptable salt thereof, wherein Y is C₁-C₃ alkylene, and wherein one methylene unit of the C₁-C₃ alkylene is optionally replaced by NH, N—(C₁-C₆ haloalkyl), or N—(C₁-C₆ alkyl).

16. The compound of any one of claims 1-15, or a single stereoisomer or mixture of stereoisomers thereof, or a pharmaceutically acceptable salt thereof, wherein Y is selected from the group consisting of —CH₂—, —CH₂NH—, —CH₂NMe-, —CH₂N(CH₂CH₂F)—, and —CH₂NEt-.

17. The compound of any one of claims 1-16, or a single stereoisomer or mixture of stereoisomers thereof, or a pharmaceutically acceptable salt thereof, wherein 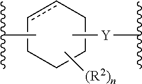
is selected from the group consisting of

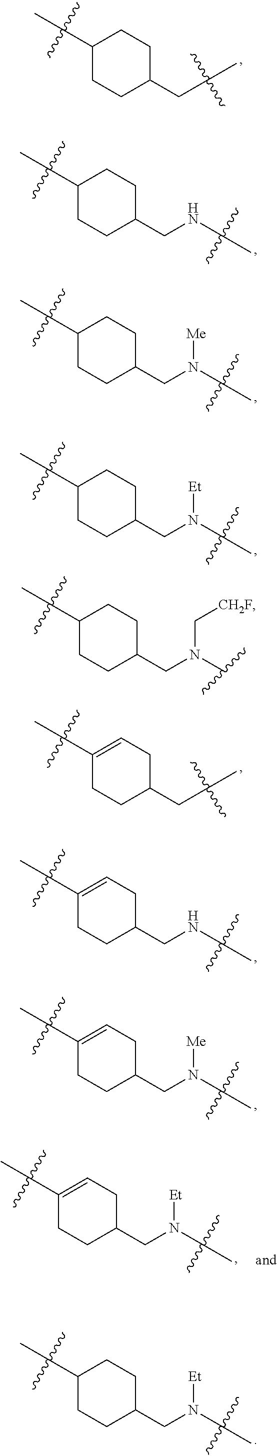

18. The compound of any one of claims 1-17, or a single stereoisomer or mixture of stereoisomers thereof, or a pharmaceutically acceptable salt thereof, wherein ring B is a 3-8 membered monocyclic cycloalkylene optionally substituted with up to three substituents independently selected from the group consisting of C₁-C₆ alkyl, C₁-C₆ alkoxy, halo, CN, C₁-C₆ haloalkyl, OH, COOH, COO(C₁-C₆ alkyl), CONH₂, CONH(C₁-C₆ alkyl), CON(C₁-C₆ alkyl)₂, and C₁-C₆ hydroxyalkyl.

19. The compound of claim 18, or a single stereoisomer or mixture of stereoisomers thereof, or a pharmaceutically acceptable salt thereof, wherein ring B is a 5-6 membered monocyclic cycloalkylene optionally substituted with up to three substituents independently selected from the group consisting of C₁-C₆ alkyl, C₁-C₆ alkoxy, halo, CN, C₁-C₆ haloalkyl, OH, and C₁-C₆ hydroxyalkyl.

20. The compound of any one of claims 1-17, or a single stereoisomer or mixture of stereoisomers thereof, or a pharmaceutically acceptable salt thereof, wherein ring B is a 3-8 membered monocyclic heterocycloalkylene optionally substituted with up to three substituents independently selected from the group consisting of C₁-C₆ alkyl, C₁-C₆ alkoxy, halo, CN, C₁-C₆ haloalkyl, OH, COOH, COO(C₁-C₆ alkyl), CONH₂, CONH(C₁-C₆ alkyl), CON(C₁-C₆ alkyl)₂, and C₁-C₆ hydroxyalkyl.

21. The compound of claim 20, or a single stereoisomer or mixture of stereoisomers thereof, or a pharmaceutically acceptable salt thereof, wherein ring B is a 4-7 membered monocyclic heterocycloalkylene optionally substituted with up to three substituents independently selected from the group consisting of C₁-C₆ alkyl, C₁-C₆ alkoxy, halo, CN, C₁-C₆ haloalkyl, OH, and C₁-C₆ hydroxyalkyl, and wherein B contains up to 2 nitrogen atoms.

22. The compound of any one of claims 1-17, or a single stereoisomer or mixture of stereoisomers thereof, or a pharmaceutically acceptable salt thereof, wherein ring B is a 6-10 membered bicyclic cycloalkylene optionally substituted with up to three substituents independently selected from the group consisting of C₁-C₆ alkyl, C₁-C₆ alkoxy, halo, C₁-C₆ haloalkyl, OH, and C₁-C₆ hydroxyalkyl.

23. The compound of claim 22, or a single stereoisomer or mixture of stereoisomers thereof, or a pharmaceutically acceptable salt thereof, wherein ring B is a 6-10 membered fused, spiro, or bridged bicyclic cycloalkylene.

24. The compound of claim 23, or a single stereoisomer or mixture of stereoisomers thereof, or a pharmaceutically acceptable salt thereof, wherein ring B is a 6-10 membered fused bicyclic cycloalkylene.

25. The compound of claim 23, or a single stereoisomer or mixture of stereoisomers thereof, or a pharmaceutically acceptable salt thereof, wherein ring B is a 6-10 membered bridged bicyclic cycloalkylene.

26. The compound of claim 23, or a single stereoisomer or mixture of stereoisomers thereof, or a pharmaceutically acceptable salt thereof, wherein ring B is a 6-10 membered spiro bicyclic cycloalkylene.

27. The compound of any one of claims 1-17, or a single stereoisomer or mixture of stereoisomers thereof, or a pharmaceutically acceptable salt thereof, wherein ring B is a 6-12 membered bicyclic heterocycloalkylene optionally substituted with up to three substituents independently selected from the group consisting of C₁-C₆ alkyl, C₁-C₆ alkoxy, halo, C₁-C₆ haloalkyl, OH, and C₁-C₆ hydroxyalkyl.

28. The compound of claim 27, or a single stereoisomer or mixture of stereoisomers thereof, or a pharmaceutically acceptable salt thereof, wherein ring B is a fused, spiro, or bridged bicyclic heterocycloalkylene containing up to 2 nitrogen atoms.

29. The compound of claim 28, or a single stereoisomer or mixture of stereoisomers thereof, or a pharmaceutically acceptable salt thereof, wherein ring B is a fused bicyclic heterocycloalkylene containing up to 2 nitrogen atoms.

30. The compound of claim 28, or a single stereoisomer or mixture of stereoisomers thereof, or a pharmaceutically acceptable salt thereof, wherein ring B is a spiro bicyclic heterocycloalkylene containing up to 2 nitrogen atoms.

31. The compound of claim 28, or a single stereoisomer or mixture of stereoisomers thereof, or a pharmaceutically acceptable salt thereof, wherein ring B is a bridged bicyclic heterocycloalkylene containing up to 2 nitrogen atoms.

32. The compound of any one of claims 1-17, or a single stereoisomer or mixture of stereoisomers thereof, or a pharmaceutically acceptable salt thereof, wherein ring B is selected from the group consisting of

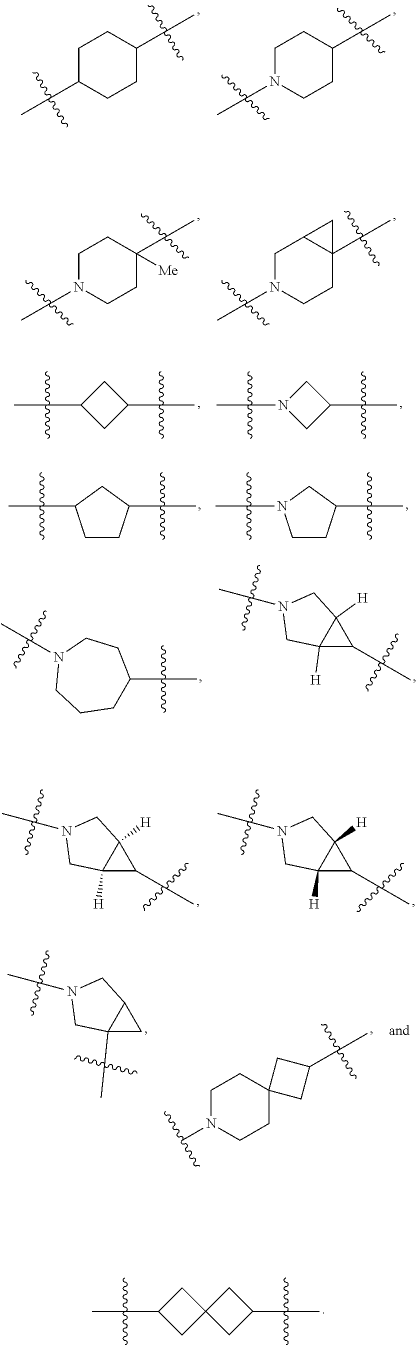

33. The compound of any one of claims 1-32, or a single stereoisomer or mixture of stereoisomers thereof, or a pharmaceutically acceptable salt thereof, wherein L is a bond.

34. The compound of any one of claims 1-32, or a single stereoisomer or mixture of stereoisomers thereof, or a pharmaceutically acceptable salt thereof, wherein L is C₁-C₆ alkylene, wherein up to two methylene units of the C₁-C₆ alkylene are optionally and independently replaced with O, NH, (C═O), NH(C═O), N—(C₁₋₆ alkyl)(C═O), (C═NH), NH(C═N), or N—(C₁₋₆ alkyl).

35. The compound of claim 34, or a single stereoisomer or mixture of stereoisomers thereof, or a pharmaceutically acceptable salt thereof, wherein L is C₁-C₆ alkylene.

36. The compound of claim 35, or a single stereoisomer or mixture of stereoisomers thereof, or a pharmaceutically acceptable salt thereof, wherein L is —CH₂— or —CH₂—CH₂—.

37. The compound of any one of claims 1-36, or a single stereoisomer or mixture of stereoisomers thereof, or a pharmaceutically acceptable salt thereof, wherein R_{x′} and R_{y′} are H.

38. The compound of any one of claims 1-37, or a single stereoisomer or mixture of stereoisomers thereof, or a pharmaceutically acceptable salt thereof, wherein 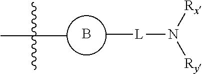
is selected from the group consisting of

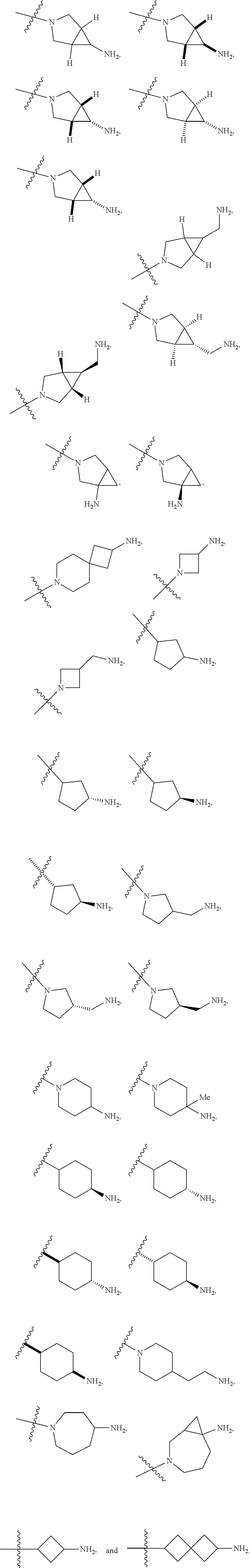

39. The compound of any one of claims 1-38, or a single stereoisomer or mixture of stereoisomers thereof, or a pharmaceutically acceptable salt thereof, wherein R¹ and R² are each independently selected from the group consisting of C₁-C₆ alkyl, halo, C₁-C₆ haloalkyl, oxo, and C₁-C₆ alkoxy, and m and n are each independently 0, 1, or 2.

40. The compound of claim 39, or a single stereoisomer or mixture of stereoisomers thereof, or a pharmaceutically acceptable salt thereof, wherein R¹ and R² are each independently C₁-C₆ alkyl, halo, oxo, or C₁-C₆ haloalkyl, and m and n are each independently 0 or 1.

41. The compound of any one of claims 1-40, or a single stereoisomer or mixture of stereoisomers thereof, or a pharmaceutically acceptable salt thereof which is a compound of formula IA:

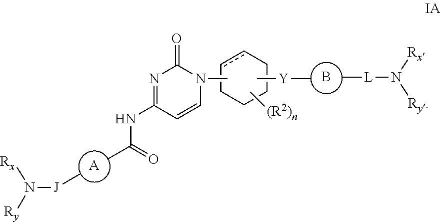

42. The compound of claim 41, or a single stereoisomer or mixture of stereoisomers thereof, or a pharmaceutically acceptable salt thereof, which is a compound of formula IA-1: 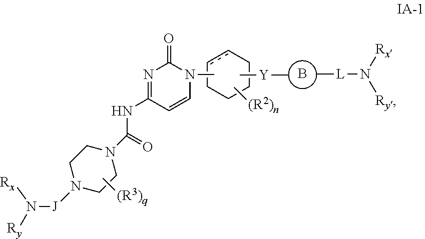
wherein each R³ is independently selected from the group consisting of H, C₁-C₆ alkyl, C₁-C₆ alkoxy, halo, CN, C₁-C₆ haloalkyl, OH, NH₂, and oxo, and wherein q is 0, 1, 2, or 3.

43. The compound of claim 42, or a single stereoisomer or mixture of stereoisomers thereof, or a pharmaceutically acceptable salt thereof, which is a compound of formula IA-2: 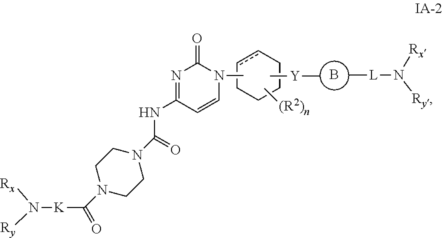
wherein K is C₁-C₄ alkylene optionally substituted with halo, hydroxyl or C₁-C₆ alkoxy group.

44. The compound of claim 43, or a single stereoisomer or mixture of stereoisomers thereof, or a pharmaceutically acceptable salt thereof, which is a compound of formula IA-3: 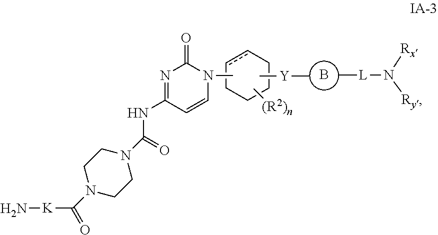
wherein K is C₁-C₃ alkylene.

45. The compound of claim 44, or a single stereoisomer or mixture of stereoisomers thereof, or a pharmaceutically acceptable salt thereof, which is a compound of formula IA-4:

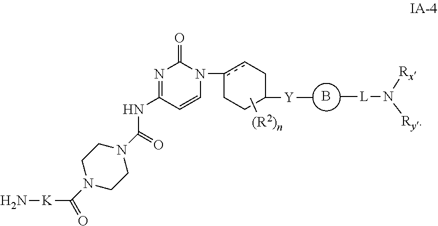

46. The compound of claim 45, or a single stereoisomer or mixture of stereoisomers thereof, or a pharmaceutically acceptable salt thereof, which is a compound of formula IA-5:

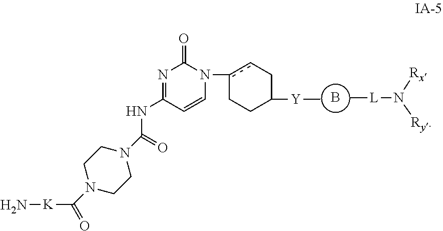

47. The compound of claim 46, or a single stereoisomer or mixture of stereoisomers thereof, or a pharmaceutically acceptable salt thereof, which is a compound of formula IA-6:

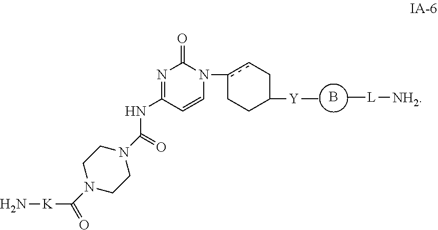

48. The compound of claim 47, or a single stereoisomer or mixture of stereoisomers thereof, or a pharmaceutically acceptable salt thereof, which is a compound of formula IA-7:

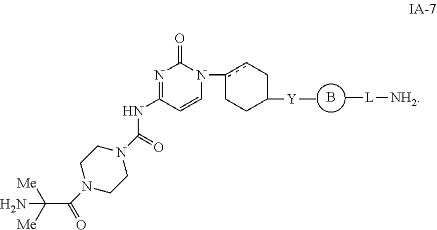

49. The compound of claim 48, or a single stereoisomer or mixture of stereoisomers thereof, or a pharmaceutically acceptable salt thereof, which is a compound of formula IA-7a, formula IA-7b, formula IA-7c, formula IA-7d, IA-7e, IA-7f, or IA-7g: 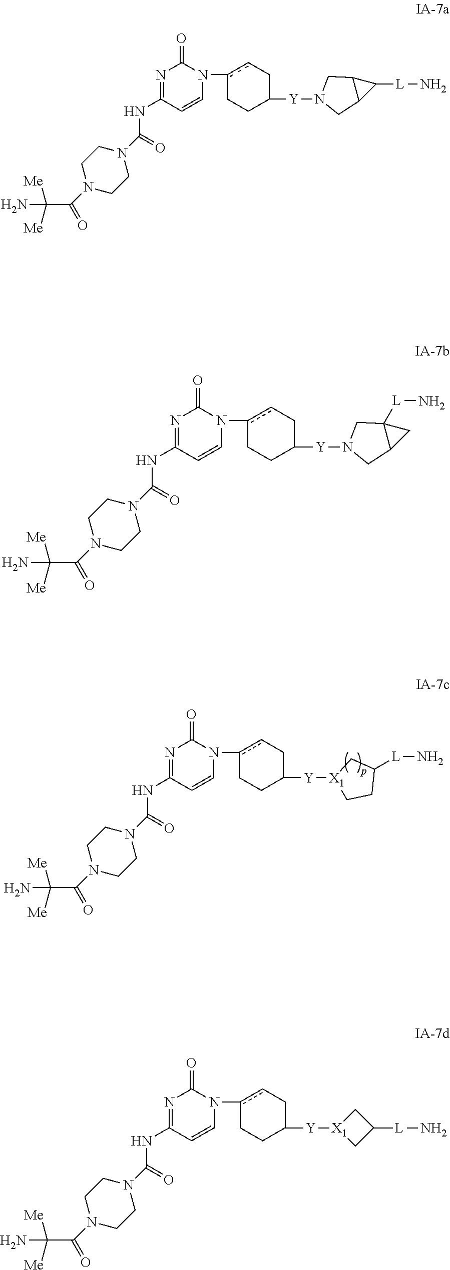 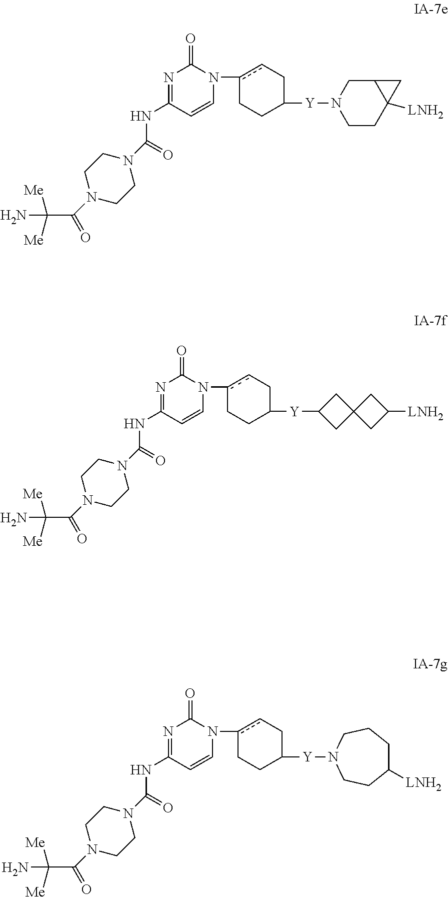
wherein each X₁ is independently CH or N, and p is 1, 2, or 3.

50. The compound of any one of claims 41-49, or a single stereoisomer or mixture of stereoisomers thereof, or a pharmaceutically acceptable salt thereof, wherein Y is selected from the group consisting of —CH₂—, —CH₂NH—, —CH₂NMe-, —CH₂NCH₂CH₂F—, and —CH₂NEt-.

51. The compound of any one of claims 41-48, or a single stereoisomer or mixture of stereoisomers thereof, or a pharmaceutically acceptable salt thereof, wherein ring B is selected from the group consisting of

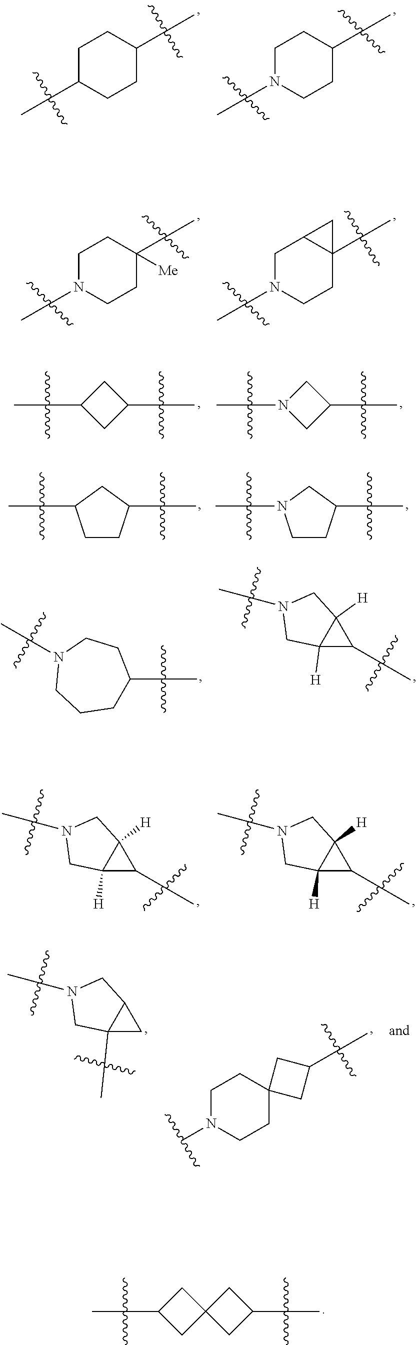

52. The compound of any one of claims 41-49, or a single stereoisomer or mixture of stereoisomers thereof, or a pharmaceutically acceptable salt thereof, wherein L is a bond or C₁-C₃ alkylene.

53. A compound, or a single stereoisomer or mixture of stereoisomers thereof, or a pharmaceutically acceptable salt thereof, which is listed in Table 1 and Table 2.

54. A compound, or a single stereoisomer or mixture of stereoisomers thereof, or a pharmaceutically acceptable salt thereof, which is listed in Table 2.

55. A pharmaceutical composition comprising a compound of any one of claims 1-54, or a pharmaceutically acceptable salt thereof, and a pharmaceutically acceptable excipient.

56. A method of treating a bacterial infection in a patient in need of such treatment, comprising administering the compound of any one of claims 1-54, or a pharmaceutically acceptable salt thereof, or the pharmaceutical composition of claim 55.

57. The method of claim 56, wherein the bacterial infection is caused by a bacterium including gram positive and gram negative bacteria.

58. The method of claim 57, wherein the bacterium includes Francisella tularensis, Burkholderia mallei, Burkholderia pseudomallei, Bacillus anthracis, Yersinia pestis, Salmonella, Clostridium difficile, Citrobacter, Enterobacter, Burkholderia genus, cepacia, Mycobacterium, Proteus, Streptococcus, Serratia, Enterobacteriaceae, Escherichia, Klebsiella, Pseudomonas, and Acinitobacter.

59. A process for preparing a compound of formula I: 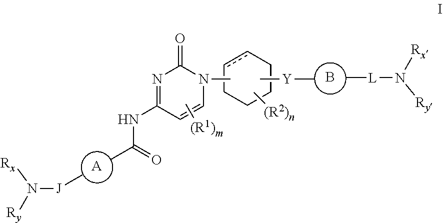
or a single stereoisomer or mixture of stereoisomers thereof, or a pharmaceutically acceptable salt thereof, the process comprising:
combining a compound of formula E: 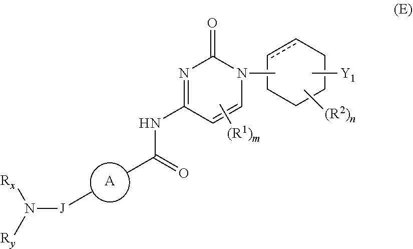
with a compound of:
formula D 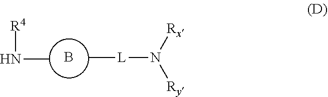
or formula D′ 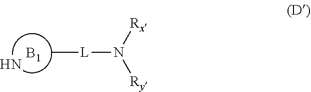
under a reductive amination condition to provide the compound of formula I,
wherein ring A, ring B, J, L, R¹, R², Rₓ, R_{y}, R_{x′}, R_{y′}, and n have the same definitions in any one of claims 1-52;
ring B₁ is a nitrogen containing 3-8 membered monocyclic heterocycloalkylene or a nitrogen containing 6-12 membered bicyclic heterocycloalkylene, each of which is optionally substituted with up to three substituents independently selected from the group consisting of C₁-C₆ alkyl, C₁-C₆ alkoxy, halo, CN, C₁-C₆ haloalkyl, OH, COOH, COO(C₁-C₆ alkyl), CONH₂, CONH(C₁-C₆ alkyl), CON(C₁-C₆ alkyl)₂, and C₁-C₆ hydroxyalkyl;
Y is C₁-C₆ alkylene, wherein one methylene unit of the C₁-C₆ alkylene is optionally replaced by NH or N—(C₁-C₆ alkyl);
Y₁ is C₁-C₆ alkylene, wherein one methylene unit of the C₁-C₆ alkylene is replaced by (C═O); and
R⁴ is H or C₁-C₆ alkyl.

60. A compound of formula E: 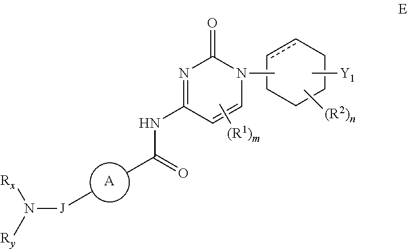
or a pharmaceutically acceptable salt thereof wherein ring A, J, R¹, R², Rₓ, R_{y}, m, and n have the same definitions in any one of claims 1-52; and Y₁ is C₁-C₆ alkylene, wherein one methylene unit of the C₁-C₆ alkylene is replaced by (C═O).

61. The compound of claim 60 or a pharmaceutically acceptable salt thereof, wherein the compound is selected from the group consisting of: [chemical structure]
